(19) United States
(12) Patent Application Publication
(10) Pub. No.: US 2026/0048143 A1
(43) Pub. Date: Feb. 19, 2026

(54) Compositions and Methods for Targeted Delivery of CRISPR-CAS Effector Polypeptides

(71) Applicant: The Regents of the University of California, Oakland, CA (US)

(72) Inventors: Jennifer A. Doudna, Berkeley, CA (US); Jennifer R. Hamilton, Berkeley, CA (US)

(21) Appl. No.: 19/103,517

(22) PCT Filed: Aug. 21, 2023
(86) PCT No.: PCT/US2023/072598

§ 371 Date: Feb. 12, 2025

Related U.S. Application Data
(60) Provisional application No. 63/400,166, filed on Aug. 23, 2022.
(60) Provisional application No. 63/432,484, filed on Dec. 14, 2022.

(51) Int. Cl. A61K 48/00; A61K 31/7088; A61K 38/00; A61K 47/68; A61P 35/00; C07K 14/145; C07K 14/475; C07K 14/705; C07K 16/28; C12N 9/22; C12N 15/113

(52) U.S. Cl. CPC A61K 48/0041; A61K 31/7088; A61K 38/00; A61K 47/68; A61P 35/00; C07K 14/145; C07K 14/475; C07K 14/70517; C07K 16/28; C12N 9/222; C12N 15/1138; C07K 2317/31; C07K 2317/569; C07K 2317/622; C07K 2319/03; C12N 2310/20

(57) ABSTRACT

The present disclosure provides enveloped delivery vehicles (EDVs) comprising a nucleic acid-binding effector polypeptide, or a nucleic acid encoding the nucleic acid-binding effector polypeptide, where the EDV comprises a fusion polypeptide comprising (i) a viral envelope protein and (ii) a targeting polypeptide that provides for binding to a target cell. The present disclosure provides methods of using an EDV of the present disclosure for delivery of, e.g., a nucleic acid-binding effector polypeptide, to a eukaryotic cell.

### CROSS-REFERENCE

This application claims the benefit of U.S. Provisional Patent Application Nos. 63/400,166 filed Aug. 23, 2022, and 63/432,484 filed Dec. 14, 2022, which applications are incorporated herein by reference in their entirety.

### STATEMENT REGARDING FEDERALLY SPONSORED RESEARCH

This invention was made with government support under Grant Number GM143461 awarded by the National Institutes of Health. The government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, “BERK-477WO_SEQ_LIST.xml” created on Aug. 21, 2023, and having a size of 348,713 bytes. The contents of the Sequence Listing XML are incorporated by reference herein in their entirety.

### INTRODUCTION

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. Genome editing can be carried out using a CRISPR/Cas system comprising a CRISPR/Cas effector polypeptide and a guide RNA. CRISPR/Cas systems are revolutionizing the field of gene editing and genome engineering. Efficient methods for delivering CRISPR-Cas genome editing components into target cells are needed, for both ex vivo and in vivo applications.

Current delivery strategies have drawbacks. For example, delivery of a recombinant virus encoding a CRISPR-Cas effector polypeptide leads to prolonged CRISPR-Cas effector polypeptide expression in target cells, thus increasing the likelihood for off-target gene editing events. Others have used a ribonucleoprotein (RNP) comprising a CRISPR-Cas effector polypeptide and guide RNA (gRNA) to deliver the genome editing components into a cell.

There is a need in the art for additional strategies for delivering CRISPR-Cas effector polypeptides into target cells.

### SUMMARY

The present disclosure provides enveloped delivery vehicles (EDVs) (also referred to as virus like particles (VLPs)) comprising a nucleic acid-binding effector polypeptide (e.g., a CRISPR Cas effector polypeptide), or a nucleic acid encoding the nucleic acid-binding effector polypeptide, where the EDV comprises a fusion polypeptide comprising (i) a viral envelope protein and (ii) a targeting polypeptide that provides for binding to a target cell. The present disclosure provides methods of delivering a CRISPR-Cas effector polypeptide into a eukaryotic cell, using an EDV of the present disclosure. The present disclosure provides in vivo gene editing methods, using an EDV of the present disclosure.

### BRIEF DESCRIPTION OF THE DRAWINGS

FIG. 1A-1C depict genome editing of on-target cells versus bystander (non-target) cells using Cas9-EDVs comprising Gag-NES-2Xp53NLS-Cas9 and a guide RNA that provides for knockout of beta-2 microglobulin (β2M) in 293T cells.

FIG. 2 depicts genome editing of primary human cells (CD34⁺ hematopoietic stem and progenitor cells and T cells) with Cas9-EDVs.

FIG. 3 depicts genome editing of primary human T cells with Cas9-EDVs comprising a guide RNA that provides for knockout of TRAC. The EDVs comprised an envelope protein comprising: i) single-chain Fv (scFv) targeting ii) CD3; iii) CD4; iv) scFv targeting CD19; v) CD3 and CD4; or vi) no scFv. Cas9-EDVs pseudotyped with HIV-1 and VSVG are presented as controls-HIV-1 Env pseudotyped EDVs mediate genome editing specifically in CD4⁺ T cells, while VSVG pseudotyped EDVs mediate genome editing in CD4⁺ and CD8⁺ T cells.

FIG. 4 depicts activation of primary human T cells with Cas9-EDV displaying anti-CD3 scFv and anti-CD28 scFv.

FIG. 5 schematically depicts targeted lentivirus delivery in vivo.

FIG. 6A-6F depict targeted lentivirus delivery in vivo. Cas9-EDV comprising envelope proteins comprising anti-CD3 scFv and anti-CD28 scFv were used, and included a construct encoding a chimeric antigen receptor (CAR) that includes an anti-CD19 scFv. The data show the % CAR-T cells (FIG. 6D), the % CD19 B cells (FIG. 6E and FIG. 6F).

FIG. 7 schematically depicts assessing T cell-targeted Cas9-EDV and lentivirus activity in vivo.

FIG. 8A-8C depict in vivo generation of CAR-T cells.

FIG. 9 depicts in vivo generation of CAR-T cells. Cas9-EDVs included a construct encoding a CAR and a guide RNA that provides for knockout of TRAC. Control lentivirus included the construct encoding a CAR but not the guide RNA.

FIG. 10 depicts in vivo generation of CAR-T cells, and depicts the proportion that are CD4⁺ T cells or CD8⁺ T cells.

FIG. 11 depicts in vivo generation of CD8⁺ CAR-T cells.

FIG. 12A-12B depict in vivo genome editing of T cells.

FIG. 13A-13B depict in vivo genome editing of CD8⁺ T cells.

FIG. 14 depicts in vivo genome editing of mCherry⁺ CAR-T cells.

FIG. 15A-15P provide amino acid sequences of CRISPR-Cas effector polypeptides.

FIG. 16A-16D provide amino acid sequences of wild-type vesicular stomatitis virus (VSV) glycoprotein (VSVG) (FIG. 16A); a mutant VSVG (FIG. 16B); wild-type measles virus hemagglutinin (HA) (FIG. 16C); and a mutant measles virus HA (FIG. 16D).

FIG. 17 provides the amino acid sequence of a reverse transcriptase.

FIG. 18 provides a table that provides descriptions of scFv polypeptides referred to in Example 2.

FIG. 19A-19F provide nucleotide sequences (FIG. 19A-19D and FIG. 19F) encoding: Gag-Cas9 (FIG. 19A); Gag-Cas9 with 3× NES (FIG. 19B); Gag-Cas9 with 2× p53 NLS (FIG. 19C); Gag-Cas9 with 3× NES and 2× p53 NLS (FIG. 19D); anti-CD19 scFv 1 (FIG. 19F); and an amino acid sequence of Gag-Cas9 with 3× NES and 2× p53 NLS (FIG. 19E).

FIG. 20A-20E depict cell-specific genome editing with antibody-targeted Cas9-virus-like particles (VLPs).

FIG. 21A-21C depict use of antibody-targeted VLPs for genome editing of specific cells.

FIG. 22A-22E. Cell-specific genome editing with antibody-targeted Cas9-EDVs. (FIG. 22A) Schematic scFv targeting molecules (blue) and VSVGmut (orange) on the exterior surface of a Cas9-EDV. Cas9-EDVs package pre-formed Cas9-single guide RNA complexes to avoid genetically encoding genome editors within a viral genome. (FIG. 22B) Experimental outline and schematic of the lentiviral vector used for engineering HEK293T EGFP cells that express heterologous ligands on the plasma membrane (e.g. CD19). To promote cellular engineering via single lentiviral integration events, engineered cell mixtures were generated via low multiplicity of infection to achieve<25% EGFP+ cells. Engineered cell mixtures were challenged with B2M-targeting Cas9-EDVs to test targeting molecule activity. (FIG. 22C-22E) Assessment of antibody-targeted Cas9-EDV activity. HEK293T and CD19 EGFP HEK293T cells were mixed at an approximate ratio of 3:1 and treated with B2M-targeting Cas9-EDVs displaying various targeting molecule pseudotypes. Cas9-EDVs were concentrated 1Ox, and cells were treated with 50 1d Cas9-EDVs (FIG. 22C, 22E) or in a dilution curve (FIG. 22D). Analysis was performed at 7 days post-treatment to assess B2M knockout in EGFP+(on-target) and EGFP-(bystander) cells by flow cytometry (FIG. 22C, 22D) and amplicon sequencing (FIG. 22E). N=3 technical replicates for all panels except for the 100 μl dose of CD19-scFv in (FIG. 22D) (N=2). Error bars represent the standard error of the mean.

FIG. 23A-23I. Optimization of Cas9-EDVs for enhanced genome editing activity in primary human cells. (FIG. 23A) Genome editing activity comparison of CD19 antibody-targeted Cas9-EDV variants packaging B2M-targeted Cas9 ribonucleoproteins (RNPs). Expression of B2M protein was assessed by flow cytometry 7 days post-treatment in CD19-expressing target cells. (FIG. 23B) Diagram of the optimized Gag-Cas9 and Gag-pol Cas9-EDV production plasmids; features updated from Hamilton & Tsuchida et al., 2021 are highlighted in teal. (FIG. 23C-23E) Genome editing activity of optimized VSVG-pseudotyped Cas9-EDVs in primary human CD34⁺ cells (FIG. 23C) and activated (FIG. 23D) and resting primary human T cells (FIG. 23E). B2M or TRAC genome editing was assessed by amplicon sequencing 7 days post-treatment. (FIG. 23F) Schematic of potential intra-particle Cas9-EDV configurations for packaged Cas9 RNPs following proteolytic maturation. (FIG. 23G) Schematic of the compound GS-CA1 inhibiting either the nuclear import and/or uncoating of an HIV-1 capsid. (FIG. 23H) An mNeonGreen lentiviral vector was used to transduce HEK293T cells at the indicated MOI in the presence of GS-CA1 or DMSO. The percent of mNeonGreen-positive cells was assessed by flow cytometry 3 days post-treatment. TU=transducing units. (FIG. 231) B2M-targeting Cas9-EDVs, pre-titered such that the highest treatment dose would result in approximately 50% cells B2M-, were used to transduce HEK293T cells in the presence of GS-CA1 or DMSO. B2M expression was assessed by flow cytometry 3 days post-treatment. Error bars represent the standard deviation. N=3 technical replicates were used in all experiments.

FIG. 24A-24E. Multiplexed antibody targeting and editing of primary human T cells. (FIG. 24A-24B) Treating resting human T cells with Cas9-EDVs co-displaying CD3 and CD28 scFvs results in cellular activation (FIG. 24A) and proliferation (FIG. 24B) as measured by flow cytometry detection of CD25 3 days post-treatment and fold expansion relative to the untreated T cell count, respectively. CD25 expression and cellular proliferation was observed for CD3/CD28 scFv Cas9-EDVs, regardless of whether they packaged Cas9 RNPs targeting PDCD1 or a non-targeting control. (FIG. 24C) Genome editing 3 days post-treatment, as detected by amplicon next-generation sequencing. For figures A-C, Cas9-EDVs were concentrated 62× and 50 μl was used to treat 30k resting T cells. CD3 scFv-1 and CD28 scFv-2 were tested. (FIG. 24D) Screening the mono-display of additional CD3 scFv targeting molecules for B2M-targeted Cas9-EDVs on the Jurkat T cell line. B2M expression was assessed by flow cytometry 3 days post-treatment. Cas9-EDVs were concentrated 15× and 50 μl was used to treat 30k Jurkat cells. (FIG. 24E) Testing a panel of T cell-targeted, B2M-targeting Cas9-EDVs, displaying single or multiplexed scFv targeting molecules. Activated primary human T cells were treated with 1.38×10⁸ Cas9-EDVs displaying one or a combination of CD3 scFv-3, CD4 scFv-2, CD28 scFv-2, and a control scFv, and were assessed for B2M expression in CD4⁺ and CD8⁺ T cells by flow cytometry 6 days post-treatment. Error bars represent the standard error of the mean. N=3 technical replicates were for all panels.

FIG. 25A-25I. Programmable human cell delivery generates gene-edited CAR T cells in vivo. (FIG. 25A) Summary of T cell-targeted Cas9-EDVs and lentivirus tested in PBMC-humanized mice. Both particles display multiplexed scFvs (CD3 scFv-3, CD4 scFv-1, and CD28 scFv-2). The Cas9-EDV vector co-packages a lentiviral-encoded CAR-2A-mCherry transgene and Cas9 RNP complexes to disrupt the T cell receptor alpha constant (TRAC) gene; the lentivirus encodes the CAR-2A-mCherry transgene. (FIG. 25B) Experimental schematic for testing T cell-targeted Cas9-EDVs and lentivirus in PBMC-humanized mice by I.V. (intravenous) retro-orbital injections. (FIG. 25C) Representative flow cytometry plots demonstrating that CAR-expressing human T cells are present in the spleens of PBMC-humanized mice 10 days post administration of 1.5×10⁹ Cas9-EDV (N=2) or lentivirus (N=3), quantified in (FIG. 25D). PBS=phosphate-buffered saline. (FIG. 25E) Gene editing is observed in CAR-negative human T cells isolated from mice treated with T cell-targeted Cas9-EDVs, and is enriched in CAR-positive human T cells, (FIG. 25F). One CAR-positive lentivirus sample was excluded in (FIG. 25F) due to failing sequencing. (FIG. 25G) CAR-expressing human T cells are detectable in the spleens of PBMC-humanized mice 10 days post administration of 6.2×10⁸ Cas9-EDV (N=8) or lentivirus (N=8) administration. (FIG. 25H) Genome editing is observed in CAR-negative human T cells isolated from mice treated with T cell-targeted Cas9-EDVs and is enriched in CAR-positive human T cells, (FIG. 251). For all plots, black lines indicate the median of the data set. LOD=limit of detection, as defined by the average modified reads from lentiviral-treated samples.

FIG. 26A-26C. Functional dynamics of cellular engineering in vivo. (FIG. 26A) Depletion of CD19⁺ B cells is observed post-administration of T cell-targeted lentivirus (experiment 2). Human CD45⁺ cells were isolated from PBMC-humanized spleens 10 days post systemic administration, and the percentage of CD19-expressing cells was assessed by flow cytometry. P=*** indicates p<0.001 (unpaired T-test). Black lines indicate the median of the data set. (FIG. 26B) Model for the in vivo generation of functional CAR-T cells, with or without simultaneous gene editing. Schematic made with Biorender. (FIG. 26C) TCR clonotypes of CAR-transduced T cells isolated from humanized mice treated with T cell-targeted Cas9-EDV (mouse numbers 2, 4, 6) or lentivirus (mouse numbers 13, 14, 17) from experiment 2. “n” indicates the unique number of clonotypes detected.

FIG. 27A-27C. Establishing antibody-targeted Cas9-EDV delivery. (FIG. 27A) Schematic of an antibody-derived single-chain variable fragment (scFv) targeting molecule for Cas9-EDV display. The scFv is fused to the stalk and transmembrane domain (TMD) of human CD8a. (FIG. 27B) Single-chain variable fragment (scFv) targeting molecule schematic. ScFv targeting molecules were constructed in either VH-VL or VL-VH orientations. Amino acid (AA) residues correspond to the CD8a protein sequence (UniProt P01732); linker (SEQ ID NO:203). TMD=transmembrane domain. S=serine. (FIG. 27C) Antibody-targeted Cas9-EDVs mediate targeted genome editing regardless of target cell frequency. CD19 EGFP HEK293T cells were mixed with HEK293T cells to achieve target cell frequencies of ˜2-92%. Heterogeneous cell mixtures were challenged with antibody-targeted Cas9-EDVs (100 μl, 2.5× concentration) and B2M knockout was assessed in EGFP+(on-target) and EGFP-(bystander) cells by flow cytometry 7 days post-treatment. N=3 technical replicates. Error bars represent the standard error of the mean.

FIG. 28A-28C. Antibody-targeted Cas9-EDVs are a programmable strategy for mediating genome editing in specific cells. (FIG. 28A) Validation of cell-surface ligand expression in engineered cells. Engineered 293T cells transduced to express either CD19, CD20, CD4, or CD28 and EGFP were assayed for successful ligand expression. Cells were stained with monoclonal antibodies (CD19-PE, CD20-PE CD4-PE-Cy7, CD28-PE) to verify ligand expression only in EGFP+ cells. (FIG. 28B) Various scFv targeting molecules were developed to target ligands expressed on human immune cells: CD19, CD20, CD4, and CD28. Cell-specific antibody-retargeted Cas9-EDVs activity was assessed for on-target, ligand+ cells (EGFP+) and off-target, bystander cells (EGFP-) by flow cytometry 7 days post-treatment. (FIG. 28C) Antibody-targeted Cas9-EDV-mediated genome editing is highly specific for cells expressing the scFv cognate ligand. scFv-Cas9-EDVs were tested on engineered cells expressing the cognate and noncognate ligands, and genome editing activity was measured by flow cytometry 3 days post-treatment. N=3 technical replicates. All error bars represent the standard error of the mean.

FIG. 29A-29J. Optimization and study of Cas9-EDV activity. (FIG. 29A) Diagram of the Gag-Cas9 plasmid indicating the site of nuclear localization signal (NLS) variant addition to the N-terminal end of Cas9. (FIG. 29B) Assessment of N-terminal Cas9 NLS additions on Cas9-EDV activity. Cas9-EDVs were used to treat a mixture of CD19⁺ and CD19-293T cells. Genome editing was assessed by flow cytometry detection of B2M expression in CD19⁺target cells and CD19-bystander cells 7 days post-treatment. BP=bipartite. (FIG. 29C-29D) Direct comparison of genome editing in CD19⁺on-target cells (FIG. 29C) and CD19-bystander cells (FIG. 29D) treated with Gag-[3× NES]-Cas9 EDVs versus Gag-Cas9 EDVs. Genome editing was assessed by flow cytometry detection of B2M expression 7 days post-treatment. (FIG. 29E) Genome editing activity comparison of CD19 antibody-targeted Cas9-EDV variants packaging B2M-targeted Cas9 RNPs. Expression of B2M protein was assessed by flow cytometry 7 days post-treatment in CD19-negative bystander cells. (FIG. 29F) Direct comparison of genome editing in on-target and bystander cells of the Gag-[3× NES]-[2× p53-NLS]-Cas9 EDV variant. The difference in genome-edited cells between the two cell types is indicated for 1.56 μl and 6.25 pl Cas9-EDV treatment doses. (FIG. 29G) Treatment of target cells with GS-CA1 does not alter genome editing by Cas9 RNP nucleofection. 293T cells were nucleofected with 50 pmol B2M-targeted Cas9 RNPs and either cultured in the presence of 25 nM GS-CA1, 0.05% DMSO, or Opti-MEM. Genome editing was assessed by flow cytometry detection of B2M expression at 3 days post-treatment. (FIG. 29H) Diagram of the Gag-TCL-Cas9 and Gag-TCL-TEV plasmids for producing TEVp-Cas9-EDVs which rely on the TEV protease to release Cas9 from Gag. TCL=TEV protease-cleavable linker. (FIG. 291) Western blot analysis of the loss of Gag proteolytic processing in TEVp-Cas9-EDVs, as indicated by the absence of p24 and other Gag proteolytic intermediates. CA=capsid, a component of the gag polypeptide. (FIG. 29J) Genome editing activity of TEVp-Cas9-EDVs packaging Cas9 RNPs that activate tdTomato expression upon genome editing of a murine neural progenitor cell line harboring a loxP-STOP-loxP-tdTomato reporter (51). Genome editing was assessed by flow cytometry detection of tdTomato reporter activation 7 days post-treatment. N=3 technical replicates were used in all experiments.

FIG. 30A-30M. Optimized Cas9-EDV characterization and in vivo biodistribution. (FIG. 30A) Dynamic light scattering analysis of Cas9-EDV and lentiviral particles. Particles were pseudotyped with either VSVG or an anti-CD19 scFv+VSVGmut and the Z-Average (Z-Ave) was measured in nanometers (d.nm). Commercially-available 100 nm gold beads were used as a control. Points indicate technical replicates. (FIG. 30B) Representative transmission electron microscopy (TEM) images of anti-CD19 scFv+VSVGmut-pseudotyped Cas9-EDVs. (FIG. 30C) Anti-myc immunogold TEM labeling of anti-CD19 scFv 1 targeting molecules on Cas9-EDVs (top). No staining is observed when the anti-CD19 scFv 1 myc tag is swapped for a strep tag (bottom). (FIG. 30D) Detection of B2M sgRNA in produced Cas9-EDVs and Cre-EDVs. A custom qPCR primer/probe set was used for B2M sgRNA detection, and fold change was calculated by normalizing to the Cre-EDV sample. Cre-EDVs were generated by swapping Cre Recombinase for Cas9 in the Gag-Cas9 V2 plasmid (see FIG. 2B). Cre-EDVs (no sgRNA) were produced analogously, but with plasmids lacking the U6-sgRNA expression cassette. N.D=not detected. Points indicate technical replicates. (FIG. 30E) To assess the unintended carry-over of plasmid from producer cells to Cas9-EDV treated cells, LentiX cells were transfected to produce Cas9-EDVs either in the absence (top) or the presence (bottom) of 1 μg of an EGFP-expressing plasmid. Flow cytometry was performed on the producer cells three days post transfection. (FIG. 30F) Flow cytometry analysis of EGFP-expression in Cas9-EDV treated cells. 293T cells were treated with B2M Cas9-EDVs produced either in the absence (top) or presence (bottom) of an EGFP-expression plasmid. Flow cytometry was performed 6 days post Cas9-EDV treatment and EGFP and B2M expression were assessed. (FIG. 30G) Standard curve quantifying synthesized B2M sgRNA via a custom qPCR primer/probe set, used to interpolate the number of B2M sgRNAs associated with Cas9-EDVs produced in one 10-cm dish. (FIG. 30H). Points indicate biological replicates of Cas9-EDVs produced in one 10-cm dish and concentrated 30× into 300 1d. (FIG. 30I) The number of Cas9-EDV particles produced per one 10-cm dish, as estimated by p24 ELISA. Points indicate biological replicates (FIG. 30J & 30K). B2M knockout in 293T cells following treatment with the indicated Cas9-EDV volumes (Cas9-EDVs were concentrated 30×) (FIG. 30J) or nucleofection with Cas9 RNP complexes (FIG. 30K). Flow cytometry was used to detect B2M expression 6 days post treatment. Four-parameter logistic curves were used to fit the data. (FIG. 30L) Genome editing comparison between Cas9-EDV treatment volume and Cas9 RNP nucleofection. Data from J and K were used to interpolate how Cas9-EDV treatment volume relates to Cas9 RNP pmol dose. B2M sgRNA molecules and particle numbers are scaled to Cas9-EDV treatment volume (FIG. 30H & 30I). (FIG. 30M) Biodistribution of VSVG and anti-CD19 scFv+VSVGmut lentivirus. 3E9 lentiviral copies, as normalized by qPCR, were systemically administered to C57BL/6 mice through retro-orbital injections. The indicated tissues/organs were harvested five minutes post-vector administration, RNA was isolated, and qPCR was performed to detect the presence of lentiviral vectors. The quantity of vector is plotted as a fold change, relative to the amount of vector in the serum. Each point represents an individual mouse. Two mice received 100 pl PBS as a vehicle control and no lentiviral vector was detected in any organ, as expected (not shown). Floating numbers (D, H, I) indicate the mean. All error bars represent standard error of the mean.

FIG. 31A-31D. Assessment of T cell-targeting molecules for Cas9-EDV delivery. (FIG. 31A) Assessment of human CD45 targeting molecules. A panel of CD45-targeted Cas9-EDVs packaging B2M-targeted Cas9 RNPs were produced and concentrated 13.8-fold. 100 pl was used to treat 30k Jurkat cells, and B2M expression was assessed by flow cytometry on day 3. (FIG. 31B) Assessment of CD4 and CD45-targeted Cas9-EDVs in activated primary human T cells. Cas9-EDVs packaging TRAC-targeted Cas9 RNPs were produced and 1.15×10⁷ Cas9-EDVs were used to treat 15k activated T cells. T cell receptor (TCR) loss, indicative of TRAC genome editing, was assessed by flow cytometry 5 days post-treatment. Representative plots are shown, and results are quantified in (FIG. 31C). CD4 scFv-1 and CD45 scFv-5 targeting molecules were used. (FIG. 31D) Assessment of multiplexing targeting molecules for delivery to activated primary human T cells. 15k activated T cells were treated with 1.38×10⁸ Cas9-EDVs packaging B2M-targeted Cas9 RNPs, and flow cytometry analysis of B2M expression was assessed on day 6. Representative flow cytometry plots are shown.CD3 scFv-3, CD4 scFv-2, and CD28 scFv-2 targeting molecules were used. Error bars represent the standard error of the mean. N=3 technical replicates were assessed (FIG. 31A & 31C).

FIG. 32A-32G. Assessment of CAR T generation in vivo using T cell-targeted vectors. (FIG. 32A) Analysis of mCherry+CAR T cells in spleens from humanized mouse experiment 1. Mice were treated with Cas9-EDV, lentivirus (LV), or PBS and flow cytometry analysis was performed 10 days post-treatment. Flow cytometry quantification of CD4⁺(FIG. 32B) and CD8⁺ CAR T cells (FIG. 32C). (FIG. 32D) Analysis of mCherry+CAR T cells in spleens from humanized mouse experiment 2. Mice were treated with Cas9-EDV, lentivirus (LV), or PBS and flow cytometry analysis was performed 10 days post-treatment. Upper left quadrant numbers are mouse identifiers. Flow cytometry quantification of CD4⁺(FIG. 32E) and CD8⁺ CAR T cells (FIG. 32F). (FIG. 32G) Quantification of body weight change in humanized mice from experiment 2. The dotted line indicates 100%. Black lines indicate the median (B-C, E-G).

FIG. 33A-33B. Analysis of delivery to off-target cells in vivo. (FIG. 33A-33B) Representative confocal microscopy images of the livers from PBMC-humanized mice 10 days post systemic administration of T-cell targeting Cas9-EDV and lentivirus demonstrating that mCherry+(red) cells co-localize with human CD3⁺(green) T cells (FIG. 33A) and F4/80⁺(green) phagocytic cells (FIG. 33B) but not β-catenin+(teal) hepatocytes. Scale bar, 65 pm.

FIG. 34A-34B. Assessment of CD19⁺ B cells and CAR T cell receptor clonality following in vivo cellular engineering. (FIG. 34A) Flow cytometry analysis of CD19⁺ B cells in Cas9-EDV- or lentivirus-treated mice 10 days post-administration. Human CD45⁺ cells were isolated from humanized mouse spleens prior to flow cytometry analysis. Upper left quadrant numbers are mouse identifiers. (FIG. 34B) Correlation between the number of in vivo-generated CAR T clonotypes versus the number of CAR T cells sorted (Pearson's correlation coefficient, R=0.938). Point labels indicate the unique mouse analyzed.

FIG. 35 provides a table that provides descriptions of scFv polypeptides referred to in Example 3. Sequences: (GGGGSGGGGSGGGGS, SEQ ID NO194; TGGGGSGGGGSGGGGS, SEQ ID NO:192; TGSTSGSGKPGSGEGSTKG, SEQ ID NO:193; GGGGSGGGGSGGGGSS, SEQ ID NO:203).

### DEFINITIONS

“Heterologous,” as used herein, means a nucleotide or polypeptide sequence that is not found in the native nucleic acid or protein, respectively. For example, relative to a CRISPR-Cas effector polypeptide, a heterologous polypeptide comprises an amino acid sequence from a protein other than the CRISPR-Cas effector polypeptide. As another example, a CRISPR-Cas effector protein (e.g., a dead CRISPR-Cas effector protein) can be fused to an active domain from a non-CRISPR-Cas effector protein (e.g., a cytidine deaminase), and the sequence of the active domain could be considered a heterologous polypeptide (it is heterologous to the CRISPR-Cas effector protein).

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “naturally-occurring” as used herein as applied to a nucleic acid, a protein, a cell, or an organism, refers to a nucleic acid, cell, protein, or organism that is found in nature.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

“Heterologous,” as used herein, refers to a nucleotide or amino acid sequence that is not found in the native nucleic acid or protein, respectively. For example, relative to a Cas9 polypeptide, a heterologous polypeptide comprises an amino acid sequence from a protein other than the Cas9 polypeptide. Thus, for example, a polymerase polypeptide is heterologous to a Cas9 polypeptide.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, nucleotide sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant nucleotide sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such artificial combination can be carried out to join together nucleic acid segments of desired functions to generate a desired combination of functions.

Similarly, the term “recombinant” polypeptide refers to a polypeptide which is not naturally occurring, e.g., is made by the artificial combination of two otherwise separated segments of amino acid sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change can be achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013);5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, evibodies, minobodies, diabodies, and fusion proteins comprising an antigen-binding (also referred to herein as antigen binding) portion of an antibody and a non-antibody protein.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids. In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VH_{H} antibody.

“Single-chain Fv” or “sFv” or “scFv” antibody fragments comprise the V_{H} and V_{L}domains of antibody, wherein these domains are present in a single polypeptide chain. In some embodiments, the Fv polypeptide further comprises a polypeptide linker between the V_{H} and V_{L}domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “antibody mimetic” refers to compounds which, like antibodies, can specifically and/or selectively bind antigens or other targets, but which are not structurally related to antibodies. Antibody mimetics are usually artificial peptides or proteins, but they are not limited to such embodiments. Typically, antibody mimetics are smaller than antibodies, with a molar mass of about 3-20 kDa (whereas antibodies are generally about 150 kDa). Non-limiting examples of antibody mimetics include peptide aptamers, affimers, affilins, affibodies, affitins, alphabodies, anticalins, avimers, DARPins, fynomers, Kunitz domain peptides, nanoCLAMPs, affinity reagents and scaffold proteins.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, non-human primates, humans, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a guide RNA” includes a plurality of such guide RNAs; reference to “a targeting polypeptide” includes a plurality of such polypeptides; and reference to “the CRISPR-Cas effector polypeptide” includes reference to one or more CRISPR-Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The terms enveloped delivery vehicle (EDV) and virus like particle (VLP) are used interchangeably herein as equivalent terms. As such, reference to one (e.g., EDV) is considered reference to the other (e.g., VLP). Therefore, all disclosures/discussions herein of EDVs are to be equally considered disclosures/discussions of VLPs and vice versa.

The present disclosure provides enveloped delivery vehicles (EDVs) comprising a nucleic acid-binding effector polypeptide, or a nucleic acid encoding the nucleic acid-binding effector polypeptide, where the EDV comprises a fusion polypeptide comprising (i) a viral envelope protein and (ii) a targeting polypeptide that provides for binding to a target cell. The present disclosure provides methods of delivering a nucleic acid-binding effector polypeptide into a eukaryotic cell, using an EDV of the present disclosure.

The present disclosure provides EDVs comprising a CRISPR-Cas effector polypeptide, or a nucleic acid encoding the CRISPR-Cas effector polypeptide, where the EDV comprises a fusion polypeptide comprising (i) a viral envelope protein and (ii) a targeting polypeptide that provides for binding to a target cell. The present disclosure provides methods of delivering a CRISPR-Cas effector polypeptide into a eukaryotic cell, using an EDV of the present disclosure.

In some cases, the EDV comprises a nucleic acid comprising a nucleotide sequence encoding a therapeutic polypeptide, such as a chimeric antigen receptor (CAR). In some cases, the EDV comprises one or more CRISPR-Cas guide nucleic acids, or one or more nucleic acids comprising nucleotide sequences encoding the one or more CRISPR-Cas guide nucleic acids, where the one or more CRISPR-Cas guide nucleic acids provide for knockout of an endogenous nucleic acid. In some cases, the EDV comprises: i) a donor template nucleic acid; and ii) one or more CRISPR-Cas guide nucleic acids, or one or more nucleic acids comprising nucleotide sequences encoding the one or more CRISPR-Cas guide nucleic acids, where contacting a target nucleic acid with a CRISPR-Cas effector polypeptide, the one or more CRISPR-Cas guide nucleic acids, and the donor template nucleic acid, results in insertion of the donor template nucleic acid into the target nucleic acid. In some cases, the donor template nucleic acid comprises a nucleotide sequence encoding a therapeutic polypeptide.

### Enveloped Delivery Vehicles (Edvs) Comprising One or More Targeting Polypeptides

The present disclosure provides enveloped delivery vehicles (EDVs) comprising a nucleic acid-binding effector polypeptide, or a nucleic acid comprising a nucleotide sequence encoding the nucleic acid-binding effector polypeptide, where the EDVs comprise a fusion polypeptide comprising (i) a viral envelope protein (e.g., a viral envelope glycoprotein) and (ii) a polypeptide (a “targeting polypeptide”) that provides for binding to a target cell.

The present disclosure provides enveloped delivery vehicles (EDVs) comprising a nucleic acid-binding effector polypeptide, or a nucleic acid comprising a nucleotide sequence encoding the nucleic acid-binding effector polypeptide, where the EDVs comprise a fusion polypeptide comprising (i) a viral envelope protein (e.g., a viral envelope glycoprotein) and (ii) one or more antibodies or antibody analogs that specifically bind to a target polypeptide on a target cell. In some cases, the EDV comprises a therapeutic polypeptide, or a nucleic acid comprising a nucleotide sequence encoding the therapeutic polypeptide, encapsidated within the EDV. The EDVs can be used in in vivo methods of genome editing, which methods are also provided. In some cases, the EDVs comprise a CRISPR-Cas effector polypeptide as the nucleic acid-binding effector polypeptide. In some cases, the EDVs comprise a nucleic acid comprising a nucleotide sequence encoding a CRISPR-Cas effector polypeptide. In some cases, an EDV of the present disclosure comprises an RNP comprising: a) a CRISPR-Cas effector polypeptide; and b) a CRISPR-Cas guide nucleic acid, where the guide nucleic acid (e.g., guide RNA)) comprises: i) a nucleotide sequence that comprises a protein-binding segment comprising a nucleotide sequence that binds to the CRISPR-Cas effector polypeptide, and a target-binding segment comprising a nucleotide sequence that is complementary to a target nucleotide sequence of a target DNA in a cell (e.g., a eukaryotic cell; e.g., a eukaryotic cell present in an individual). In some cases, an EDV of the present disclosure comprises: a nucleic acid (e.g., a recombinant expression vector) comprising a nucleotide sequence encoding a CRISPR-Cas effector polypeptide and a nucleotide sequence encoding a CRISPR-Cas guide RNA. In some cases, an EDV of the present disclosure comprises a donor nucleic acid. Viral envelope proteins

As noted above, an EDV of the present disclosure comprises a fusion polypeptide comprising: (i) a viral envelope protein (e.g., a viral envelope glycoprotein) and (ii) polypeptide that provides for binding to a target cell.

Suitable viral envelope proteins include, e.g., a vesicular stomatitis virus (VSV) glycoprotein (VSV-G protein), a Measles virus hemagglutinin (HA) protein and/or a measles virus fusion glycoprotein, an Influenza virus neuraminidase (NA) protein, a Measles virus F protein, an Influenza virus HA protein, a Moloney virus MLV-A protein, a Moloney virus MLV-E protein, a Baboon Endogenous retrovirus (BAEV) envelope protein, an Ebola virus glycoprotein, a foamy virus envelope protein, and a combination or two or more of the foregoing viral envelope proteins.

In some cases, the viral envelope protein is a VSV-G protein. In some cases, the viral envelope protein is a measles virus hemagglutinin protein. In some cases, the viral envelope protein is a measles virus F protein. In some cases, the viral envelope protein is an influenza virus hemagglutinin protein. In some cases, the viral envelope protein is a Moloney virus MLV-A protein. In some cases, the viral envelope protein is a Moloney virus MLV-E protein. In some cases, the viral envelope protein is a baboon endogenous retrovirus envelope protein. In some cases, the viral envelope protein is an Ebola virus glycoprotein. In some cases, the viral envelope protein is a foamy virus envelope protein.

In some cases, the viral envelope protein is a VSV-G protein. A suitable VSV-G protein comprises an amino acid sequence having at least 80%, at least 85%, at least 90%, at least 95%, at least 98%, at least 99%, or 100%, amino acid sequence identity to the following amino acid sequence:

(SEQ ID NO: 18)
IMKCLLYLAFLFIGVNCKFTIVFPHNQKGNWKNVPSNYHYCPSSSDLNWHNDLIGTALQ
VKMPKSHKAIQADGWMCHASKWVTTCDFRWYGPKYITHSIRSFTPSVEQCKESIEQTKQGTWL
NPGFPPQSCGYATVIDAEAVIVQVTPHHVLVDEYTGEWVDSQFINGKCSNYICPTVHNSTTWHS
DYKVKGLCDSNLISMDITFFSEDGELSSLGKEGTGFRSNYFAYETGGKACKMQYCKHWGVRLP
SGVWFEMADKDLFAAARFPECPEGSSISAPSQTSVDVSLIQDVERILDYSLCQETWSKIRAGLPIS
PVDLSYLAPKNPGTGPAFTIINGTLKYFETRYIRVDIAAPILSRMVGMISGTTTERELWDDWAPY
EDVEIGPNGVLRTSSGYKFPLYMIGHGMLDSDLHLSSKAQVFEHPHIQDAASQLPDDESLFFGDT
GLSKNPIELVEGWFSSWKSSIASFFFIIGLIIGLFLVLRVGIHLCIKLKHTKKRQIYTDIEMNRLGK.

In some cases, a suitable VSV-G protein comprises an amino acid sequence having at least 80%, at least 85%, at least 90%, at least 95%, at least 98%, at least 99%, or 100%, amino acid sequence identity to the following amino acid sequence:

(SEQ ID NO: 19)
MKCLLYLAFLFIGVNCKFTIVFPHNQKGNWKNVPSNYHYCPSSSDLNWHNDLIGTALQ
VKMPKSHKAIQADGWMCHASKWVTTCDFRWYGPKYITHSIRSFTPSVEQCKESIEQTKQGTWL
NPGFPPQSCGYATVTDAEAVIVQVTPHHVLVDEYTGEWVDSQFINGKCSNYICPTVHNSTTWHS
DYKVKGLCDSNLISMDITFFSEDGELSSLGKEGTGFRSNYFAYETGGKACKMQYCKHWGVRLP
SGVWFEMADKDLFAAARFPECPEGSSISAPSQTSVDVSLIQDVERILDYSLCQETWSKIRAGLPIS
PVDLSYLAPKNPGTGPAFTIINGTLKYFETRYIRVDIAAPILSRMVGMISGTTTERELWDDWAPY
EDVEIGPNGVLRTSSGYKFPLYMIGHGMLDSDLHLSSKAQVFEHPHIQDAASQLPDDESLFFGDT
GLSKNPIELVEGWFSSWKSSIASFFFIIGLIIGLFLVLRVGIHLCIKLKHTKKRQIYTDIEMNRLGK.

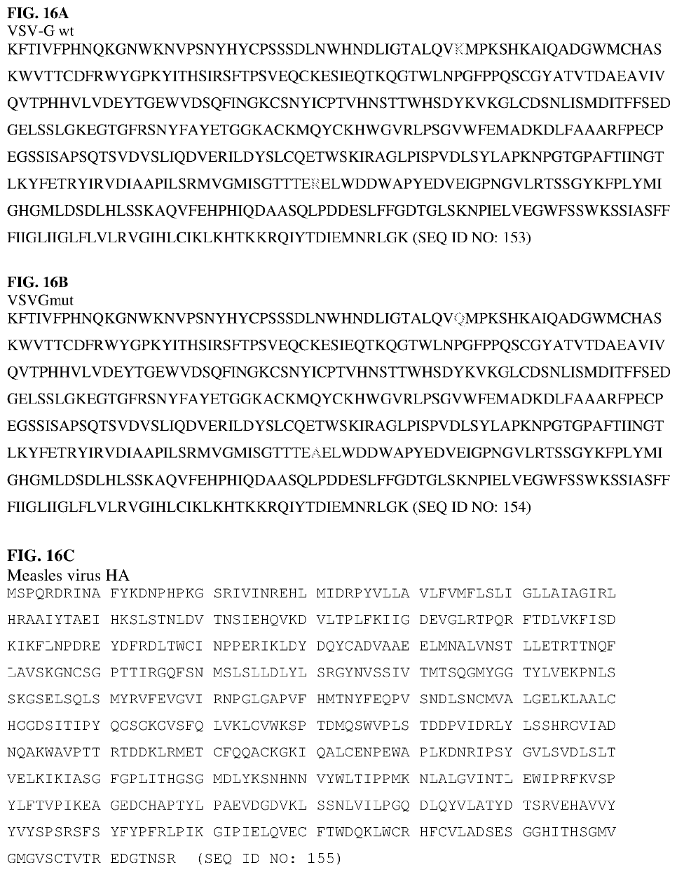

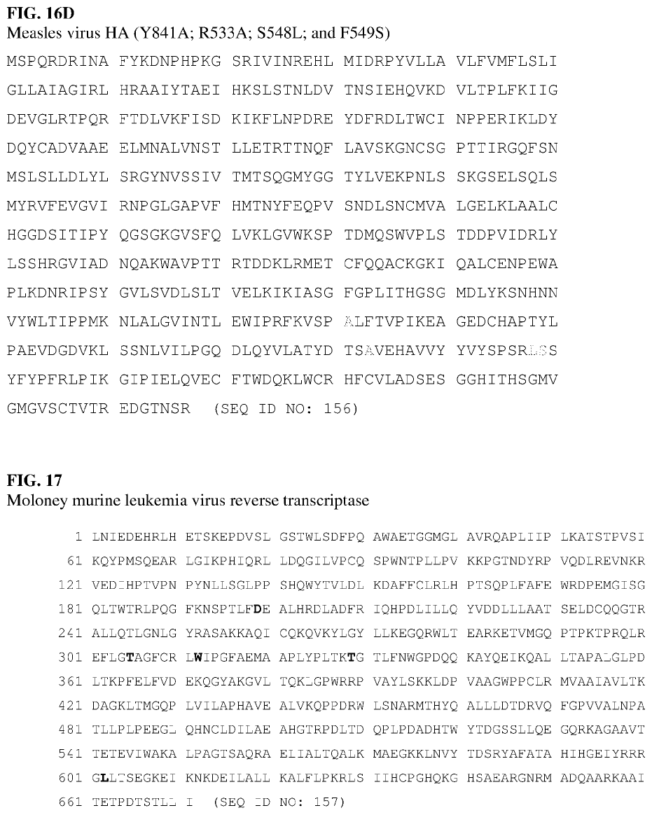

In some cases, a suitable VSV-G protein comprises an amino acid sequence having at least 80%, at least 85%, at least 90%, at least 95%, at least 98%, at least 99%, or 100%, amino acid sequence identity to the amino acid sequence depicted in FIG. 16A.

In some cases, the viral envelope protein is a BAEV-G protein. A suitable BAEV-G protein comprises an amino acid sequence having at least 80%, at least 85%, at least 90%, at least 95%, at least 98%, at least 99%, or 100%, amino acid sequence identity to the following amino acid sequence:

(SEQ ID NO: 49)
MGFTTKIIFLYNLVLVYAGFDDPRKAIELVQKRYGRPCDCSGGQVSEPPSDRVSQVTCS
GKTAYLMPDQRWKCKSIPKDTSPSGPLQECPCNSYQSSVHSSCYTSYQQCRSGNKTYYTATLLK
TQTGGTSDVQVLGSTNKLIQSPCNGIKGQSICWSTTAPIHVSDGGGPLDTTRIKSVQRKLEEIHKA
LYPELQYHPLAIPKVRDNLMVDAQTLNILNATYNLLLMSNTSLVDDCWLCLKLGPPTPLAIPNF
LLSYVTRSSDNISCLIIPPLLVQPMQFSNSSCLFSPSYNSTEEIDLGHVAFSNCTSITNVTGPICAVN
GSVFLCGNNMAYTYLPTNWTGLCVLATLLPDIDIIPGDEPVPIPAIDHFIYRPKRAIQFIPLLAGLG
ITAAFTTGATGLGVSVTQYTKLSNQLISDVQILSSTIQDLQDQVDSLAEVVLQNRRGLDLLTAEQ
GGICLALQEKCCFYVNKSGIVRDKIKTLQEELERRRKDLASNPLWTGLQGLLPYLLPFLGPLLTL
LLLLTIGPCIFNRLTAFINDKLNIIHAMVLTQQYQVLRTDEEAQD.

In some cases, the viral envelope protein is an influenza virus HIN1 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MKAILVVLLY TFATANADTL CIGYHANNST DTVDTVLEKN VTVTHSVNLL EDKHNGKLCK LRGVAPLHLG KCNIAGWILG NPECESLSTA SSWSYIVETP SSDNGTCYPG DFIDYEELRE QLSSVSSFER FEIFPKTSSW PNHDSNKGVT AACPHAGAKS FYKNLIWLVK KGNSYPKLSK SYINDKGKEV LVLWGIHHPS TSADQQSLYQ NADAYVFVGS SRYSKKFKPE IAIRPKVRXX EGRMNYYWTL VEPGDKITFE ATGNLVVPRY AFAMERNAGS GIIISDTPVH DCNTTCQTPK GAINTSLPFQ NIHPITIGKC PKYVKSTKLR LATGLRNIPS IQSRGLFGAI AGFIEGGWTG MVDGWYGYHH QNEQGSGYAA DLKSTQNAID EITNKVNSVI EKMNTQFTAV GKEFNHLEKR IENLNKKVDD GFLDIWTYNA ELLVLLENER TLDYHDSNVK NLYEKVRSQL KNNAKEIGNG CFEFYHKCDN TCMESVKNGT YDYPKYSEEA KLNREEIDGV KLESTRIYQI LAIYSTVASS LVLVVSLGAI SFWMCSNGSL QCRICI (SEQ ID NO:50; GenBank Accession No: ACP44189). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and natural killer (NK) cells.

In some cases, the viral envelope protein is an influenza virus H3N2 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MKTIIALSYI LCLVFAQKLP GNDNSTATLC LGHHAVPNGT IVKTITNDQI EVTNATELVQ SSSTGGICDS PHQILDGENC TLIDALLGDP QCDGFQNKKW DLFVERSKAY SNCYPYDVPD YASLRSLVAS SGTLEFNNES FNWTGVTQNG TSSACKRRSN NSFFSRLNWL THLKFKYPAL NVTMPNNEKF DKLYIWGVHH PGTDNDQISL YAQASGRITV STKRSQQTVI PSIGSRPRIR DVPSRISIYW TIVKPGDILL INSTGNLIAP RGYFKIRSGK SSIMRSDAPI GKCNSECITP NGSIPNDKPF QNVNRITYGA CPRYVKQNTL KLATGMRNVP EKQTRGIFGA IAGFIENGWE GMVDGWYGFR HQNSEGTGQA ADLKSTQAAI NQINGKLNRL IGKTNEKFHQ IEKEFSEVEG RIQDLEKYVE DTKIDLWSYN AELLVALENQ HTIDLTDSEM NKLFERTKKQ LRENAEDMGN GCFKIYHKCD NACIGSIRNG TYDHDVYRDE ALNNRFQIKG VELKSGYKDW ILWISFAISC FLLCVALLGF IMWACQKGNI RCNICI (SEQ ID NO:51; GenBank Accession No: YP_308839). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and natural killer (NK) cells.

In some cases, the viral envelope protein is an influenza virus A H5N1 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MEKIVLLLAI VSLVKSDQIC IGYHANNSTE QVDTIMEKNV TVTHAQDILE KTHNGKLCDL NGVKPLILRD CSVAGWLLGN PMCDEFINVP EWSYIVEKAS PANDLCYPGD FNDYEELKHL LSRTNHFEKI QIIPKSSWSN HDASSGVSSA CPYHGRSSFF RNVVWLIKKN SAYPTIKRSY NNTNQEDLLV LWGIHHPNDA AEQTKLYQNP TTYISVGTST LNQRLVPEIA TRPKVNGQSG RMEFFWTILK PNDAINFESN GNFIAPEYAY KIVKKGDSAI MKSELEYGNC NTKCQTPMGA INSSMPFHNI HPLTIGECPK YVKSNRLVLA TGLRNTPQRE RRRKKRGLFG AIAGFIEGGW QGMVDGWYGY HHSNEQGSGY AADKESTQKA IDGVTNKVNS IIDKMNTQFE AVGREFNNLE RRIENLNKQM EDGFLDVWTY NAELLVLMEN ERTLDFHDSN VKNLYDKVRL QLRDNAKELG NGCFEFYHKC DNECMESVKN GTYDYPQYSE EARLNREEIS GVKLESMGTY QILSIYSTVA SSLALAIMVA GLSLWMCSNG SLQCRICI (SEQ ID NO:52; GenBank Accession No: YP_308669). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an influenza virus H7N9 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MNTQILVFAL IAIIPTNADK ICLGHHAVSN GTKVNTLTER GVEVVNATET VERTNIPRIC SKGKRTVDLG QCGLLGTITG PPQCDQFLEF SADLIIERRE GSDVCYPGKF VNEEALRQIL RESGGIDKEA MGFTYSGIRT NGATSACRRS GSSFYAEMKW LLSNTDNAAF PQMTKSYKNT RKSPALIVWG IHHSVSTAEQ TKLYGSGNKL VTVGSSNYQQ SFVPSPGARP QVNGLSGRID FHWLMLNPND TVTFSFNGAF IAPDRASFLR GKSMGIQSGV QVDANCEGDC YHSGGTIISN LPFQNIDSRA VGKCPRYVKQ RSLLLATGMK NVPEIPKGRG LFGAIAGFIE NGWEGLIDGW YGFRHQNAQG EGTAADYKST QSAIDQITGK LNRLIEKTNQ QFELIDNEFN EVEKQIGNVI NWTRDSITEV WSYNAELLVA MENQHTIDLA DSEMDKLYER VKRQLRENAE EDGTGCFEIF HKCDDDCMAS IRNNTYDHSK YREEAMQNRI QIDPVKLSSG YKDVILWFSF GASCFILLAI VMGLVFICVK NGNMRCTICI (SEQ ID NO:53; GenBank Accession No: YP_009118475). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a Hepatitis B Virus (HBV) S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MENTTSGFLG PLLVLQAGFF LLTRNLTIPQ SLDSWWTSLN FLGGAPTCPG QNSQSPTSNH SPTSCPPICP GYRWMCLRRF IIFLFILLLC LIFLLVLLDY QGMLPVCPLL PGTSTTSTGP CKTCTIPAQG TSMFPSCCCT KPSDGNCTCI PIPSSWAFAR FLWEWASVRF SWLSLLVPFV QWFVGLSPTV WLSVIWMMWY WGPSLYNILS PFLPLLPIFF CLWVYI (SEQ ID NO:54; GenBank Accession No: ABV02793). Such a heterologous glycoprotein may be useful in directing an EDV of the present disclosure to a liver cell.

In some cases, the viral envelope protein is a Hepatitis B Virus (HBV) middle S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MQWNSTAFHQ ALQDPKVRGL YFPAGGSSSG TVNPAPNIAS HISSISARTG DPVTNMENIT SGFLGPLLVL QAGFFLLTRI LTIPQSLDSW WTSLNFLGGS PVCLGQNSQS PTSNHSPTSC PPICPGYRWM CLRRFIIFLF ILLLCLIFLL VLLDYQGMLP VCPLIPGSTT TSTGPCKTCT TPAQGNSMFP SCCCTKPTDG NCTCIPIPSS WAFAKYLWEW ASVRFSWLSL LVPFVQWFVG LSPTVWLSAI WMMWYWGPSL YSIVSPFIPL LPIFFCLWVY I (SEQ ID NO:55; GenBank Accession No: ACJ66136). Such a heterologous glycoprotein may be useful in directing an EDV of the present disclosure to a liver cell.

In some cases, the viral envelope protein is a Hepatitis B Virus (HBV) large S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MGLSWTVPLE WGKNHSTTNP LGFFPDHQLD PAFRANTRNP DWDHNPNKDH WTEANKVGVG AFGPGFTPPH GGLLGWSPQA QGMLKTLPAD PPPASTNRQS GRQPTPITPP LRDTHPQAMQ WNSTTFHQAL QDPKVSALYL PAGGSSSGTV NPVPTTASLI SSIFSRIGDP APNMESITSG FLGPLLVLQA GFFLLTKILT IPQSLDSWWT SLNFLGGAPV CLGQNSQSPT SSHSPTSCPP ICPGYRWMCL RRFIIFLFIL LLCLIFLLVL LDYQGMLPVC PLIPGSSTTS TGPCRTCTTL AQGTSMFPSC CCSKPSDGNC TCIPIPSSWA FGKFLWEWAS ARFSWLSLLV PFVQWFAGLS PTVWLSVIWM MWYWGPSLYN ILSPFIPLLP IFFCLWVYI (SEQ ID NO:56; GenBank Accession No: AGR65633). Such a heterologous glycoprotein may be useful in directing an EDV of the present disclosure to a liver cell.

In some cases, the viral envelope protein is a Hepatitis B Virus (HBV) small S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MENITSGFLG PLLVLQAGFF LLTRILTIPQ SLDSWWTSLN FLGGTTVCLG QNSQSPTSNH SPTSCPPTCP GYRWMCLRRF IIFLFILLLC LIFLLVLLDY QGMLPVCPLI PGSSTTSTGP CRTCTTPAQG TSMYPSCCCT KPSDGNCTCI PIPSSWAFGK FLWEWASARF SWLSLLVPFV QWFVGLSPTV WLSVIWMMWY WAPNLHNILS PFLPLLPIFL CLWVYI (SEQ ID NO:57; GenBank Accession No: AHC69850. Such a heterologous glycoprotein may be useful in directing an EDV of the present disclosure to a liver cell.

In some cases, the viral envelope protein is a Hepatitis B Virus (HBV) pre S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MGGWSSKPRK GMGTNLAVPN PLGFFPDHQL DPAFKANSDN PDWDLNTHKD YWPDAWKVGV GAFGPGFTPP HGGLLGWSPQ AQGLLTTVPA APPPASTNRQ SGRQPTPLSP PLRDTHPQAM KWNSTTFHQT LQDPRVRALY LPAGGSSSGT VSPAQNTVSA ISSILSKTGD PVPNMESIAS GLLGPLLVLQ AGFFLLTKIL TIPQSLDSWW TSLNFLGGTP VCLGQNSQSQ ISSHSPTCCP PTCPGYRWMC LRRFIIFLCI LLLCLIFLLV LLDYQGMLPV CPLIPGSSTT STGPCKTCTA PAQGTSMFPS CCCTKPTDGN CTCIPIPSSW AFAKYLWEWA SVRFSWLSLL VPFVQWFVGL SPTVWLSVIW MMWFWGPSLY NILSPFIPLL PIFFCLWVYI (SEQ ID NO:58; GenBank Accession No: CAA66700). Such a heterologous glycoprotein may be useful in directing an EDV of the present disclosure to a liver cell.

In some cases, the viral envelope protein is a Hepatitis B Virus (HBV) preS2 glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MQWNSTTFHQ TLQDPRVRGL YFPAGGSSSG TVNPVPTTVS HISSIFSRIG DPALNMENIT SGFLGPLLVL QAGFFLLTRI LTIPQSLDSW WTSLNFLGGT TVCLGQNSQS PTSNHSPTSC PPTCPGYRWM CLRRFIIFLF ILLLCLIFLL VLLDYQGMLS VCPLIPGSTT TSTGPCKTCTTPAQGTSIHP SCCCTKPSDG NCTWIPIPSS WAFGKFLWEW ASARFSWLSL LVPFVQWFVG LSPTVWLSVI WIMWYWGPSL YSILSPFLPL LPIFFCLWVY I (SEQ ID NO:59; GenBank Accession No: AA012662). Such a heterologous glycoprotein may be useful in directing an EDV of the present disclosure to a liver cell.

In some cases, the viral envelope protein is a Rabies virus. A suitable Rabies virus protein comprises an amino acid sequence having at least 80%, at least 85%, at least 90%, at least 95%, at least 98%, at least 99%, or 100%, amino acid sequence identity to the following amino acid sequence: MVPQALLFVP LLVFPLCFGK FPIYTIPDKL GPWSPIDIHH LSCPNNLVVE DEGCTNLSGF SYMELKVGYI SAIKVNGFTC TGVVTEAETY TNFVGYVTTT FKRKHFRPTP DACRSAYNWK MAGDPRYEES LHNPYPDYHW LRTVKTTKES LVIISPSVAD LDPYDKSLHS RVFPSGKCSG ITVSSTYCST NHDYTIWMPE NLRLGTSCDI FINSRGKRAS KGSQTCGFID ERGLYKSLKG ACKLKLCGVL GLRLMDGTWV AMQTSDETKW CPPDQLVNLH DFRSDEIEHL VVEELVKKRE ECLDALESIM TTKSVSFRRL SHLRKLVPGF GKAYTIFNKT LMEADAHYKS VRTWNEIIPS KGCLRVGGRC HPHVNGVFFN GIILGPEGHV LIPEMQSSLL QQHMELLESS VIPLMHPLAD PSTVFKEGDE AEDFVEVHLP DVHKQVSGVN LGLPNWGKYV LLSAGALIAL MLIIFLLTCC RRVNRPESTQ HSLGGKRRKV SITSQSGKII SSWESYKSGG ETRL (SEQ ID NO:60; GenBank Accession No: AWR88358). Such a glycoprotein may be useful for targeting an EDV of the present disclosure to neurons, astrocytes, oligodendrocyctes, glia, and other cells of the of the central nervous system.

In some cases, the viral envelope protein is a Mokola virus glycoprotein. A suitable Mokola virus protein comprises an amino acid sequence having at least 80%, at least 85%, at least 90%, at least 95%, at least 98%, at least 99%, or 100%, amino acid sequence identity to the following amino acid sequence: MNIPCFVVIL SLATTHSLGE FPLYTIPEKI EKWTPIDMIH LSCPNNLLSE EEGCNAESSF TYFELKSGYL AHQKVPGFTC TGVVNEAETY TNFVGYVTTT FKRKHFRPTV AACRDAYNWK VSGDPRYEES LHTPYPDSSW LRTVTTTKES LLIISPSIVE MDIYGRTLHS PMFPSGVCSN VYPSVPSCET NHDYTLWLPE DPSLSLVCDI FTSSNGKKAM NGSRICGFKD ERGFYRSLKG ACKLTLCGRP GIRLFDGTWV SFTKPDVHVW CTPNQLINIH NDRLDEIEHL IVEDIIKKRE ECLDTLETIL MSQSVSFRRL SHFRKLVPGY GKAYTILNGS LMETNVYYKR VDKWADILPS KGCLKVGQQC MEPVKGVLFN GIIKGPDGQI LIPEMQSEQL KQHMDLLKAA VFPLRHPLIS REAVFKKDGD ADDFVDLHMP DVHKSVSDVD LGLPHWGFWM LIGATIVAFV VLVCLLRVCC KRVRRRRSGR ATQEIPLSFP SAPVPRAKVV SSWESYKGLP GT (SEQ ID NO:61; GenBank Accession No: AAB26292). Such a glycoprotein may be useful for targeting an EDV of the present disclosure to neurons, astrocytes, oligodendrocyctes, glia, and other cells of the of the central nervous system.

In some cases, the viral envelope protein is a lymphocytic choriomeningitis virus (LCMV) glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIIIT SIKAVYNFAT CGILALISFL LLAGRSCGLY GLDGPDIYKG IYQFKSVEFD MSHLNLTMPN ACSANNSHHY ISMGNSGLEL TFTNDSIISH NFCNLTSAFN KKTFDHTLMS IVSSLHLSIR GNSNYKAVSC DFNSGITIQY NLSFSDAQSA LSQCKTFRGR VLDMFRTAFG GKYMRSGWGW TGSDGKTTWC SQTSYQYLII QNRTWENHCR YAGPFGMARI LFAQEKTKFL TRRLAGTFTW TLSDSSGVDN PGGYCLTRWM ILAADLKCFG NTAVAKCNMN HDEEFCDMLR LIDYNKAALS KFKEDVESAL HLFKVTVNSL VSDQLLMRNH LRDLMGVPYC NYSRFWYLEH TKTGETSVPK CWLVTNGSYL NETHFSDQIE QEADNMITDM LRKDYIKRQG STPLALMDLL MFSTSAYLVS VFLHLVKIPT HRHIKGGSCP KPHRLTNKGI CSCGAFKVPG VKTVWKRR (SEQ ID NO:62; GenBank Accession No: AIW66623).

In some cases, the viral envelope protein is a lymphocytic choriomeningitis virus (LCMV) glycoprotein C. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIIIT SIKAVYNFAT CGILALVSFL FLAGRSCGMY GLNGPDIYKG VYQFKSVEFD MSHLNLTMPN ACSANNSHHY ISMGSSGLEL TFTNDSILNH NFCNLTSAFN KKTFDHTLMS IVSSLHLSIR GNSNHKAVSC DFNNGITIQY NLSFSDPQSA ISQCRTFRGR VLDMFRTAFG GKYMRSGWGW AGSDGKTTWC SQTSYQYLII QNRTWENHCR YAGPFGMSRI LFAQEKTKFL TRRLAGTFTW TLSDSSGVEN PGGYCLTKWM ILAAELKCFG NTAVAKCNVN HDEEFCDMLR LIDYNKAALS KFKQDVESAL HVFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLIS IFLHLVKIPT HRHIKGGSCP KPHRLTNKGI CSCGAFKVPG VKTIWKRR (SEQ ID NO:63; GenBank Accession No: CAC01231).

In some cases, the viral envelope protein is a lymphocytic choriomeningitis virus (LCMV) glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIVIT GIKAVYNFAT CGIFALISFL LLAGRSCGMY GLKGPDIYKG VYQFKSVEFD MSHLNLTMPN ACSANNSHHY ISMGTSGLEL TFTNDSIISH NFCNLTSAFN KKTFDHTLMS IVSSLHLSIR GNSNYKAVSC DFNNGITIQY NLTFSDAQSA QSQCRTFRGR VLDMFRTAFG GKYMRSGWGW TGSDGKTTWC SQTSYQYLII QNRTWENHCT YAGPFGMSRI LLSQEKTKFF TRRLAGTFTW TLSDSSGVEN PGGYCLTKWM ILAAELKCFG NTAVAKCNVN HDAEFCDMLR LIDYNKAALS KFKEDVESAL HLFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLVS IFLHLVKIPT HRHIKGGSCP KPHRLTNKGI CSCGAFKVPG VKTVWKRR (SEQ ID NO:64; GenBank Accession No: P09991).

In some cases, the viral envelope protein is a lymphocytic choriomeningitis virus (LCMV) Gi glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MYGLKGPDIYKG VYQFKSVEFD MSHLNLTMPN ACSANNSHHY ISMGTSGLEL TFTNDSIISH NFCNLTSAFN KKTFDHTLMS IVSSLHLSIR GNSNYKAVSC DFNNGITIQY NLTFSDAQSA QSQCRTFRGR VLDMFRTAFG GKYMRSGWGW TGSDGKTTWC SQTSYQYLII QNRTWENHCT YAGPFGMSRI LLSQEKTKFF TRRLA (SEQ ID NO:65; GenBank Accession No: P09991).

In some cases, the viral envelope protein is a lymphocytic choriomeningitis virus (LCMV) G2 glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: GTFTW TLSDSSGVEN PGGYCLTKWM ILAAELKCFG NTAVAKCNVN HDAEFCDMLR LIDYNKAALS KFKEDVESAL HLFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLVS IFLHLVKIPT HRHIKGGSCP KPHRLTNKGI CSCGAFKVPG VKTVWKRR (SEQ ID NO:66; GenBank Accession No: P09991).

In some cases, the viral envelope protein is a Ross River virus El glycoprotein. A suitable Ross River virus protein comprises an amino acid sequence having at least 80%, at least 85%, at least 90%, at least 95%, at least 98%, at least 99%, or 100%, amino acid sequence identity to the following amino acid sequence: YEHTATIPNV VGFPYKAHIE RNXFSPMTLQ LEVVXXSLEP TLNLEYITCE YKTVVPSPFI KCCGTSECSS KEQPDYQCKV YTGVYPFMWG GAYCFCDSEN TQLSEAYVDR SDVCKHDHAL AYKAHTASLK ATIRISYGTI NQTTEAFVNG EHAVNVGGSK FIFGPISTAW SPFDNKIVVY KDDVYNQDFP PYGSGQPGRF GDIQSRTVES KDLYANTALK LSRPSPGVVH VPYTQTPSGF KYWLKEKGSS LNTKAPFGCK IKTNPVRAMD CAVGSIPVSM DIPDSAFTRV VDAPAVTDLS CQVAVCTHSS DFGXVATLSY KTDKPGKCAV HSHSNVATLQ EATVDVKEDG KVTVHFSXXS ASPAFKVSVC DAKTTCTAAC EPPKDHIVPY GASHNNQVFP DMSGTAMTWV QRMASGLGGL ALIAVVVLVL VTCITMRR (SEQ ID NO:67; GenBank Accession No: NP_740686). Such a glycoprotein may be useful for targeting an EDV of the present disclosure to skeletal muscle, and cells that make up the joints, joint-associated connective tissue, bone, neurons, and lymphatic cells.

In some cases, the viral envelope protein is a Ross River virus E2 glycoprotein. A suitable Ross River virus protein comprises an amino acid sequence having at least 80%, at least 85%, at least 90%, at least 95%, at least 98%, at least 99%, or 100%, amino acid sequence identity to the following amino acid sequence: SVIEHFNVYK ATRPYLAXCA DCGDGYFCYS PVATEKIRDE ASDGMLKIQV SAQIGLDKAG THAHTKMRYM AGHDVQESKR DSLRVYTSAA CSIHGTMGHF IVAHCPPGDY LKXSFEDANS HVKACKVQYK HDPLPVGREK FVVRPHFGVE LPCTSYQLTT APTDEEIDMH TPPDIPDRTL LSQTAGNVKI TAGGRTIRYN CTCGRDNVGT TSTDKTINTC KIDQCHAAVT SHDKWXFTSP FVPRADQTAR KGKVHVPFPL TNVTCRVPLA RAPDVTYGKK EVTLRLHPDH PTXFSYRSLG AVPHPYEEWV DKFSERIIPV TEEGIEYQWG NNPPVRLWAQ LTTEGKPHGW PHEIIQYYYG LYPAATIAAV SGASLMALLT LAATCCMLAT ARRKCLTPYA LTPGAVVPLT LGLLXCAPRA NA (SEQ ID NO:68; GenBank Accession No: NP_740684). Such a glycoprotein may be useful for targeting an EDV of the present disclosure to skeletal muscle, and cells that make up the joints, joint-associated connective tissue, bone, neurons, and lymphatic cells.

In some cases, the viral envelope protein is a Semliki Forest virus El glycoprotein. A suitable Semliki Forest virus protein comprises an amino acid sequence having at least 80%, at least 85%, at least 90%, at least 95%, at least 98%, at least 99%, or 100%, amino acid sequence identity to the following amino acid sequence: YEHSTVMPNV VGFPYKAHIE RPGYSPLTLQ MQVVETSLEP TLNLEYITCE YKTVVPSPYV KCCGASECST KEKPDYQCKV YTGVYPFMWG GAYCFCDSEN TQLSEAYVDR SDVCRHDHAS AYKAHTASLK AKVRVMYGNV NQTVDVYVNG DHAVTIGGTQ FIFGPLSSAW TPFDNKIVVY KDEVFNQDFP PYGSGQPGRF GDIQSRTVES NDLYANTALK LARPSPGMVH VPYTQTPSGF KYWLKEKGTA LNTKAPFGCQ IKTNPVRAMN CAVGNIPVSM NLPDSAFTRI VEAPTIIDLT CTVATCTHSS DFGGVLTLTY KTNKNGDCSV HSHSNVATLQ EATAKVKTAG KVTLHFSTAS ASPSFVVSLC SARATCSASC EPPKDHIVPY AASHSNVVFP DMSGTALSWV QKISGGLGAF AIGAILVLVV VTCIGLRR (SEQ ID NO:69; GenBank Accession No: NP_819008). Such a glycoprotein may be useful for targeting an EDV of the present disclosure to muscle, pancreas, neurons, astrocytes, oligodendrocytes, glia, and other cells of the of the central nervous system.

In some cases, the viral envelope protein is a Semliki Forest virus E2 glycoprotein. A suitable Semliki Forest virus protein comprises an amino acid sequence having at least 80%, at least 85%, at least 90%, at least 95%, at least 98%, at least 99%, or 100%, amino acid sequence identity to the following amino acid sequence: SVSQHFNVYK ATRPYIAYCA DCGAGHSCHS PVAIEAVRSE ATDGMLKIQF SAQIGIDKSD NHDYTKIRYA DGHAIENAVR SSLKVATSGD CFVHGTMGHF ILAKCPPGEF LQVSIQDTRN AVRACRIQYH HDPQPVGREK FTIRPHYGKE IPCTTYQQTT AETVEEIDMH MPPDTPDRTL LSQQSGNVKI TVGGKKVKYN CTCGTGNVGT TNSDMTINTC LIEQCHVSVT DHKKWQFNSP FVPRADEPAR KGKVHIPFPL DNITCRVPMA REPTVIHGKR EVTLHLHPDH PTLFSYRTLG EDPQYHEEWV TAAVERTIPV PVDGMEYHWG NNDPVRLWSQ LTTEGKPHGW PHQIVQYYYG LYPAATVSAV VGMSLLALIS IFASCYMLVA ARSKCLTPYA LTPGAAVPWT LGILCCAPRA HA (SEQ ID NO:48; GenBank Accession No: NP_819006). Such a glycoprotein may be useful for targeting an EDV of the present disclosure to muscle, pancreas, neurons, astrocytes, oligodendrocyctes, glia, and other cells of the of the central nervous system.

In some cases, the viral envelope protein is a Sindbis virus El glycoprotein. A suitable Sindbis virus protein comprises an amino acid sequence having at least 80%, at least 85%, at least 90%, at least 95%, at least 98%, at least 99%, or 100%, amino acid sequence identity to the following amino acid sequence: YEHATTVPNV PQIPYKALVE RAGYAPLNLE ITVMSSEVLP STNQEYITCK FTTVVPSPKI KCCGSLECQP AAHADYTCKV FGGVYPFMWG GAQCFCDSEN SQMSEAYVEL SADCASDHAQ AIKVHTAAMK VGLRIVYGNT TSFLDVYVNG VTPGTSKDLK VIAGPISASF TPFDHKVVIH RGLVYNYDFP EYGAMKPGAF GDIQATSLTS KDLIASTDIR LLKPSAKNVH VPYTQASSGF EMWKNNSGRP LQETAPFGCK IAVNPLRAVD CSYGNIPISI DIPNAAFIRT SDAPLVSTVK CEVSECTYSA DFGGMATLQY VSDREGQCPV HSHSSTATLQ ESTVHVLEKG AVTVHFSTAS PQANFIVSLC GKKTTCNAEC KPPADHIVST PHKNDQEFQA AISKTSWSWL FALFGGASSL LIIGLMIFAC SMMLTSTRR (SEQ ID NO:70; GenBank Accession No: NP_740677). Such a glycoprotein may be useful for targeting an EDV of the present disclosure to muscle, pancreas, neurons, astrocytes, oligodendrocytes, glia, and other cells of the of the central nervous system.

In some cases, the viral envelope protein is a Sindbis virus E2 glycoprotein. A suitable Sindbis virus protein comprises an amino acid sequence having at least 80%, at least 85%, at least 90%, at least 95%, at least 98%, at least 99%, or 100%, amino acid sequence identity to the following amino acid sequence: SVIDDFTLTS PYLGTCSYCH HTVPCFSPVK IEQVWDEADD NTIRIQTSAQ FGYDQSGAAS ANKYRYMSLK QDHTVKEG™ DDIKISTSGP CRRLSYKGYF LLAKCPPGDS VTVSIVSSNS ATSCTLARKI KPKFVGREKY DLPPVHGKKI PCTVYDRLKE TTAGYITMHR PRPHAYTSYL EESSGKVYAK PPSGKNITYE CKCGDYKTGT VSTRTEITGC TAIKQCVAYK SDQTKWVFNS PDLIRHDDHT AQGKLHLPFK LIPSTCMVPV AHAPNVIHGF KHISLQLDTD HLTLLTTRRL GANPEPTTEW IVGKTVRNFT VDRDGLEYIW GNHEPVRVYA QESAPGDPHG WPHEIVQHYY HRHPVYTILA VASATVAMMI GVTVAVLCAC KARRECLTPY ALAPNAVIPT SLALLCCVRS ANA (SEQ ID NO:71; GenBank Accession No: NP_740675). Such a glycoprotein may be useful for targeting an EDV of the present disclosure to skeletal muscle, and cells that make up the joints, joint-associated connective tissue, bone, neurons, and lymphatic cells.

In some cases, the viral envelope protein is an Ebola Zaire virus glycoprotein. A suitable Ebola Zaire virus protein comprises an amino acid sequence having at least 80%, at least 85%, at least 90%, at least 95%, at least 98%, at least 99%, or 100%, amino acid sequence identity to the following amino acid sequence: MGVTGILQLP RDRFKRTSFF LWVIILFQRT FSIPLGVIHN STLQVSDVDK LVCRDKLSST NQLRSVGLNL EGNGVATDVP SATKRWGFRS GVPPKVVNYE AGEWAENCYN LEIKKPDGSE CLPAAPDGIR GFPRCRYVHK VSGTGPCAGD FAFHKEGAFF LYDRLASTVI YRGTTFAEGV VAFLILPQAK KDFFSSHPLR EPVNATEDPS SGYYSTTIRY QATGFGTNET EYLFEVDNLT YVQLESRFTP QFLLQLNETI YTSGKRSNTT GKLIWKVNPE IDTTIGEWAF WETKKNLTRK IRSEELSFTV VSNGAKNISG QSPARTSSDP GTNTTTEDHK IMASENSSAM VQVHSQGREA AVSHLTTLAT ISTSPQSLTT KPGPDNSTHN TPVYKLDISE ATQVEQHHRR TDNDSTASDT PSATTAAGPP KAENTNTSKS TDFLDPATTT SPQNHSETAG NNNTHHQDTG EESASSGKLG LITNTIAGVA GLITGGRRTR REAIVNAQPK CNPNLHYWTT QDEGAAIGLA WIPYFGPAAE GIYIEGLMHN QDGLICGLRQ LANETTQALQ LFLRATTELR TFSILNRKAI DFLLQRWGGT CHILGPDCCI EPHDWTKNIT DKIDQIIHDF VDKTLPDQGD NDNWWTGWRQ WIPAGIGVTG VIIAVIALFC ICKFVF (SEQ ID NO:72; GenBank Accession No: AAB81004). Such a glycoprotein may be useful for targeting an EDV of the present disclosure to hepatocytes, endothelial cells, dendritic cells, macrophages, and monocytes.

In some cases, the viral envelope protein is an Ebola Zaire virus glycoprotein. A suitable Ebola Zaire virus protein comprises an amino acid sequence having at least 80%, at least 85%, at least 90%, at least 95%, at least 98%, at least 99%, or 100%, amino acid sequence identity to the following amino acid sequence: IPLGVIHN STLQVSDVDK LVCRDKLSST NQLRSVGLNL EGNGVATDVP SATKRWGFRS GVPPKVVNYE AGEWAENCYN LEIKKPDGSECLPAAPDGIR GFPRCRYVHK VSGTGPCAGD FAFHKEGAFF LYDRLASTVI YRGTTFAEGV VAFLILPQAK KDFFSSHPLR EPVNATEDPS SGYYSTTIRY QATGFGTNET EYLFEVDNLT YVQLESRFTP QFLLQLNETI YTSGKRSNTT GKLIWKVNPE IDTTIGEWAF WETKKNLTRK IRSEELSFTV VSNGAKNISG QSPARTSSDP GTNTTTEDHK IMASENSSAM VQVHSQGREA AVSHLTTLAT ISTSPQSLTT KPGPDNSTHN TPVYKLDISE ATQVEQHHRR TDNDSTASDT PSATTAAGPP KAENTNTSKS TDFLDPATTT SPQNHSETAG NNNTHHQDTG EESASSGKLG LITNTIAGVA GLITGGRRTR REAIVNAQPK CNPNLHYWTT QDEGAAIGLA WIPYFGPAAE GIYIEGLMHN QDGLICGLRQ LANETTQALQ LFLRATTELR TFSILNRKAI DFLLQRWGGT CHILGPDCCI EPHDWTKNIT DKIDQIIHDF VDKTLPDQGD NDNWWTGWRQ WIPAGIGVTG VIIAVIALFC ICKFVF (SEQ ID NO:73). Such a glycoprotein may be useful for targeting an EDV of the present disclosure to hepatocytes, endothelial cells, dendritic cells, macrophages, and monocytes.

In some cases, the viral envelope protein is an Ebola Reston virus glycoprotein. A suitable Ebola Reston virus protein comprises an amino acid sequence having at least 80%, at least 85%, at least 90%, at least 95%, at least 98%, at least 99%, or 100%, amino acid sequence identity to the following amino acid sequence: MGSGYQLLQL PRERFRKTSF LVWVIILFQR AISMPLGIVT NSTLKATEID QLVCRDKLSS TSQLKSVGLN LEGNGIATDV PSATKRWGFR SGVPPKVVSY EAGEWAENCY NLEIKKSDGS ECLPLPPDGV RGFPRCRYVH KVQGTGPCPG DLAFHKNGAF FLYDRLASTV IYRGTTFAEG VVAFLILSEP KKHFWKATPA HEPVNTTDDS TSYYMTLTLS YEMSNFGGNE SNTLFKVDNH TYVQLDRPHT PQFLVQLNET LRRNNRLSNS TGRLTWTLDP KIEPDVGEWA FWETKKNFSQ QLHGENLHFQ IPSTHTNNSS DQSPAGTVQG KISYHPPANN SELVPTDSPP VVSVLTAGRT EEMSTQGLTN GETITGFTAN PMTTTIAPSP TMTSEVDNNV PSEQPNNTAS IEDSPPSASN ETIYHSEMDP IQGSNNSAQS PQTKTTPAPT TSPMTQDPQE TANSSKPGTS PGSAAGPSQP GLTINTVSKV ADSLSPTRKQ KRSVRQNTAN KCNPDLYYWT AVDEGAAVGL AWIPYFGPAA EGIYIEGVMH NQNGLICGLR QLANETTQAL QLFLRATTEL RTYSLLNRKA IDFLLQRWGG TCRILGPSCC IEPHDWTKNI TDEINQIKHD FIDNPLPDHG DDLNLWTGWR QWIPAGIGII GVIIAIIALL CICKILC (SEQ ID NO:74; GenBank Accession No: NP_690583). Such a glycoprotein may be useful for targeting an EDV of the present disclosure to hepatocytes, endothelial cells, dendritic cells, macrophages, and monocytes.

In some cases, the viral envelope protein is a Marburg virus glycoprotein. A suitable Marburg virus protein comprises an amino acid sequence having at least 80%, at least 85%, at least 90%, at least 95%, at least 98%, at least 99%, or 100%, amino acid sequence identity to the following amino acid sequence: MKTTCFLISL ILIQGTKNLP ILEIASNNQP QNVDSVCSGT LQKTEDVHLM GFTLSGQKVA DSPLEASKRW AFRTGVPPKN VEYTEGEEAK TCYNISVTDP SGKSLLLDPP TNIRDYPKCK TIHHIQGQNP HAQGIALHLW GAFFLYDRIA STTMYRGKVF TEGNIAAMIV NKTVHKMIFS RQGQGYRHMN LTSTNKYWTS SNGTQTNDTG CFGALQEYNS TKNQTCAPSK IPPPLPTARP EIKLTSTPTD ATKLNTTDPS SDDEDLATSG SGSGEREPHT TSDAVTKQGL SSTMPPTPSP QPSTPQQGGN NTNHSQDAVT ELDKNNTTAQ PSMPPHNTTT ISTNNTSKHN FSTLSAPLQN TTNDNTQSTI TENEQTSAPS ITTLPPTGNP TTAKSTSSKK GPATTAPNTT NEHFTSPPPT PSSTAQHLVY FRRKRSILWR EGDMFPFLDG LINAPIDFDP VPNTKTIFDE SSSSGASAEE DQHASPNISL TLSYFPNINE NTAYSGENEN DCDAELRIWS VQEDDLAAGL SWIPFFGPGI EGLYTAVLIK NQNNLVCRLR RLANQTAKSL ELLLRVTTEE RTFSLINRHA IDFLLTRWGG TCKVLGPDCC IGIEDLSKNI SEQIDQIKKD EQKEGTGWGL GGKWWTSDWG VLTNLGILLL LSIAVLIALS CICRIFTKYI G (SEQ ID NO:75); GenBank Accession No: CAA78117). Such a glycoprotein may be useful for targeting an EDV of the present disclosure to hepatocytes, endothelial cells, dendritic cells, macrophages, and monocytes.

In some cases, the viral envelope protein is a murine leukemia virus (MLV) glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MESTTLSKPF KNQVNPWGPL IVLLILGGVN PVALGNSPHQ VFNLTWEVTN GDRETVWAIA GNHPLWTWWP DLTPDLCMLA LHGPSYWGLE YRAPFSPPPG PPCCSGSSDS TPGCSRDCEE PLTSYTPRCN TAWNRLKLSK VTHAHNEGFY VCPGPHRPRW ARSCGGPESF YCASWGCETT GRASWKPSSS WDYITVSNNL TSDQATPVCK GNEWCNSLTI RFTSFGKQAT SWVTGHWWGL RLYVSGHDPG LIFGIRLKIT DSGPRVPIGP NPVLSDRRPP SRPRPTRSPP PSNSTPTETP LTLPEPPPAG VENRLLNLVK GAYQALNLTS PDKTQECWLC LVSGPPYYEG VAVLGTYSNH TSAPANCSVA SQHKLTLSEV TGQGLCIGAV PKTHQVLCNT TQKTSDGSYY LAAPTGTTWA CSTGLTPCIS TTILDLTTDY CVLVELWPRV TYHSPSYVYH QFEGRAKYKR EPVSLTLALL LGGLTMGGIA AGVGTGTTAL VATQQFQQLQ AAMHDDLKEV EKSITNLEKS LTSLSEVVLQ NRRGLDLLFL KEGGLCAALK EECCFYADHT GLVRDSMAKL RERLSQRQKL FESQQGWFEG LFNKSPWFTT LISTIMGPLI ILLLILLFGP CILNRLVQFI KDRISVVQAL VLTQQYHQLK TIRDCKSRE (SEQ ID NO:76; GenBank Accession No: AAA51037).

In some cases, the viral envelope protein is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MESTTLSKPF KNQVNPWGPL IVLLILRGVN PVTLGNSPHQ VFNLTWEVTN GDRETVWAIT GNHPLWTWWP DLTPDLCMLA LHGPSYWGLE YRAPFSPPPG PPCCSGSSDS TPGCSRDCEE PLTSYTPRCN TAWNRLKLSK VTHAHNGGFY VCPGPHRPRW ARSCGGPESF YCASWGCETT GRASWKPSSS WDYITVSNNL TSDQATPVCK GNKWCNSLTI RFTSFGKQAT SWVTGHWWGL RLYVSGHDPG LIFGIRLKIT DSGPRVPIGP NPVLSDRRPP SRPRPTRSPP PSNSTPTETP LTLPEPPPAG VENRLLNLVK GAYQALNLTS PDKTQECWLC LVSGPPYYEG VAVLGTYSNH TSAPANCSVA SQHKLTLSEV TGQGLCIGAV PKTHQVLCNT TQKTSDGSYY LAAPTGTTWA CSTGLTPCIS TTILDLTTDY CVLVELWPRV TYHSPSYVYH QFERRAKYKR EPVSLTLALL LGGLTMGGIA AGVGTGTTAL VATQQFQQLQ AAMHDDLKEV EKSITNLEKS LTSLSEVVLQ NRRGLDLLFL KEGGLCAALK EECCFYADHT GLVRDSMAKL RERLSQRQKL FESQQGWFEG LFNKSPWFTT LISTIMGPLI 1LLLILLFGP CILNRLVQFI KDRISVVQAL VLTQQYHQLK IIEDCKSRE (SEQ ID NO:77; GenBank Accession No: AID54959).

In some cases, the viral envelope protein is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence:

(SEQ ID NO: 79; GenBank Accession No: AAA46514)
MEGPAFSKPL KDKINPWKSL MVMGVYLRVG MAESPHQVEN VTWRVTNLMT GRTANATSLL
GTVQDAFPRL YFDLCDLVGE EWDPSDQEPY VGYGCKYPGG RKRTRTFDFY VCPGHTVKSG
CGGPREGYCG EWGCETTGQA YWKPTSSWDL ISLKRGNTPW DTGCSKMACG PCYDLSKVSN
SFQGATRGGR CNPLVLEFTD AGKKANWDGP KSWGLRLYRT GTDPITMFSL TRQVLNIGPR
IPIGPNPVIT GQLPPSRPVQ IRLPRPPQPP PTGAASIVPE TAPPSQQPGT GDRLLNLVEG
AYQALNLTNP DKTQECWLCL VSGPPYYEGV AVVGTYTNHS TAPASCTATS QHKLTLSEVT
GQGLCMGALP KTHQALCNTT QSAGSGSYYL AAPAGTMWAC STGLTPCLST TMLNLTTDYC
VLVELWPRII YHSPDYMYGQ LEQRTKYKRE PVSLTLALLL GGLTMGGIAA GIGTGTTALI
KTQQFEQLHA AIQTDLNEVE KSITNLEKSL TSLSEVVLQN RRGLDLLFLK EGGLCAALKE
ECCFYADHTG LVRDSMAKLR ERLNQRQKLF ESGQGWFEGQ FNRSPWFTTL ISTIMGPLIV
LLLILLFGPC ILNRLVQFVK DRISVVQALV LTQQYHQLKP IEYEP.

In some cases, the viral envelope protein is an MLV glycoprotein. A suitable MLV

(SEQ ID NO: 78; GenBank Accession No: AAA46515)
MARSTLSKPP QDKINPWKPL IVMGVLLGVG MAESPHQVEN VTWRVTNLMT GRTANATSLL
GTVQDAFPKL YFDLCDLVGE EWDPSDQEPY VGYGCKYPAG RQRTRTFDFY VCPGHTVKSG
CGGPGEGYCG KWGCETTGQA YWKPTSSWDL ISLKRGNTPW DTGCSKVACG PCYDLSKVSN
SFQGATRGGR CNPLVLEFTD AGKKANWDGP KSWGLRLYRT GTDPITMFSL TRQVLNVGPR
VPIGPNPVLP DQRLPSSPIE IVPAPQPPSP LNTSYPPSTT STPSTSPTSP SVPQPPPGTG
DRLLALVKGA YQALNLTNPD KTQECWLCLV SGPPYYEGVA VVGTYTNHST APANCTATSQ
HKLTLSEVTG QGLCMGAVPK THQALCNTTQ SAGSGSYYLA APAGTMWACS TGLTPCLSTT
VLNLTTDYCV LVELWPRVIY HSPDYMYGQL EQRTKYKREP VSLTLALLLG GLTMGGIAAG
IGTGTTALIK TQQFEQLHAA IQTDLNEVEK SITNLEKSLT SLSEVVLQNR RGLDLLFLKE
GGLCAALKEE CCFYADHTGL VRDSMAKLRE RLNQRQKLFE TGQGWFEGLF NRSPWFTTLI
STIMGPLIVL LLILLFGPCI LNRLVQFVKD RISVVQALVL TQQYHQLKPI EYEP.

protein comprises an amino acid sequence having at least 80%, at least 85%, at least 90%, at least 95%, at least 98%, at least 99%, or 100%, amino acid sequence identity to the following amino acid sequence: MEGPAFSKPL KDKINPWKSL MVMGVYLRVG MAESPHQVFN VTWRVTNLMT GRTANATSLL GTVQDAFPRL YFDLCDLVGE EWDPSDQEPY VGYGCKYPGG RKRTRTFDFY VCPGHTVKSG CGGPREGYCG EWGCETTGQA YWKPTSSWDL ISLKRGNTPW DTGCSKMACG PCYDLSKVSN SFQGATRGGR CNPLVLEFTD AGKKANWDGP KSWGLRLYRT GTDPITMFSL TRQVLNIGPR IPIGPNPVIT GQLPPSRPVQ IRLPRPPQPP PTGAASIVPE TAPPSQQPGT GDRLLNLVEG

AYQALNLTNP DKTQECWLCL VSGPPYYEGV AVVGTYTNHS TAPASCTATS QHKLTLSEVT GQGLCMGALP KTHQALCNTT QSAGSGSYYL AAPAGTMWAC STGLTPCLST TMLNLTTDYC VLVELWPRII YHSPDYMYGQ LEQRTKYKRE PVSLTLALLL GGLTMGGIAA GIGTGTTALI KTQQFEQLHA AIQTDLNEVE KSITNLEKSL TSLSEVVLQN RRGLDLLFLK EGGLCAALKE ECCFYADHTG LVRDSMAKLR ERLNQRQKLF ESGQGWFEGQ FNRSPWFTTL ISTIMGPLIV LLLILLFGPC ILNRLVQFVK DRISVVQALV LTQQYHQLKP IEYEP (SEQ ID NO:79; GenBank Accession No: AAA46514).

In some cases, the viral envelope protein is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence:

(SEQ ID NO: 80; GenBank Accession No: AAA46531)
MEGSAFSKPL KDKINPWGPL IVMGILVRAG ASVQRDSPHQ IFNVTWRVTN LMTGQTANAT
SLLGTMTDTF PKLYFDLCDL VGDYWDDPEP DIGDGCRTPG GRRRTRLYDF YVCPGHTVPI
GCGGPGEGYC GKWGCETTGQ AYWKPSSSWD LISLKRGNTP KDQGPCYDSS VSSGVQGATP
GGRCNPLVLE FTDAGRKASW DAPKVWGLRL YRSTGADPVT RFSLTRQVLN VGPRVPIGPN
PVITDQLPPS QPVQIMLPRP PHPPPSGTVS MVPGAPPPSQ QPGTGDRLLN LVEGAYQALN
LTSPDKTQEC WLCLVSGPPY YEGVAVLGTY SNHTSAPANC SVASQHKLTL SEVTGQGLCV
GAVPKTHQAL CNTTQKTSDG SYYLAAPAGT IWACNTGLTP CLSTTVLNLT TDYCVLVELW
PKVTYHSPDY VYGQFEKKTK YKREPVSLTL ALLLGGLTMG GIAAGVGTGT TALVATKQFE
QLQAAIHTDL GALEKSVSAL EKSLTSLSEV VLQNRRGLDL LFLKEGGLCA ALKEECCFYA
DHTGVVRDSM AKLRERLNQR QKLFESGQGW FEGLFNRSPW FTTLISTIMG PLIVLLLILL
LGPCILNRLV QFVKDRISVV QALILTQQYH QLKSIEPEEV ESRE.

In some cases, the viral envelope protein is a polytropic mink cell focus-forming virus glycoprotein. A suitable polytropic mink cell focus-forming virus protein comprises an amino acid sequence having at least 80%, at least 85%, at least 90%, at least 95%, at least 98%, at least 99%, or 100%, amino acid sequence identity to the following amino acid sequence: VQHDSPHQVF NVTWRVTNLM TGQTANATSL LGTMTDAFPK LYFDLCDLIG DDWDETGLGC RTPGGRKRAR TFDFYVCPGH TVPTGCGGPR EGYCGKWGCE TTGQAYWKPS SLWDLISLKR GNTPQNQGPC YDSSAVSSDI KGATPGGRCN PLVLEFTDAG KKASWDGPKV WGLRLYRSTG TDPVTRFSLT RRVLNIGPRV PIGPNPVIID QLPPSRPVQI MLPRPPQPPP PGAASIVPET APPSNQPGTG DRLLNLVDGA YQALNLTSPD KTQECWLCLV AEPPYYEGVA VLGTYSNHTS APANCSVASQ HKLTLSEVTG RGLCIGTVPK THQALCNTTL KTNKGSYYLV APAGTTWACN TGLTPCLSAT VLNRTTDYCV LVELWPRVTY HPPSYVYSQF EKSYRHKR (SEQ ID NO:81; GenBank Accession No: 2016415A).

In some cases, the viral envelope protein is a gibbon ape leukemia virus (GALV) glycoprotein. A suitable GALV protein comprises an amino acid sequence having at least 80%, at least 85%, at least 90%, at least 95%, at least 98%, at least 99%, or 100%, amino acid sequence identity to the following amino acid sequence: MVLLPGSMLL TSNLHHLRHQ MSPGSWKRLI ILLSCVFGGG GTSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKRE AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL VLRQKYQALE NEGNL (SEQ ID NO:82; GenBank Accession No: P21415).

In some cases, the viral envelope protein is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence:

(SEQ ID NO: 83)
TSLQNKNPH QPMTLTWQVL SQTGDVVWDT KAVQPPWTWW PTLKPDVCAL AASLESWDIP
GTDVSSSKRV RPPDSDYTAA YKQITWGAIG CSYPRARTRM ASSTFYVCPR DGRTLSEARR
CGGLESLYCK EWDCETTGTG YWLSKSSKDL ITVKWDQNSE WTQKFQQCHQ TGWCNPLKID
FTDKGKLSKD WITGKTWGLR FYVSGHPGVQ FTIRLKITNM PAVAVGPDLV LVEQGPPRTS
LALPPPLPPR EAPPPSLPDS NSTALATSAQ TPTVRKTIVT LNTPPPTTGD RLFDLVQGAF
LTLNATNPGA TESCWLCLAM GPPYYEAIAS SGEVAYSTDL DRCRWGTQGK LTLTEVSGHG
LCIGKVPFTH QHLCNQTLSI NSSGDHQYLL PSNHSWWACS TGLTPCLSTS VFNQTRDFCI
QVQLIPRIYY YPEEVLLQAY DNSHPRTKRE AVSLTLAVLL GLGITAGIGT GSTALIKGPI
DLQQGLTSLQ IAIDADLRAL QDSVSKLEDS LTSLSEVVLQ NRRGLDLLFL KEGGLCAALK
EECCFYIDHS GAVRDSMKKL KEKLDKRQLE RQKSQNWYEG WENNSPWFTT LLSTIAGPLL
LLLLLLILGP CIINKLVQFI NDRISAVKIL VLRQKYQALE NEGNL.

In some cases, the viral envelope protein is a GALV glycoprotein. A suitable GALV

(SEQ ID NO: 83)
TSLQNKNPH QPMTLTWQVL SQTGDVVWDT KAVQPPWTWW PTLKPDVCAL AASLESWDIP
GTDVSSSKRV RPPDSDYTAA YKQITWGAIG CSYPRARTRM ASSTFYVCPR DGRTLSEARR
CGGLESLYCK EWDCETTGTG YWLSKSSKDL ITVKWDQNSE WTQKFQQCHQ TGWCNPLKID
FTDKGKLSKD WITGKTWGLR FYVSGHPGVQ FTIRLKITNM PAVAVGPDLV LVEQGPPRTS
LALPPPLPPR EAPPPSLPDS NSTALATSAQ TPTVRKTIVT LNTPPPTTGD RLFDLVQGAF
LTLNATNPGA TESCWLCLAM GPPYYEAIAS SGEVAYSTDL DRCRWGTQGK LTLTEVSGHG
LCIGKVPFTH QHLCNQTLSI NSSGDHQYLL PSNHSWWACS TGLTPCLSTS VFNQTRDFCI
QVQLIPRIYY YPEEVLLQAY DNSHPRTKRE AVSLTLAVLL GLGITAGIGT GSTALIKGPI
DLQQGLTSLQ IAIDADLRAL QDSVSKLEDS LTSLSEVVLQ NRRGLDLLFL KEGGLCAALK
EECCFYIDHS GAVRDSMKKL KEKLDKRQLE RQKSQNWYEG WENNSPWFTT LLSTIAGPLL
LLLLLLILGP CIINKLVQFI NDRISAVKIL VLRQKYQALE NEGNL.

protein comprises an amino acid sequence having at least 80%, at least 85%, at least 90%, at least 95%, at least 98%, at least 99%, or 100%, amino acid sequence identity to the following amino acid sequence: TSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR 33

CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKRE AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL VLRQKYQALE NEGNL (SEQ ID NO:83).

In some cases, the viral envelope protein is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence: TSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKR (SEQ ID NO:84).

In some cases, the viral envelope protein is a GALV glycoprotein. A suitable GALV

(SEQ ID NO: 84)
TSLQNKNPH QPMTLTWQVL SQTGDVVWDT KAVQPPWTWW PTLKPDVCAL AASLESWDIP
GTDVSSSKRV RPPDSDYTAA YKQITWGAIG CSYPRARTRM ASSTFYVCPR DGRTLSEARR
CGGLESLYCK EWDCETTGTG YWLSKSSKDL ITVKWDQNSE WTQKFQQCHQ TGWCNPLKID
FTDKGKLSKD WITGKTWGLR FYVSGHPGVQ FTIRLKITNM PAVAVGPDLV LVEQGPPRTS
LALPPPLPPR EAPPPSLPDS NSTALATSAQ TPTVRKTIVT LNTPPPTTGD RLFDLVQGAF
LTLNATNPGA TESCWLCLAM GPPYYEAIAS SGEVAYSTDL DRCRWGTQGK LTLTEVSGHG
LCIGKVPFTH QHLCNQTLSI NSSGDHQYLL PSNHSWWACS TGLTPCLSTS VFNQTRDFCI
QVQLIPRIYY YPEEVLLQAY DNSHPRTKR.

protein comprises an amino acid sequence having at least 80%, at least 85%, at least 90%, at least 95%, at least 98%, at least 99%, or 100%, amino acid sequence identity to the following amino acid sequence:

(SEQ ID NO: 85)
E AVSLTLAVLL GLGITAGIGT GSTALIKGPI DLQQGLTSLQ IAIDADLRAL QDSVSKLEDS
LTSLSEVVLQ NRRGLDLLFL KEGGLCAALK EECCFYIDHS GAVRDSMKKL KEKLDKRQLE
RQKSQNWYEG WENNSPWFTT LLSTIAGPLL LLLLLLILGP CIINKLVQFI NDRISAVKIL.

In some cases, the viral envelope protein is a RD1 14 retrovirus glycoprotein. A suitable RD 114 retrovirus protein comprises an amino acid sequence having at least 80%, at least 85%, at least 90%, at least 95%, at least 98%, at least 99%, or 100%, amino acid sequence identity to the following amino acid sequence: MKLPTGMVIL CSLIIVRAGF DDPRKAIALV QKQHGKPCEC SGGQVSEAPP NSIQQVTCPG KTAYLMTNQK WKCRVTPKNL TPSGGELQNC PCNTFQDSMH SSCYTEYRQC RANNKTYYTA TLLKIRSGSL NEVQILQNPN QLLQSPCRGS INQPVCWSAT APIHISDGGG PLDTKRVWTV QKRLEQIHKA MHPELQYHPL ALPKVRDDLS LDARTFDILN TTFRLLQMSN FSLAQDCWLC LKLGTPTPLA IPTPSLTYSL ADSLANASCQ IIPPLLVQPM QFSNSSCLSS PFINDTEQID LGAVTFTNCT SVANVSSPLC ALNGSVFLCG NNMAYTYLPQ NWTGLCVQAS LLPDIDIIPG DEPVPIPAID HYIHRPKRAV QFIPLLAGLG ITAAFTTGAT GLGVSVTQYT KLSHQLISDV QVLSGTIQDL QDQVDSLAEV VLQNRRGLDL LTAEQGGICL ALQEKCCFYA NKSGIVRNKI RTLQEELQKR RESLASNPLW TGLQGFLPYL LPLLGPLLTL LLILTIGPCV FSRLMAFIND RLNVVHAMVL AQQYQALKAE EEAQD (SEQ ID NO:86; GenBank Accession No: YP_001497149).

In some cases, the viral envelope protein is a Sendai virus (SeV) glycoprotein. A suitable SeV protein comprises an amino acid sequence having at least 80%, at least 85%, at least 90%, at least 95%, at least 98%, at least 99%, or 100%, amino acid sequence identity to the following amino acid sequence: MTAYIQRSQC ISTSLLVVLT TLVSCQIPRD RLSNIGVIVD EGKSLKIAGS HESRYIVLSL VPGVDFENGC GTAQVIQYKS LLNRLLIPLR DALDLQEALI TVTNDTTQNA GAPQSRFFGA VIGTIALGVA TSAQITAGIA LAEAREAKRD IALIKESMTK THKSIELLQN AVGEQILALK TLQDFVNDEI KPAISELGCE TAALRLGIKL TQHYSELLTA FGSNFGTIGE KSLTLQALSS LYSANITEIM TTIKTGQSNI YDVIYTEQIK GTVIDVDLER YMVTLSVKIP ILSEVPGVLI HKASSISYNI DGEEWYVTVP SHILSRASFL GGADITDCVE SRLTYICPRD PAQLIPDSQQ KCILGDTTRC PVTKVVDSLI PKFAFVNGGV VANCIASTCT CGTGRRPISQ DRSKGVVFLT HDNCGLIGVN GVELYANRRG HDATWGVQNL TVGPAIAIRP IDISLNLADA TNFLQDSKAE LEKARKILSE VGRWYNSRET VITIIVVMVV ILVVIIVIII VLYRLRRSML MGNPDDRIPR DTYTLEPKIR HMYTNGGFDA MAKER (SEQ ID NO:87; GenBank Accession No: P04855).

In some cases, the viral envelope protein is an SeV FO glycoprotein. A suitable SeV protein comprises an amino acid sequence having at least 80%, at least 85%, at least 90%, at least 95%, at least 98%, at least 99%, or 100%, amino acid sequence identity to the following amino acid sequence:

(SEQ ID NO: 88; GenBank Accession No: P04855)
QIPRD RLSNIGVIVD EGKSLKIAGS HESRYIVLSL VPGVDFENGC GTAQVIQYKS
LLNRLLIPLR DALDLQEALI TVINDTTQNA GAPQSRFFGA VIGTIALGVA TSAQITAGIA
LAEAREAKRD IALIKESMTK THKSIELLQN AVGEQILALK TLQDFVNDEI KPAISELGCE
TAALRLGIKL TQHYSELLTA FGSNFGTIGE KSLTLQALSS LYSANITEIM TTIKTGQSNI
YDVIYTEQIK GTVIDVDLER YMVTLSVKIP ILSEVPGVLI HKASSISYNI DGEEWYVTVP
SHILSRASFL GGADITDCVE SRLTYICPRD PAQLIPDSQQ KCILGDTTRC PVTKVVDSLI
PKFAFVNGGV VANCIASTCT CGTGRRPISQ DRSKGVVFLT HDNCGLIGVN GVELYANRRG
HDATWGVQNL TVGPAIAIRP IDISLNLADA TNFLQDSKAE LEKARKILSE VGRWYNSRET
VITIIVVMVV ILVVIIVIII VLYRLRRSML MGNPDDRIPR DTYTLEPKIR HMYTNGGFDA
MAEKR.

In some cases, the viral envelope protein is an SeV F2 glycoprotein. A suitable SeV protein comprises an amino acid sequence having at least 80%, at least 85%, at least 90%, at least 95%, at least 98%, at least 99%, or 100%, amino acid sequence identity to the following amino acid sequence:

(SEQ ID NO: 89; GenBank Accession No: P04855)
QIPRD RLSNIGVIVD EGKSLKIAGS HESRYIVLSL VPGVDFENGC GTAQVIQYKS
LLNRLLIPLR DALDLQEALI TVINDTTQNA GAPQSR.

In some cases, the viral envelope protein is an SeV F1 glycoprotein. A suitable SeV protein comprises an amino acid sequence having at least 80%, at least 85%, at least 90%, at least 95%, at least 98%, at least 99%, or 100%, amino acid sequence identity to the following amino acid sequence:

(SEQ ID NO: 90; GenBank Accession No: P04855)
FFGA VIGTIALGVA TSAQITAGIA LAEAREAKRD IALIKESMTK THKSIELLQN AVGEQILALK
TLQDFVNDEI KPAISELGCE TAALRLGIKL TQHYSELLTA FGSNFGTIGE KSLTLQALSS
LYSANITEIM TTIKTGQSNI YDVIYTEQIK GTVIDVDLER YMVTLSVKIP ILSEVPGVLI
HKASSISYNI DGEEWYVTVP SHILSRASFL GGADITDCVE SRLTYICPRD PAQLIPDSQQ
KCILGDTTRC PVTKVVDSLI PKFAFVNGGV VANCIASTCT CGTGRRPISQ DRSKGVVFLT
HDNCGLIGVN GVELYANRRG HDATWGVQNL TVGPAIAIRP IDISLNLADA TNFLQDSKAE
LEKARKILSE VGRWYNSRET VITIIVVMVV ILVVIIVIII VLYRLRRSML MGNPDDRIPR
DTYTLEPKIR HMYTNGGFDA MAKER.

In some cases, the viral envelope protein is an SeV hemagglutinin-neuraminidase glycoprotein. A suitable SeV protein comprises an amino acid sequence having at least 80%, at least 85%, at least 90%, at least 95%, at least 98%, at least 99%, or 100%, amino acid sequence identity to the following amino acid sequence: MDGDRSKRDS YWSTSPGGST TKLVSDSERS GKVDTWLLIL AFTQWALSIA TVIICIVIAA RQGYSMERYS MTVEALNTSN KEVKESLTSL IRQEVITRAA NIQSSVQTGI PVLLNKNSRD VIRLIEKSCN RQELTQLCDS TIAVHHAEGI APLEPHSFWR CPAGEPYLSS DPEVSLLPGP SLLSGSTTIS GCVRLPSLSI GEAIYAYSSN LITQGCADIG KSYQVLQLGY ISLNSDMFPD LNPVVSHTYD INDNRKSCSV VATGTRGYQL CSMPIVDERT DYSSDGIEDL VLDILDLKGR TKSHRYSNSE IDLDHPFSAL YPSVGSGIAT EGSLIFLGYG GLTTPLQGDT KCRIQGCQQV SQDTCNEALK ITWLGGKQVV SVLIQVNDYL SERPRIRVTT IPITQNYLGA EGRLLKLGDQ VYIYTRSSGW HSQLQIGVLD VSHPLTISWT PHEALSRPGN EDCNWYNTCP KECISGVYTD AYPLSPDAAN VATVTLYANT SRVNPTIMYS NTTNIINMLR IKDVQLEAAY TTTSCITHFG KGYCFHIIEI NQKSLNTLQP MLFKTSIPKL CKAES (SEQ ID NO:91; GenBank Accession No: BAA24391).

In some cases, the viral envelope protein is a Jaagsiekte sheep retrovirus (JSRV) glycoprotein. A suitable JSRV protein comprises an amino acid sequence having at least 80%, at least 85%, at least 90%, at least 95%, at least 98%, at least 99%, or 100%, amino acid sequence identity to the following amino acid sequence: MPKRRAGFRK GWYARQRNSL THQMQRMTLS EPTSELPTQR QIEALMRYAW NEAHVQPPVT PTNILIMLLL LLQRIQNGAA ATFWAYIPDP PMLQSLGWDK ETVPVYVNDT SLLGGKSDIH ISPQQANISF YGLTTQYPMC FSYQSQHPHC IQVSADISYP RVTISGIDEK TGMRSYRDGT GPLDIPFCDK HLSIGIGIDT PWTLCRARIA SVYNINNANT TLLWDWAPGG TPDFPEYRGQ HPPISSVNTA PIYQTELWKL LAAFGHGNSL YLQPNISGSK YGDVGVTGFL YPRACVPYPF MVIQGHMEIT PSLNIYYLNC SNCILTNCIR GVAKGEQVII VKQPAFVMLP VEITEEWYDE TALELLQRIN TALSRPKRGL SLIILGIVSL ITLIATAVTA SVSLAQSIQV AHTVDSLSSN VTKVMGTQEN IDKKIEDRLP ALYDVVRVLG EQVQSINFRM KIQCHANYKW ICVTKKPYNT SDFPWDKVKK HLQGIWFNTT VSLDLLQLHN EILDIENSPK ATLNIADTVD NFLQNLFSNF PSLHSLWRSI IAMGAVLTFV LIIICLAPCL IRSIVKEFLH MRVLIHKNML QHQHLMELLN NKERGAAGDD P (SEQ ID NO:92; GenBank Accession No: ABI50237).

In some cases, the viral envelope protein is a baculovirus gp64 glycoprotein. A suitable baculovirus protein comprises an amino acid sequence having at least 80%, at least 85%, at least 90%, at least 95%, at least 98%, at least 99%, or 100%, amino acid sequence identity to the following amino acid sequence: MFHLLTLLLL LFINMNLYLA GEHCNVQMKN GPYRIKNLAI TPPRETLKKD VTVTIVETDY EENVLIGYKG YYQAYGYNGG SLDANTRLEE TMESLPLTKE DLLTWTYRQE CEVGEELIDR WGSDSDDCYR NKDGRGVWVK TKELVKRQNN NHFAHHTCNR SWRCGFSTAK MYSKLVCDDE TNDCKVFILD NTGKPINITT NEVLYRDGVN MMLKSKPTFT RREEKVACLL VKDELNPDKT REHCLIDSDI YDLSNNNWFC MFNKCIKRNV DSVVKKRPNK WMHNLAPKYS EGATATKGDM MHIQEELMYE NDLLKMNIEL VHAHMNKLNN IIHDLIVSIA KVDERLIGNL MNISVSSVFL SDDTFLLMPC TNPPQHTSNC YNNSIYREGR WVFNEDTSEC IDFNNYRELS IDDDIEFWIP TIGNTTYHDS WKDASGWSFV AQQKSNLIMT MENTKFGGVG TSLSDITSMS EGELTAKLTT FVFSHIVTFI LIIILIILCI CLLKK (SEQ ID NO:93; GenBank Accession No: YP_009182316).

In some cases, the viral envelope protein is a baculovirus gp64 glycoprotein. A suitable baculovirus protein comprises an amino acid sequence having at least 80%, at least 85%, at least 90%, at least 95%, at least 98%, at least 99%, or 100%, amino acid sequence identity to the following amino acid sequence: MLRITLLILF LVRFVSGAEH CNAQMKSGPW RIKNLPIAPP KETLQKDVDV EIVETDLDEN VIIGYKGYYQ AYAYNGGSLD PNTSVDETTQ TLNIDKDDLI TWGDRRKCEV GEELIDQWGS DSDSCFKDKL GRGVWVAGKE LVKRKNNNHF AHHTCNRSWR CGVSTAKMYT RLECDNETDD CKVTILDING TVINVTENEV LHRDGVSMIL KQKSTFTRRT EKVACLLIKD DKSDPYSITR EHCLIDNDIF DLSKNTWNCK FNRCIKRRSE NVVKKRPPTW RHNEPPKHSE GTTATKGDLM HIQEELMYEN DLLRMNLELL HAHINKLNNM MHDLIVSVAK VDERLIGNLM NNSVSSTFLS DDTFLLMPCT NPPPHTSNCY NNSIYKEGRW VANTDSSQCI DFRNYKELAI DDDIEFWIPT IGNTSYHESW KDASGWSFIA QQKSNLIS™ ENTKFGGHTT SLSDIGDMAK GELNATLYSF MLGHGFSFFL IIGVIVFLIC MVRSRVRAF (SEQ ID NO:94; GenBank Accession No: YP_473216).

In some cases, the viral envelope protein is a Chandipura virus glycoprotein. A suitable Chandipura virus protein comprises an amino acid sequence having at least 80%, at least 85%, at least 90%, at least 95%, at least 98%, at least 99%, or 100%, amino acid sequence identity to the following amino acid sequence: MTSSVTISVI LLISFIAPSY SSLSIAFPEN TKLDWKPVTK NTRYCPMGGE WFLEPGLQEE SFLSSTPIGA TPSKSDGFLC HAAKWVTTCD FRWYGPKYIT HSIHNIKPTR SDCDTALASY KSGTLVSPGF PPESCGYASV TDSEFLVIMI TPHHVGVDDY RGHWVDPLFV GGECDQSYCD TIHNSSVWIP ADQTKKNICG QSFTPLTVTV AYDKTKEIAA GAIVFKSKYH SHMEGARTCR LSYCGRNGIK FPNGEWVSLD VKTKIQEKPL LPLFKECPAG TEVRSTLQSD GAQVLTSEIQ RILDYSLCQN TWDKVERKEP LSPLDLSYLA SKSPGKGLAY TVINGTLSFA HTRYVRMWID GPVLKEMKGK RESPSGISSD IWTQWFKYGD MEIGPNGLLK TAGGYKFPWH LIGMGIVDNE LHELSEANPL DHPQLPHAQS IADDSEEIFF GDTGVSKNPV ELVTGWFTSW KESLAAGVVL ILVVVLIYGV LRCFPVLCTT CRKPKWKKGV ERSDSFEMRI FKPNNMRARV (SEQ ID NO:95; GenBank Accession No: YP_007641380).

In some cases, viral envelope protein is a Venezuelan equine encephalitis virus glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: MFPFQPMYPM QPMPYRNPFA APRRPWFPRT DPFLAMQVQE LTRSMANLTF KQRRDAPPEG PSAKKPKKEA SQKQKGGGQG KKKKNQGKKK AKTGPPNPKA QNGNKKKTNK KPGKRQRMVM KLESDKTFPI MLEGKINGYA CVVGGKLFRP MHVEGKIDND VLAALKTKKA SKYDLEYADV PQNMRADTFK YTHEKPQGYY SWHHGAVQYE NGRFTVPKGV GAKGDSGRPI LDNQGRVVAI VLGGVNEGSR TALSVVMWNE KGVTVKYTPE NCEQWSLVTT MCLLANVTFP CAQPPICYDR KPAETLAMLS VNVDNPGYDE LLEAAVKCPG STEELFKEYK LTRPYMARCI RCAVGSCHSP IAIEAVKSDG HDGYVRLQTS SQYGLDSSGN LKGRTMRYDM HGTIKEIPLH QVSLHTSRPC HIVDGHGYFL LARCPAGDSI TMEFKKDSVT HSCSVPYEVK FNPVGRELYT HPPEHGVEQA CQVYAHDAQN RGAYVEMHLP GSEVDSSLVS LSGSSVTVTP PVGTSALVEC ECGGTKISET INKTKQFSQC TKKEQCRAYR LQNDKWVYIS DKLPKAAGAT LKGKLHVPFL LADGKCTVPL APEPMITFGF RSVSLKLHPK NPTYLTTRQL ADEPHYTHEL ISEPAVRNFT VTGKGWEFVW GNHPPKRFWA QETAPGNPHG LPHEVITHYY HRYPMSTILG LSICAAIATV SVAASTWLFC RSRVACLTPY RLTPNARIPF CLAVLCCART ARAETTWESL DHLWNNNQQM FWIQLLIPLA ALIVVTRLLR CVCCVVPFLV MAGAAGAGAY EHATTMPSQA GISYNTIVNR AGYAPLPISI TPTKIKLIPT VNLEYVTCHY KTGMDSPAIK CCGSQECTPT YRPDEQCKVF TGVYPFMWGG AYCFCDTENT QVSKAYVMKS DDCLADHAEA YKAHTASVQA FLNITVGEHS IVTTVYVNGE TPVNFNGVKL TAGPLSTAWT PFDRKIVQYA GEIYNYDFPE YGAGQPGAFG DIQSRTVSSS DLYANTNLVL QRPKAGAIHV PYTQAPSGFE QWKKDKAPSL KSTAPFGCEI YTNPIRAENC AVGSIPLAFD IPDALFTRVS ETPTLSAAEC TLNECVYSSD FGGIATVKYS ASKSGKCAVH VPSGTATLKE AAVELTEQGS ATIHFSTANI HPEFRLQICT SYVTCKGDCH PPKDHIVTHP QYHAQTFTAA VSKTAWTWLT SLLGGSAVII IIGLVLATIV AMYVLTNQKH N (SEQ ID NO:96; GenBank Accession No: AAU89534). Such a glycoprotein may be useful for targeting an EDV of the present disclosure to dendritic cells, macrophages, and cells of the spleen, lymph node, thymus, pancreas, skeletal muscle, and central nervous system.

In some cases, the viral envelope protein is a Venezuelan equine encephalitis virus E2 glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: STEELFKEYK LTRPYMARCI RCAVGSCHSP IAIEAVKSDG HDGYVRLQTS SQYGLDSSGN LKGRTMRYDM HGTIKEIPLH QVSLHTSRPC HIVDGHGYFL LARCPAGDSI TMEFKKDSVT HSCSVPYEVK FNPVGRELYT HPPEHGVEQA CQVYAHDAQN RGAYVEMHLP GSEVDSSLVS LSGSSVTVTP PVGTSALVEC ECGGTKISET INKTKQFSQC TKKEQCRAYR LQNDKWVYIS DKLPKAAGAT LKGKLHVPFL LADGKCTVPL APEPMITFGF RSVSLKLHPK NPTYLTTRQL ADEPHYTHEL ISEPAVRNFT VTGKGWEFVW GNHPPKRFWA QETAPGNPHG LPHEVITHYY HRYPMSTILG LSICAAIATV SVAASTWLFC RSRVACLTPY RLTPNARIPF CLAVLCCART ARA (SEQ ID NO:97; GenBank Accession No: AAU89534). Such a glycoprotein may be useful for targeting an EDV of the present disclosure to dendritic cells, macrophages, and cells of the spleen, lymph node, thymus, pancreas, skeletal muscle, and central nervous system.

In some cases, the viral envelope protein is a Venezuelan equine encephalitis virus El glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: Y EHATTMPSQA GISYNTIVNR AGYAPLPISI TPTKIKLIPT VNLEYVTCHY KTGMDSPAIK CCGSQECTPT YRPDEQCKVF TGVYPFMWGG AYCFCDTENT QVSKAYVMKS DDCLADHAEA YKAHTASVQA FLNITVGEHS IVTTVYVNGE TPVNFNGVKL TAGPLSTAWT PFDRKIVQYA GEIYNYDFPE YGAGQPGAFG DIQSRTVSSS DLYANTNLVL QRPKAGAIHV PYTQAPSGFE QWKKDKAPSL KSTAPFGCEI YTNPIRAENC AVGSIPLAFD IPDALFTRVS ETPTLSAAEC TLNECVYSSD FGGIATVKYS ASKSGKCAVH VPSGTATLKE AAVELTEQGS ATIHFSTANI HPEFRLQICT SYVTCKGDCH PPKDHIVTHP QYHAQTFTAA VSKTAWTWLT SLLGGSAVII IIGLVLATIV AMYVLTNQKH N (SEQ ID NO:98; GenBank Accession No: AAU89534). Such a glycoprotein may be useful for targeting an EDV of the present disclosure to dendritic cells, macrophages, and cells of the spleen, lymph node, thymus, pancreas, skeletal muscle, and central nervous system.

In some cases, the viral envelope protein is a Lassa virus glycoprotein. A suitable Lassa virus protein comprises an amino acid sequence having at least 80%, at least 85%, at least 90%, at least 95%, at least 98%, at least 99%, or 100%, amino acid sequence identity to the following amino acid sequence: MGQIVTFFQE VPHVIEEVMN IVLIALSVLA VLKGLYNFAT CGLVGLVTFL LLCGRSCTTS LYKGVYELQT LELNMETLNM TMPLSCTKNN SHHYIMVGNE TGLELTLTNT SIINHKFCNL SDAHKKNLYD HALMSIISTF HLSIPNFNQY EAMSCDFNGG KISVQYNLSH SYAGDAANHC GTVANGVLQT FMRMAWGGSY IALDSGRGNW DCIMTSYQYL IIQNTTWEDH CQFSRPSPIG YLGLLSQRTR DIYISRRLLG TFTWTLSDSE GKDTPGGYCL TRWMLIEAEL KCFGNTAVAK CNEKHDEEFC DMLRLFDFNK QAIQRLKAEA QMSIQLINKA VNALINDQLI MKNHLRDIMG IPYCNYSKYW YLNHTTTGRT SLPKCWLVSN GSYLNETHFS DDIEQQADNM ITEMLQKEYM ERQGKTPLGL VDLFVFSTSF YLISIFLHLV KIPTHRHIVG KSCPKPHRLN HMGICSCGLY KQPGVPVKWK R (SEQ ID NO:99; GenBank Accession No: ADY11070).

In some cases, the viral envelope protein is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKMRRA LFLQAFLTGR PGKASKKDPK KNPLATSKKD PEKTPLLPTR VNYILIIGVL VLCEVTGVRA DVHLLEQPGN LWITWANRTG QTDFCLSTQS ATSPFQTCLI GIPSPISEGD FKGYVSDNCT TLGTDRLVSS ASITGGPDNS TTLTYRKVSC LLLKLNVSMW NEPPELQLLG SQSLPNITDI TQISGVAGGC VGFRPKGVPW YLGWSQGEAT RFLLRHPSFS NLTGPFTVVT ADRHNLFMGS EYCGAYGYRF WEIYNCSQEG QQYRCGKARR PRPQSPETQC TRQGGIWVNR SKEINETEPF SFTVNCTASN LGNASGCCGK AGTILPGIWV DSTQGNFTKP KALPPAIFLI CGDRAWQGIP SRPVGGPCYL GKLTMLAPNH TDILKILANS SRTGIRRRRS VSHLDDTCSD EVQLWGPTAR IFASILAPGV AAAQALREIE RLACWSVKQA NLTTSLLGDL LDDVTSIRHA VLQNRAAIDF LLLAHGHGCE DIAGMCCFNL SDHSESIQKK FQLMKEHVNK IGVDSDPIGS WLRGLFGGIG GWAVHLLKGL LLGLVVILLL VVCLPCFLQF VSSSIRKMIN NSVSYHTEYR KMQGGAV (SEQ ID NO:100; GenBank Accession No: AD034853).

In some cases, the viral envelope protein is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKMRRA LFLQAFLTGH PGKVSKKDSK KKPPATGKRD PEKTPLLPTR VNYILIIGVL VLCEVTGVRA DVHLLEQPGN LWITWANRTG QTDFCLSTQS ATSPFQTCLI GIPSPISEGD FKGYVSGNCT ALGTHRLVSS GIHGGPDNST TLTYRKVSCL LLKLNVSLLD EPSELQLLGS QSLPNITNIT QIPSVAGGCI GFTPYGSPAG VYGWDRRQVT HILLTDPGSN PFFNKASNSS KPFTVVTADR HNLFMGSEYC GAYGYRFWEM YNCSQMRQNW SICMDVWGRG LPESWCTSTG GIWVNQSKEI NETEPFSFTA NCTGSNLGNV SGCCGESITI LPPGAWVDST QGSFTKPKAL PPGIFLICGD RAWQGIPSRP VGGPCYLGKL TMLAPNHTDI LKILANSSQT GVRHKRSVTH LDDTCSDEVQ LWGPTARIFA SILAPGVAAA QALREIERLA CWSVKQANLT TSLLGDLLDD VTSIRHAVLQ NRAAIDFLLL AHGHGCEDIA GMCCFNLSDH SESIQKKFQL MKEHVNKIGV DSDPIGSWLR GLFGGIGEWA VHLLKGLLLG LVVILLLVVC LPCFLQFVSS SIRKMINNSI SYHTEYRKMQ GGAV (SEQ ID NO:101; GenBank Accession No: AEF97639).

In some cases, the viral envelope protein is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKAFLT GHPGKVSKKD SKKKPPATSK KDPEKTPLLP SRGYFFFPTI LVCVVIISVV PGVGGVHLLR QPGNVWVTWA NKTGRTDFCL SLQSATSPFR TCLIGIPQYP LNTFKGYVTN VTACDNDADL ASQTACLIKA LNTTLPWDPQ ELDILGSQMI KNGTTRTCVT FGSVCYKENN RSRVCHNFDG NFNGTGGAEA ELRDFIAKWK SDDLLIRPYV NQSWTMVSPI NVESFSISRR YCGFTSNETR YYRGDLSNWC GSKRGKWSAG YSNRTKCSSN TTGCGGNCTT EWNYYAYGFT FGKQPEVLWN NGTAKALPPG IFLICGDRAW QGIPRNALGG PCYLGQLTML SPNFTTWITY GPNITGHRRS RRAIRGLSPD CSDEVQLWSA TARIFASFFA PGVAAAQALK EIERLACWSV KQANLTSLIL NAMLEDMNSI RHAVLQNRAA IDFLLLAQGH GCQDVEGMCC FNLSDHSESI HKALQAMKEH TEKIQVEDDP IGDWFTRTFG DLGRWLAKGV KTLLFALLVI VCLLAIIPCI IKCFQDCLSR TMNQFMDERI RYHRIREQL (SEQ ID NO:102; GenBank Accession No: AWM62167).

In some cases, the viral envelope protein is a human T-lymphotropic virus 1 (HTLV-1) glycoprotein. A suitable HTLV-1 protein comprises an amino acid sequence having at least 80%, at least 85%, at least 90%, at least 95%, at least 98%, at least 99%, or 100%, amino acid sequence identity to the following amino acid sequence: MGKFLATLIL FFQFCPLILG DYSPSCCTLT VGVSSYHSKP CNPAQPVCSW TLDLLALSAD QALQPPCPNL VSYSSYHATY SLYLFPHWIK KPNRNGGGYY SASYSDPCSL KCPYLGCQSW TCPYTGAVSS PYWKFQQDVN FTQEVSHLNI NLHFSKCGFP FSLLVDAPGY DPIWFLNTEP SQLPPTAPPL LSHSNLDHIL EPSIPWKSKL LTLVQLTLQS TNYTCIVCID RASLSTWHVL YSPNVSVPSL SSTPLLYPSL ALPAPHLTLP FNWTHCFDPQ IQAIVSSPCH NSLILPPFSL SPVPTLGSRS RRAVPVAVWL VSALAMGAGV AGGITGSMSL ASGKSLLHEV DKDISQLTQA IVKNHKNLLK IAQYAAQNRR GLDLLFWEQG GLCKALQEQC CFLNITNSHV SILQERPPLE NRVLTGWGLN WDLGLSQWAR EALQTGITLV ALLLLVILAG PCILRQLRHL PSRVRYPHYS LINPESSL (SEQ ID NO:103; GenBank Accession No: AAU04884). Such a glycoprotein may be useful for targeting an EDV of the present disclosure to CD4⁺ and CD8⁺ T cells.

In some cases, the viral envelope protein is a human foamy virus gp130 glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: MAPPMTLQQW IIWKKMNKAH EALQNTTTVT EQQKEQIILD IQNEEVQPTR RDKFRYLLYT CCATSSRVLA WMFLVCILLI IVLVSCFVTI SRIQWNKDIQ VLGPVIDWNV TQRAVYQPLQ TRRIARSLRM QHPVPKYVEV NMTSIPQGVY YEPHPEPIVV KERVLGLSQI LMINSENIAN NANLTQEVKK LLTEMVNEEM QSLSDVMIDF EIPLGDPRDQ EQYIHRKCYQ EFANCYLVKY KEPKPWPKEG LIADQCPLPG YHAGLTYNRQ SIWDYYIKVE SIRPANWTTK SKYGQARLGS FYIPSSLRQI NVSHVLFCSD QLYSKWYNIE NTIEQNERFL LNKLNNLTSG TSVLKKRALP KDWSSQGKNA LFREINVLDI CSKPESVILL NTSYYSFSLW EGDCNFTKDM ISQLVPECDG FYNNSKWMHM HPYACRFWRS KKNEKEETKC RDGETKRCLY YPLWDSPEST YDFGYLAYQK NFPSPICIEQ QKIRDQDYEV YSLYQERKIA SKAYGIDTVL FSLKNFLNYT GTPVNEMPNA RAFVGLIDPK FPPSYPNVTR EHYTSCNNRK RRSVDNNYAK LRSMGYALTG AVQTLSQISD INDENLQQGI YLLRDHVITL MEATLHDISV MEGMFAVQHL HTHLNHLK™ LLERRIDWTY MSSTWLQQQL QKSDDEMKVI KRIARSLVYY VKQTHSSPTA TAWEIGLYYE LVIPKHIYLN NWNVVNIGHL VKSAGQLTHV TIAHPYEIIN KECVETIYLH LEDCTRQDYV ICDVVKIVQP CGNSSDTSDC PVWAEAVKEP FVQVNPLKNG SYLVLASSTD CQIPPYVPSI VTVNETTSCF GLDFKRPLVA EERLSFEPRL PNLQLRLPHL VGIIAKIKGI KIEVTSSGES IKEQIERAKA ELLRLDIHEG DTPAWIQQLA AATKDVWPAA ASALQGIGNF LSGTAQGIFG TAFSLLGYLK PILIGVGVIL LVILIFKIVS WIPTKKKNQ (SEQ ID NO:104; GenBank Accession No: P14351).

In some cases, the viral envelope protein is a human foamy virus glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: SLRM QHPVPKYVEV NMTSIPQGVY YEPHPEPIVV KERVLGLSQI LMINSENIAN NANLTQEVKK LLTEMVNEEM QSLSDVMIDF EIPLGDPRDQ EQYIHRKCYQ EFANCYLVKY KEPKPWPKEG LIADQCPLPG YHAGLTYNRQ SIWDYYIKVE SIRPANWTTK SKYGQARLGS FYIPSSLRQI NVSHVLFCSD QLYSKWYNIE NTIEQNERFL LNKLNNLTSG TSVLKKRALP KDWSSQGKNA LFREINVLDI CSKPESVILL NTSYYSFSLW EGDCNFTKDM ISQLVPECDG FYNNSKWMHM HPYACRFWRS KKNEKEETKC RDGETKRCLY YPLWDSPEST YDFGYLAYQK NFPSPICIEQ QKIRDQDYEV YSLYQERKIA SKAYGIDTVL FSLKNFLNYT GTPVNEMPNA RAFVGLIDPK FPPSYPNVTR EHYTSCNNRK RR (SEQ ID NO:105).

In some cases, the viral envelope protein is a human foamy virus glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: SVDNNYAK LRSMGYALTG AVQTLSQISD INDENLQQGI YLLRDHVITL MEATLHDISV MEGMFAVQHL HTHLNHLK™ LLERRIDWTY MSSTWLQQQL QKSDDEMKVI KRIARSLVYY VKQTHSSPTA TAWEIGLYYE LVIPKHIYLN NWNVVNIGHL VKSAGQLTHV TIAHPYEIIN KECVETIYLH LEDCTRQDYV ICDVVKIVQP CGNSSDTSDC PVWAEAVKEP FVQVNPLKNG SYLVLASSTD CQIPPYVPSI VTVNETTSCF GLDFKRPLVA EERLSFEPRL PNLQLRLPHL VGIIAKIKGI KIEVTSSGES IKEQIERAKA ELLRLDIHEG DTPAWIQQLA AATKDVWPAA ASALQGIGNF LSGTAQGIFG TAFSLLGYLK PILIGVGVIL LVILIFKIVS WIPTKKKNQ (SEQ ID NO:106).

In some cases, the viral envelope protein is a visna-maedi virus gp160 glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: MASKESKPSR TTRRGMEPPL RETWNQVLQE LVKRQQQEEE EQQGLVSGKK KSWVSIDLLG TEGKDIKKVN IWEPCEKWFA QVVWGVLWVL QIVLWGCLMW EVRKGNQCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO:107; GenBank Accession No: P35954).

In some cases, the viral envelope protein is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPW11MG1 VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO:108).

In some cases, the viral envelope protein is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO:108).

In some cases, the viral envelope protein is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKR (SEQ ID NO:109).

In some cases, the viral envelope protein is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: GIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO:110).

In some cases, the viral envelope protein is a severe acute respiratory syndrome-associated coronavirus (SARS-CoV) spike glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: MFIFLLFLTL TSGSDLDRCT TFDDVQAPNY TQHTSSMRGV YYPDEIFRSD TLYLTQDLFL PFYSNVTGFH TINHTFGNPV IPFKDGIYFA ATEKSNVVRG WVFGSTMNNK SQSVIIINNS TNVVIRACNF ELCDNPFFAV SKPMGTQTHT MIFDNAFNCT FEYISDAFSL DVSEKSGNFK HLREFVFKNK DGFLYVYKGY QPIDVVRDLP SGFNTLKPIF KLPLGINITN FRAILTAFSP AQDIWGTSAA AYFVGYLKPT TFMLKYDENG TITDAVDCSQ NPLAELKCSV KSFEIDKGIY QTSNFRVVPS GDVVRFPNIT NLCPFGEVFN ATKFPSVYAW ERKKISNCVA DYSVLYNSTF FSTFKCYGVS ATKLNDLCFS NVYADSFVVK GDDVRQIAPG QTGVIADYNY KLPDDFMGCV LAWNTRNIDA TSTGNYNYKY RYLRHGKLRP FERDISNVPF SPDGKPCTPP ALNCYWPLND YGFYTTTGIG YQPYRVVVLS FELLNAPATV CGPKLSTDLI KNQCVNFNFN GLTGTGVLTP SSKRFQPFQQ FGRDVSDFTD SVRDPKTSEI LDISPCSFGG VSVITPGTNA SSEVAVLYQD VNCTDVSTAI HADQLTPAWR IYSTGNNVFQ TQAGCLIGAE HVDTSYECDI PIGAGICASY HTVSLLRSTS QKSIVAYTMS LGADSSIAYS NNTIAIPTNF SISITTEVMP VSMAKTSVDC NMYICGDSTE CANLLLQYGS FCTQLNRALS GIAAEQDRNT REVFAQVKQM YKTPTLKYFG GFNFSQILPD PLKPTKRSFI EDLLFNKVTL ADAGFMKQYG ECLGDINARD LICAQKFNGL TVLPPLLTDD MIAAYTAALV SGTATAGWTF GAGAALQIPF AMQMAYRFNG IGVTQNVLYE NQKQIANQFN KAISQIQESL TTTSTALGKL QDVVNQNAQA LNTLVKQLSS NFGAISSVLN DILSRLDKVE AEVQIDRLIT GRLQSLQTYV TQQLIRAAEI RASANLAATK MSECVLGQSK RVDFCGKGYH LMSFPQAAPH GVVFLHVTYV PSQERNFTTA PAICHEGKAY FPREGVFVFN GTSWFITQRN FFSPQIITTD NTFVSGNCDV VIGIINNTVY DPLQPELDSF KEELDKYFKN HTSPDVDLGD ISGINASVVN IQKEIDRLNE VAKNLNESLI DLQELGKYEQ YIKWPWYVWL GFIAGLIAIV MVTILLCCMT SCCSCLKGAC SCGSCCKFDE DDSEPVLKGV KLHYT (SEQ ID NO:111; GenBank Accession No: ABA02260). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a SARS-CoV S2 glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: CDI PIGAGICASY HTVSLLRSTS QKSIVAYTMS LGADSSIAYS NNTIAIPTNF SISITTEVMP VSMAKTSVDC NMYICGDSTE CANLLLQYGS FCTQLNRALS GIAAEQDRNT REVFAQVKQM YKTPTLKYFG GFNFSQILPD PLKPTKRSFI EDLLFNKVTL ADAGFMKQYG ECLGDINARD LICAQKFNGL TVLPPLLTDD MIAAYTAALV SGTATAGWTF GAGAALQIPF AMQMAYRFNG IGVTQNVLYE NQKQIANQFN KAISQIQESL TTTSTALGKL QDVVNQNAQA LNTLVKQLSS NFGAISSVLN DILSRLDKVE AEVQIDRLIT GRLQSLQTYV TQQLIRAAEI RASANLAATK MSECVLGQSK RVDFCGKGYH LMSFPQAAPH GVVFLHVTYV PSQERNFTTA PAICHEGKAY FPREGVFVFN GTSWFITQRN FFSPQIITTD NTFVSGNCDV VIGIINNTVY DPLQPELDSF KEELDKYFKN HTSPDVDLGD ISGINASVVN IQKEIDRLNE VAKNLNESLI DLQELGKYEQ YIKWPWYVWL GFIAGLIVIV MVTILLCCMT SCCSCLKGAC SCGSCCKFDE DDSEPVLKGV KL (SEQ ID NO:112; GenBank Accession No: ABD73002). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a SARS-CoV spike receptor binding domain glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: PNIT NLCPFGEVFN ATKFPSVYAW ERKKISNCVA DYSVLYNSTF FSTFKCYGVS ATKLNDLCFS NVYADSFVVK GDDVRQIAPG QTGVIADYNY KLPDDFMGCV LAWNTRNIDA TSTGNYNYKY RYLRHGKLRP FERDISNVPF SPDGKPCTPP ALNCYWPLND YGFYTTTGIG YQPYRVVVLS FELLNAPATV CGPKLSTDLI KNQCVNFNFN GLTGTGVLTP SSKRFQPFQQ FGRDVSDFTD SVRDPKTSE (SEQ ID NO:113; GenBank Accession No: ABD73002). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a respiratory syncytial virus (RSV) glycoprotein G. A suitable RSV protein comprises an amino acid sequence having at least 80%, at least 85%, at least 90%, at least 95%, at least 98%, at least 99%, or 100%, amino acid sequence identity to the following amino acid sequence: MSKNKDQRTA KTLERTWDTL NHLLFISSCL YKLNLKSVAQ ITLSILAMII STSLIIAAII FIASANHKVT PTTAIIQDAT SQIKNTTPTY LTQNPQLGIS PSNPSEITSQ ITTILASTTP GVKSTLQSTT VKTKNTTTTQ TQPSKPTTKQRQNKPPSKPN NDFHFEVFNF VPCSICSNNP TCWAICKRIP NKKPGKKTTTKPTKKPTLKT TKKDPKPQTT KSKEVPTTKP TEEPTINTTK TNIITTLLTS NTTGNPELTS QMETFHSTSS EGNPSPSQVS TTSEYPSQPS SPPNTPRQ (SEQ ID NO:114; UniProtKB: P03423-1). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an RSV glycoprotein F. A suitable RSV protein comprises an amino acid sequence having at least 80%, at least 85%, at least 90%, at least 95%, at least 98%, at least 99%, or 100%, amino acid sequence identity to the following amino acid sequence: MELLILKANA ITTILTAVTF CFASGQNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCD™ NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 115; GenBank Accession No: P03420). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an RSV glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCD™ NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO:116). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an RSV FO glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCD™ NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO:116; GenBank Accession No: P03420). Such a glycoprotein may be useful for targeting an EDV of the present disclosure to cells of respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an RSV F2 glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRR (SEQ ID NO: 117; GenBank Accession No: P03420). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is an RSV Fl glycoprotein. A suitable RSV protein comprises an amino acid sequence having at least 80%, at least 85%, at least 90%, at least 95%, at least 98%, at least 99%, or 100%, amino acid sequence identity to the following amino acid sequence: FLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCD™ NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO:118; GenBank Accession No: P03420). Such a glycoprotein may be useful for targeting an EDV of the present disclosure to cells of the lung/respiratory tract.

In some cases, the viral envelope protein is an RSV glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCD™ NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO:116). Such a glycoprotein may be useful for targeting an EDV of the present disclosure to cells of the lung/respiratory tract.

In some cases, the viral envelope protein is a human parainfluenza virus type 3 hemagglutinin-neuraminidase glycoprotein. A suitable human parainfluenza virus type 3 protein comprises an amino acid sequence having at least 80%, at least 85%, at least 90%, at least 95%, at least 98%, at least 99%, or 100%, amino acid sequence identity to the following amino acid sequence: MEYWKHTNHG KDAGNELETS MATHGNKLTN KITYILWTII LVLLSIVFII VLINSIKSEK AHESLLQNIN NEFMEITEKI QMASDNTNDL IQSGVNTRLL TIQSHVQNYI PISLTQQMSD LRKFISEITI RNDNQEVLPQ RITHDVGIKP LNPDDFWRCT SGLPSLMKTP KIRLMPGPGL LAMPTTVDGC IRTPSLVIND LIYAYTSNLI TRGCQDIGKS YQVLQIGIIT VNSDLVPDLN PRISHTFNIN DNRKSCSLAL LNTDVYQLCS TPKVDERSDY ASPGIEDIVL DIVNYDGSIS TTRFKNNNIS FDQPYAALYP SVGPGIYYKG KIIFLGYGGL EHPINENVIC NTTGCPGKTQ RDCNQASHSP WFSDRRMVNS IIVVDKGLNS IPKLKVWTIS MRQNYWGSEG RLLLLGNKIY IYTRSTSWHS KLQLGIIDIT DYSDIRIKWT WHNVLSRPGN NECPWGHSCP DGCITGVYTD AYPLNPTGSI VSSVILDSQK SRVNPVITYS TATERVNELA ILNRTLSAGY TTTSCITHYN KGYCFHIVEI NHKSLNTLQP MLFKTEIPKS CS (SEQ ID NO:119; GenBank Accession No: AAP35240). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a human parainfluenza virus type 3 glycoprotein FO. A suitable human parainfluenza virus type 3 protein comprises an amino acid sequence having at least 80%, at least 85%, at least 90%, at least 95%, at least 98%, at least 99%, or 100%, amino acid sequence identity to the following amino acid sequence: MPISILLIIT TMIMASHCQI DITKLQHVGV LVNSPKGMKI SQNFETRYLI LSLIPKIDDS NSCGDQQIKQ YKRLLDRLII PLYDGLRLQK DVIVANQESN ENTDPRTERF FGGVIGTIAL GVATSAQITA AVALVEAKQA RSDIEKLKEA IRDTNKAVQS VQSSVGNLIV AIKSVQDYVN KEIVPSIARL GCEAAGLQLG IALTQHYSEL TNIFGDNIGS LQEKGIKLQG IASLYRTNIT EIFTTSTVDK YDIYDLLFTE SIKVRVIDVD LNDYSITLQV RLPLLTRLLN TQIYKVDSIS YNIQNREWYI PLPSHIMTKG AFLGGADVKE CIEAFSSYIC PSDPGFVLNH EMESCLSGNI SQCPRTTVTS DIVPRYAFVN GGVVANCITT TCTCNGIGNR INQPPDQGVK IITHKECNTI GINGMLFNTN KEGTLAFYTP ADITLNNSVA LDPIDISIEL NKAKSDLEES KEWIRRSNQK LDSIGSWHQS STTIIVILIM MIILFIINIT IITIAIKYYR IQKRNRVDQN DKPYVLTNK (SEQ ID NO:120; GenBank Accession No: AXA52708). Such a glycoprotein may be useful for targeting an EDV of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the viral envelope protein is a Hepatitis C virus (HCV) El glycoprotein. A suitable HCV protein comprises an amino acid sequence having at least 80%, at least 85%, at least 90%, at least 95%, at least 98%, at least 99%, or 100%, amino acid sequence identity to the following amino acid sequence: YQVRNSSGLY HVTNDCPNSS IVYEAADAIL HTPGCVPCVR EGNASRCWVA VTPTVATRDG KLPTTQLRRH IDLLVGSATL CSALYVGDLC GSVFLVGQLF TFSPRRHWTT QDCNCSIYPG HITGHRMAWD MMMNWSPTAA LVVAQLLRIP QAIMDMIAGA HWGVLAGIAY FSMVGNWAKV LVVLLLFAGV DA (SEQ ID NO:121; GenBank Accession No: NP_751920). Such a glycoprotein may be useful for targeting an EDV of the present disclosure to a liver cell.

In some cases, the viral envelope protein is an HCV E2 glycoprotein. A suitable HCV protein comprises an amino acid sequence having at least 80%, at least 85%, at least 90%, at least 95%, at least 98%, at least 99%, or 100%, amino acid sequence identity to the following amino acid sequence: ETHVTGGSAG RTTAGLVGLL TPGAKQNIQL INTNGSWHIN STALNCNESL NTGWLAGLFY QHKFNSSGCP ERLASCRRLT DFAQGWGPIS YANGSGLDER PYCWHYPPRP CGIVPAKSVC GPVYCFTPSP VVVGTTDRSG APTYSWGAND TDVFVLNNTR PPLGNWFGCT WMNSTGFTKV CGAPPCVIGG VGNNTLLCPT DCFRKHPEAT YSRCGSGPWI TPRCMVDYPY RLWHYPCTIN YTIFKVRMYV GGVEHRLEAA CNWTRGERCD LEDRDRSELS PLLLSTTQWQ VLPCSFTTLP ALSTGLIHLH QNIVDVQYLY GVGSSIASWA IKWEYVVLLF LLLADARVCS CLWMMLLISQ AEA (SEQ ID NO:122; GenBank Accession No: NP_751921). Such a glycoprotein may be useful for targeting an EDV of the present disclosure to a liver cell.

In some cases, the viral envelope protein is a fowl plague virus glycoprotein. A suitable fowl plague virus protein comprises an amino acid sequence having at least 80%, at least 85%, at least 90%, at least 95%, at least 98%, at least 99%, or 100%, amino acid sequence identity to the following amino acid sequence: MNTQILVFAL VAVIPTNADK ICLGHHAVSN GTKVNTLTER GVEVVNATET VERTNIPKIC SKGKRTTDLG QCGLLGTITG PPQCDQFLEF SADLIIERRE GNDVCYPGKF VNEEALRQIL RGSGGIDKET MGFTYSGIRT NGTTSACRRS GSSFYAEMEW LLSNTDNASF PQMTKSYKNT RRESALIVWG IHHSGSTTEQ TKLYGSGNKL ITVGSSKYHQ SFVPSPGTRP QINGQSGRID FHWLILDPND TVTFSFNGAF IAPNRASFLR GKSMGIQSDV QVDANCEGEC YHSGGTITSR LPFQNINSRA VGKCPRYVKQ ESLLLATGMK NVPEPSKKRE KRGLFGAIAG FIENGWEGLV DGWYGFRHQN AQGEGTAADY KSTQSAIDQI TGKLNRLIEK TNQQFELIDN EFTEVEKQIG NLINWTKDFI TEVWSYNAEL LVAMENQHTI DLADSEMNKL YERVRKQLRE NAEEDGTGCF EIFHKCDDDC MASIRNNTYD HSKYREEAMQ NRIQIDPVKL SSGYKDVILW FSFGASCFLL LAIAVGLVFI CVKNGNMRCT ICI (SEQ ID NO:123; GenBank Accession No: 0601245A).

In some cases, the viral envelope protein is an Autographa californica nuclear polyhedrosis virus (AcMNPV) major envelope glycoprotein gp64. A suitable AcMNPV protein comprises an amino acid sequence having at least 80%, at least 85%, at least 90%, at least 95%, at least 98%, at least 99%, or 100%, amino acid sequence identity to the following amino acid sequence:

(SEQ ID NO: 158; UniProt Accession No: P17501-1)
MVSAIVLYVL LAAAAHSAFA AEHCNAQMKT GPYKIKNLDI TPPKETLQKD VEITIVETDY
NENVIIGYKG YYQAYAYNGG SLDPNTRVEE TMKTLNVGKE DLLMWSIRQQ CEVGEELID
WGSDSDDCFR DNEGRGQWVK GKELVKRQNN NHFAHHTCNK SWRCGISTSK
MYSRLECQDD TDECQVYILD AEGNPINVTV DTVLHRDGVS MILKQKSTFT TRQIKAACLL
IKDDKNNPES VTREHCLIDN DIYDLSKNTW NCKFNRCIKR KVEHRVKKRP PTWRHNVRAI
YTEGDTATKG DLMHIQEELM YENDLLKMNI ELMHAHINKL NNMLHDLIVS VAKVDERLI
NLMNNSVSST FLSDDTFLLM PCTNPPAHTS NCYNNSIYKE GRWVANTDSS QCIDFSNYKE
LAIDDDVEFW IPTIGNTTYH DSWKDASGWS FIAQQKSNLI TTMENTKFGG VGTSLSDITS
MAEGELAAKL TSFMFGHVVN FVIILIVILF LYCMIRNRNR QY.

In some cases, the viral envelope protein is an AcMNPV glycoprotein. A suitable AcMNPV protein comprises an amino acid sequence having at least 80%, at least 85%, at least 90%, at least 95%, at least 98%, at least 99%, or 100%, amino acid sequence identity to the following amino acid sequence: AEHCNAQMKT GPYKIKNLDI TPPKETLQKD VEITIVETDY NENVIIGYKG YYQAYAYNGG SLDPNTRVEE TMKTLNVGKE DLLMWSIRQQ CEVGEELIDR WGSDSDDCFR DNEGRGQWVK GKELVKRQNN NHFAHHTCNK SWRCGISTSK MYSRLECQDD TDECQVYILD AEGNPINVTV DTVLHRDGVS MILKQKSTFT TRQIKAACLL IKDDKNNPES VTREHCLIDN DIYDLSKNTW NCKFNRCIKR KVEHRVKKRP PTWRHNVRAK YTEGDTATKG DLMHIQEELM YENDLLKMNI ELMHAHINKL NNMLHDLIVS VAKVDERLIG NLMNNSVSST FLSDDTFLLM PCTNPPAHTS NCYNNSIYKE GRWVANTDSS QCIDFSNYKE LAIDDDVEFW IPTIGNTTYH DSWKDASGWS FIAQQKSNLI TTMENTKFGG VGTSLSDITS MAEGELAAKL TSFMFGHVVN FVIILIVILF LYCMIRNRNR QY (SEQ ID NO:124).

In some cases, the viral envelope protein is a measles virus hemagglutinin (H) polypeptide. See, e.g., Levy et al. (2017) BloodAAdv. 1:2088. A suitable measles virus H polypeptide comprises an amino acid sequence having at least 80%, at least 85%, at least 90%, at least 95%, at least 98%, at least 99%, or 100%, amino acid sequence identity to the following amino acid sequence: MSPQRDRINA FYKDNPHPKG SRIVINREHL MIDRPYVLLA VLFVMFLSLI GLLAIAGIRL HRAAIYTAEI HKSLSTNLDV TNSIEHQVKD VLTPLFKIIG DEVGLRTPQR FTDLVKFISD KIKFLNPDRE YDFRDLTWCI NPPERIKLDY DQYCADVAAE ELMNALVNST LLETRTTNQF LAVSKGNCSG PTTIRGQFSN MSLSLLDLYL SRGYNVSSIV TMTSQGMYGG TYLVEKPNLS SKGSELSQLS MYRVFEVGVI RNPGLGAPVF HMTNYFEQPV SNDLSNCMVA LGELKLAALC HGGDSITIPY QGSGKGVSFQ LVKLGVWKSP TDMQSWVPLS TDDPVIDRLY LSSHRGVIAD NQAKWAVPTT RTDDKLRMET CFQQACKGKI QALCENPEWA PLKDNRIPSY GVLSVDLSLT VELKIKIASG FGPLITHGSG MDLYKSNHNN VYWLTIPPMK NLALGVINTL EWIPRFKVSP YLFTVPIKEA GEDCHAPTYL PAEVDGDVKL SSNLVILPGQ DLQYVLATYD TSRVEHAVVY YVYSPSRSFS YFYPFRLPIK GIPIELQVEC FTWDQKLWCR HFCVLADSES GGHITHSGMV GMGVSCTVTR EDGTNSR (SEQ ID NO:155). Such a glycoprotein may be useful for targeting an EDV of the present disclosure to T cells, B cells, monocytes, macrophages, dendritic cells, and hematopoietic stem cells (e.g., CD34* cells).

In some cases, the viral envelope protein is a measles virus fusion (F) polypeptide. A suitable measles virus F polypeptide comprises an amino acid sequence having at least 80%, at least 85%, at least 90%, at least 95%, at least 98%, at least 99%, or 100%, amino acid sequence identity to the following amino acid sequence: MSIMGLKVNV SAIFMAVLLT LQTPTGQIHW GNLSKIGVVG IGSASYKVMT RSSHQSLVIK LMPNITLLNN CTRVEIAEYR RLLRTVLEPI RDALNAMTQN IRPVQSVASS RRHKRFAGVV LAGAALGVAT AAQITAGIAL HQSMLNSQAI DNLRASLETT NQAIETIRQA GQEMILAVQG VQDYINNELI PSMNQLSCDL IGQKLGLKLL RYYTEILSLF GPSLRDPISA EISIQALSYA LGGDINKVLE KLGYSGGDLL GILESGGIKA RITHVDTESY FIVLSIAYPT LSEIKGVIVH RLEGVSYNIG SQEWYTTVPK YVATQGYLIS NFDESSCTFM PEGTVCSQNA LYPMSPLLQE CLRGYTKSCA RTLVSGSFGN RFILSQGNLI ANCASILCKC YTTGTIINQD PDKILTYIAA DHCPVVEVNG VTIQVGSRRY PDAVYLHRID LGPPISLERL DVGTNLGNAI AKLEDAKELL ESSDQILRSM KGLSSTSIVY ILIAVCLGGL IGIPALICCC RGRCNKKGEQ VGMSRPGLKP DLTGTSKSYV RSL (SEQ ID NO:126). Such a glycoprotein may be useful for targeting an EDV of the present disclosure to T cells, B cells, monocytes, macrophages, dendritic cells, and hematopoietic stem cells (e.g., CD34⁺ cells).

### Variant Viral Envelope Proteins

In some cases, an EDV of the present disclosure comprises a fusion polypeptide comprising (i) a variant viral envelope protein and (ii) a targeting polypeptide that provides for binding to a target cell, where the variant viral envelope protein comprises one or more amino acid substitutions compared to a wild-type viral envelope protein, and where the variant viral envelope protein exhibits reduced binding to its native receptor, compared to the binding of the wild-type viral envelope protein to the native receptor. In some cases, the variant viral envelope protein retains endosomal fusion activity.

In some cases, the viral envelope protein is a variant VSV-G protein comprising an amino acid sequence having at least 80%, at least 85%, at least 90%, at least 95%, at least 98%, or at least 99%, amino acid sequence identity to the VSV-G amino acid sequence depicted in FIG. 16A; where the variant VSV-G protein exhibits comprises one or more amino acid substitutions compared to a wild-type viral envelope protein (compared to the amino acid sequence depicted in FIG. 16A), and where the variant viral envelope protein exhibits reduced binding to its native receptor, compared to the binding of a VSV-G polypeptide comprising the amino acid sequence depicted in FIG. 16A to its native receptor. The native receptor for VSV-G is the low-density lipoprotein receptor (LDLR).

In some cases, the VSV-G polypeptide comprises one or more amino acid substitutions that reduce binding to the native receptor for VSV-G, while retaining the endosomal fusion function of the VSV-G polypeptide. In some cases, the viral envelope protein is a VSV-G protein comprising an amino acid sequence having at least 80%, at least 85%, at least 90%, at least 95%, at least 98%, at least 99%, or 100%, amino acid sequence identity to the VSV-G amino acid sequence depicted in FIG. 16A, where amino acid 47 is other than a Lys. In some cases, the viral envelope protein is a VSV-G protein comprising an amino acid sequence having at least 80%, at least 85%, at least 90%, at least 95%, at least 98%, at least 99%, or 100%, amino acid sequence identity to the VSV-G amino acid sequence depicted in FIG. 16A, where amino acid 354 is other than an Arg. In some cases, the viral envelope protein is a VSV-G protein comprising an amino acid sequence having at least 80%, at least 85%, at least 90%, at least 95%, at least 98%, at least 99%, or 100%, amino acid sequence identity to the VSV-G amino acid sequence depicted in FIG. 16A, where amino acid 47 is other than a Lys and amino acid 354 is other than an Arg. In some cases, the Lys at amino acid 47 is substituted with an Ala. In some cases, the Lys at amino acid 47 is substituted with a Gln. In some cases, the Arg at amino acid 354 is substituted with an Ala. In some cases, the Arg at amino acid 354 is substituted with a Gln.

In some cases, the viral envelope protein is a VSV-G protein comprising an amino acid sequence having at least 80%, at least 85%, at least 90%, at least 95%, at least 98%, at least 99%, or 100%, amino acid sequence identity to the VSV-G amino acid sequence depicted in FIG. 16B, where the VSV-G protein comprises a Gln at position 47 and an Ala at position 354.

In some cases, the viral envelope protein is a variant measles hemagglutinin (HA) protein comprising an amino acid sequence having at least 80%, at least 85%, at least 90%, at least 95%, at least 98%, or at least 99%, amino acid sequence identity to the measles HA amino acid sequence depicted in FIG. 16C; where the variant measles HA protein exhibits comprises one or more amino acid substitutions compared to a wild-type viral envelope protein (compared to the amino acid sequence depicted in FIG. 16C), and where the variant viral envelope protein exhibits reduced binding to its native receptor, compared to the binding of a measles HA protein comprising the amino acid sequence depicted in FIG. 16C to its native receptor. In some cases, the variant measles HA protein comprises a substitution of one or more of Y481, R533, S548, and F549, based on the amino acid numbering set out in FIG. 16C. CD46 is a native receptor for measles virus HA.

In some cases, the viral envelope protein is a variant measles HA protein comprising an amino acid sequence having at least 80%, at least 85%, at least 90%, at least 95%, at least 98%, or at least 99%, amino acid sequence identity to the measles HA amino acid sequence depicted in FIG. 16D, where amino acid 841 is other than a Tyr, amino acid 533 is other than an Arg, amino acid 548 is other than a Ser, and amino acid 549 is other than a Phe. In some cases, the viral envelope protein is a variant measles HA protein comprising an amino acid sequence having at least 80%, at least 85%, at least 90%, at least 95%, at least 98%, or at least 99%, amino acid sequence identity to the measles HA amino acid sequence depicted in FIG. 16D, where amino acid 841 is Ala, amino acid 533 is Ala, amino acid 548 is Len, and amino acid 549 is Ser.

### Targeting Polypeptides

An EDV of the present disclosure comprises a fusion polypeptide that comprises: i) a viral glycoprotein; and ii) a targeting polypeptide (i.e., one or more targeting polypeptides) that provides for binding to a target cell or target cell type. Targeting polypeptides include antibodies and antibody mimetics (also referred to as antibody analogs). Suitable antibody analogs include, e.g., an affibody, an affilin, an affimer, an affitin, an alphabody, an anticalin, an avimer, a DARPin, a Fynomer, a Kunitz domain peptide, a monobody, a repebody, a VLR, and a nanoCLAMP. Suitable antibodies include a single chain Fv (scFv) polypeptide, a diabody, a triabody, and a nanobody. In some cases, the antibody is a single-chain Fv polypeptide. In some cases, the antibody is a nanobody. In some cases, the antibody is a bispecific antibody. In some cases, an EDV of the present disclosure comprises a fusion polypeptide that comprises: i) a viral envelop protein; and ii) one or more antibodies or antibody analogs that bind specifically to a target polypeptide on a target cell. In some cases, an EDV of the present disclosure comprises a fusion polypeptide that comprises: i) a viral envelop protein; and ii) two different antibodies (e.g., a first antibody and a second antibody), where the first antibody specifically binds to a first target polypeptide on a target cell and the second antibody specifically binds to a second target polypeptide on the same target cell.

In some instances, EDVs comprising two or more different targeting polypeptides are provided. In some cases, EDVs comprising a bispecific targeting polypeptide is provided wherein the bispecific targeting polypeptide binds to two different targets on the targeted cell type. In some cases, the bispecific targeting polypeptide is a bispecific antibody or derivative thereof.

In some cases, the targeting polypeptide provides for selective binding to an organ such as kidney, liver, bone, pancreas, brain, lung, heart, and the like. In some cases, the targeting polypeptide provides for selective binding to a particular cell type. For example, in some cases, the targeting polypeptide provides for selective binding to a cell such as a skeletal muscle cell, a cardiomyocyte, an adipocyte, an epithelial cell, an endothelial cell, a macrophage, a beta islet cell, or an immune cell (e.g., a T cell, a B cell, a monocyte, a natural killer cell, a dendritic cell, etc.). In some cases, the targeting polypeptide provides for selective binding to a diseased cell, relative to a non-diseased cell of the same cell type. In some cases, the antibody provides for selective binding to a CAR-T cell, i.e., a T cell that is modified to express a chimeric antigen receptor (CAR) on its surface.

### Fusion Polypeptides

In some cases, e.g., where the antibody is a scFv or a nanobody, the antibody itself is a fusion polypeptide comprising: (i) the antibody; and (ii) a heterologous polypeptide (a “fusion partner”). The fusion partner can be a polypeptide that enhances accessibility of the antibody to a target cell. Suitable fusion partners include, but are not limited to, the stalk portion of a polypeptide; the stalk and transmembrane domain of a polypeptide; an immunoglobulin hinge polypeptide; a linker polypeptide; and the like.

In some cases, the fusion partner is the stalk and transmembrane domain of a transmembrane protein. A “transmembrane domain”, as used herein, is a portion of a transmembrane protein that contains a hydrophobic portion that can insert into or span a cell membrane. Transmembrane components or domains have a three-dimensional structure that is thermodynamically stable in a cell membrane and generally range in length from about 15 amino acids to about 30 amino acids. The structure of a transmembrane component or domain may comprise an alpha helix, a beta barrel, a beta sheet, a beta helix, or any combination thereof. In certain embodiments, a transmembrane component or domain comprises or is derived from a known transmembrane protein (e.g., a CD4 transmembrane domain, a CD8 transmembrane domain, a CD27 transmembrane domain, a CD28 transmembrane domain, or any combination thereof).

In some cases, the fusion partner is the stalk and transmembrane domain of a CD8a polypeptide. As an example, the stalk and transmembrane domain can comprise the following amino acid sequence: TTTPAPRPPTPAPTIASQPLSLRPEACRPAAGGAVIHTRGLDFACDIYIWAPLAGTCGVLLLSLVIT LYC (SEQ ID NO:20), where the stalk has the amino acid sequence TTTPAPRPPTPAPTIASQPLSLRPEACRPAAGGAVHTRGLDFACD (SEQ ID NO:21) and the TMD has the sequence IYIWAPLAGTCGVLLLSLVITLYC (SEQ ID NO:22). As another example, the fusion partner is the stalk domain of a CD8a polypeptide; e.g., the fusion partner comprises the amino acid sequence: TTTPAPRPPTPAPTIASQPLSLRPEACRPAAGGAVHTRGLDFACD (SEQ ID NO:21).

In some cases, the fusion partner is the stalk and transmembrane domain of a platelet-derived growth factor receptor (PDGFR).

In some cases, the fusion partner is a glycine-rich polypeptide having a length of from 5 amino acids to about 50 amino acids; e.g., where the fusion partner comprises the sequence (GGGGS)n (SEQ ID NO:38), where n is an integer from 1 to 10, e.g., where n is 1, 2, 3, 4, 5, 6, 7, 8, 9, or 10, e.g., where n is 3.

In some cases, the fusion partner is an immunoglobulin (Ig) hinge polypeptide. As used herein, a “hinge polypeptide,” “hinge region,” or a “hinge” refers to (a) an immunoglobulin hinge sequence (made up of, for example, upper and core regions of an immunoglobulin hinge) or a functional fragment or variant thereof, (b) a type II C-lectin interdomain (stalk) region or a functional fragment or variant thereof, or (c) a cluster of differentiation (CD) molecule stalk region or a functional variant thereof. As used herein, a “wild-type immunoglobulin hinge region” refers to a naturally occurring upper and middle hinge amino acid sequences interposed between and connecting the CHI and CH2 domains (for IgG, IgA, and IgD) or interposed between and connecting the CHI and CH3 domains (for IgE and IgM) found in the heavy chain of an antibody.

### T Cell-Targeting Antibodies

In some cases, the targeting polypeptide is an antibody that targets (binds specifically to) an antigen expressed on the surface of a T cell, thereby targeting the EDV to the T cell. In some cases, the T cell is a CD4⁺ T cell. In some cases, the T cell is a CD8⁺ T cell. In some cases, the antibody is a scFv or a nanobody that binds to CD4. In some cases, the antibody is a scFv or a nanobody that binds to CD3. In some cases, the antibody is a scFv or a nanobody that binds to CD8. In some cases, the antibody is a scFv or a nanobody that binds to CD28. In some cases, the targeting polypeptide comprises one or more antibodies, e.g., one or a combination of anti-CD3 (e.g., CD3 scFv-3), anti-CD4 (e.g., CD4 scFv-2), and anti-CD28 (e.g., CD28 scFv-2) (e.g., an anti-CD3 and an anti-CD4 antibody; an anti-CD3 and an anti-CD28 antibody; an anti-CD3, an anti-CD4, and an anti-CD28 antibody; and the like).

In some cases, an EDV comprises a fusion polypeptide comprising: i) a viral envelop protein; ii) an anti-CD3 antibody; and iii) an anti-CD8 antibody. In some cases, an EDV comprises a fusion polypeptide comprising: i) a viral envelop protein; ii) an anti-CD3 antibody; and iii) an anti-CD28 antibody. In some cases, an EDV comprises a fusion polypeptide comprising: i) a viral envelop protein; ii) an anti-CD3 antibody; and iii) an anti-CD4 antibody. In some cases, an EDV comprises a fusion polypeptide comprising: i) a viral envelop protein; ii) an anti-CD3 antibody; and iii) an anti-CD28 antibody. In some cases, an EDV comprises a fusion polypeptide comprising: i) a viral envelop protein; ii) an anti-CD3 nanobody; and iii) an anti-CD8 nanobody. In some cases, an EDV comprises a fusion polypeptide comprising: i) a viral envelop protein; ii) an anti-CD3 nanobody; and iii) an anti-CD28 nanobody. In some cases, an EDV comprises a fusion polypeptide comprising: i) a viral envelop protein; ii) an anti-CD3 nanobody; and iii) an anti-CD4 nanobody. In some cases, an EDV comprises a fusion polypeptide comprising: i) a viral envelop protein; ii) an anti-CD3 nanobody; and iii) an anti-CD28 nanobody. In some cases, an EDV comprises a fusion polypeptide comprising: i) a viral envelop protein; ii) an anti-CD3 scFv; and iii) an anti-CD8 scFv. In some cases, an EDV comprises a fusion polypeptide comprising: i) a viral envelop protein; ii) an anti-CD3 scFv; and iii) an anti-CD28 scFv. In some cases, an EDV comprises a fusion polypeptide comprising: i) a viral envelop protein; ii) an anti-CD3 scFv; and iii) an anti-CD4 scFv. In some cases, an EDV comprises a fusion polypeptide comprising: i) a viral envelop protein; ii) an anti-CD3 scFv; and iii) an anti-CD28 scFv.

### Cancer Cell-Targeting Antibodies

In some cases, the targeting polypeptide is an antibody that targets a cancer antigen, thereby targeting the EDV to a cancerous cell that displays the cancer antigen on its cell surface.

Suitable antigens bound by an antibody present in an EDV of the present disclosure include, e.g., CD3, epidermal growth factor receptor (EGFR), CA-125 (highly expressed on epithelial ovarian cancer cells), CD80, CD86, glycoprotein IIb/IIIa receptor, CD51, TNF-α, epithelial adhesion molecule EpcAM (CD326), vascular endothelial growth factor receptor-2 (VEGFR-2), CD52, mesothelin, activin receptor-like kinase 1 (ALK-1), phosphatidyl serine, CD19, vascular endothelial growth factor A (VEGF-A), IL-6 receptor, CDIIa, CD25, CD2, CD3 receptor, and the like.

Suitable antigens bound by an antibody present in an EDV of the present disclosure include, e.g., carbonic anhydrase IX, alpha-fetoprotein (AFP), a-actinin-4, A3, ART-4, B7, Ba 733, BAGE, BrE3-antigen, CA125, CAMEL, CAP-1, CASP-8/m, CCL19, CCL21, CD1, CDia, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, HIF-Ia, colon-specific antigen-p (CSAp), CEACAM5, CEACAM6, c-Met, DAM, epidermal growth factor receptor (EGFR), EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-P, HLA-DR, HM1.24, human chorionic gonadotropin (HCG) and its subunits, HER2/neu, histone H2B, histone 113, histone H4, HMGB-1, hypoxia inducible factor (HIF-1), HSP70-2M, HST-2, insulin-like growth factor-1 receptor (IGF-1R), IFN-γ IFN-α, IFN-β, IFN-X, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-IA, MIP-1B, MIF, MUC1, MUC2, MUC3, MUC4, MUC5ac, MUC13, MUC16, MUM-1/2, MUM-3, NCA66, NCA95, NCA90, PAM4 antigen, PD-1, PD-L1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn antigen, tumor necrosis antigens, VEGFR, ED-B fibronectin, WT-1, 17-IA-antigen, complement factors C3, C3a, C3b, C5a, C5; and the like.

In some cases, the cancer-associated antigen is an antigen associated with a hematological cancer. Examples of such antigens include, but arc not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin aV03, IGF-R1, IL6, KIR, PD-1, PD-Li, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin aV03, integrin α5β1, Le^{gamma} , Liv1, mesothelin, mucins, NaPi2b, PD-1, PD-Li, PD-1 receptor, pgA33, PSMA, RANKL, ROR1, TAG-72, tenascin, TRAILRI, TRAILR2, VEGF, VEGFR, and others listed above.

In some cases, the cancer-associated antigen is an antigen associated with a cancer stem cell. Examples of such antigens include, but are not limited to, SSEA3, SSEA4, TRA-1-60, TRA-1-81, CD133, CD90, CD326, Cripto-1, PODXL-1, ABCG2, CD24, CD49f, Notch2, CD146, CD10, CD117, and CD26 (Kim & Ryu (2017) BMB Rep 50(6): 285-298).

Examples of suitable antibodies include, e.g., abciximab (anti-glycoprotein IIb/IIIa), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), trastuzumab (anti-ErbB2), lambrolizumab (anti-PD-1 receptor), nivolumab (anti-PD-1 receptor), ipilimumab (anti-CTLA-4), abagovomab (anti-CA-125), adecatumumab (anti-EpCAM), atlizumab (anti-IL-6 receptor), benralizumab (anti-CD125), obinutuzumab (GA101, anti-CD20), CC49 (anti-TAG-72), tocilizumab (anti-IL-6 receptor), basiliximab (anti-CD25), daclizumab (anti-CD25), efalizumab (anti-CD11a), GA101 (anti-CD20; Glycart Roche), muromonab-CD3 (anti-CD3 receptor), natalizumab (anti-α-4 integrin), and the like.

Non-limiting examples of cancer-associated antigen-targeted antibodies that can be targeted include, but are not limited to, abituzumab (anti-CD51), LL1 (anti-CD74), LL2 or RFB4 (anti-CD22), veltuzumab (hA20, anti-CD20), rituxumab (anti-CD20), obinutuzumab (GA101, anti-CD20), ibalizumab (anti-CD4), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PGi-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

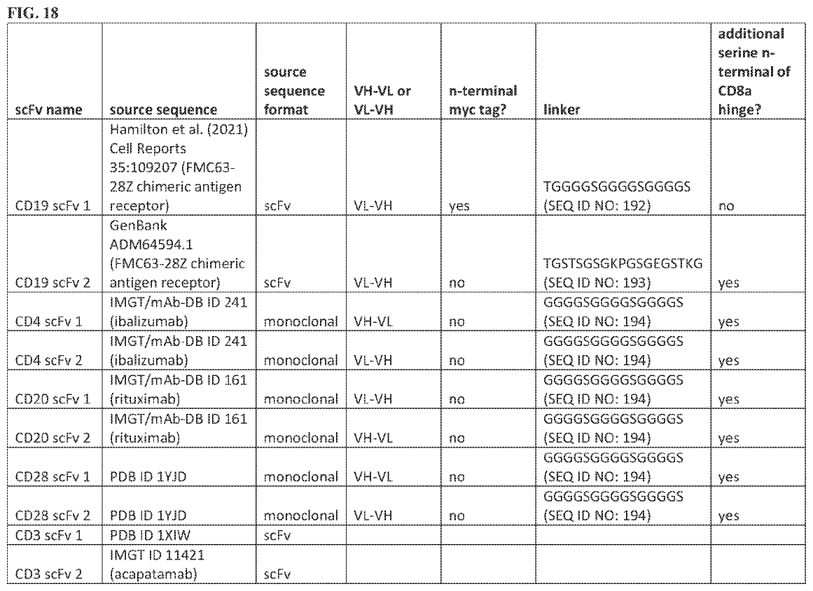

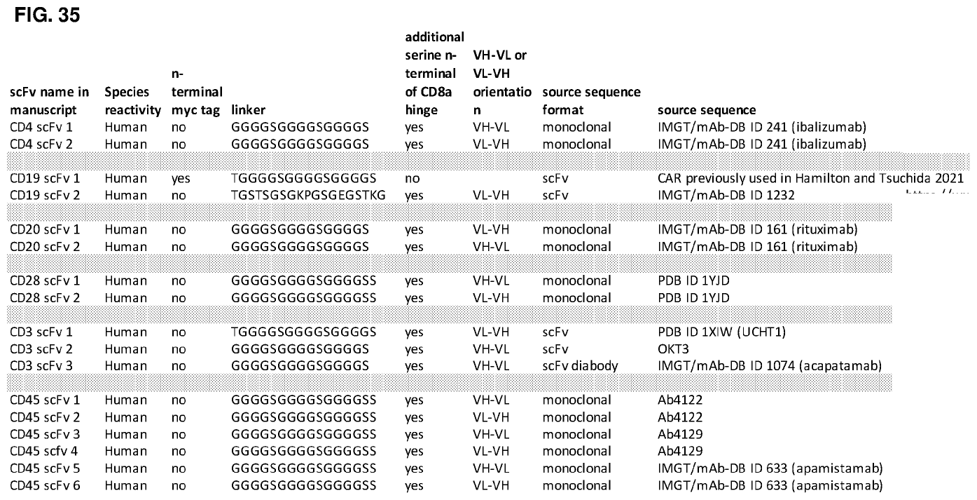

VH and VL amino acid sequences of various cancer antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of antibodies that can be used as part of a targeting polypeptide of a subject EDV. In some cases, an antibody includes the CDR sequences from the anti-CD19 scFv FMC63. In some cases, an antibody includes the CDR sequences from the anti-CD4 antibody ibalizumab (IMGT/mAb-DB ID 241). In some cases, an antibody includes the CDR sequences from the anti-CD20 antibody rituximab (IMGT/mAb-DB ID 161). In some cases, an antibody includes the CDR sequences from the anti-CD3 antibody acapatamab (IMGT/mAb-DB ID 1074). In some cases, an antibody includes the CDR sequences from the anti-CD3 antibody OKT3. In some cases, an antibody includes the CDR sequences from the anti-CD28 antibody PDB ID IYJD. In some cases, an antibody includes the CDR sequences from the anti-CD19 antibody IMGT/mAb-DB ID 1232. In some cases, an antibody includes the CDR sequences from the anti-CD45 antibody Ab4122. In some cases, an antibody includes the CDR sequences from the anti-CD45 antibody Ab4129. In some cases, an antibody includes the CDR sequences from the anti-CD45 antibody apamistamab (IMGT/mAb-DB ID 633). See, e.g., FIG. 18 and FIG. 35. The following are non-limiting examples of cancer antigen-binding antibodies. Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence:

DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:159); and b) a heavy chain comprising an amino acid sequence having at least 90%, at least 95%, at least 98%, at least 99%, or 100%, amino acid sequence identity to the following amino acid sequence:

(SEQ ID NO: 160)
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYWGQGTL
VTVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEAL
HNHYTQKSLSLSPGK.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:161); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:162). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:162); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:161). Suitable linkers are described elsewhere herein and include, e.g., (GGGGS)n (SEQ ID NO: 38), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above.

For example, an anti-Her2 antibody can comprise a VL CDR1 having the amino acid sequence RASQDVNTAVA (SEQ ID NO:164); a VL CDR2 having the amino acid sequence SASFLY (SEQ ID NO:165); a VL CDR3 having the amino acid sequence QQHYTTPP (SEQ ID NO:166); a VH CDR1 having the amino acid sequence GFNIKDTY (SEQ ID NO:167); a VH CDR2 having the amino acid sequence IYPTNGYT (SEQ ID NO:168); and a VH CDR3 having the amino acid sequence SRWGGDGFYAMDY (SEQ ID NO:169).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 170)
EVQLVESGGGLVQPGGSLRLSCAASGFNIKDTYIHWVRQAPGKGLEWVARIYPTNGYTRYADS
VKGRFTISADTSKNTAYLQMNSLRAEDTAVYYCSRWGGDGFYAMDYWGQGTLVTVSSGGGG
SGGGGSGGGGSDIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:

DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:171); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 172)
EVQLVESGGGLVQPGGSLRLSCAASGFTFTDYTMDWVRQAPGKGLEWVADVNP
NSGGSIYNQRFKGRFTLSVDRSKNTLYLQMNSLRAEDTAVYYCARNLGPSFYFDYWGQGTLVT
VSSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALH
NHYTQKSLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:173); and b) a VH comprising an amino acid sequence having at least 90%, at least 95%, at least 98%, at least 99%, or 100%, amino acid sequence identity to the amino acid sequence:

(SEQ ID NO: 174)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. For example, an anti-HER2 antibody can comprise a VL CDR1 having the amino acid sequence KASQDVSIGVA (SEQ ID NO:175); a VL CDR2 having the amino acid sequence SASYRY (SEQ ID NO:176); a VL CDR3 having the amino acid sequence QQYYIYPY (SEQ ID NO:177); a VH CDR1 having the amino acid sequence GFTFTDYTMD (SEQ ID NO:178); a VH CDR2 having the amino acid sequence ADVNPNSGGSIYNQRFKG (SEQ ID NO:179); and a VH CDR3 having the amino acid sequence ARNLGPSFYFDY (SEQ ID NO:180).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 170)
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYWGQGTL
VTVSSGGGGSGGGGSGGGGSDIQMTQSPSSLSASVGDRVTITCRASQDVNTAVAWYQQKPGKA
PKLLIYSASFLYSGVPSRFSGSRSGTDFTLTISSLQPEDFATYYCQQHYTTPPTFGQGTKVEIK.

### Anti-CD19

Anti-CD19 antibodies are known in the art; and the VH and VL, or the VH and VL CDRs, of any anti-CD19 antibody can be used. See e.g., WO 2005/012493.

In some cases, an anti-CD19 antibody includes a VL CDR1 comprising the amino acid sequence KASQSVDYDGDSYLN (SEQ ID NO:181); a VL CDR2 comprising the amino acid sequence DASNLVS (SEQ ID NO:182); and a VL CDR3 comprising the amino acid sequence QQSTEDPWT (SEQ ID NO:183). In some cases, an anti-CD19 antibody includes a VH CDR1 comprising the amino acid sequence SYWMN (SEQ ID NO:184); a VH CDR2 comprising the amino acid sequence QIWPGDGDTNYNGKFKG (SEQ ID NO:185); and a VH CDR3 comprising the amino acid sequence RETTTVGRYYYAMDY (SEQ ID NO:186). In some cases, an anti-CD19 antibody includes a VL CDR1 comprising the amino acid sequence KASQSVDYDGDSYLN (SEQ ID NO:181); a VL CDR2 comprising the amino acid sequence DASNLVS (SEQ ID NO:182); a VL CDR3 comprising the amino acid sequence QQSTEDPWT (SEQ ID NO:183); a VH CDR1 comprising the amino acid sequence SYWMN (SEQ ID NO:184); a VH CDR2 comprising the amino acid sequence QIWPGDGDTNYNGKFKG (SEQ ID NO:185); and a VH CDR3 comprising the amino acid sequence RETTTVGRYYYAMDY (SEQ ID NO:186).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 187)
DIQLTQSPASLAVSLGQRATISCKASQSVDYDGDSYLNWYQQIPGQPPKLLIYDASNLVSGIPPRF
SGSGSGTDFTLNIHPVEKVDAATYHCQQSTEDPWTFGGGTKLEIKGGGGSGGGGSGGGGSQVQ
LQQSGAELVRPGSSVKISCKASGYAFSSYWMNWVKQRPGQGLEWIGQIWPGDGDTNYNGKFK
GKATLTADESSSTAYMQLSSLASEDSAVYFCARRETTTVGRYYYAMDYWGQGTTVTVS.

### Anti-Mesothelin

Anti-mesothelin antibodies are known in the art, see, e.g., U.S. 2019/0000944; WO 2009/045957; WO 2014/031476; U.S. Pat. No. 8,460,660; US 2013/0066055; and WO 2009/068204.

In some cases, an anti-mesothelin antibody comprises: a) a light chain comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSW YQQHPGKAPKLMIYGVNNRPSGVSNRFS GSKSGNTASLTISGLQAEDEADYYCSSYDIESATPVFGGGTKLTVLGQPKAAPSVTLFPPSSEELQ ANKATLVCLISDFYPGAVTVAWKGDSSPVKAGVETTTPSKQSNNKYAASSYLSLTPEQWKSHR SYSCQVTHEGSTVEKTVAPTESS (SEQ ID NO:215); and
b) a heavy chain comprising an amino acid sequence having at least 90%, at least 95%, at least 98%, at least 99%, or 100%, amino acid sequence identity to the following amino acid sequence:

(SEQ ID NO: 216)
QVELVQSGAEVKKPGESLKISCKGSGYSFTSYWIGWVRQAPGKGLEWMGIIDPG
DSRTRYSPSFQGQVTISADKSISTAYLQWSSLKASDTAMYYCARGQLYGGTYMDGWGQGTLV
TVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVKG
FYPSDIAEWESNGQPENNYKTTPPVLDSDGSFFLYSKLTVDKSRWQQGNVFSCSVMHEALHN
HYTQKSLSLSPGK.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO:217); and b) a VH comprising an amino acid sequence having at least 90%, at least 95%, at least 98%, at least 99%, or 100%, amino acid sequence identity to the amino acid sequence: QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:218).

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above.

For example, an anti-mesothelin antibody can comprise a VL CDR1 having the amino acid sequence TGTSSDIGGYNSVS (SEQ ID NO:219); a VL CDR2 having the amino acid sequence LMIYGVNNRPS (SEQ ID NO:220); a VL CDR3 having the amino acid sequence SSYDIESATP (SEQ ID NO:221); a VH CDR1 having the amino acid sequence GYSFTSYWIG (SEQ ID NO:222); a VH CDR2 having the amino acid sequence WMGIIDPGDSRTRYSP (SEQ ID NO:223); and a VH CDR3 having the amino acid sequence GQLYGGTYMDG (SEQ ID NO:224).

An anti-mesothelin antibody can be a scFv. As one non-limiting example, an anti-mesothelin scFv can comprise the following amino acid sequence: QVQLQQSGAEVKKPGASVKVSCKASGYTFTGYYMHWVRQAPGQGLEWMGRINPNSGGTNYA QKFQGRVTMTRDTSISTAYMELSRLRSEDTAVYYCARGRYYGMDVWGQGTMVTVSSGGGGS GGGGSGGGGSGGGGSEIVLTQSPATLSLSPGERATISCRASQSVSSNFAWYQQRPGQAPRLLIYD ASNRATGIPPRFSGSGSGTDFTLTISSLEPED FAAYYCHQRSNWLYTFGQGTKVDIK (SEQ ID NO:225), where VH CDR1, CDR2, and CDR3 are underlined; and VL CDR1, CDR2, and CDR3 are bolded and underlined.

As one non-limiting example, an anti-mesothelin scFv can comprise the following amino acid sequence: QVQLVQSGAEVKKPGASVKVSCKASGYTFTGYYMHWVRQAPGQGLEWMGWINPNSGGTNY AOKFOGRVTMTRDTSISTAYMELSRLRSDDTAVYYCARDLRRTVVTPRAYYGMDVWGQGTTV TVSSGGGGSGGGGSGGGGSGGGGSDIQLTQSPSTLSASVGDRVTITCOASQDISNSLNWYQQKA GKAPKLLIYDASTLETGVPSRFSGSGSGTDFSF TISSLQPEDIATYYCQQHDNLPLTFGQGTKVEIK (SEQ ID NO:226), where VH CDR1, CDR2, and CDR3 are underlined; and VL CDR1, CDR2, and CDR3 are bolded and underlined.

### Anti-CD22

CD22 (also known as B-Lymphocyte Cell Adhesion Molecule, Sialic Acid-Binding Ig-Like Lectin 2, or SIGLEC2) is a sialic acid-binding adhesion molecule largely restricted to the B cell lineage and expressed on most B-lineage malignancies.

Anti-CD22 antibodies are known in the art; and the VH and VL, or the VH and VL CDRs, of any anti-CD22 antibody can be used. See, e.g., Xiao et al. (2009) Mabs 1:297 (describing the fully human anti-CD22 m971 scFv); and U.S. Patent Publication No. 2020/0147134. Examples of anti-CD22 antibodies include epratuzumab and inotuzumab. See, e.g., Lenoard et al. (2007) Oncogene 26:3704 and U.S. Pat. No. 5,789,554 (describing epratuzumab); and DiJoseph et al. (2007) Leukemia 21:2240 (describing inotuzumab).

For example, an anti-CD22 antibody can comprise: i) a heavy chain variable region (VH) CDR1 having the amino acid sequence: GDSVSSNSAA (SEQ ID NO:227); ii) a VH CDR2 having the amino acid sequence: TYYRSKWYN (SEQ ID NO:228); iii) a VH CDR3 having the amino acid sequence: AREVTGDLEDAFDI (SEQ ID NO:229); iv) a light chain variable region (VL) CDR1 having the amino acid sequence: QTIWSY (SEQ ID NO:230); v) a VL CDR2 having the amino acid sequence: AAS (Ala-Ala-Ser); and vi) a VL CDR3 having the amino acid sequence: QQSYSIPQT (SEQ ID NO:231).

### Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene.

Anti-TROP-2 antibodies are known in the art; and the VH and VL, or the VH and VL CDRs, of any anti-TROP-2 antibody can be used. See, e.g., U.S. Pat. No. 7,238,785). In some cases, an anti-TROP-2 antibody comprises: i) light chain CDR sequences CDR1 (KASQDVSIAVA; SEQ ID NO:232); CDR2 (SASYRYT; SEQ ID NO:233); and CDR3 (QQHYITPLT; SEQ ID NO:234); and ii) heavy chain CDR sequences CDR1 (NYGMN; SEQ ID NO:235); CDR2 (WINTYTGEPTYTDDFKG; SEQ ID NO:236); and CDR3 (GGFGSSYWYFDV; SEQ ID NO:237).

In some cases, an anti-TROP-2 antibody comprises: i) heavy chain CDR sequences CDR1 (TAGMQ; SEQ ID NO:238); CDR2 (WINTHSGVPKYAEDFKG (SEQ ID NO:239); and CDR3 (SGFGSSYWYFDV; SEQ ID NO:240); and ii) light chain CDR sequences CDR1 (KASQDVSTAVA; SEQ ID NO:241); CDR2 (SASYRYT; SEQ ID NO:233); and CDR3 (QQHYITPLT; SEQ ID NO:234).

In some cases, an anti-TROP2 antibody comprises: a) VL CDR1, VL CDR2, and VL CDR3 present in a light chain variable region (VL) comprising the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:244); and b) VH CDR1, CDR2, and CDR3 present in a heavy chain variable region (VH) comprising the following amino acid sequence:

(SEQ ID NO: 245)
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSS.

In some cases, an anti-TROP-2 antibody comprises: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:244); and b) a VH region comprising an amino acid sequence having at least 90%, at least 95%, at least 98%, at least 99%, or 100%, amino acid sequence identity to the following amino acid sequence:

(SEQ ID NO: 245)
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSS.

### Anti-BCMA

Anti-BCMA (B-cell maturation antigen) antibodies are known in the art; and the VH and VL, or the VH and VL CDRs, of any anti-BCMA antibody can be used. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:

QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLGQPKAAPSV TLFPPSSEELQANKATLVCLISDFYPGAVTVAWKADSSPVKAGVETTTPDSKQSNNKYAASSYL SLTPEQWKSHRSYSCQVTHEGSTVEKTVAPTECS (SEQ ID NO:248); and

b) a heavy chain comprising an amino acid sequence having at least 90%, at least 95%, at least 98%, at least 99%, or 100%, amino acid sequence identity to the following amino acid sequence:

(SEQ ID NO: 249)
EVQLVESGGGLVKPGGSLRLSCAASGFTFGDYALSWFRQAPGKGLEWVGVSRSKAYGGTTDY
AASVKGRFTISRDDSKSTAYLQMNSLKTEDTAVYYCASSGYSSGWTPFDYWGQGTLVTVSSAS
TKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQ
KSLSLSPGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:

QSVLTQPPSASGTPGQRVTISCSGSSSNIGSNTVNWYQQLPGTAPKLLIFNYHQRP SGVPDRFSGSKSGSSASLAiSGLQSEDEADYYCAAWDDSLNGWVFGGGTKLTVLG (SEQ ID NO:250); and b) a VH comprising an amino acid sequence having at least 90%, at least 95%, at least 98%, at least 99%, or 100%, amino acid sequence identity to the amino acid sequence:

(SEQ ID NO: 251)
EVQLVESGGGLVKPGGSLRLSCAASGFTFGDYALSWFRQAPGKGLEWVG
VSRSKAYGGTTDYAASVKGRFTISRDDSKSTAYLQMNSLKTEDTAVYYC
ASSGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above.

For example, an anti-BCMA antibody can comprise a VL CDR1 having the amino acid sequence SSNIGSNT (SEQ ID NO:252), a VL CDR2 having the amino acid sequence NYH, a VL CDR3 having the amino acid sequence AAWDDSLNGWV (SEQ ID NO:253)), a VH CDR1 having the amino acid sequence GFTFGDYA (SEQ ID NO:254), a VH CDR2 having the amino acid sequence SRSKAYGGTT (SEQ ID NO:255), and a VH CDR3 having the amino acid sequence ASSGYSSGWTPFDY (SEQ ID NO:256).

An anti-BCMA antibody can be a scFv. As one non-limiting example, an anti-BCMA scFv can comprise the following amino acid sequence:

(SEQ ID NO: 257)
QVQLVQSGAEVKKPGSSVKVSCKASGGTFSNYWMHWVRQAPGQGLEWMGATYRGHSDTYY
NQKFKGRVTITADKSTSTAYMELSSLRSEDTAVYYCARGAIYNGYDVLDNWGQGTLVTVSSGG
GGSGGGGSGGGGSGGGGSDIQMTQSPSSLSASVGDRVTITCSASQDISNYLNWYQQKPGKAPKL
LIYYTSNLHSGVPSRFSGSGSGTDFTLTISSLQPEDFATYYCQQYRKLPWTFGQGTKLEIKR.

As another example, an anti-BCMA scFv can comprise the following amino acid sequence:

(SEQ ID NO: 258)
DIQMTQSPSSLSASVGDRVTITCSASQDISNYLNWYQQKPGKAPKLLIYYTSNLHSGVPSRFSGS
GSGTDFTLTISSLQPEDFATYYCQQYRKLPWTFGQGTKLEIKRGGGGSGGGGSGGGGSGGGGSQ
VQLVQSGAEVKKPGSSVKVSCKASGGTFSNYWMHWVRQAPGQGLEWMGATYRGHSDTYYN
QKFKGRVTITADKSTSTAYMELSSLRSEDTAVYYCARGAIYNGYDVLDNWGQGTLVTVSS.

In some cases, an anti-BCMA antibody can comprise a VL CDR1 having the amino acid sequence SASQDISNYLN (SEQ ID NO:259); a VL CDR2 having the amino acid sequence YTSNLHS (SEQ ID NO:260); a VL CDR3 having the amino acid sequence QQYRKLPWT (SEQ ID NO:261); a VH CDR1 having the amino acid sequence NYWMH (SEQ ID NO:262); a VH CDR2 having the amino acid sequence ATYRGHSDTYYNQKFKG (SEQ ID NO:263); and a VH CDR3 having the amino acid sequence GAIYNGYDVLDN (SEQ ID NO:264).

In some cases, an anti-BCMA antibody comprises: a) a light chain comprising an amino acid sequence having at least 90%, at least 95%, at least 98%, at least 99%, or 100%, amino acid sequence identity to the following amino acid sequence: DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:265).

In some cases, an anti-BCMA antibody comprises: a) a heavy chain comprising an amino

(SEQ ID NO: 265)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKR.

acid sequence having at least 90%, at least 95%, at least 98%, at least 99%, or 100%, amino acid sequence identity to the following amino acid sequence:

(SEQ ID NO: 266)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

### Anti-MUC16

In some cases, an antibody is specific for MUC16 (also known as CA125). See, e.g., Yin et al. (2002) Int. J. Cancer 98:737. For example, an antibody can be specific for a MUC16 polypeptide present on a cancer cell. See, e.g., US 2018/0118848; and US 2018/0112008. In some cases, a MUC16-specific antibody is a scFv. In some cases, a MUC16-specific antibody is a nanobody.

As one example, an anti-MUC16 antibody can comprise a VH CDR1 having the amino acid sequence GFTFSNYY (SEQ ID NO:267); a VH CDR2 having the amino acid sequence ISGRGSTI (SEQ ID NO:268); a VH CDR3 having the amino acid sequence VKDRGGYSPY (SEQ ID NO:269); a VL CDR1 having the amino acid sequence QSISTY (SEQ ID NO:270); a VL CDR2 having the amino acid sequence TAS; and a VL CDR3 having the amino acid sequence QQSYSTPPIT (SEQ ID NO:271). See, e.g., US 2018/0118848.

### Anti-Claudin-18.2

In some cases, an antibody is specific for claudin-18 isoform 2 (“claudin-18.2”). See, e.g., WO 2013/167259. In some cases, a claudin-18.2-specific antibody is a scFv. In some cases, a claudin-18.2-specific antibody is a nanobody.

As one example, an anti-claudin-18.2 antibody can comprise a VH CDR1 having the amino acid sequence GYTFTDYS (SEQ ID NO:272); a VH CDR2 having the amino acid sequence INTETGVP (SEQ ID NO:273); a VH CDR3 having the amino acid sequence ARRTGFDY (SEQ ID NO:274); a VL CDR1 having the amino acid sequence KNLLHSDGITY (SEQ ID NO:275); a VL CDR2 having the amino acid sequence RVS; and a VL CDR3 having the amino acid sequence VQVLELPFT (SEQ ID NO:276).

As another example, an anti-claudin-18.2 antibody can comprise a VH CDR1 having the amino acid sequence GFTFSSYA (SEQ ID NO:277); a VH CDR2 having the amino acid sequence ISDGGSYS (SEQ ID NO:278); a VH CDR3 having the amino acid sequence ARDSYYDNSYVRDY (SEQ ID NO:279); a VL CDR1 having the amino acid sequence QDINTF (SEQ ID NO:280); a VL CDR2 having the amino acid sequence RTN; and a VL CDR3 having the amino acid sequence LQYDEFPLT (SEQ ID NO:281).

Examples of antibodies include, but are not limited to: Natalizumab (Tysabri®; Biogen Idec/Elan) targeting a4 subunit of a41 andu407 integrins (used in the treatment of MS and Crohn's disease); Vedolizumab (MLN2; Millennium Pharmaceuticals/Takeda) targeting a47 integrin (as used in the treatment of UC and Crohn's disease); Belimumab (Benlysta; Human Genome Sciences/GlaxoSmithKline) targeting BAFF (as used in the treatment of SLE); Atacicept (TACI-Ig; Merck/Serono) targeting BAFF and APRIL (as used in the treatment of SLE); Alefacept (Amevive®; Astellas) targeting CD2 (as used in the treatment of Plaque psoriasis, GVHD); Otelixizumab (TRX4; Tolerx/GlaxoSmithKline) targeting CD3 (as used in the treatment of T1D); Teplizumab (MGA031; MacroGenics/Eli Lilly) targeting CD3 (as used in the treatment of T1D); Rituximab (Rituxan®/Mabthera; Genentech/Roche/Biogen Idec) targeting CD20 (as used in the treatment of Non-Hodgkin's lymphoma, RA (in patients with inadequate responses to TNF blockade) and CLL); Ofatumumab (Arzerra®; Genmab/GlaxoSmithKline) targeting CD20 (as used in the treatment of CLL, RA); Ocrelizumab (2H7; Genentech/Roche/Biogen Idec) targeting CD20 (as used in the treatment of RA and SLE); Epratuzumab (hLL2; Immunomedics/UCB) targeting CD22 (as used in the treatment of SLE and non-Hodgkin's lymphoma); Alemtuzumab (Campath®/MabCampath; Genzyme/Bayer) targeting CD52 (as used in the treatment of CLL, MS); Abatacept (Orencia®; Bristol-Myers Squibb) targeting CD80 and CD86 (as used in the treatment of RA and JIA, UC and Crohn's disease, SLE); Eculizumab (Soliris®; Alexion pharmaceuticals) targeting C5 complement protein (as used in the treatment of Paroxysmal nocturnal haemoglobinuria); Omalizumab (Xolair®; Genentech/Roche/Novartis) targeting IgE (as used in the treatment of Moderate to severe persistent allergic asthma); Canakinumab (Ilaris®; Novartis) targeting IL-I1 (as used in the treatment of Cryopyrin-associated periodic syndromes, Systemic JIA, neonatal-onset multisystem inflammatory disease and acute gout); Mepolizumab (Bosatria; GlaxoSmithKline) targeting IL-5 (as used in the treatment of Hyper-eosinophilic syndrome); Reslizumab (SCH55700; Ception Therapeutics) targeting IL-5 (as used in the treatment of Eosinophilic oesophagitis); Tocilizumab (Actemra®/RoActemra®; Chugai/Roche) targeting IL-6R (as used in the treatment of RA, JIA); Ustekinumab (Stelara®; Centocor) targeting IL-12 and IL-23 (as used in the treatment of Plaque psoriasis, Psoriatic arthritis, Crohn's disease); Briakinumab (ABT-874; Abbott) targeting IL-12 and IL-23 (as used in the treatment of Psoriasis and plaque psoriasis); Etanercept (Enbrel®; Amgen/Pfizer) targeting TNF (as used in the treatment of RA, JIA, psoriatic arthritis, AS and plaque psoriasis); Infliximab (Remicade®; Centocor/Merck) targeting TNF (as used in the treatment of Crohn's disease, RA, psoriatic arthritis, UC, AS and plaque psoriasis); Adalimumab (Humira®/Trudexa; Abbott) targeting TNF (as used in the treatment of RA, JIA, psoriatic arthritis, Crohn's disease, AS and plaque psoriasis); Certolizumab pegol (Cimnzia®; UCB) targeting TNF (as used in the treatment of Crohn's disease and RA); Golimumab (Simponi®; Centocor) targeting TNF (as used in the treatment of RA, psoriatic arthritis and AS); and the like. In some cases, the antibody whose production is induced by the intracellular domain of a synNotch polypeptide of the present disclosure is a therapeutic antibody for the treatment of cancer. Such antibodies include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MED14736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, chl4.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

Examples of antibodies include, but are not limited to: Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexclizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Scribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBSO7, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like Antibody mimetics

In some cases, an EDV of the present disclosure comprises an antibody mimetic (also referred to as an “antibody analog”). Non-limiting examples of antibody mimetics include peptide aptamers, affimers, affilins, affibodies, affitins, alphabodies, anticalins, avimers, DARPins, fynomers, Kunitz domain peptides, nanoCLAMPs, affinity reagents, and scaffold proteins.

### Nucleic Acid-Bindings Polypeptides

As noted above, the present disclosure provides EDVs comprising a nucleic acid-binding effector polypeptide, or a nucleic acid encoding the nucleic acid-binding effector polypeptide, where the EDV comprises a fusion polypeptide comprising (i) a viral envelope protein and (ii) a targeting polypeptide that provides for binding to a target cell. The present disclosure provides methods of delivering a nucleic acid-binding effector polypeptide into a eukaryotic cell, using an EDV of the present disclosure.

Suitable nucleic acid binding effector polypeptides include nucleases. Suitable nucleases include, but are not limited to, a homing nuclease polypeptide; a Foki polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

CRISPR-Cas effector polypeptides

As noted above, in some cases, an EDV of the present disclosure comprises a CRISPR-Cas effector polypeptide, or a nucleic acid (e.g., a recombinant expression vector) comprising a nucleotide sequence encoding a CRISPR-Cas effector polypeptide. The CRISPR-Cas effector polypeptide can be any of a variety of CRISPR-Cas effector polypeptides. Suitable CRISPR-Cas effector polypeptides are described in detail below. For example, in some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Casl2e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a CasI4a polypeptide, a Casl4b polypeptide, or a Casl4c polypeptide. Also suitable for use is a variant CRISPR-Cas effector polypeptide, where the variant CRISPR-Cas effector polypeptide has reduced nucleic acid cleavage activity. Also suitable for use is a CRISPR-Cas effector fusion polypeptide comprising: i) a CRISPR-Cas effector polypeptide is a variant that has reduced nucleic acid cleavage activity; and ii) a heterologous fusion polypeptide. In some cases, the heterologous fusion polypeptide is a protein modifying enzyme. In some cases, the heterologous fusion polypeptide is a nucleic acid modifying enzyme. In some cases, the heterologous fusion polypeptide is a reverse transcriptase. In some cases, the heterologous fusion polypeptide is a cytidine deaminase. In some cases, the heterologous fusion polypeptide is an adenine deaminase. In some cases, the heterologous fusion polypeptide is a transcription factor. In some cases, the heterologous fusion polypeptide is a transcription activator. In some cases, the heterologous fusion polypeptide is a transcription repressor. Suitable protein-modifying enzymes and nucleic acid modifying enzymes are described in detail below. For example, in some cases, the nucleic acid modifying enzyme is a cytidine deaminase. In some cases, the nucleic acid modifying enzyme is an adenosine deaminase. In some cases, the nucleic acid modifying enzyme is a prime editor. As described in more detail below, in some cases, the CRISPR-Cas effector polypeptide comprises one or more nuclear localization signals.

Examples of CRISPR-Cas effector polypeptides are CRISPR-Cas endonucleases (e.g., class 2 CRISPR-Cas effector polypeptide such as a type II, type V, or type VI CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have reduced or undetectable endonuclease activity. A CRISPR-Cas effector polypeptide can also be a fusion CRISPR-Cas effector polypeptide comprising a heterologous fusion partner. In some cases, a suitable CRISPR-Cas effector polypeptide is a class 2 CRISPR-Cas effector polypeptide. In some cases, a suitable CRISPR-Cas effector polypeptide is a class 2 type 11 CRISPR-Cas effector polypeptide (e.g., a Cas9 protein). In some cases, a suitable CRISPR-Cas effector polypeptide is a class 2 type V CRISPR-Cas endonuclease (e.g., a Cpf1 protein, a C2cl protein, or a C2c3 protein). In some cases, a suitable CRISPR-Cas effector polypeptide is a class 2 type VI CRISPR-Cas effector polypeptide (e.g., a C2c2 protein; also referred to as a “Casl3a” protein). Also suitable for use is a CasX protein. Also suitable for use is a CasY protein.

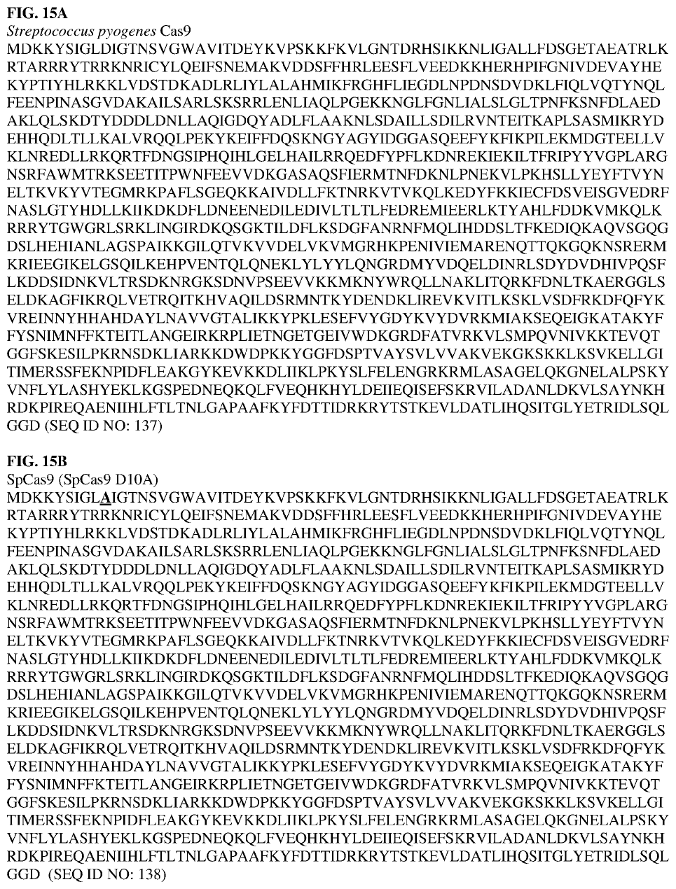

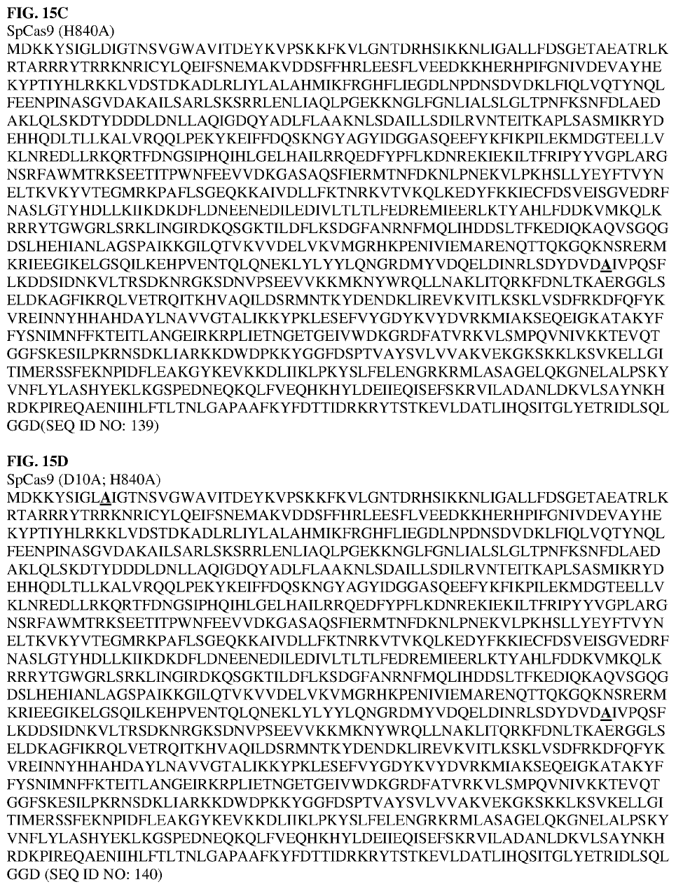

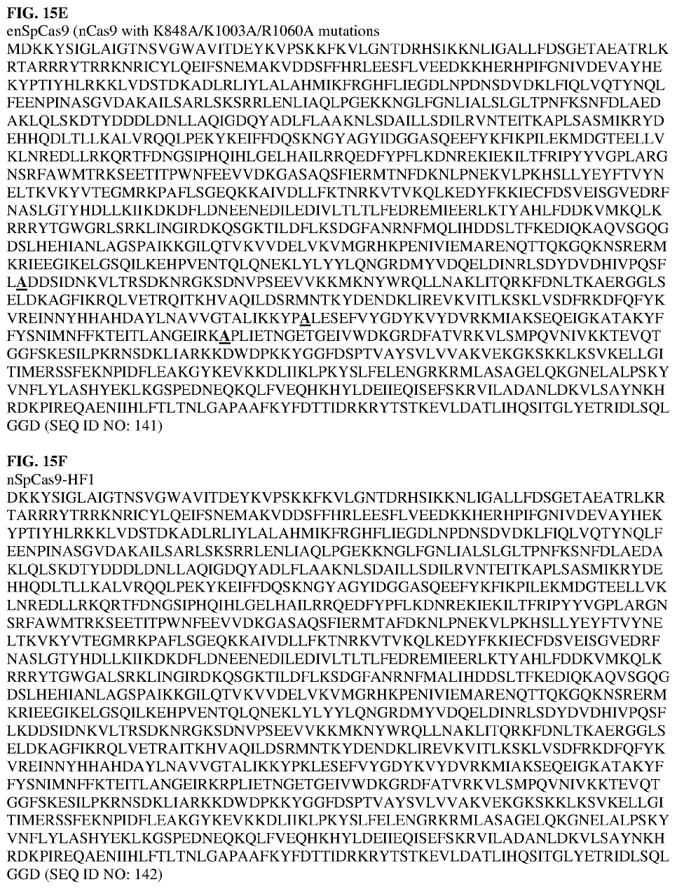

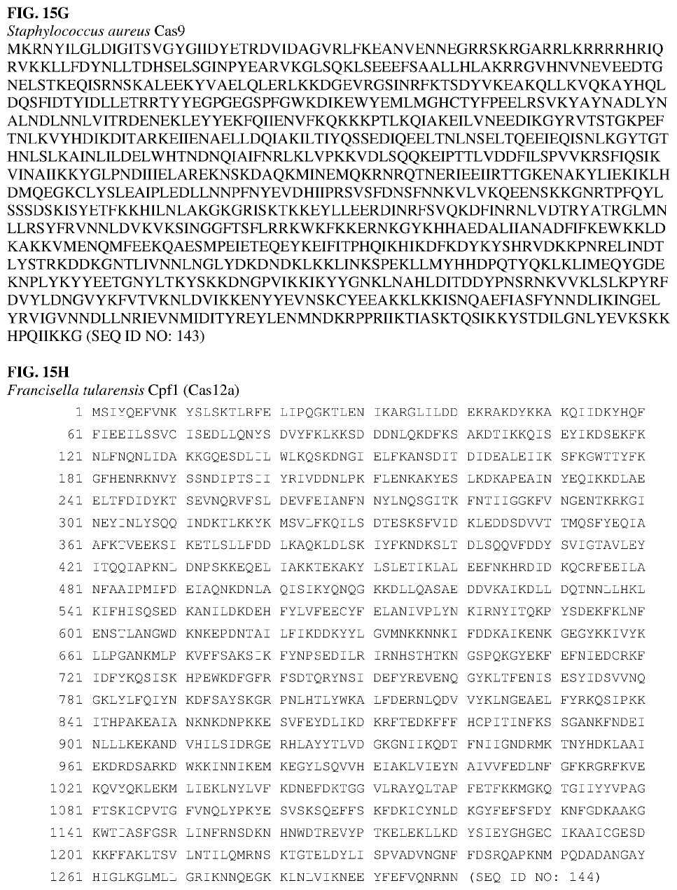

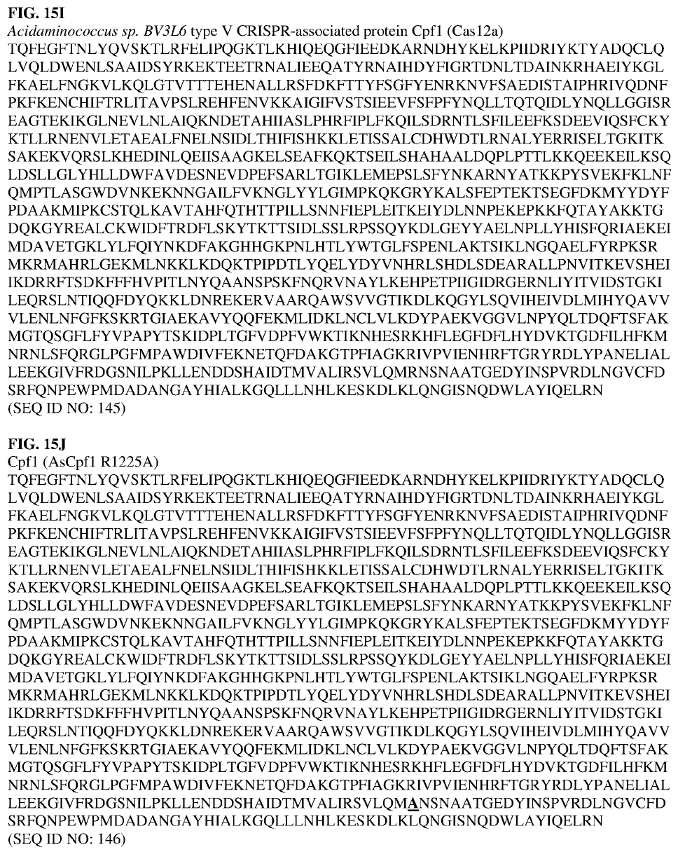

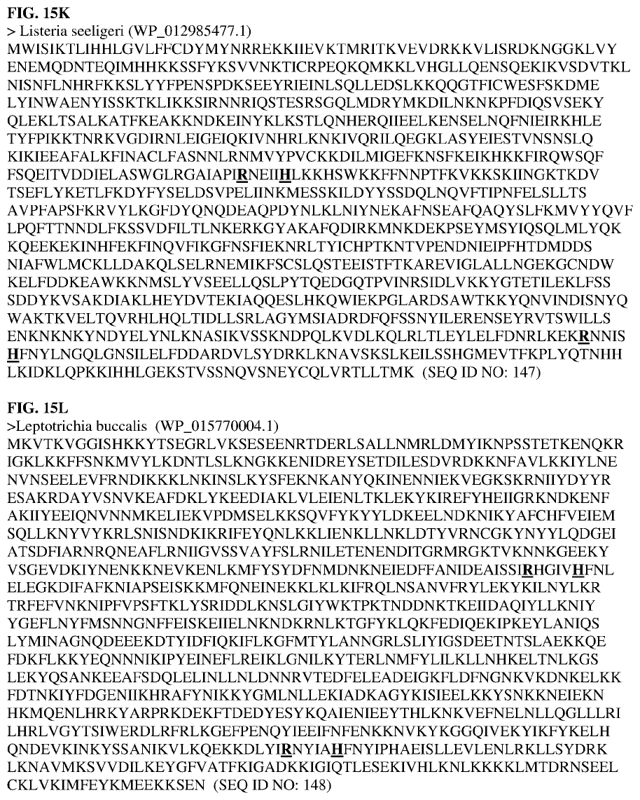

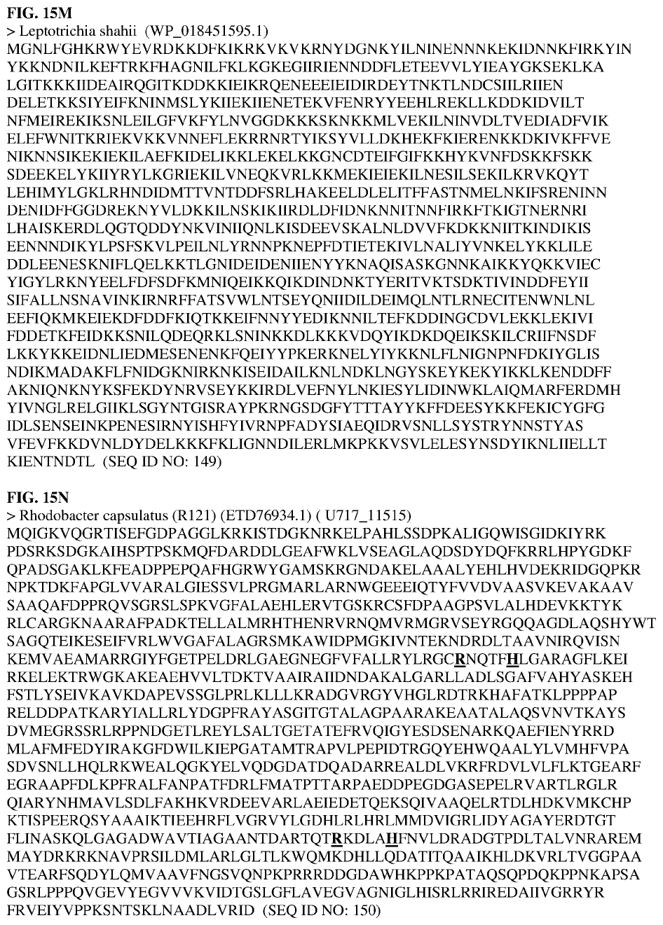

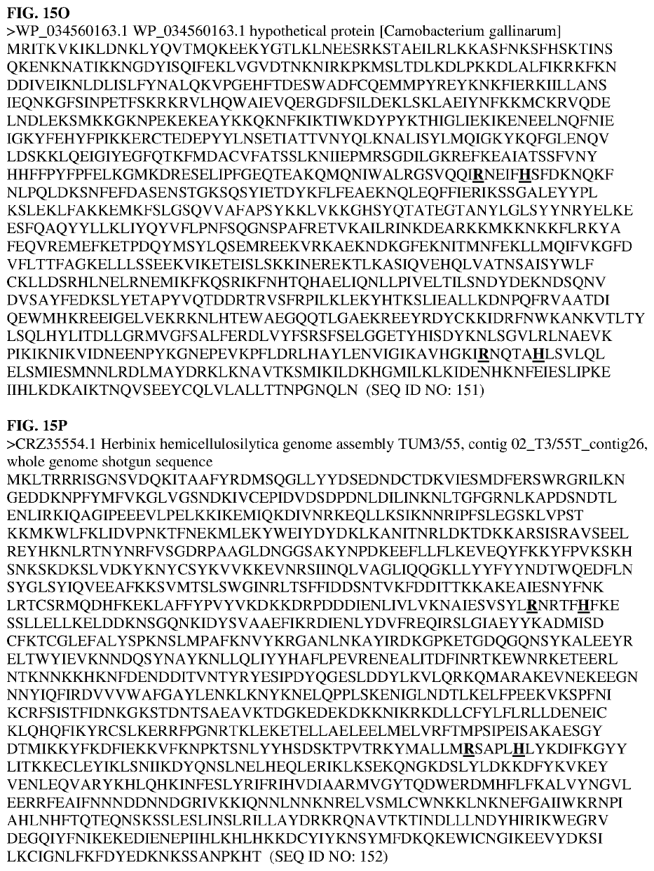

In some cases, a suitable CRISPR-Cas effector polypeptide comprises an amino acid sequence having at least 85%, at least 90%, at least 95%, at least 98%, at least 99%, or 100%, amino acid sequence identity to the amino acid sequence depicted in any one of FIG. 15A-15P.

In some cases, the CRISPR-Cas effector polypeptide is a Type 11 CRISPR-Cas effector polypeptide. In some cases, the CRISPR-Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 15A.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 15G.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 15A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 15A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable CRISPR-Cas effector polypeptide is a type V CRISPR-Cas effector polypeptide. In some cases, a type V CRISPR-Cas effector polypeptide is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 15H, FIG. 15I, or FIG. 15J.

In some cases, a suitable CRISPR-Cas effector polypeptide is a CasX or a CasY polypeptide. CasX and CasY polypeptides are described in Burstein et al. (2017) Nature 542:237.

In some cases, a suitable CRISPR-Cas effector polypeptide is a fusion protein comprising a CRISPR-Cas effector polypeptide that is fused to a heterologous polypeptide (also referred to as a “fusion partner”). In some cases, a CRISPR-Cas effector polypeptide is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

A nucleic acid that binds to a class 2 CRISPR-Cas effector polypeptide (e.g., a Cas9 protein; a type V or type VI CRISPR-Cas protein; a Cpf1 protein; etc.) and targets the complex to a specific location within a target nucleic acid is referred to herein as a “guide RNA” or “CRISPR-Cas guide nucleic acid” or “CRISPR-Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR-Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, an EDV of the present disclosure comprises a CRISPR-Cas effector polypeptide, or both a CRISPR-Cas effector polypeptide and a guide RNA. In some cases, e.g., where a target nucleic acid comprises a deleterious mutation in a defective allele (e.g., a deleterious mutation in a retinal cell target nucleic acid), the CRISPR-Cas effector polypeptide/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), can be used to correct the deleterious mutation, e.g., via homology-directed repair (HDR).

In some cases, an EDV of the present disclosure comprises: i) a CRISPR-Cas effector polypeptide; and ii) one guide RNA. In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, an EDV of the present disclosure comprises: i) a CRISPR-Cas effector polypeptide; and ii) 2 or more gRNAs, where the two or more gRNAs provide for multiplexed gene knockout, e.g., each of the 2 or more guide RNAs is targeted to a different gene. In some cases, the guide RNAs are sgRNAs. In some cases, the guide RNAs are dgRNAs.

In some cases, an EDV of the present disclosure comprises: i) a CRISPR-Cas effector polypeptide; and ii) 2 separate sgRNAs, where the 2 separate sgRNAs provide for deletion (“knockout”) of a target nucleic acid via non-homologous end joining (NHEJ). In some cases, the guide RNAs are sgRNAs. In some cases, the guide RNAs are dgRNAs.

Class 2 CRISPR-Cas effector polypeptides

In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR-Cas protein” is used herein to encompass the CRISPR-Cas effector polypeptide (e.g., the target nucleic acid cleaving protein) from class 2 CRISPR systems. Thus, the term “class 2 CRISPR-Cas effector polypeptide” as used herein encompasses type II CRISPR-Cas effector polypeptides (e.g., Cas9); type V-A CRISPR-Cas effector polypeptides (e.g., Cpf1 (also referred to a “Cas12a”)); type V-B CRISPR-Cas effector polypeptides (e.g., C2cl (also referred to as “Cas12b”)); type V-C CRISPR-Cas effector polypeptides (e.g., C2c3 (also referred to as “Casl2c”)); type V-U1 CRISPR-Cas effector polypeptides (e.g., C2c4); type V-U2 CRISPR-Cas effector polypeptides (e.g., C2c8); type V-U5 CRISPR-Cas effector polypeptides (e.g., C2c5); type V-U4 CRISPR-Cas proteins (e.g., C2c9); type V-U3 CRISPR-Cas effector polypeptides (e.g., C2c10); type VI-A CRISPR-Cas effector polypeptides (e.g., C2c2 (also known as “Casl3a”)); type VI-B CRISPR-Cas effector polypeptides (e.g., Casl3b (also known as C2c4)); and type VI-C CRISPR-Cas effector polypeptides (e.g., Casl3c (also known as C2c7)). To date, class 2 CRISPR-Cas effector polypeptides encompass type II, type V, and type VI CRISPR-Cas effector polypeptides, but the term is also meant to encompass any class 2 CRISPR-Cas effector polypeptide suitable for binding to a corresponding guide RNA and forming an RNP complex.

In some cases, a CRISPR-Cas effector polypeptide is a fusion polypeptide comprising: i) a CRISPR-Cas effector polypeptide; and 1i) one or more heterologous fusion partners (one or more heterologous fusion polypeptides). In some cases, a fusion CRISPR-Cas effector polypeptide comprises one or more localization signal peptides. In some cases, a fusion CRISPR-Cas effector polypeptide comprises one or more localization signal peptides. Suitable localization signals (“subcellular localization signals”) include, e.g., a nuclear localization signal (NLS) for targeting to the nucleus; a sequence to keep the fusion protein out of the nucleus, e.g., a nuclear export sequence (NES); a sequence to keep the fusion protein retained in the cytoplasm; a mitochondrial localization signal for targeting to the mitochondria; a chloroplast localization signal for targeting to a chloroplast; an endoplasmic reticulum (ER) retention signal; and ER export signal; and the like. In some cases, a fusion CRISPR-Cas effector polypeptide does not include a NLS so that the protein is not targeted to the nucleus (which can be advantageous, e.g., when the target nucleic acid is an RNA that is present in the cytosol). In some cases, a fusion CRISPR-Cas effector polypeptide comprises both an NES and one or more NLSs. A suitable NES comprises hydrophobic amino acid residues, e.g., LXXXLXXLXL (SEQ ID NO:207), where L is a hydrophobic amino acid residue (e.g., Len) and X is any other amino acid. Suitable NESs are known in the art; see, e.g., Xu et al. (2012) Mol. Biol. Cell 23:3677. Non-limiting examples of suitable NESs include: LPPLERLTL (SEQ ID NO:188); LALKLAGLDL (SEQ ID NO:189); LSQALASSFSV (SEQ ID NO:190); NELALKLAGLDI (SEQ ID NO:191). In some cases, the NES comprises the amino acid sequence LPPLERLTL (SEQ ID NO:188).

In some cases, a fusion CRISPR-Cas effector polypeptidc includes (is fused to) a nuclear localization signal (NLS) (e.g., in some cases 2 or more, 3 or more, 4 or more, or 5 or more NLSs). Thus, in some cases, a fusion polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

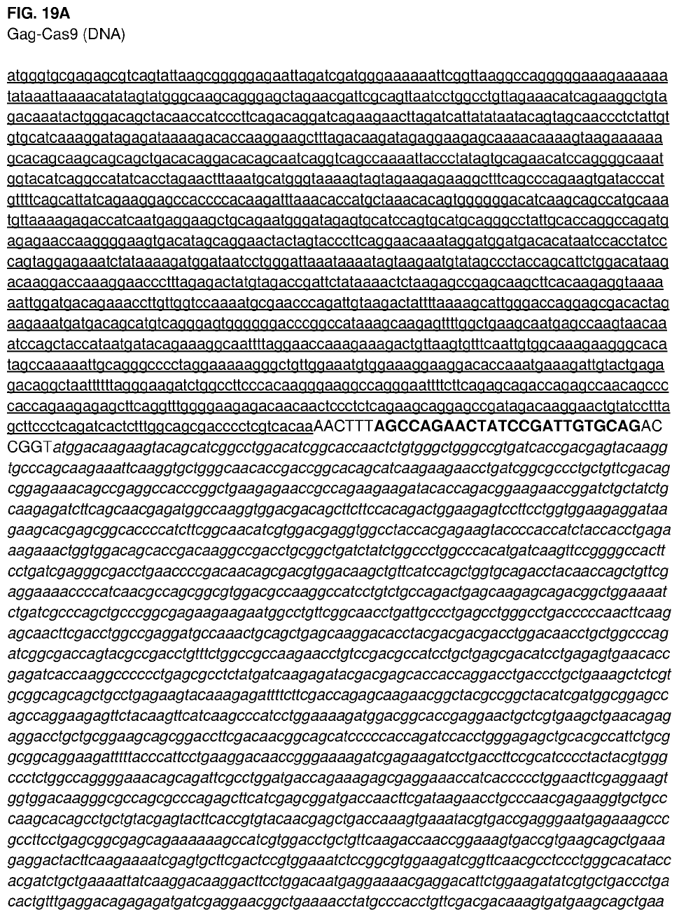

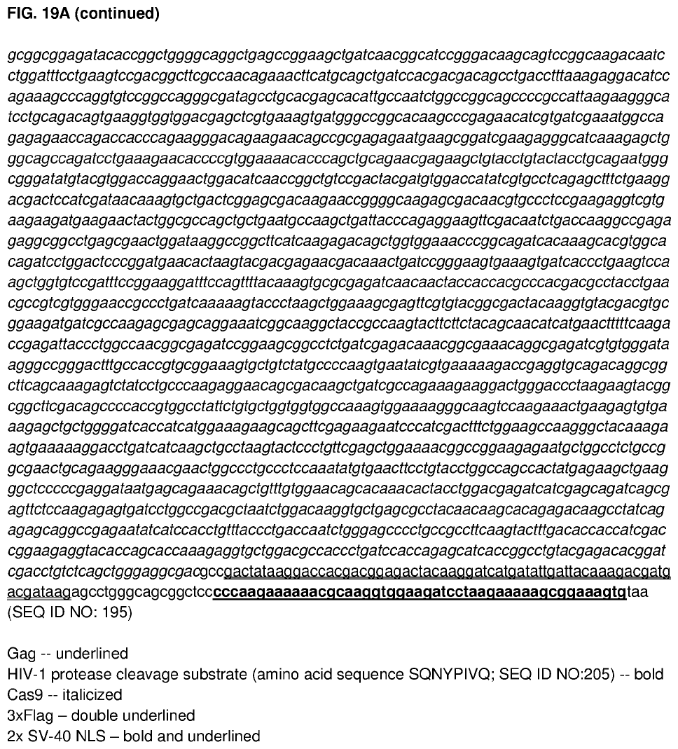

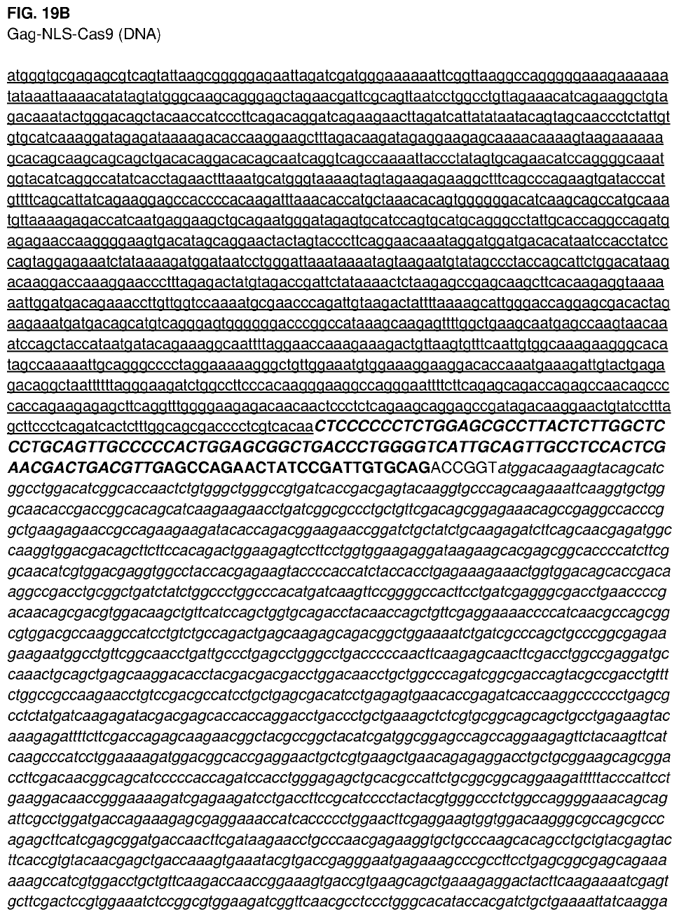

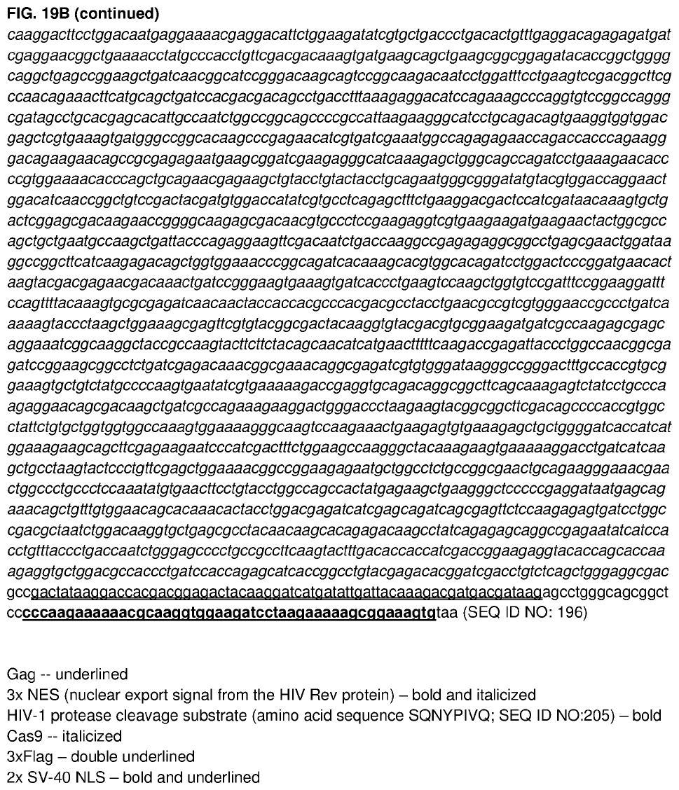

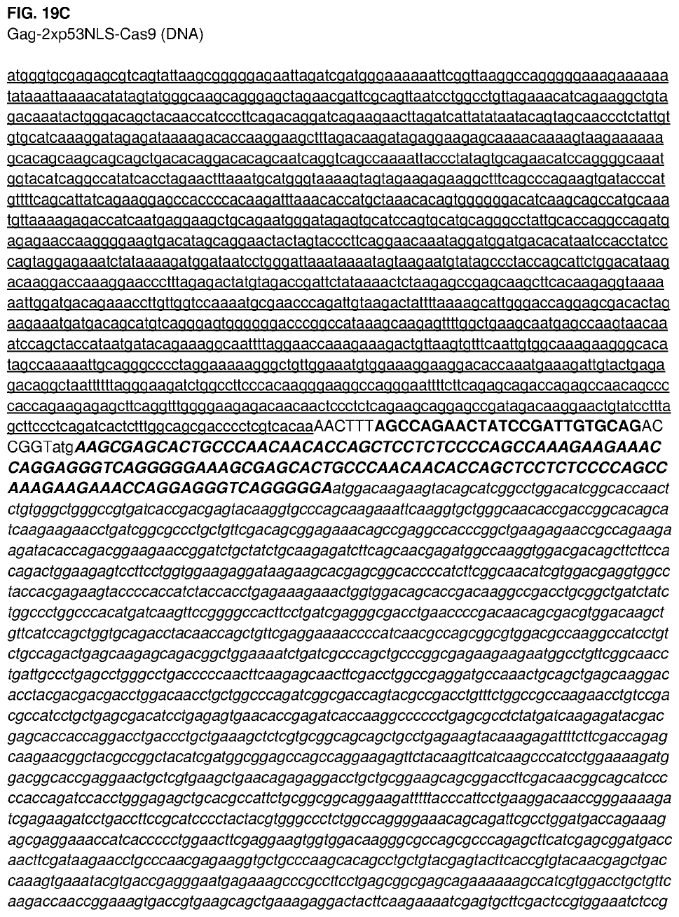

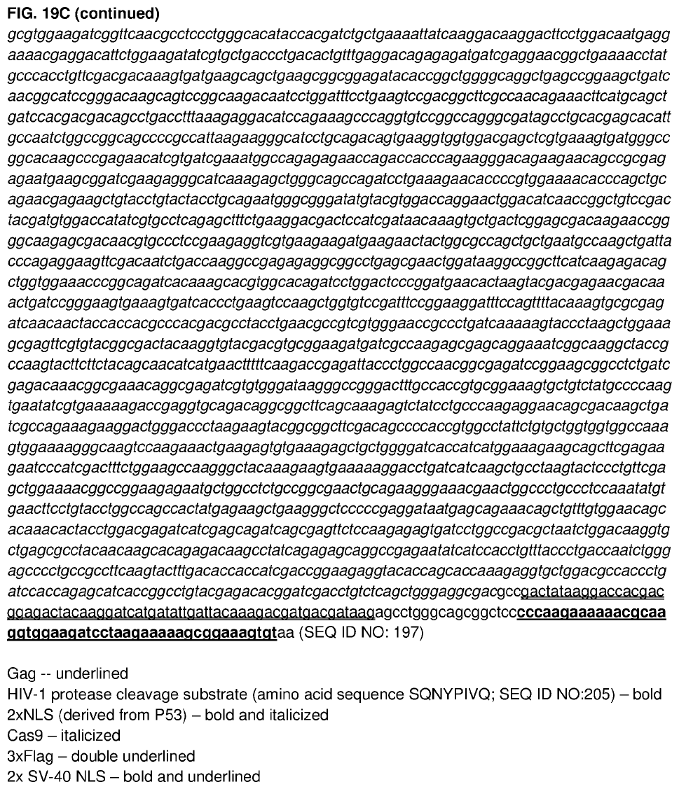

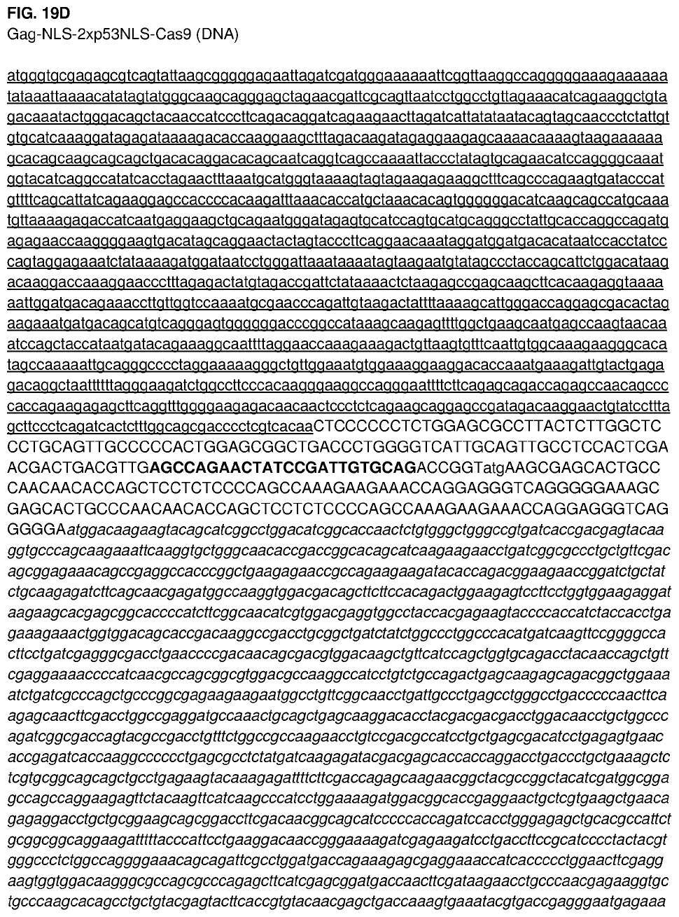

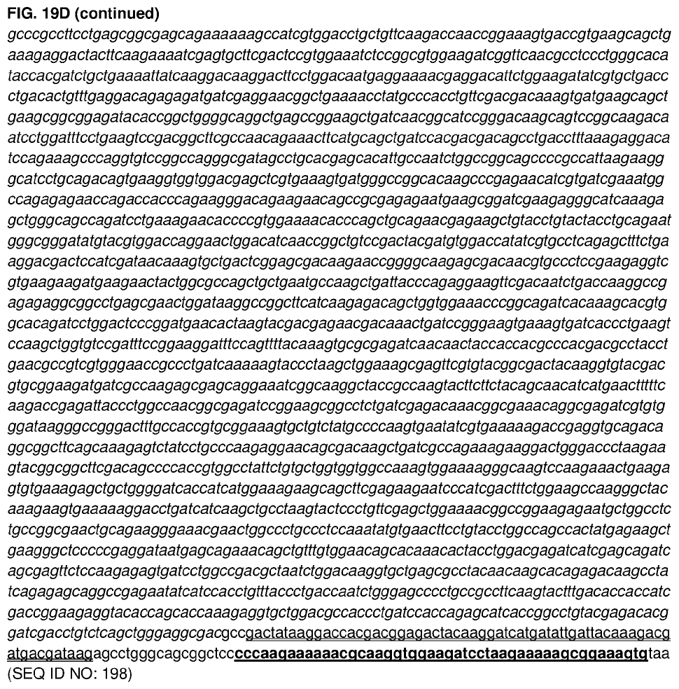

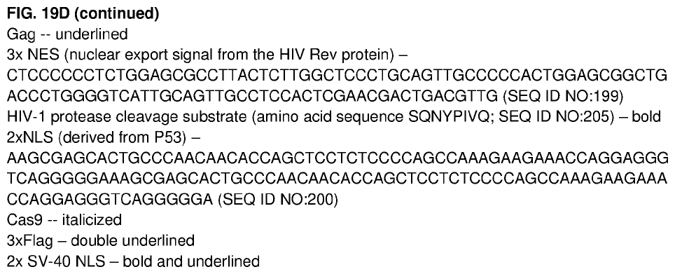

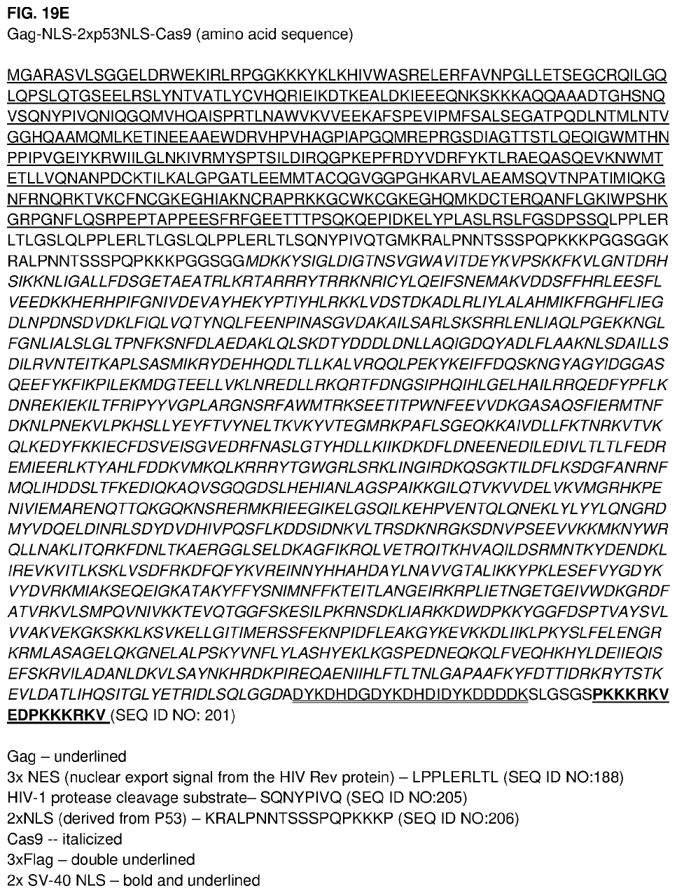

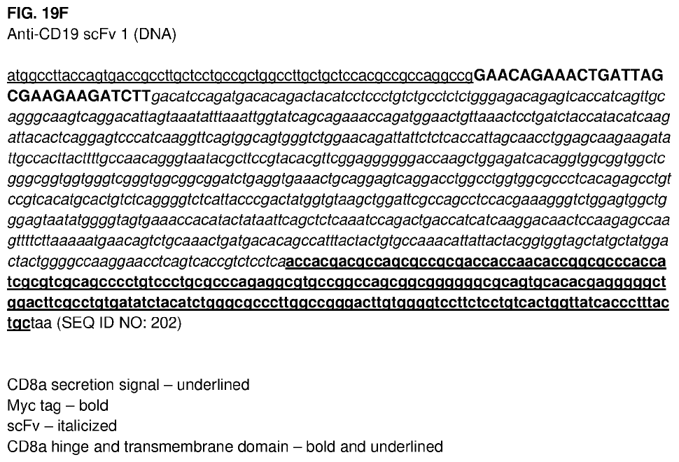

In some cases, a fusion CRISPR-Cas effector polypeptidc comprises: i) a lentiviral Gag polypeptide; ii) a nuclear export signal peptide; iii) 2 copies of an NLS; and iv) a CRISPR-Cas effector polypeptide. Non-limiting examples of nucleotide sequences encoding Gag-Cas9 fusion polypeptides with NES and/or NLS are provided in FIG. 19A-19D. FIG. 19E provides an example of a Gag-Cas9 fusion polypeptide with 3 NES and 2 NLS.

Non-limiting examples of NLSs include an NLS sequence derived from: the NLS of the SV40 virus large T-antigen, having the amino acid sequence PKKKRKV (SEQ ID NO:1); the NLS from nucleoplasmin (e.g., the nucleoplasmin bipartite NLS with the sequence KRPAATKKAGQAKKKK (SEQ ID NO:2)); the c-myc NLS having the amino acid sequence PAAKRVKLD (SEQ ID NO:3) or RQRRNELKRSP (SEQ ID NO:4); the hRNPA1 M9 NLS having the sequence NQSSNFGPMKGGNFGGRSSGPYGGGGQYFAKPRNQGGY (SEQ ID NO:5); the sequence RMRIZFKNKGKDTAELRRRRVEVSVELRKAKKDEQILKRRNV (SEQ ID NO:6) of the IBB domain from importin-alpha; the sequences VSRKRPRP (SEQ ID NO:7) and PPKKARED (SEQ ID NO:8) of the myoma T protein; the sequence PQPKKKPL (SEQ ID NO:9) of human p53; the sequence SALIKKKKKMAP (SEQ ID NO:10) of mouse c-abl IV; the sequences DRLRR (SEQ ID NO:11) and PKQKKRK (SEQ ID NO:16) of the influenza virus NS1; the sequence RKLKKKIKKL (SEQ ID NO:12) of the Hepatitis virus delta antigen; the sequence REKKKFLKRR (SEQ ID NO:13) of the mouse Mxl protein; the sequence KRKGDEVDGVDEVAKKKSKK (SEQ ID NO:14) of the human poly(ADP-ribose) polymerase; and the sequence RKCLQAGMNLEARKTKK (SEQ ID NO:15) of the steroid hormone receptors (human) glucocorticoid. In some cases, an NLS comprises the amino acid sequence MDSLLMNRRKFLYQFKNVRWAKGRRETYLC (SEQ ID NO:17). In general, NLS (or multiple NLSs) are of sufficient strength to drive accumulation of the fusion polypeptide in a detectable amount in the nucleus of a eukaryotic cell. Detection of accumulation in the nucleus may be performed by any suitable technique. For example, a detectable marker may be fused to the fusion polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

### Fusion Polypeptides

As noted above, in some cases, an EDV of the present disclosure comprises a fusion polypeptide comprising: (i) a CRISPR-Cas effector polypeptide; and (ii) one or more heterologous polypeptides. A heterologous polypeptide is also referred to herein as a “fusion partner.” In some cases, a CRISPR-Cas effector polypeptide is fused to one or more heterologous polypeptides that has/have an activity of interest (e.g., a catalytic activity of interest, subcellular localization activity, etc.) to form a fusion protein.

In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like). In some cases, the fusion partner is a reverse transcriptase. In some cases, the fusion partner is a base editor. In some cases, the fusion partner is a deaminase.

In some cases, a CRISPR-Cas fusion polypeptide includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity).

In some cases, a CRISPR-Cas fusion polypeptide includes a heterologous polypeptide that has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used in increase transcription include but are not limited to: transcriptional activators such as VP16, VP64, VP48, VP160, p⁶⁵ subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysinc demcthylases such as JHDM2a/b, UTX, JMJD3, and the like; histone acetyltransferases such as GCN5, PCAF, CBP, p300, TAFI, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROSI, and the like.

Examples of proteins (or fragments thereof) that can be used in decrease transcription include but are not limited to: transcriptional repressors such as the Kruppel associated box (KRAB or SKD); KOXI repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARIDJB/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDACl1, and the like; DNA methylases such as Hhal DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., Foki nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (lET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBECI), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMTIC and EHMT2), SUV39H2, ESET/SETDB1, and the like, SET1A, SET1B, MLL1 to 5, ASH1, SYMD2, NSD1, DOT1L, Pr-SET7/8, SUV4-20H1, EZH2, RIZI), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAFT, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDACT, HDAC2, HDAC3, HDAC8, HDAC4, HDACS, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of a suitable fusion partners are dihydrofolate reductase (DHFR) destabilization domain (e.g., to generate a chemically controllable fusion polypeptide), and a chloroplast transit peptide.

In some case, a CRISPR-Cas fusion polypeptide comprises: a) a CRISPR-Cas effector polypeptide; and b) a chloroplast transit peptide. Thus, for example, a ribonucleoprotein (RNP) complex, comprising a CRISPR-Cas effector polypeptide of the present disclosure and a guide RNA, can be targeted to the chloroplast. In some cases, this targeting may be achieved by the presence of an N-terminal extension, called a chloroplast transit peptide (CTP) or plastid transit peptide. Chromosomal transgenes from bacterial sources must have a sequence encoding a CTP sequence fused to a sequence encoding an expressed polypeptide if the expressed polypeptide is to be compartmentalized in the plant plastid (e.g. chloroplast). Accordingly, localization of an exogenous polypeptide to a chloroplast is often 1 accomplished by means of operably linking a polynucleotide sequence encoding a CTP sequence to the 5′ region of a polynucleotide encoding the exogenous polypeptide. The CTP is removed in a processing step during translocation into the plastid. Processing efficiency may, however, be affected by the amino acid sequence of the CTP and nearby sequences at the amino terminus of the peptide. Other options for targeting to the chloroplast which have been described are the maize cab-m7 signal sequence (U.S. Pat. No. 7,022,896, WO 97/41228) a pea glutathione reductase signal sequence (WO 97/41228) and the CTP described in US2009029861.

In some cases, a CRISPR-Cas fusion polypeptide comprises: a) a CRISPR-Cas effector polypeptide of the present disclosure; and b) an endosomal escape peptide. In some cases, an endosomal escape polypeptide comprises the amino acid sequence GLFXALLXLLXSLWXLLLXA (SEQ ID NO:24), wherein each X is independently selected from lysine, histidine, and arginine. In some cases, an endosomal escape polypeptide comprises the amino acid sequence GLFHALLHLLHSLWHLLLHA (SEQ ID NO:25).

Additional suitable heterologous polypeptides include, but are not limited to, a polypeptide that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). Non-limiting examples of heterologous polypeptides to accomplish increased or decreased transcription include transcription activator and transcription repressor domains. In some such cases, a CRISPR-Cas fusion polypeptide is targeted by the guide nucleic acid (guide RNA) to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some cases, the changes are transient (e.g., transcription repression or activation). In some cases, the changes are inheritable (e.g., when epigenetic modifications are made to the target nucleic acid or to proteins associated with the target nucleic acid, e.g., nucleosomal histones).

Non-limiting examples of heterologous polypeptides for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

The heterologous polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP S1, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI D1 and terminal uridylate transferase); proteins and protein domains responsible for RNA localization (for example from IMPI, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP Al); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as heterologous polypeptides for a fusion polypeptide of the present disclosure have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c6>-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to, proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pill/Abyl, etc.). Nucleases

In some cases, a CRISPR-Cas fusion polypeptide comprises: i) a CRISPR-Cas effector polypeptide; and ii) a heterologous polypeptide (a “fusion partner”), where the heterologous polypeptide is a nuclease. In some cases, a CRISPR-Cas fusion polypeptide comprises a nucleic acid binding effector polypeptide. Suitable nucleic acid binding effector polypeptides can be nucleases including, but not limited to, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

Reverse transcriptases

In some cases, a CRISPR-Cas fusion polypeptide comprises: i) a CRISPR-Cas effector polypeptide; and ii) a heterologous polypeptide (a “fusion partner”), where the heterologous polypeptide is a reverse transcriptase polypeptide. Reverse transcriptases are known in the art; see, e.g., Cot6 and Roth (2008) Virus Res. 134:186. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; a transcription xenopolymerase (RTX); avian myeloblastosis virus reverse transcriptase (AMV-RT); a Eubacterium rectale maturase reverse transcriptase (Marathon®; and the like. The reverse transcriptase fusion partner can include one or more mutations. For example, in some cases, the reverse transcriptase is a M-MLV reverse transcriptase polypeptide that comprises one or more mutations selected from the group consisting of D200N, T306K, W313F, T330P and L603W. In some cases, the reverse transcriptase is a pentamutant of M-MLV RT (e.g., comprising the following substitutions: D200N/L603W/T330P/T306K/W313F) (where D200, L603, T330, T306, and W313 correspond to D199, L602, T329, T305, and W312 of the M-MLV RT amino acid sequence depicted in FIG. 17). Base editors

In some cases, a CRISPR-Cas fusion polypeptide comprises: i) a CRISPR-Cas effector polypeptide; and ii) one or more heterologous polypeptides (a “fusion partner”), where at least one of the one or more heterologous polypeptides is a deaminase. Suitable deaminases include, e.g., an adenosine deaminase; a cytidine deaminase (e.g., an activation-induced cytidine deaminase (AID)); APOBEC3G; and the like); and the like.

A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence: MSEVEFSHEYWMRHALTLAKRAWDEREVPVGAVLVHNNRVIGEGWNRPIGRHDPTAHAEIMA

(SEQ ID NO: 26)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 27)
MRRAFITGVFFLSEVEFSHEYWMRHALTLAKRAWDEREVPVGAVLVHNN
RVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGI
LADECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 28)
MGSHMTNDIYFMTLAIEEAKKAAQLGEVPIGAIITKDDEVIARAHNLRE
TLQQPTAHAEHIAIERAAKVLGSWRLEGCTLYVTLEPCVMCAGTIVMSR
IPRVVYGADDPKGGCSGSLMNLLQQSNFNHRAIVDKGVLKEACSTLLTT
FFKNLRANKKSTN.

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 29)
MTQDELYMKEAIKEAKKAEEKGEVPIGAVLVINGEIIARAHNLRETEQR
SIAHAEMLVIDEACKALGTWRLEGATLYVTLEPCPMCAGAVVLSRVEKV
VFGAFDPKGGCSGTLMNLLQEERFNHQAEVVSGVLEEECGGMLSAFFRE
LRKKKKAARKNLSE.

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 30)
MPPAFITGVTSLSDVELDHEYWMRHALTLAKRAWDEREVPVGAVLVHNH
RVIGEGWNRPIGRHDPTAHAEIMALRQGGLVLQNYRLLDTTLYVTLEPC
VMCAGAMVHSRIGRVVFGARDAKTGAAGSLIDVLHHPGMNHRVEIIEGV
LRDECATLLSDFFRMRRQEIKALKKADRAEGAGPAV.

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 31)
MDEYWMQVAMQMAEKAEAAGEVPVGAVLVKDGQQIATGYNLSISQHDPT
AHAEILCLRSAGKKLENYRLLDATLYITLEPCAMCAGAMVHSRIARVVY
GARDEKTGAAGTVVNLLQHPAFNHQVEVTSGVLAEACSAQLSRFFKRRR
DEKKALKLAQRAQQGIE.

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 32)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAI
LHSRIKRLVFGASDYKTGAIGSRFHFFDDYKMNHTLEITSGVLAEECSQ
KLSTFFQKRREEKKIEKALLKSLSDK.

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 33)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAI
SHARIGRVVFGADDPKGGAVVHGPKFFAQPTCHWRPEVTGGVLADESAD
LLRGFFRARRKAKI.

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 34)
MSSLKKTPIRDDAYWMGKAIREAAKAAARDEVPIGAVIVRDGAVIGRGH
NLREGSNDPSAHAEMIAIRQAARRSANWRLTGATLYVTLEPCLMCMGAI
ILARLERVVFGCYDPKGGAAGSLYDLSADPRLNHQVRLSPGVCQEECGT
MLSDFFRDLRRRKKAKATPALFIDERKVPPEP.

Cytidine deaminases suitable for inclusion in a CRISPR-Cas effector polypeptide fusion polypeptide of the present disclosure include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 35)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTFKDYFYC
WNTFVENHERTFKAWEGLHENSVRLSRQLRRILLPLYEVDDLRDAFRTL
GL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:36).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO:35).

### Transcription Factors

In some cases, a CRISPR-Cas fusion polypeptide comprises: i) a CRISPR-Cas effector polypeptide; and ii) a heterologous polypeptide (a “fusion partner”), where the heterologous polypeptide is a transcription factor. A transcription factor can include: i) a DNA binding domain; and ii) a transcription activator. A transcription factor can include: i) a DNA binding domain; and ii) a transcription repressor. Suitable transcription factors include polypeptides that include a transcription activator or a transcription repressor domain (e.g., the Kruppel associated box (KRAB or SKD); the Mad mSIN3 interaction domain (SID); the ERF repressor domain (ERD), etc.); zinc-finger-based artificial transcription factors (see, e.g., Sera (2009) Adv. Drug Deliv. 61:513); TALE-based artificial transcription factors (see, e.g., Liu et al. (2013) Nat. Rev. Genetics 14:781); and the like. In some cases, the transcription factor comprises a VP64 polypeptide (transcriptional activation). In some cases, the transcription factor comprises a KrUppel-associated box (KRAB) polypeptide (transcriptional repression). In some cases, the transcription factor comprises a Mad mSIN3 interaction domain (SID) polypeptide (transcriptional repression). In some cases, the transcription factor comprises an ERF repressor domain (ERD) polypeptide (transcriptional repression). For example, in some cases, the transcription factor is a transcriptional activator, where the transcriptional activator is GAL4-VP16.

### Recombinases

In some cases, a CRISPR-Cas fusion polypeptide comprises: i) a CRISPR-Cas effector polypeptide; and ii) a heterologous polypeptide (a “fusion partner”), where the heterologous polypeptide is a recombinase. Suitable recombinases include, e.g., a Cre recombinase; a Hin recombinase; a Tre recombinase; a FLP recombinase; and the like.

### NLS

In some cases, a CRISPR-Cas fusion polypeptide comprises: i) a CRISPR-Cas effector polypeptide; and ii) a heterologous polypeptide (a “fusion partner”), where the heterologous polypeptide provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some cases, a CRISPR-Cas fusion polypeptide does not include an NLS so that the protein is not targeted to the nucleus (which can be advantageous, e.g., when the target nucleic acid is an RNA that is present in the cytosol). In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6XHis tag; a hemagglutinin (HA) tag; a FLAG tag; a Myc tag; and the like).

In some cases, a CRISPR-Cas fusion polypeptide comprises: a) a CRISPR-Cas effector polypeptide; and b) one or more nuclear localization signals (NLSs) (e.g., in some cases 2 or more, 3 or more, 4 or more, or 5 or more NLSs). Thus, in some cases, a fusion polypeptide of the present disclosure includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a CRISPR-Cas fusion polypeptide comprises: a) a CRISPR-Cas effector polypeptide; and b) from 1 to 10 NLSs (e.g., 1-9, 1-8, 1-7, 1-6, 1-5, 2-10, 2-9, 2-8, 2-7, 2-6, or 2-5 NLSs). In some cases, a CRISPR-Cas fusion polypeptide comprises: a) a CRISPR-Cas effector polypeptide; and b) from 2 to 5 NLSs (e.g., 2-4 NLSs, or 2-3 NLSs).

Non-limiting examples of NLSs include an NLS sequence derived from: the NLS of the SV40 virus large T-antigen, having the amino acid sequence PKKKRKV (SEQ ID NO:1); the NLS from nucleoplasmin (e.g., the nucleoplasmin bipartite NLS with the sequence KRPAATKKAGQAKKKK (SEQ ID NO:2)); the c-myc NLS having the amino acid sequence PAAKRVKLD (SEQ ID NO:3) or RQRRNELKRSP (SEQ ID NO:4); the hRNPAJ M9 NLS having the sequence NQSSNFGPMKGGNFGGRSSGPYGGGGQYFAKPRNQGGY (SEQ ID NO:5); the sequence RMRIZFKNKGKDTAELRRRRVEVSVELRKAKKDEQILKRRNV (SEQ ID NO:6) of the IBB domain from importin-alpha; the sequences VSRKRPRP (SEQ ID NO:7) and PPKKARED (SEQ ID NO: 8) of the myoma T protein; the sequence PQPKKKPL (SEQ ID NO:9) of human p53; the sequence SALIKKKKKMAP (SEQ ID NO:10) of mouse c-abl IV; the sequences DRLRR (SEQ ID NO:11) and PKQKKRK (SEQ ID NO:16) of the influenza virus NS1; the sequence RKLKKKIKKL (SEQ ID NO:12) of the Hepatitis virus delta antigen; the sequence REKKKFLKRR (SEQ ID NO:13) of the mouse Mxl protein; the sequence KRKGDEVDGVDEVAKKKSKK (SEQ ID NO:14) of the human poly(ADP-ribose) polymerase; and the sequence RKCLQAGMNLEARKTKK (SEQ ID NO:15) of the steroid hormone receptors (human) glucocorticoid. In general, NLS (or multiple NLSs) are of sufficient strength to drive accumulation of the CRISPR-Cas effector polypeptide in a detectable amount in the nucleus of a eukaryotic cell. Detection of accumulation in the nucleus may be performed by any suitable technique. For example, a detectable marker may be fused to the CRISPR-Cas effector polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

### PTD

In some cases, a CRISPR-Cas fusion polypeptide includes a “Protein Transduction Domain” or PTD (also known as a CPP-cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some cases, a PTD is covalently linked to the amino terminus of a CRISPR-Cas effector polypeptide. In some cases, a PTD is covalently linked to the carboxyl terminus of a CRISPR-Cas effector polypeptide. In some cases, the PTD is inserted internally in a CRISPR-Cas effector polypeptide (i.e., is not at the N- or C-terminus of the CRISPR-Cas effector polypeptide) at a suitable insertion site. In some cases, a CRISPR-Cas fusion polypeptide includes: a) a a CRISPR-Cas fusion polypeptide; and b) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a CRISPR-Cas fusion polypeptide includes one or more NLSs (e.g., 2 or more, 3 or more, 4 or more, or 5 or more NLSs). In some cases, a PTD is covalently linked to a nucleic acid (e.g., a CRISPR-Cas guide nucleic acid, a polynucleotide encoding a CRISPR-Cas guide nucleic acid, a polynucleotide encoding a fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO: 127); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 128); Transportan GWTLNSAGYLLGKINLKALAALAKKIL (SEQ ID NO: 129); KALAWEAKLAKALAKALAKHLAKALAKALKCEA (SEQ ID NO: 130); and RQIKIWFQNRRMKWKK (SEQ ID NO: 131). Exemplary PTDs include but are not limited to, YGRKKRRQRRR (SEQ ID NO: 127), RKKRRQRRR (SEQ ID NO: 132); an arginine homopolymer of from 3 arginine residues to 50 arginine residues; Exemplary PTD domain amino acid sequences include, but are not limited to, any of the following: YGRKKRRQRRR (SEQ ID NO: 127); RKKRRQRR (SEQ ID NO: 133); YARAAARQARA (SEQ ID NO: 134); THRLPRRRRRR (SEQ ID NO: 135); and GGRRARRRRRR (SEQ ID NO: 136). In some cases, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Linkers (e.g., for Fusion Partners)

In some cases, a CRISPR-Cas polypeptide can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)˜where n is an integer of at least one; glycine-scrine polymers (including, for example, (GS), (GSGGS)U (SEQ ID NO: 40), (GGSGGS)˜(SEQ ID NO: 41), (GGGGS)n (SEQ ID NO:38), and (GGGS)˜(SEQ ID NO: 42), where n is an integer of at least one; e.g., where n is an integer from 1 to 10); glycine-alanine polymers; and alanine-serine polymers. Exemplary linkers can comprise amino acid sequences including, but not limited to, GGSG (SEQ ID NO: 39), GGSGG (SEQ ID NO: 43), GSGSG (SEQ ID NO: 44), GSGGG (SEQ ID NO: 45), GGGSG (SEQ ID NO: 46), GSSSG (SEQ ID NO: 47), GGGGS (SEQ ID NO:38), and the like. The ordinarily skilled artisan will recognize that design of a peptide conjugated to any desired element can include linkers that are all or partially flexible, such that the linker can include a flexible linker as well as one or more portions that confer less flexible structure.

### Guide Nucleic Acid

As noted above, an EDV of the present disclosure comprises a CRISPR-Cas effector polypeptide guide nucleic acid (e.g., RNA) or a nucleic acid comprising a nucleotide sequence encoding a CRISPR-Cas effector polypeptide guide RNA.

A nucleic acid molecule that binds to a CRISPR-Cas effector polypeptide protein and targets the complex to a specific location within a target nucleic acid is referred to herein as a “CRISPR-Cas effector polypeptide guide RNA” or simply a “guide RNA.”

A guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA. In some cases, the guide RNA is a Cas9 guide RNA.

The first segment (targeting segment) of a guide RNA includes a nucleotide sequence (a guide sequence) that is complementary to (and therefore hybridizes with) a specific sequence (a target site) within a target nucleic acid (e.g., a target DNA, e.g., ssDNA, dsDNA, or a target RNA), such as the complementary strand of a double stranded target DNA, etc. The protein-binding segment (or “protein-binding sequence”) interacts with (binds to) a CRISPR-Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the guide RNA (the guide sequence of the guide RNA) and the target nucleic acid.

A guide RNA and a CRISPR-Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid). The CRISPR-Cas effector polypeptide of the complex provides the site-specific activity (e.g., cleavage activity or an activity provided by the CRISPR-Cas effector polypeptide when the CRISPR-Cas effector polypeptide is a CRISPR-Cas effector polypeptide fusion polypeptide, i.e., has a fusion partner). In other words, the CRISPR-Cas effector polypeptide is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the guide RNA.

The “guide sequence” also referred to as the “targeting sequence” of a guide RNA can be modified so that the guide RNA can target a CRISPR-Cas effector polypeptide to any desired sequence of any desired target nucleic acid, with the exception that the protospacer adjacent motif (PAM) sequence can be taken into account. Thus, for example, a guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some embodiments, a guide RNA includes two separate nucleic acid molecules: an “activator” and a “targeter” and is referred to herein as a “dual guide RNA”, a “double-molecule guide RNA”, or a “two-molecule guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) comprises both the targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a guide RNA. The exact sequence of a given crRNA or tracrRNA molecule is characteristic of the species in which the RNA molecules are found. A dual guide RNA can include any corresponding activator and targeter pair.

The term “activator” or “activator RNA” is used herein to mean a tracrRNA-like molecule (tracrRNA: “trans-acting CRISPR RNA”) of a dual guide RNA (and therefore of a single guide RNA when the “activator” and the “targeter” are linked together by, e.g., intervening nucleotides). Thus, for example, a guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases, the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a dual guide RNA (and therefore of a single guide RNA when the “activator” and the “targeter” are linked together, e.g., by intervening nucleotides). Thus, for example, a guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator) comprises a stretch of nucleotides (a duplex-forming segment) that forms the other half of the dsRNA duplex of the protein-binding segment of the guide RNA. In other words, a stretch of nucleotides of the targeter is complementary to and hybridizes with a stretch of nucleotides of the activator to form the dsRNA duplex of the protein-binding segment of a guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a guide RNA. The particular sequence of a given naturally existing crRNA or tracrRNA molecule is characteristic of the species in which the RNA molecules are found. Examples of suitable activator and targeter are well known in the art.

### Knockouts

In some cases, as noted above, a guide RNA present in an EDV of the present disclosure, or a guide RNA encoded by a guide RNA-encoded nucleic acid present in an EDV of the present disclosure, provides for deletion (“knockout”) of a target nucleic acid.

For example, in some cases, an EDV of the present disclosure provides for: i) delivery of a therapeutic protein; and ii) knockout of a target nucleic acid. As one non-limiting example, an EDV of the present disclosure can both: i) provide for delivery of a therapeutic protein (such as a chimeric antigen receptor (CAR)); and ii) knock out an endogenous nucleic acid encoding a beta-2 microglobulin (02M) polypeptide, where the guide RNA present in the EDV (or encoded by a nucleic acid present in the EDV) would comprise a nucleotide sequence targeting a p32M-encoding nucleic acid in a target cell. Such an EDV would be useful for generating T cells that express a CAR (“CAR-T cells”) that do not express endogenous major histocompatibility complex (MHC) class I antigens on their cell surface and thus could be useful for delivery of allogeneic CAR-T cells.

As another example, in some cases, an EDV comprises a guide RNA, or a nucleic acid comprising a nucleotide sequence encoding the guide RNA, where the guide RNA provides for knockout of the endogenous T-cell receptor alpha constant (TRAC) gene, such that a TRAC polypeptide is not produced in the cell.

A TRAC polypeptide can comprise the following amino acid sequence:

(SEQ ID NO: 204)
IQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKL
VEKSFETDTNLNFQNLSVIGFRILLLKVAGFNLLMTLRLWSS.

As another example, in some cases, an EDV comprises a guide RNA, or a nucleic acid comprising a nucleotide sequence encoding the guide RNA, where the guide RNA provides for knockout of an endogenous gene encoding an immune checkpoint. Immune checkpoints include, e.g., PD-1, PD-L1, CTLA4, and TIGIT.

### Donor Nucleic Acid

In some cases, an EDV of the present disclosure comprises a donor nucleic acid. By a “donor nucleic acid” or “donor sequence” or “donor polynucleotide” or “donor template” it is meant a nucleic acid sequence to be inserted at the site cleaved by a CRISPR-Cas effector protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair or a non disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

In some cases, the donor sequence is provided to the cell as single-stranded DNA. In some cases, the donor sequence is provided to the cell as double-stranded DNA. It may be introduced into a cll in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance.

### Therapeutic Proteins

As noted above, in some cases, an EDV of the present disclosure comprises a nucleic acid comprising a nucleotide sequence encoding a therapeutic polypeptide.

A therapeutic polypeptide encoded by a nucleic acid present in a EDV of the present disclosure can have a length of from about 250 amino acids to about 3000 amino acids. For example, in some cases, a therapeutic polypeptide encoded by a nucleic acid present in a EDV of the present disclosure has a length of from about 250 amino acids to about 500 amino acids, from about 500 amino acids to about 1000 amino acids, from about 500 amino acids to about 750 amino acids, from about 750 amino acids to about 1500 amino acids, from about 750 amino acids to about 1000 amino acids, from about 1000 amino acids to about 1250 amino acids, from about 1000 amino acids to about 1500 amino acids, from about 1250 amino acids to about 1500 amino acids, from about 1250 amino acids to about 1750 amino acids, from about 1500 amino acids to about 1750 amino acids, from about 1500 amino acids to about 2000 amino acids, from about 1500 amino acids to about 2500 amino acids, from about 2000 amino acids to about 2500 amino acids, from about 2000 amino acids to about 3000 amino acids, or from about 2500 amino acids to about 3000 amino acids.

Suitable therapeutic proteins include, but are not limited to, a chimeric antigen receptor (CAR), a T cell receptor (TCR), a natural killer cell receptor (NKR), a synNotch polypeptide, an antibody, a Modular Extracellular Sensor Architecture (MESA) receptor, and the like. In some cases, a therapeutic protein is a functional version of a protein, e.g., a cystic fibrosis transmembrane conductance (CFTR) protein, a globin polypeptide (e.g., 0-globin), and the like.

### Antibodies

In some cases, the therapeutic protein is an antibody. Suitable antibodies include, e.g., therapeutic antibodies. In some cases, the antibody is a single-chain Fv (scFv). In some cases, the antibody is a nanobody.

Suitable antibodies include, e.g., Natalizumab (Tysabri®; Biogen Idec/Elan) targeting a4 subunit of α4β1 and α4β7 integrins (as used in the treatment of MS and Crohn's disease); Vedolizumab (MLN2; Millennium Pharmaceuticals/Takeda) targeting a4P7 integrin (as used in the treatment of UC and Crohn's disease); Belimumab (Benlysta; Human Genome Sciences/GlaxoSmithKline) targeting BAFF (as used in the treatment of SLE); Atacicept (TACI-Ig; Merck/Serono) targeting BAFF and APRIL (as used in the treatment of SLE); Alefacept (Amevive®; Astellas) targeting CD2 (as used in the treatment of Plaque psoriasis, GVHD); Otelixizumab (TRX4; Tolerx/GlaxoSmithKline) targeting CD3 (as used in the treatment of TID); Teplizumab (MGA031; MacroGenics/Eli Lilly) targeting CD3 (as used in the treatment of T1D); Rituximab (Rituxan®/Mabthera; Genentech/Roche/Biogen Idec) targeting CD20 (as used in the treatment of Non-Hodgkin's lymphoma, RA (in patients with inadequate responses to TNF blockade) and CLL); Ofatumumab (Arzerra®; Genmab/GlaxoSmithKline) targeting CD20 (as used in the treatment of CLL, RA); Ocrelizumab (2H7; Genentech/Roche/Biogen Idec) targeting CD20 (as used in the treatment of RA and SLE); Epratuzumab (hLL2; Immunomedics/UCB) targeting CD22 (as used in the treatment of SLE and non-Hodgkin's lymphoma); Alemtuzumab (Campath®/MabCampath; Genzyme/Bayer) targeting CD52 (as used in the treatment of CLL, MS); Abatacept (Orencia®; Bristol-Myers Squibb) targeting CD80 and CD86 (as used in the treatment of RA and JIA, UC and Crohn's disease, SLE); Eculizumab (Soliris®; Alexion pharmaceuticals) targeting C5 complement protein (as used in the treatment of Paroxysmal nocturnal haemoglobinuria); Omalizumab (Xolair®; Genentech/Roche/Novartis) targeting IgE (as used in the treatment of Moderate to severe persistent allergic asthma); Canakinumab (Ilaris®; Novartis) targeting IL-1 (as used in the treatment of Cryopyrin-associated periodic syndromes, Systemic JIA, neonatal-onset multisystem inflammatory disease and acute gout); Mepolizumab (Bosatria; GlaxoSmithKline) targeting IL-5 (as used in the treatment of Hyper-eosinophilic syndrome); Reslizumab (SCH55700; Ception Therapeutics) targeting IL-5 (as used in the treatment of Eosinophilic oesophagitis); Tocilizumab (Actemra®/RoActemra®; Chugai/Roche) targeting IL-6R (as used in the treatment of RA, JIA); Ustekinumab (Stelara®; Centocor) targeting IL-12 and IL-23 (as used in the treatment of Plaque psoriasis, Psoriatic arthritis, Crohn's disease); Briakinumab (ABT-874; Abbott) targeting IL-12 and IL-23 (as used in the treatment of Psoriasis and plaque psoriasis); Etanercept (Enbrel®; Amgen/Pfizer) targeting TNF (as used in the treatment of RA, JIA, psoriatic arthritis, AS and plaque psoriasis); Infliximab (Remicade®; Centocor/Merck) targeting TNF (as used in the treatment of Crohn's disease, RA, psoriatic arthritis, UC, AS and plaque psoriasis); Adalimumab (Humira®/Trudexa®; Abbott) targeting TNF (as used in the treatment of RA, JIA, psoriatic arthritis, Crohn's disease, AS and plaque psoriasis); Certolizumab pegol (Cimzia®; UCB) targeting TNF (as used in the treatment of Crohn's disease and RA); Golimumab (Simponi®; Centocor) targeting TNF (as used in the treatment of RA, psoriatic arthritis and AS); and the like. In some cases, the antibody whose production is induced by the intracellular domain of a synNotch polypeptide of the present disclosure is a therapeutic antibody for the treatment of cancer. Such antibodies include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-Li (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin _5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETRI) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81 C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

Suitable antibodies include, e.g., Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomilixirnab, Gusetkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumnretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumah, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBSO7, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

### Chimeric Antigen Receptors (CARs)

A CAR generally comprises: a) an extracellular domain comprising an antigen-binding domain (antigen-binding polypeptide); b) a transmembrane region; and c) a cytoplasmic domain comprising an intracellular signaling domain (intracellular signaling polypeptide). In some cases, a CAR comprises: a) an extracellular domain comprising the antigen-binding domain; b) a transmembrane region; and c) a cytoplasmic domain comprising: i) one or more co-stimulatory polypeptides; and ii) an intracellular signaling domain. In some cases, a CAR comprises hinge region between the extracellular antigen-binding domain and the transmembrane domain. Thus, in some cases, a CAR comprises: a) an extracellular domain comprising the antigen-binding domain; b) a hinge region; c) a transmembrane region; and d) a cytoplasmic domain comprising an intracellular signaling domain. In some cases, a CAR comprises: a) an extracellular domain comprising the antigen-binding domain; b) a hinge region; c) a transmembrane region; and d) a cytoplasmic domain comprising: i) one or more co-stimulatory polypeptides; and ii) an intracellular signaling domain.

Exemplary CAR structures are known in the art (See e.g., WO 2009/091826; US 20130287748; WO 2015/142675; WO 2014/055657; WO 2015/090229; and U.S. Pat. No. 9,587,020. In some cases, a CAR is a single polypeptide chain. In some cases, a CAR comprises two polypeptide chains. Generally, any CAR structure known to those skilled in the art can be used.

CARs specific for a variety of tumor antigens are known in the art; for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J. Natl Cancer Inst (2014) 107(1):364), carbonic anhydrase IX-specific CARs (Lamers et al., Biochem Soc Trans (2016) 44(3):951-959), folate receptor-α (FR-α)-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL-13Ra2-specific CARs (Brown et al., Clin Cancer Res (2015) 21(18):4062-4072), ganglioside GD2-specific CARs (Louis et al., Blood (2011) 118(23):6050-6056; Caruana et al., Nat Med (2015) 21(5):524-529; Yu et al. (2018) J. Hematol. Oncol. 11:1), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2): 154-166), mesothelin (MSLN)-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (YescartaT^{M}) and Tisagenlecleucel (KymriahT^{M}). See also, Li et al., JHematol and Oncol (2018) 11:22, reviewing clinical trials of tumor-specific CARs; Heyman and Yan (2019) Cancers 11:pii:E191; Baybutt et al. (2019) Clin. Pharmacol. Ther. 105:71.

As noted above, a CAR comprises an extracellular domain comprising an antigen-binding domain. The antigen-binding domain present in a CAR can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable. In some cases, the antigen-binding domain is a nanobody.

In some cases, the antigen bound by the antigen-binding domain of a CAR is selected from: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PRI) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE Al) polypeptide, a cytochrome P450 1B1 (CYP1B1) polypeptide, a placenta-specific protein 1 (PLACI) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B713; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFP) polypeptide, a MAD-CT-2 polypeptide, or a Fos-related antigen-1 (FOSL) polypeptide.

The antigen-binding polypeptide of a CAR can bind any of a variety of cancer-associated antigens, including, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), B-cell maturation antigen (BCMA), high molecular weight-melanoma associated antigen (HMW-MAA), MAGE-Ai, IL-13R-a2, GD2, and the like. Cancer-associated antigens also include, e.g., 4-1BB, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, Li-CAM, IL-13, IL-6, insulin-like growth factor I receptor, integrin a5p1, integrin avP3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDCi, SLAMF7, TAG-72, tenascin C, TGF beta 2, TGF-β, TRAIL-Ri, TRAIL-R2, tumor antigen CTAA16.88, VEGF-A, VEGFR-1, VEGFR2, and vimentin.

VH and VL amino acid sequences of various cancer-associated antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of antibodies that bind cancer-associated antigens.

As one non-limiting example, in some cases, a CAR comprises an anti-CD19 antibody (e.g., an anti-CD19 scFv or an anti-CD19 nanobody). Anti-CD19 antibodies are known in the art; and the VH and VL, or the VH and VL CDRs, of any anti-CD19 antibody can be included in a CAR. See e.g., WO 2005/012493.

In some cases, an anti-CD19 antibody includes a VL CDR1 comprising the amino acid sequence KASQSVDYDGDSYLN (SEQ ID NO:181); a VL CDR2 comprising the amino acid sequence DASNLVS (SEQ ID NO:182); and a VL CDR3 comprising the amino acid sequence QQSTEDPWT (SEQ ID NO:183). In some cases, an anti-CD19 antibody includes a VH CDR1 comprising the amino acid sequence SYWMN (SEQ ID NO:184); a VH CDR2 comprising the amino acid sequence QIWPGDGDTNYNGKFKG (SEQ ID NO:185); and a VH CDR3 comprising the amino acid sequence RETTTVGRYYYAMDY (SEQ ID NO:186). In some cases, an anti-CD19 antibody includes a VL CDR1 comprising the amino acid sequence KASQSVDYDGDSYLN (SEQ ID NO:181); a VL CDR2 comprising the amino acid sequence DASNLVS (SEQ ID NO:182); and a VL CDR3 comprising the amino acid sequence QQSTEDPWT (SEQ ID NO:183); a VH CDR1 comprising the amino acid sequence SYWMN (SEQ ID NO:184); a VH CDR2 comprising the amino acid sequence QIWPGDGDTNYNGKFKG (SEQ ID NO:185); and a VH CDR3 comprising the amino acid sequence RETTTVGRYYYAMDY (SEQ ID NO:186).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 187)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGGGGSGGGGSGGGGSQVQLQQSGAELVRPGSSVKI
SCKASGYAFSSYWMNWVKQRPGQGLEWIGQIWPGDGDTNYNGKFKGKAT
LTADESSSTAYMQLSSLASEDSAVYFCARRETTTVGRYYYAMDYWGQGT
TVTVS.

### Compositions Comprising an Edv

The present disclosure provides compositions, including pharmaceutical compositions, comprising an EDV of the present disclosure. The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, ₁₉t Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H.C. Ansel et al., eds 7′ ed., Lippincott, Williams, & Wilkins; and Handbook of Pharmaceutical Excipients (2000) A.H. Kibbe et al., eds., 3^{rd} ed. Amer. Pharmaceutical Assoc.

A composition of the present disclosure can include: a) an EDV of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

In some cases, the composition is sterile. In some cases, the composition is suitable for administration to a human subject, e.g., where the composition is sterile and is free of detectable pyrogens and/or other toxins.

In some cases, a composition of the present disclosure comprises: i) an EDV that does not include a donor template nucleic acid; and ii) a donor template nucleic acid (provided separately from the EDV).

### Nucleic Acids

As noted above, in some cases, an EDV of the present disclosure comprises a nucleic acid comprising a nucleotide sequence encoding a CRISPR-Cas effector polypeptide. In some cases, an EDV of the present disclosure comprises a nucleic acid comprising a nucleotide sequence encoding a guide RNA. In some cases, an EDV of the present disclosure comprises a nucleic acid comprising a nucleotide sequence encoding a therapeutic polypeptide.

A coding sequence (e.g., a nucleotide sequence encoding a CRISPR-Cas effector polypeptide; a nucleotide sequence encoding a CRISPR-Cas guide RNA; a nucleotide sequence encoding a therapeutic protein) present in an EDV of the present disclosure can be operably linked to a transcriptional control element (e.g., a promoter). The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human Hi promoter (H1), and the like.

In some cases, a coding nucleotide sequence is operably linked to (under the control of) a promoter operable in a cukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA in a eukaryotic cell it may sometimes be necessary to modify the sequence encoding the guide RNA to eliminate runs of Ts. In some cases, a nucleotide sequence encoding guide RNA is operably linked to a promoter operable in a eukaryotic cell (e.g., a CMV promoter, an EF1αpromoter, an estrogen receptor-regulated promoter, and the like).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothioncin promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

### Methods of Delivering a Nucleic Acid-Binding Effector Polypeptide

The present disclosure provides methods of delivering a nucleic acid-binding effector polypeptide to a target eukaryotic cell. The methods generally involve contacting the cell with a EDV of the present disclosure or administering a EDV to an organism. In some cases, the target cell is in vitro. In some cases, the target cell is in vivo and the method comprises administering the EDV to an individual.

The present disclosure provides methods of delivering a CRISPR-Cas polypeptide to a target eukaryotic cell. The methods generally involve contacting the cell with a EDV of the present disclosure or administering a EDV to an organism. In some cases, the target cell is in vitro. In some cases, the target cell is in vivo and the method comprises administering the EDV to an individual.

Where a EDV of the present disclosure comprises a guide RNA, in some instances, the guide RNA provides for knockout of a nucleic acid targeted by the guide RNA. Thus, in some cases, a EDV of the present disclosure provides for: i) delivery of a therapeutic protein; and ii) knockout of a target nucleic acid. As one non-limiting example, a EDV of the present disclosure can both: i) provide for delivery of a therapeutic protein (such as a chimeric antigen receptor (CAR)); and ii) knock out an endogenous nucleic acid encoding a beta-2 microglobulin (02M) polypeptide, where the guide RNA present in the EDV (or encoded by a nucleic acid present in the EDV) would comprise a nucleotide sequence targeting a P2M-encoding nucleic acid in a target cell. Such a EDV would be useful for generating T cells that express a CAR (“CAR-T cells”) that do not express endogenous major histocompatibility complex (MHC) class I antigens on their cell surface and thus could be useful for delivery of allogeneic CAR-T cells. As another non-limiting example, a EDV of the present disclosure can both: i) provide for delivery of a therapeutic protein (such as an antibody, e.g., a cancer-specific antibody or other therapeutic antibody); and ii) knock out an endogenous nucleic acid encoding an antibody light chain (e.g., a kappa light chain) or an immunoglobulin (Ig) Fe polypeptide (e.g., an Ig Fe polypeptide of a particular isotype such as IgG1). Such a EDV would be useful for generating B cells that produce a therapeutic antibody.

In some cases, a EDV of the present disclosure provides for homology directed repair (HDR) of a defective target nucleic acid. In some cases, a EDV of the present disclosure provides for non-homologous end joining (NHEJ) of a target nucleic acid, e.g., to provide for a knockout of a target nucleic acid.

A cell that serves as a recipient for a EDV of the present disclosure can be any of a variety of eukaryotic cells, including, e.g., in vitro cells; in vivo cells; ex vivo cells; primary cells; cancer cells; animal cells; plant cells; algal cells; fungal cells; etc. A cell that serves as a recipient for a EDV of the present disclosure is referred to as a “host cell” or a “target cell.”

In some cases, the target cell is in vitro. In some cases, cells are removed from an individual, contacted with a EDV of the present disclosure in vitro, such that the cells are modified to produce the therapeutic protein encoded by a nucleic acid present in the EDV; and returning the modified cells to the individual from whom the cells were obtained. In some cases, cells are removed from an individual, contacted with a EDV of the present disclosure in vitro, such that the cells are modified to produce the therapeutic protein encoded by a nucleic acid present in the EDV; and administering the modified cells to an individual other than the individual from whom the cells were obtained.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells. In some cases, the cell is a tissue-resident stem cell.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some cases, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3-. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other instances, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other instances, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some cases, the target cell is a lung cell. In some cases, the EDV comprises a guide RNA, or a nucleic acid comprising a nucleotide sequence encoding a guide RNA, where the guide RNA comprises a targeting sequence that targets a CFTR (cystic fibrosis transmembrane conductance regulator) gene. For example, targeting a CFTR gene can treat cystic fibrosis. Where the target gene comprises a defect that leads to pathology, a donor nucleic acid comprising a nucleotide sequence without the defect can be included in the EDV, such that the defect is corrected.

In some cases, the target cell is a CD34⁺ cell. In some cases, the EDV comprises a guide RNA, or a nucleic acid comprising a nucleotide sequence encoding a guide RNA, where the guide RNA comprises a targeting sequence that targets an HbF (fetal hemoglobin) gene. For example, targeting an HbF gene can treat sickle cell disease or heta-thalassemia. Where the target gene comprises a defect that leads to pathology, a donor nucleic acid comprising a nucleotide sequence without the defect can be included in the EDV, such that the defect is corrected.

In some cases, the target cell is a CD8⁺ T cell. In some cases, the EDV comprises a guide RNA, or a nucleic acid comprising a nucleotide sequence encoding a guide RNA, where the guide RNA comprises a targeting sequence that targets a gene selected from PD1 (programmed cell death 1), CTLA4 (cytotoxic T-lymphocyte-associated protein 4), and TCR (T-cell receptor). For example, targeting a PD-1 gene, a CTLA-4 gene, or a TCR gene, can be used in the generation of chimeric antigen receptor (CAR)-T cells.

In some cases, the target cell is a CD4⁺ T cell. In some cases, the EDV comprises a guide RNA, or a nucleic acid comprising a nucleotide sequence encoding a guide RNA, where the guide RNA comprises a targeting sequence that targets a CCR5 gene, or targets an integrated and proviral HIV-1. Targeting a CCR5 gene can be used to enhance resistance to HIV. Targeting an integrated and proviral HIV-1 can be used to reduce the pool of T cells that are reservoirs for latent HIV.

In some cases, the target cell is a skeletal muscle cell. In some cases, the EDV comprises a guide RNA, or a nucleic acid comprising a nucleotide sequence encoding a guide RNA, where the guide RNA comprises a targeting sequence that targets a Duchenne muscular dystrophy (DMD) gene. Targeting a DMD gene can be used to treat Duchenne muscular dystrophy. Where the target gene comprises a defect that leads to pathology, a donor nucleic acid comprising a nucleotide sequence without the defect can be included in the EDV, such that the defect is corrected.

In some cases, the target cell is an ocular cell (e.g., in a retinal cell, a photoreceptor cell, etc.). In some cases, the EDV comprises a guide RNA, or a nucleic acid comprising a nucleotide sequence encoding a guide RNA, and wherein the guide RNA comprises a targeting sequence that targets a CEP290 (centrosomal protein 290) gene. Targeting a CEP290 gene can be used to treat Leber congenital amaurosis 10 (LCA10). Where the target gene comprises a defect that leads to pathology, a donor nucleic acid comprising a nucleotide sequence without the defect can be included in the EDV, such that the defect is corrected.

In some cases, target cell is an auditory cell (e.g., hair cells, cochlear cells, etc.). In some cases, the EDV comprises a guide RNA, or a nucleic acid comprising a nucleotide sequence encoding a guide RNA, where the guide RNA comprises a targeting sequence that targets a USH2A (Usher syndrome 2A) gene. Targeting a USH2A gene can be used to treat Usher Syndrome type 2A. Where the target gene comprises a defect that leads to pathology, a donor nucleic acid comprising a nucleotide sequence without the defect can be included in the EDV, such that the defect is corrected.

In some cases, the target cell is a central nervous system cell (e.g., neurons (e.g., excitatory and inhibitory neurons); and glial cells (e.g., oligodendrocytes, astrocytes and microglia)). In some cases, the EDV comprises a guide RNA, or a nucleic acid comprising a nucleotide sequence encoding a guide RNA, and wherein the guide RNA comprises a targeting sequence that targets a gene selected from Tau/MAPT-1, HTT (Huntingtin), SOD1 (superoxide dismutase 1), SOCS3 (suppressor of cytokine signaling 3), USP8 (ubiquitin specific peptidase 8), DOT1L (DOTI-like histone lysine methyltransferase), UFM1 (ufmylation; ubiquitin fold modifier 1), SOCS2 (suppressor of cytokine signaling 2), SOCS9 (suppressor of cytokine signaling 9), SOCS13 (suppressor of cytokine signaling 13), SOCSII (suppressor of cytokine signaling 11), and SOCS5 (suppressor of cytokine signaling 5). For example, targeting a Tau gene can treat Alzheimer's disease. As another example, targeting an HTT gene can treat Huntington Disease. As another example, targeting a SOD1 gene can treat amyotrophic lateral sclerosis. As another example, targeting a Ufmylation, USP8, DOT1L, SOCS2, SOCS3, SOCS9, SOCS13, SOCS11, or SOCS5 gene can treat glioblastoma. Where the target gene comprises a defect that leads to pathology, a donor nucleic acid comprising a nucleotide sequence without the defect can be included in the EDV, such that the defect is corrected.

In some cases, a single dose of a composition comprising a EDV of the present disclosure comprises from about 102 EDVs to about 109 EDVs. For example, a single dose of a composition comprising a EDV of the present disclosure comprises from about 10² EDVs to about 10 EDVs, from about 10³ EDVs to about 10⁴ EDVs, from about 10⁴ EDVs to about 105 EDVs, from about 10 EDVs to about 10⁶ EDVs, from about 10⁶ EDVs to about 10?EDVs, from about 10⁷ EDVs to about 10 EDVs, from about 10 EDVs to about 109 EDVs, or from about 109 EDVs to about 10¹⁰ EDVs. A composition comprising a EDV of the present disclosure can be administered via any of a variety of parenteral and non-parenteral routes of administration. For example, a composition comprising a EDV of the present disclosure can be administered intravenously, intramuscularly, intratumorally, peritumorally, subcutaneously, intraperitoneally, and the like. A EDV of the present disclosure can be administered via convection enhanced delivery (CED) injection.

### Methods of In Vivo Genome Editing

The present disclosure provides a method of modifying a target nucleic acid in a target eukaryotic cell in vivo, the method comprising administering to an individual in need thereof an effective amount of an EDV of the present disclosure, or a composition comprising an EDV of the present disclosure. The EDV enters the target eukaryotic cell in the individual and modifies the target nucleic acid in the target eukaryotic cell. In some cases, the target cell is a CD4⁺ T cell. In some cases, the target cell is a CD8⁺ T cell.

In some cases, the target cell is an immune cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg). In some cases, the target cell is a CD4⁺ T cell. In some cases, the target cell is a CD8⁺ T cell.

In some cases, the EDV comprises one or more guide RNAs (or one or more nucleic acids comprising nucleotide sequences encoding the one or more guide RNAs) that provide for one or more of: a) insertion of a nucleic acid comprising a nucleotide sequence encoding a therapeutic polypeptide into the genome of the target cell; b) deletion of one or more endogenous nucleic acids in the target cell.

In some cases, the EDV comprises a nucleic acid comprising a nucleotide sequence encoding a CAR. In some cases, the EDV comprises: a) a nucleic acid comprising a nucleotide sequence encoding a CAR; and b) a guide RNA that provides for knockout of an TRAC-encoding nucleic acid in the target cell. In some cases, the EDV comprises a nucleic acid comprising a nucleotide sequence encoding a CAR. In some cases, the EDV comprises: a) a nucleic acid comprising a nucleotide sequence encoding a CAR; and b) a guide RNA that provides for knockout of an immune checkpoint in the target cell. In some of these embodiments, the target cell is a CD8⁺ T cell.

In some cases, a single dose of a composition comprising an EDV of the present disclosure comprises from about 102 EDVs to about 10 EDVs. For example, a single dose of a composition comprising an EDV of the present disclosure comprises from about 102 EDVs to about 10 EDVs, from about 10′ EDVs to about 10⁴ EDVs, from about 10⁴ EDVs to about 10′ EDVs, from about 10 EDVs to about 10⁶ EDVs, from about 10⁶ EDVs to about 10⁷ EDVs, from about 10⁷ EDVs to about 10′ EDVs, from about 10′ EDVs to about 10 EDVs, or from about 10 EDVs to about 10¹⁰ EDVs.

A composition comprising an EDV of the present disclosure can be administered via any of a variety of parenteral and non-parenteral routes of administration. For example, a composition comprising an EDV of the present disclosure can be administered intravenously, intramuscularly, intratumorally, peritumorally, subcutaneously, intraperitoneally, and the like. An EDV of the present disclosure can be administered via convection enhanced delivery (CED) injection.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below (see, e.g., SET A, SET B, and SET C). As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Set A

Aspect 1. A virus-like particle (VLP) comprising: a) a nucleic acid-binding effector polypeptide; and b) one or more fusion polypeptides comprising: i) a viral envelope protein; and ii) a targeting polypeptide.
Aspect 2. The VLP of aspect 1, wherein the targeting polypeptide is one or more antibodies or one or more antibody analogs.
Aspect 3. The VLP of aspect 2, wherein the one or more antibody analogs is an affibody, an affilin, an affimer, an affitin, an alphabody, an anticalin, an avimer, a DARPin, a Fynomer, a Kunitz domain peptide, a monobody, a repebody, a VLR, or a nanoCLAMP.
Aspect 4. The VLP of aspect 2, wherein the one or more antibodies is a single chain Fv (scFv) polypeptide, a diabody, a bispecific antibody, a triabody, or a nanobody.
Aspect 5. The VLP of aspect 1, wherein the targeting polypeptide is a fusion polypeptide comprising: (i) an antibody or antibody analog; and (ii) one or more heterologous polypeptides.
Aspect 6. The VLP of aspect 5, wherein the antibody is a single chain Fv (scFv) polypeptide, a diabody, a triabody, or a nanobody.
Aspect 7. The VLP of aspect 5, wherein the antibody analog is an affibody, an affilin, an affimer, an affitin, an alphabody, an anticalin, an avimer, a DARPin, a Fynomer, a Kunitz domain peptide, a monobody, a repebody, a VLR, or a nanoCLAMP.
Aspect 8. The VLP of any one of aspects 5-7, wherein the one of more heterologous polypeptides comprises a stalk portion of a transmembrane polypeptide.
Aspect 9. The VLP of any one of aspects 5-7, wherein the one of more heterologous polypeptides comprises a stalk portion and a transmembrane portion of a transmembrane polypeptide.
Aspect 10. The VLP of aspect 9, wherein the transmembrane polypeptide is a CD8a chain polypeptide or a platelet-derived growth factor polypeptide.
Aspect 11. The VLP of aspect 8, wherein the one or more heterologous polypeptides comprises the stalk portion of a CD8a chain polypeptide.
Aspect 12. The VLP of aspect 8, wherein the one or more heterologous polypeptides comprises the stalk portion and the transmembrane domain of a CD8a chain polypeptide.
Aspect 13. The VLP of aspect 12, wherein the stalk portion and the transmembrane domain comprises the amino acid sequence TTTPAPRPPTPAPTIASQPLSLRPEACRPAAGGAVHTRGLDFACDIYIWAPLAGTCGVL LLSLVITLYC (SEQ ID NO:20).
Aspect 14. The VLP of any one of aspects 1-13, wherein the viral envelope protein is selected from a Hepatitis B virus (HBV) glycoprotein, a Hepatitis C virus (HCV) glycoprotein, a Marburg virus glycoprotein, an Ebola virus glycoprotein, a vesicular stomatitis virus (VSV) glycoprotein, an influenza virus hemagglutinin, a SARS-CoV glycoprotein, a respiratory syncytial virus (RSV) glycoprotein, a human parainfluenza virus glycoprotein, a measles virus hemagglutinin and/or a measles virus fusion glycoprotein, an HTLV-1 glycoprotein, a Ross river virus glycoprotein, a rabies virus glycoprotein, a Mokola virus glycoprotein, a Semliki Forest virus glycoprotein, a Sindbis virus glycoprotein, a Venezuelan equine encephalitis virus glycoprotein.
Aspect 15. The VLP of any one of aspects 1-14, wherein the viral envelope protein is a variant viral envelope protein that comprises one or more amino acid substitutions that reduce binding of the viral envelope protein to its receptor.
Aspect 16. The VLP of aspect 15, wherein the viral glycoprotein is a variant vesicular stomatitis virus glycoprotein (VSVG) that comprises a substitution of K47 and/or R354, wherein the amino acid numbering is based on the amino acid sequence depicted in FIG. 16A.
Aspect 17. The VLP of any one of aspect 1-16, wherein the targeting polypeptide binds to a cancer cell, a hematopoietic stem cell, a lung cell, a neuron, an adipocyte, a hepatocyte, an endothelial cell, a muscle cell, a cardiomyocyte, a retinal cell, or a T cell.
Aspect 18. The VLP of any one of aspects 1-16, wherein the targeting polypeptide binds to a CD8⁺ T cell or a CD4⁺ T cell.
Aspect 19. The VLP of any one of aspects 1-16, wherein the targeting polypeptide binds selectively to a cancer cell.
Aspect 20. The VLP of any one of aspects 1-19, wherein the nucleic acid binding effector polypeptide is a CRISPR-Cas effector polypeptide, a Zinc Finger Nuclease (ZFN) or a Transcription activator-like effector nuclease. CRISPR-Cas effector polypeptide is a type II CRISPR-Cas effector polypeptide, a type V CRISPR-Cas effector polypeptide, or a type VI CRISPR-Cas effector polypeptide.
Aspect 21. The VLP of aspect 20, wherein the CRISPR-Cas effector polypeptide is a type II CRISPR-Cas effector polypeptide, a type V CRISPR-Cas effector polypeptide, or a type VI CRISPR-Cas effector polypeptide.
Aspect 22. The VLP of any one of aspects 1-21, wherein the nucleic acid-binding effector polypeptide is a fusion polypeptide comprising: i) a CRISPR-Cas effector polypeptide; and ii) one or more heterologous polypeptides.
Aspect 23. The VLP of aspect 22, wherein the CRISPR-Cas effector polypeptide exhibits reduced catalytic activity compared to a wild-type CRISPR-Cas effector polypeptide, wherein the CRISPR-Cas effector polypeptide retains the ability to bind to a target nucleic acid when the CRISPR-Cas effector polypeptide is complexed with a guide nucleic acid.
Aspect 24. The VLP of aspect 22 or 23, wherein at least one of the one or more heterologous polypeptides comprises a deaminase, a reverse transcriptase, a transcription modulator, or an epigenetic modulator.
Aspect 25. The VLP of any one of aspects 22-24, wherein the one or more heterologous polypeptides comprises one or more nuclear localization signals.
Aspect 26. The VLP of any one of aspects 1-25, comprising a CRISPR-Cas guide RNA, or a nucleic acid comprising a nucleotide sequence encoding the CRISPR-Cas guide RNA. Aspect 27. The VLP of any one of aspects 1-26, further comprising a donor template nucleic acid, or a nucleotide sequence encoding the donor template nucleic acid.
Aspect 28. The VLP of any one of aspects 1-27, further comprising a therapeutic polypeptide, or a nucleic acid comprising a nucleotide sequence encoding a therapeutic polypeptide. Aspect 29. A composition comprising the VLP of any one of aspects 1-18.
Aspect 30. The composition of aspect 29, comprising a pharmaceutically acceptable excipient. Aspect 31. A method of delivering a nucleic acid-binding effector polypeptide to a eukaryotic cell, the method comprising contacting the cell with the VLP of any one of aspects 1-28, or the composition of aspect 29 or aspect 30.
Aspect 32. The method of aspect 31, wherein the eukaryotic cell is in vivo.
Aspect 33. The method of aspect 31, wherein the eukaryotic cell is in vitro.
Aspect 34. The method of any one of aspects 31-33, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, or a T cell.
Aspect 35. A method for modifying a target nucleic acid in a eukaryotic cell, the method comprising contacting the cell with the VLP of any one of aspects 1-28, or the composition of aspect 29 or aspect 30, wherein said contacting results in delivery of the nucleic acid-binding effector polypeptide into the cell and modification of the target nucleic acid.
Aspect 36. The method of aspect 35, wherein the eukaryotic cell is in vivo.
Aspect 37. The method of aspect 35, wherein the eukaryotic cell is in vitro.
Aspect 38. The method of any one of aspects 35-37, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, or a T cell.

### Set B

Aspect 1. An enveloped delivery vehicle (EDV) comprising: a) a nucleic acid-binding effector polypeptide; and b) one or more fusion polypeptides comprising: i) a viral envelop protein; and ii) one or more targeting polypeptides, optionally wherein the one or more targeting polypeptides comprise one or more antibodies or antibody analogs that bind specifically to a target polypeptide on a target cell.
Aspect 2. The EDV of aspect 1, wherein the one or more antibodies or antibody analogs is an affibody, an affilin, an affimer, an affitin, an alphabody, an anticalin, an avimer, a DARPin, a Fynomer, a Kunitz domain peptide, a monobody, a repebody, a VLR, or a nanoCLAMP.
Aspect 3. The EDV of aspect 2, wherein the one or more antibodies is a single chain Fv (scFv) polypeptide, a diabody, a bispecific antibody, a triabody, or a nanobody Aspect 4. The EDV of any one of aspects 1-3, wherein the one or more antibodies or antibody analogs bind specifically to a CD8⁺ T cell or a CD4⁺ T cell.
Aspect 5. The EDV of aspect 4, wherein the one or more antibodies comprise an anti-CD3 antibody and an anti-CD8 antibody.
Aspect 6. The EDV of aspect 5, wherein the anti-CD3 antibody and the anti-CD8 antibody are scFv polypeptides or nanobodies.
Aspect 7. The EDV of any one of aspects 1-6, wherein the viral envelope protein is selected from a Hepatitis B virus (HBV) glycoprotein, a Hepatitis C virus (HCV) glycoprotein, a Marburg virus glycoprotein, an Ebola virus glycoprotein, a vesicular stomatitis virus (VSV) glycoprotein, an influenza virus hemagglutinin, a SARS-CoV glycoprotein, a respiratory syncytial virus (RSV) glycoprotein, a human parainfluenza virus glycoprotein, a measles virus hemagglutinin and/or a measles virus fusion glycoprotein, an HTLV-1 glycoprotein, a Ross river virus glycoprotein, a rabies virus glycoprotein, a Mokola virus glycoprotein, a Semliki Forest virus glycoprotein, a Sindbis virus glycoprotein, a Venezuelan equine encephalitis virus glycoprotein.
Aspect 8. The EDV of any one of aspects 1-6, wherein the viral envelope protein is a variant viral envelope protein that comprises one or more amino acid substitutions that reduce binding of the viral envelope protein to its receptor.
Aspect 9. The EDV of aspect 8, wherein the viral glycoprotein is a variant vesicular stomatitis virus glycoprotein (VSVG) that comprises a substitution of K47 and/or R354, wherein the amino acid numbering is based on the amino acid sequence depicted in FIG. 16A.
Aspect 10. The EDV of any one of aspects 1-9, wherein the nucleic acid binding effector polypeptide is a CRISPR-Cas effector polypeptide, a Zinc Finger Nuclease (ZFN) or a Transcription activator-like effector nuclease. CRISPR-Cas effector polypeptide is a type II CRISPR-Cas effector polypeptide, a type V CRISPR-Cas effector polypeptide, or a type VI CRISPR-Cas effector polypeptide.
Aspect 11. The EDV of aspect 10, wherein the CRISPR-Cas effector polypeptide is a type II CRISPR-Cas effector polypeptide, a type V CRISPR-Cas effector polypeptide, or a type VI CRISPR-Cas effector polypeptide.
Aspect 12. The EDV of any one of aspects 1-11, wherein the nucleic acid-binding effector polypeptide is a fusion polypeptide comprising: i) a CRISPR-Cas effector polypeptide; and ii) one or more heterologous polypeptides.
Aspect 13. The EDV of aspect 12, wherein the CRISPR-Cas effector polypeptide exhibits reduced catalytic activity compared to a wild-type CRISPR-Cas effector polypeptide, wherein the CRISPR-Cas effector polypeptide retains the ability to bind to a target nucleic acid when the CRISPR-Cas effector polypeptide is complexed with a guide nucleic acid.
Aspect 14. The EDV of aspect 12 or 13, wherein at least one of the one or more heterologous polypeptides comprises a deaminase, a reverse transcriptase, a transcription modulator, or an epigenetic modulator.
Aspect 15. The EDV of any one of aspects 12-14, wherein at least one of the one or more heterologous polypeptides is a lentiviral Gag polypeptide.
Aspect 16. The EDV of any one of aspects 12-15, wherein the one or more heterologous polypeptides comprises one or more nuclear localization signals.
Aspect 17. The EDV of any one of aspects 12-16, wherein the one or more heterologous polypeptides comprises a nuclear export signal (NES) polypeptide.
Aspect 18. The EDV of any one of aspects 1-17, comprising one or more CRISPR-Cas guide RNAs, or one or more nucleic acids comprising nucleotide sequences encoding the one or more CRISPR-Cas guide RNAs.
Aspect 19. The EDV of any one of aspects 1-18, further comprising a donor template nucleic acid, or a nucleotide sequence encoding the donor template nucleic acid.
Aspect 20. The EDV of any one of aspects 1-19, further comprising a therapeutic polypeptide, or a nucleic acid comprising a nucleotide sequence encoding a therapeutic polypeptide.
Aspect 21. The EDV of aspect 20, wherein the therapeutic polypeptide is a chimeric antigen receptor (CAR).
Aspect 22. The EDV of aspect 21, wherein the CAR comprises one or more scFv or one or more nanobodies specific for a cancer-associated antigen.
Aspect 23. The EDV of aspect 22, wherein:
   a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Ra2, CD171, MUC16, PSMA, AXL, CD20, CD80/86, c-MET, DLL-3, DR5, EpHA2, FR-α, gp100, MAGE-Ai, MAGE-A3, MAGE-A4, and LMP1; or
   b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-Li, TRAILR1, TRAILR2, transferrin receptor, and VEGF.
Aspect 24. The EDV of any one of aspects 18-23, wherein at least one of the one or more guide RNAs comprises a nucleotide sequence that hybridizes with a target nucleic acid and provides for knockout of the target nucleic acid.
Aspect 25. The EDV of aspect 23, wherein the target nucleic acid that is knocked out encodes an immune checkpoint.
Aspect 26. The EDV of aspect 25, wherein the immune checkpoint is PD-1.
Aspect 27. The EDV of aspect 24, wherein the target nucleic acid that is knocked out encodes a T-cell receptor alpha constant (TRAC) polypeptide.
Aspect 28. A composition comprising the EDV of any one of aspects 1-27.
Aspect 29. The composition of aspect 28, comprising a pharmaceutically acceptable excipient.
Aspect 30. A method of modifying a target nucleic acid in a target eukaryotic cell in vivo, the method comprising administering to an individual in need thereof an effective amount of the EDV of any one of aspects 1-27, or the composition of aspect 28 or aspect 29, wherein the EDV enters the target eukaryotic cell in the individual and modifies the target nucleic acid in the target eukaryotic cell.
Aspect 31. The method of aspect 30, wherein the target cell is a CD4⁺ T cell or a CD8⁺ T cell.
Aspect 32. The method of aspect 31, wherein the fusion polypeptide in the EDV comprises: i) the viral envelop protein; and ii) an anti-CD3 antibody and an anti-CD8 antibody as the targeting polypeptides.
Aspect 33. The method of aspect 32, wherein the EDV comprises: a) a CRISPR-Cas effector polypeptide, or a nucleic acid comprising a nucleotide sequence encoding the CRISPR-Cas effector polypeptide; b) one or more CRISPR-Cas guide RNAs, or one or more nucleic acids comprising nucleotide sequences encoding the one or more CRISPR-Cas guide RNAs.
Aspect 34. The method of aspect 30, wherein the targeting polypeptide is an antibody, antibody analog, single chain Fv, diabody, triabody, nanobody or a bi-specific antibody.
Aspect 35. The method of aspect 34, wherein the targeting polypeptide binds to a surface antigen on a T-cell.
Aspect 36. The method of aspect 35, wherein the T cell surface antigen is CD4, CD8, or CD19.
Aspect 37. The method of any one of aspects 30-36, wherein the EDV comprises a nucleic acid comprising a nucleotide sequence encoding a chimeric antigen receptor (CAR).
Aspect 38. The method of aspect 37, wherein the CAR comprises one or more scFv or one or more nanobodies specific for a cancer-associated antigen.
Aspect 39. The method of aspect 38, wherein:
   a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Ra2, CD171, MUC16, PSMA, AXL, CD20, CD80/86, c-MET, DLL-3, DR5, EpHA2, FR-α, gp100, MAGE-A1, MAGE-A3, MAGE-A4, and LMPI; or
   b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin aV03, IGF-R1, IL6, KIR, PD-1, PD-Li, TRAILR1, TRAILR2, transferrin receptor, and VEGF.
Aspect 40. The method of any one of aspects 33-39, wherein at least one of the one or more guide RNAs comprises a nucleotide sequence that hybridizes with a target nucleic acid and provides for knockout of the target nucleic acid.
Aspect 41. The method of aspect 40, wherein the target nucleic acid that is knocked out encodes an immune checkpoint.
Aspect 42. The method of aspect 41, wherein the immune checkpoint is PD-1.
Aspect 43. The method of aspect 40, wherein the target nucleic acid that is knocked out encodes a T-cell receptor alpha constant (TRAC) polypeptide.
Aspect 44. The method of any one of aspects 38-43, wherein said administering treats a cancer in the individual, wherein the cancer comprises cells that express the cancer-associated antigen.
Aspect 45. The method of any one of aspects 33-44, wherein said administering is via intravenous administration.

### Set C

1. An enveloped delivery vehicle (EDV) comprising:
   a) a nucleic acid-binding effector polypeptide; and
   b) one or more fusion polypeptides comprising:
      i) a viral envelop protein; and
      ii) a targeting polypeptide that provides for binding to a target cell.
2. The VLP of 1, wherein the targeting polypeptide comprises one or more antibodies or antibody analogs.
3. The VLP of 2, wherein the one or more antibody analogs is an affibody, an affilin, an affimer, an affitin, an alphabody, an anticalin, an avimer, a DARPin, a Fynomer, a Kunitz domain peptide, a monobody, a repebody, a VLR, or a nanoCLAMP.
4. The EDV of 2, wherein the one or more antibodies is a single chain Fv (scFv) polypeptide, a diabody, a bispecific antibody, a triabody, or a nanobody.
5. The EDV of any one of 1-4, wherein the target cell is a cancer cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.
6. The EDV of any one of 1-4, wherein the target cell is a cancer cell.
7. The EDV of any one of 1-4, wherein the target cell is a CD8⁺ T cell or a CD4⁺ T cell.
8. The EDV of any one of 1-4, wherein the targeting polypeptide comprises an anti-CD19, anti-CD20, anti-CD4, anti-CD28, or anti-CD3 antibody.
9. The EDV of any one of 1-4, wherein the targeting polypeptide comprises: (a) an anti-CD3 and an anti-CD4 antibody; (b) an anti-CD3 and an anti-CD28 antibody; or (c) an anti-CD3, an anti-CD4, and an anti-CD28 antibody.
10. The EDV of any one of 2-9, wherein the targeting polypeptide is a fusion polypeptide comprising:
   (i) the one or more antibodies or antibody analogs; and
   (ii) one or more heterologous polypeptides.
11. The EDV of 10, wherein the one of more heterologous polypeptides comprises a stalk portion of a transmembrane polypeptide.
12. The EDV of 10, wherein the one of more heterologous polypeptides comprises a stalk portion and a transmembrane portion of a transmembrane polypeptide.
13. The EDV of 12, wherein the transmembrane polypeptide is a CD8a chain polypeptide or a platelet-derived growth factor polypeptide.
14. The EDV of 11, wherein the one or more heterologous polypeptides comprises the stalk portion of a CD8a chain polypeptide.
15. The EDV of 11, wherein the one or more heterologous polypeptides comprises the stalk portion and the transmembrane domain of a CD8a chain polypeptide.
16. The EDV of 15, wherein the stalk portion and the transmembrane domain comprises the amino acid sequence TTTPAPRPPTPAPTIASQPLSLRPEACRPAAGGAVHTRGLDFACDIYIWAPLAGTCGVLLL SLVITLYC (SEQ ID NO:20).
17. The EDV of any one of 1-16, wherein the viral envelope protein is selected from a Hepatitis B virus (HBV) glycoprotein, a Hepatitis C virus (HCV) glycoprotein, a Marburg virus glycoprotein, an Ebola virus glycoprotein, a vesicular stomatitis virus (VSV) glycoprotein, an influenza virus hemagglutinin, a SARS-CoV glycoprotein, a respiratory syncytial virus (RSV) glycoprotein, a human parainfluenza virus glycoprotein, a measles virus hemagglutinin and/or a measles virus fusion glycoprotein, an HTLV-1 glycoprotein, a Ross river virus glycoprotein, a rabies virus glycoprotein, a Mokola virus glycoprotein, a Semliki Forest virus glycoprotein, a Sindbis virus glycoprotein, a Venezuelan equine encephalitis virus glycoprotein.
18. The EDV of any one of 1-16, wherein the viral envelope protein is a variant viral envelope protein that comprises one or more amino acid substitutions that reduce binding of the viral envelope protein to its receptor.
19. The EDV of 18, wherein the viral glycoprotein is a variant vesicular stomatitis virus glycoprotein (VSVG) that comprises a substitution of K47 and/or R354, wherein the amino acid numbering is based on the amino acid sequence depicted in FIG. 16A.
20. The EDV of any one of 1-19, wherein the nucleic acid binding effector polypeptide is a CRISPR-Cas effector polypeptide, a Zinc Finger Nuclease (ZFN) or a Transcription activator-like effector nuclease. CRISPR-Cas effector polypeptide is a type II CRISPR-Cas effector polypeptide, a type V CRISPR-Cas effector polypeptide, or a type VI CRISPR-Cas effector polypeptide.
21. The EDV of 20, wherein the CRISPR-Cas effector polypeptide is a type TT CRISPR-Cas effector polypeptide, a type V CRISPR-Cas effector polypeptide, or a type VI CRISPR-Cas effector polypeptide.
22. The EDV of any one of 1-21, wherein the nucleic acid-binding effector polypeptide is a fusion polypeptide comprising: i) a CRISPR-Cas effector polypeptide; and ii) one or more heterologous polypeptides.
23. The EDV of 22, wherein the CRISPR-Cas effector polypeptide exhibits reduced catalytic activity compared to a wild-type CRISPR-Cas effector polypeptide, wherein the CRISPR-Cas effector polypeptide retains the ability to bind to a target nucleic acid when the CRISPR-Cas effector polypeptide is complexed with a guide nucleic acid.
24. The EDV of 22 or 23, wherein at least one of the one or more heterologous polypeptides comprises a deaminase, a reverse transcriptase, a transcription modulator, or an epigenetic modulator.
25. The EDV of any one of 22-24, wherein at least one of the one or more heterologous polypeptides is a lentiviral Gag polypeptide.
26. The EDV of any one of 22-25, wherein the one or more heterologous polypeptides comprises one or more nuclear localization signals.
27. The EDV of any one of 22-26, wherein the one or more heterologous polypeptides comprises a nuclear export signal (NES) polypeptide.
28. The EDV of any one of 1-27, comprising one or more CRISPR-Cas guide RNAs, or one or more nucleic acids comprising nucleotide sequences encoding the one or more CRISPR-Cas guide RNAs.
29. The EDV of any one of 1-28, further comprising a donor template nucleic acid, or a nucleotide sequence encoding the donor template nucleic acid.
30. The EDV of any one of 1-29, further comprising a therapeutic polypeptide, or a nucleic acid comprising a nucleotide sequence encoding a therapeutic polypeptide.
31. The EDV of 30, wherein the therapeutic polypeptide is a chimeric antigen receptor (CAR).
32. The EDV of 31, wherein the CAR comprises one or more scFv or one or more nanobodies specific for a cancer-associated antigen.
33. The EDV of 32, wherein:
   a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRII, IL-13Ra2, CD171, MUC16, PSMA, AXL, CD20, CD80/86, c-MET, DLL-3, DR5, EpHA2, FR-α, gp100, MAGE-Al, MAGE-A3, MAGE-A4, and LMP1; or
   b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin aV03, IGF-R1, IL6, KIR, PD-1, PD-L1, TRAILR1, TRAILR2, transferrin receptor, and VEGF.
34. The EDV of any one of 28-33, wherein at least one of the one or more guide RNAs comprises a nucleotide sequence that hybridizes with a target nucleic acid and provides for knockout of the target nucleic acid.
35. The EDV of 34, wherein the target nucleic acid that is knocked out encodes an immune checkpoint.
36. The EDV of 35, wherein the immune checkpoint is PD-1.
37. The EDV of 34, wherein the target nucleic acid that is knocked out encodes a T-cell receptor alpha constant (TRAC) polypeptide.
38. A composition comprising the EDV of any one of 1-37.
39. The composition of 38, comprising a pharmaceutically acceptable excipient.
40. A method of delivering a nucleic acid-binding effector polypeptide to a eukaryotic cell, the method comprising contacting a eukaryotic cell with the EDV of any one of 1-37, or the composition of 38 or 39.
41. The method of 40, wherein the eukaryotic cell is in vivo.
42. The method of 40, wherein the eukaryotic cell is in vitro.
43. The method of any one of 40-42, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.
44. A method for modifying a target nucleic acid in a eukaryotic cell, the method comprising contacting a eukaryotic cell with the EDV of any one of 1-37, or the composition of
38 or 39, wherein said contacting results in delivery of the nucleic acid-binding effector polypeptide into the cell and modification of a target nucleic acid within the cell.
45. The method of 44, wherein the eukaryotic cell is in vivo.
46. The method of 44, wherein the eukaryotic cell is in vitro.
47. The method of any one of 44-46, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.
48. A method of modifying a target nucleic acid in a target eukaryotic cell in vivo, the method comprising administering to an individual in need thereof an effective amount of the EDV of any one of 1-37, or the composition of 38 or 39, wherein the EDV enters a target eukaryotic cell in the individual and modifies a target nucleic acid in the target eukaryotic cell.
49. The method of 48, wherein the target eukaryotic cell is a CD4⁺ T cell or a CD8′ T cell.
50. The method of 49, wherein the targeting polypeptide comprises an anti-CD3 antibody and an anti-CD28 antibody.
51. The method of 50, wherein the EDV comprises:
   a) a CRISPR-Cas effector polypeptide, or a nucleic acid encoding the CRISPR-Cas effector polypeptide; and
   b) one or more CRISPR-Cas guide RNAs, or one or more nucleic acids encoding the one or more CRISPR-Cas guide RNAs.
52. The method of 48, wherein the targeting polypeptide is an antibody, antibody analog, single chain Fv, diabody, triabody, nanobody or a bi-specific antibody.
53. The method of 52, wherein the targeting polypeptide binds to a surface antigen on a T-cell.
54. The method of 52, wherein the targeting polypeptide binds to CD19, CD20, CD4, CD28, or CD3.
55. The method of any one of 48-54, wherein the EDV comprises a nucleic acid comprising a nucleotide sequence encoding a chimeric antigen receptor (CAR).
56. The method of 55, wherein the CAR comprises one or more scFv or one or more nanobodies specific for a cancer-associated antigen.
57. The method of 56, wherein:
   a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Ra2, CD171, MUC16, PSMA, AXL, CD20, CD80/86, c-MET, DLL-3, DRS, EpHA2, FR-α, gp100, MAGE-Al, MAGE-A3, MAGE-A4, and LMP1; or
   b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin aV03, IGF-R1, 1L6, KIR, PD-1, PD-L1, TRAILR1, TRAILR2, transferrin receptor, and VEGF.
58. The method of any one of 51-57, wherein at least one of the one or more guide RNAs comprises a nucleotide sequence that hybridizes with a target nucleic acid and provides for knockout of the target nucleic acid.
59. The method of 58, wherein the target nucleic acid that is knocked out encodes an immune checkpoint.
60. The method of 59, wherein the immune checkpoint is PD-1.
61. The method of 58, wherein the target nucleic acid that is knocked out encodes a T-cell receptor alpha constant (TRAC) polypeptide.
62. The method of any one of 57-61, wherein said administering treats a cancer in the individual, wherein the cancer comprises cells that express the cancer-associated antigen.
63. The method of any one of 51-62, wherein said administering is via intravenous administration.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

The antibodies used in the working examples below are shown in FIG. 18 and FIG. 35.

### Example 1

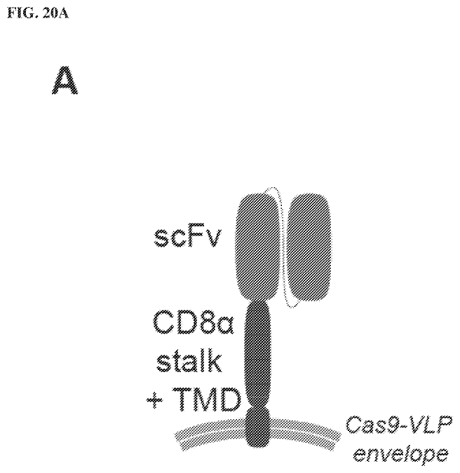

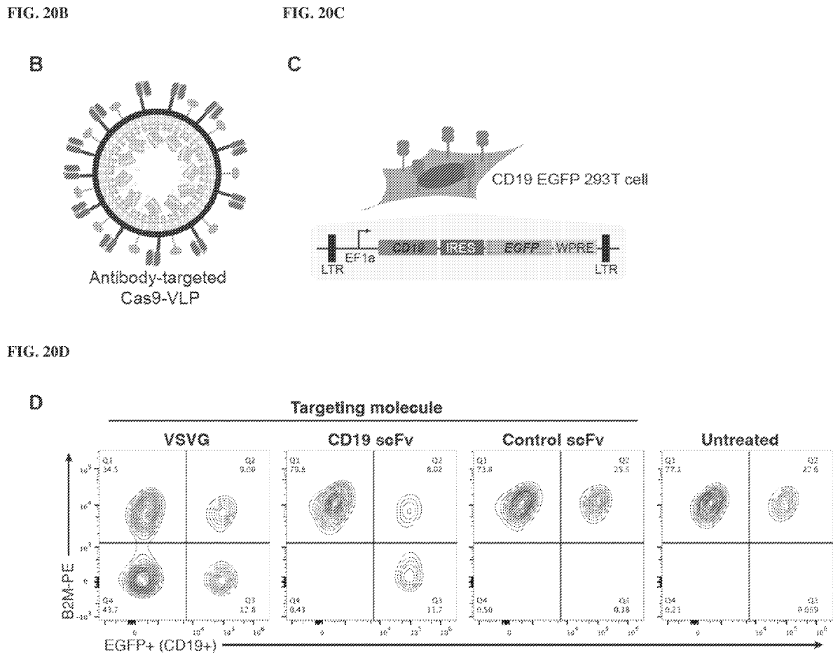

To test whether Cas9-VLP activity could be directed by antibody fragments, a CD19 antibody was cloned as a single-chain variable fragment (scFv) fused to the stalk and transmembrane domain of CD8a (FIG. 20A). CD19 scFv-targeted Cas9-VLPs were generated by co-expressing this CD19-scFv targeting molecule and VSVGmut during Cas9-VLP production. Cas9-VLPs bud from the plasma membrane of transfected producer cells. The outside of Cas9-VLPs were decorated with both scFv targeting molecules and VSVGmut (FIG. 20B). To simplify the study of cell-targeted genome editing by Cas9-VLPs, a 293T cell line co-expressing the B cell ligand CD19 and EGFP was generated, which allowed assessment of genome editing in on-target, EGFP+ cells and off-target, EGFP-bystander cells (FIG. 20C).

A-3:1 mixture of 293T and CD19 EGFP 293T cells was treated with B2M-targeted Cas9-VLPs and genome editing was assessed via B2M protein disruption at 7 days post-treatment by flow cytometry. While the VSVG-pseudotyped Cas9-VLPs mediated genome editing in both CD19⁺ and CD19-populations, treatment with CD19-scFv Cas9-VLPs resulted in the knockout of B2M specifically in CD19⁺ cells. This was specific to the CD19-scFv, as a mismatched control scFv did not result in detectable B2M knockout in either the CD19⁺ or CD19-cells (FIG. 20D). Cell-type-specific genome editing was observed across all Cas9-VLP doses tested, with up to 70% of target cells genome-edited and no editing detected in bystander, non-target cells (FIG. 20E). To investigate the possibility that genome editing was occurring at levels below the sensitivity of flow cytometry analysis, CD19⁺(EGFP+) and CD19-(EGFP-) cells were isolated by FACS and further analyzed by next-generation amplicon sequencing. For cells treated with the CD19-scFv Cas9 VLPs,-74% of reads were modified in on-target EGFP+ cells compared to-3% in bystander cells (FIG. 20F). This bystander genome editing may be attributable to the non-specific uptake of targeted-VLPs; once in the endosome, VSVGmut may mediate endosomal disruption in the absence of targeted cell uptake.

Delivery of genome editing tools inside the body may require selective delivery to rare cell types. The ability of antibody-targeted Cas9-VLPs to mediate cell-type-specific genome editing, when target cells were either abundant or rare within a mixed cell population, was assessed. CD19⁺/EGFP+ cells were diluted with unmodified cells to generate cell mixtures with target cell frequencies ranging from-˜1 to 100%. Antibody-targeted Cas9-VLP treatment resulted in genome-edited target cells across all mixtures tested, even when target cells were as rare as 1% of the population (FIG. 20G).

The modularity and programmability of antibody-targeted Cas9-VLPs to direct genome editing to cells expressing ligands besides CD19 was investigated. A panel of 293T cells was constructed, displaying various plasma membrane proteins expressed by human immune cells: CD20, CD4 and CD28 (FIG. 21). Ligand-expressing 293T cells were generated through lentiviral transduction that would generate cell mixtures where <25% of cells expressed the cognate ligand). In parallel, an additional CD19 scFv targeting molecule, as well as scFvs targeting molecules from monoclonal antibodies specific for CD20, CD4, and CD28, were constructed. Cas9-VLPs displaying candidate scFv targeting molecules were produced and tested on cell mixtures expressing cognate ligands. Genome editing was titratable and selective for ligand+over ligand-bystander cells (FIG. 21B). In all engineered cell mixtures, ligand+ and ligand-cells were similarly susceptible to VSVG-pseudotyped Cas9-VLP genome editing activity. Lastly, it was tested whether the cell-selectivity of antibody-targeted Cas9-VLPs was dependent on antibody-antigen interactions. A panel of CD19, CD20, and CD4 antibody-targeted VLPs as produced; each panel was tested on cell mixtures expressing the cognate ligands. Antibody-targeted Cas9-VLPs only mediated genome editing in cells expressing their matched ligand and not in mismatched ligand-expressing cells (FIG. 21C).

FIG. 20-Cell-specific genome editing with antibody-targeted Cas9-VLPs. (A) Schematic of an antibody-derived single-chain variable fragment (scFv) targeting molecule for Cas9-VLP display. The scFv is fused to the stalk and transmembrane domain (TMD) of human CD8a. (B) Schematic of a Cas9-VLP decorated with a scFv targeting molecule (blue) and VSVGmut (orange). (C) Schematic of the lentiviral vector used for engineering 293T cells that express human CD19 and EGFP. (D-F) Assessment of antibody-targeted Cas9-VLP activity. 293T and CD19 EGFP 293T cells were mixed at an approximate ratio of 3:1 and treated with B2M-targeting Cas9-VLPs displaying various targeting molecule pseudotypes. Analysis was performed at 7 days post treatment to assess B2M knockout in EGFP+(on-target) and EGFP-(off-target) cells by flow cytometry (D, E) and amplicon sequencing (F). Cas9-VLPs were concentrated 10× and cells were treated with 50g1 Cas9-VLPs (D, F) or in a dilution curve (E). (G) Antibody-targeted Cas9-VLPs mediate targeted genome editing regardless of target cell frequency. CD19 EGFP 293T cells were mixed with 293T cells to achieve target cell frequencies of ˜1-100%. Heterogeneous cell mixtures were challenged with antibody-targeted Cas9-VLPs (100 1, 2.5× concentration) and B2M knockout was assessed in EGFP+(on-target) and EGFP-(off-target) bystander cells by flow cytometry. All error bars represent standard error of the mean.

FIG. 21-Antibody-targeted Cas9-VLPs are a programmable, modular strategy for mediating genome editing of specific cells (A) Experimental outline and schematic of the lentiviral vector used for engineering 293T EGFP cells that express various ligands on the plasma membrane. To promote cellular engineering via single lentiviral integration events, engineered cell mixtures were generated via low MOI transduction to achieve<25% EGFP+ cells. Engineered cell mixtures were challenged with B2M-targeting Cas9-VLPs displaying various targeting molecule pseudotypes. (B) Various scFv targeting molecules were developed to target ligands expressed on human immune cells: CD19, CD20, CD4, and CD28. Cell-specific antibody-retargeted Cas9-VLP activity was assessed for on-target, ligand+ cells (EGFP+) and off-target, bystander cells (EGFP-). (C) Antibody-targeted Cas9-VLP-mediated genome editing is highly specific for cells expressing the scFv cognate ligand. scFv-Cas9-VLPs were tested on engineered cells expressing the cognate and noncognate ligands and genome editing activity was measured via flow cytometry at 3 days post treatment. All error bars represent standard error of the mean.

### Example 2: In Vivo Genome Editing

### Materials and Methods

### Plasmid Construction

VSVGmut (K47A R354A VSVG) sequence was human codon-optimized and synthesized as a gBlock (Integrated DNA Technologies) and cloned into the pCAGGS expression plasmid. To generate the CD19 scFv-1 expression plasmid, the sequence encoding the CD8a signal peptide, myc epitope tag, scFv, and CD8a stalk and transmembrane domain of a-CD19-4-1BBi;-P2A-mCherrywas subcloncd into pCAGGS. This plasmid was subsequently used as an entry plasmid for cloning all other scFv antibody fragments: the CD8a signal peptide, myc tag, and scFv sequences were dropped out by EcoRI/Esp3I restriction digest (New England Biolabs) and new DNA sequences encoding CD8a signal peptide and scFv were inserted. This cloning strategy resulted in the removal of the n-terminal myc epitope tag and the addition of a serine amino acid residue between the scFv and CD8a hinge domains. A flexible linker (GGGGSGGGGSGGGGSS; SEQ ID NO:203) was used to link VH and VL domains of source monoclonal antibody sequences. If the antibody source sequence was already a scFv, the linker from the source sequence was used. Except for CD19 scFv-1, all antibody fragment sequences were human codon optimized and synthesized as eBlock Gene Fragments (Integrated DNA Technologies). The panel of Gag-Cas9 variants were cloned using gBlock Gene Fragments (Integrated DNA Technologies). To generate the “optimized guide expression” plasmid set, a U6 promoter-driven guide RNA expression cassette was inserted into the Gag-Cas9 and psPax2 plasmid backbones through SalI-HF (New England Biolabs) restriction digestion and insertion of DNA encoding a PCR-amplified U6-guide RNA expression cassette. InFusion cloning (Takara Bio) was used to generate all plasmids. Additional information on the scFv targeting molecules and source sequences can be found in the table provided in FIG. 18.

A second-generation lentiviral transfer plasmid encoding expression of EFl a promoter-CAR-P2A-mCherry was digested with XbaI & MIuI (New England Biolabs) to drop out the CAR-P2A-mCherry transgene. Human CD19 (Uniprot #Q71UWO) DNA was ordered as a gBlock (Integrated DNA Technologies) and IRES-EGFP (amplified from the Xlone TRE3G MCS-TEV-Halo-3XF IRES EGFP-Nuc-Puro plasmid, a gift from the Darzacq/Tijan Lab) sequences were inserted using InFusion cloning (Takara Bio). This cloning strategy inserted a MluI restriction digest site 3′ of the CD19 stop codon and removed the MluI restriction digest site 3′ of the EGFP stop codon. Human CD4 (Uniprot #P01730), CD20 (Uniprot #P11836), and CD28 (Uniprot #P10747) amino acid sequences were human codon optimized for synthesis and ordered as an eBlock (CD28) or gBlocks (CD20, CD4) (Integrated DNA Technologies). Ligand-encoding sequences were subsequently cloned by restriction digest removal of CD19-encoding sequence from the EFla-CD19 IRES-EGFP lentiviral plasmid using XbaI & MluI (New England Biolabs) and inserted with InFusion cloning (Takara Bio). VSVGmut and scFv targeting plasmids were prepared using the HiSpeed Plasmid Maxi or Plasmid Plus Midi kits (QIAGEN). Lentiviral plasmids were prepared with the QIAprep Spin Miniprep Kit (QIAGEN). All plasmids were sequence-confirmed (UC Berkeley DNA Sequencing Facility, Quintara Bio, or Primordium Labs) prior to use.

### Tissue Culture and Cell Line Generation

Lenti-X and 293T cells (UC Berkeley Cell Culture Facility) were cultured in DMEM (Corning) supplemented with 10% fetal bovine serum (VWR) and 100 U/ml penicillin-streptomycin (Gibco). To generate lentiviruses encoding EFla-ligand IRES-EGFP, 3.5-4 million Lenti-X cells were plated in a 10 cm tissue culture dish (Corning) and transfected with 1 μg pCMV-VSV-G (Addgene plasmid #8454), 10pg psPax2 (Addgene plasmid #12260), and 10 μg of EFla-ligand IRES-EGFP lentiviral transfer plasmid using polyethylenimine (Polysciences Inc.) at a 3:1 PEI:plasmid ratio. Lentiviral-containing supernatants were harvested two days post-transfection and passed through a 0.45 pm PES syringe filter (VWR). Ligand-expressing 293T cells were generated by transducing 293T cells (100,000 per well in a 12-well dish) with lentivirus (0.15-1 ml) in a total well volume of 1 ml. Four days post-transduction, flow cytometry was used to identify cell mixtures where <25% of cells were expressing EGFP. Following expansion, CD19 EGFP 293T cells were additionally sorted for EGFP expression using an SH800S cell sorter (Sony Biotechnology) to generate a population of cells-100% CD19⁺/EGFP+ cells.

Granulocyte-colony stimulating factor (G-CSF)-mobilized human CD34⁺ cells (AllCells) were cultured in SFEMII (StemCell) with 100 U/ml penicillin-streptomycin (Gibco), supplemented with Flt3 ligand, thrombopoietin (TPO), and stem cell factor (Peprotech). Cells were thawed and treated with 8 uM cyclosporine H (Sigma Aldrich) for 18-24 hours prior to Cas9-EDV treatment. Primary human T cells were isolated from cryopreserved peripheral blood mononuclear cells (PBMCs) (AllCells) using the EasySep T cell negative isolation kit (17951, StemCell). Cells were cultured in XVivol5 (Lonza) supplemented with 5% fetal bovine serum, 50 μM 2-mercaptoethanol, and 10 mM N-acetyl L-cysteine (Sigma Aldrich). T cell activation was performed by treating cells with Human T-Activator CD3/CD28 Dynabeads (ThermoFisher) according to the manufacturer's instructions in the presence of 500 U/ml IL-2, 5 ng/ml IL-7, and 5 ng/ml IL-15 (Peprotech). Post 48-72 hr stimulation, T cells were debeaded (EasyEights magnet, StemCell) and cultured in T cell media supplemented with 500U/ml IL-2 (Peprotech).

### Cas9-EDV Production and Quantification

VSVG Cas9-EDVs (formerly known as “Cas9-VLPs”) were produced, packaging B2M-targeted Cas9 RNPs with the guide RNA spacer sequence 5′-GAGTAGCGCGAGCACAGCTA (SEQ ID NO:208), or TRAC-targeted with the guide RNA spacer sequence 5′-AGAGTCTCTCAGCTGGTACA (SEQ ID NO:209), or PDCD1-targeted with the guide RNA spacer sequence 5′-CGACTGGCCAGGGCGCCTGT (SEQ ID NO:210). Briefly, 3.5-4 million Lenti-X cells (Takara Bio) were seeded into 10 cm tissue culture dishes (Corning) and transfected the next day with 1 μg pCMV-VSV-G (Addgene plasmid #8454), 6.7 μg Gag-Cas9 (Addgene plasmid #171060) (or the optimized, guide-expressing Gag-NES-2xp53NLS-Cas9 plasmid), 3.3 μg psPax2 (Addgene plasmid #12260) (or the optimized, guide-expressing psPax2), and 10g U6-B2M (Addgene plasmid #171635) using polyethylenimine (Polysciences Inc.) at a 3:1 PEI:plasmid ratio. For use in mice, Cas9-EDVs were produced with the inverse ratio of 3.3 g optimized Gag-Cas9 and 6.7 gg optimized psPax2 plasmids. Supernatant was switched into Opti-MEM 6-18 hours post transfection when producing Cas9-EDV or lentiviral samples that would be used to treat primary cells or mice. Two days post-transfection, (or media change) Cas9-EDV-containing supernatants were harvested and passed through a 0.45 pm PES syringe filter (VWR) and concentrated with Lenti-X Concentrator (Takara Bio) according to the manufacturer's instructions. Concentrated Cas9-EDVs were resuspended in Opti-MEM (Gibco), SFEM II (StemCell) or T cell media at a final concentration of 10× unless otherwise noted in the figure legend. For use in mice, Cas9-EDV and lentiviral samples were concentrated using ultracentrifugation (25,000 RPM, 2 hours, using a SW28 rotor) and resuspended in sterile phosphate buffered saline (PBS) (Gibco).

Cas9-EDVs were either stored at 4° C. or frozen at −80° C. within an isopropanol-filled freezing container until use.

Antibody-targeted Cas9-EDVs were produced in the same way as VSVG Cas9-EDVs, except that the pCMV-VSV-G plasmid was omitted and 7.5 μg of the scFv targeting plasmid and 2.5 μg of VSVGmut were included during transfection.

Cas9-EDV and lentiviral samples were titered using a QuickTiter™ Lentivirus Titer Kit (Lentivirus-Associated HIV p24) (CellBio Labs) according to the manufacturer's instructions.

### Mouse Experiments

Human PBMC-engrafted NSG mice (“humanized mice”) were purchased from Jackson Laboratories. mice were anesthetized with isoflurane and retro-orbitally administered 100ul Cas9-EDVs or lentivirus resuspended in sterile PBS (doses are indicated in the figure legends). At the experimental endpoint, mice were euthanized via CO₂ inhalation, spleens were pressed through a 100 um filter to generate a single cell suspension, and red blood cells were isolated. Human immune cells were analyzed by flow cytometry, either by first gating on human CD45⁺ or by pre-isolating human immune cells prior to analysis using the EasySep™ Release Human CD45 Positive Selection Kit for humanized mouse samples (100-1007, StemCell).

### Flow Cytometry

To assess genome editing via flow cytometry, cells were stained with anti-human B2M-APC (316312, Biolegend) or human B2M-PE (316306, Biolegend) or human TCR-APC (306718, Biolegend) in PBS containing 1% bovine serum albumin and an Attune N×T flow cytometer with 96-well autosampler (Thermo Fisher Scientific) was used for flow cytometry analysis. Ligand expression was confirmed for engineered 293T cells using anti-human CD28-PE (302907, Biolegend), anti-human CD20-PE (302306, Biolegend), anti-human CD4-PE/Cyanine7 (300512, Biolegend) and anti-human CD19-PE (302208, Biolegend). Immune cell immuophenotyping was performed with human CD4-FITC (300538, 557746 Biolegend), human CD8-PE-Cy7 (557746 Biolegend), human CD45-PE-Cy7 (368531, Biolegend), and CD19-FITC (302206, Biolegend). Data analysis was performed using FlowJo v10 10.7.1 (FlowJo, LLC, Ashland OR). APC: allophycocyanin. PE: phycoerythrin. FITC: fluorescein isothiocyanate.

### Amplicon Sequencing

Next-generation sequencing was used for detection of on-target genome editing in EGFP+ and EGFP-sorted 293T cells. Genomic DNA was extracted using QuickExtract (Lucigen) as previously described. PrimeStar GXL DNA polymerase (Takara Bio) was used to amplfy the B2M Cas9-RNP target site using primers 5′-GCTCTTCCGATCTaagctgacagcattcgggc (SEQ ID NO:211) and 5′-GCTCTTCCGATCTgaagtcacggagcgagagag (SEQ ID NO:212). The resulting PCR products were cleaned up using magnetic SPRI beds (UC Berkeley DNA Sequencing Facility). Library preparation and sequencing was performed by the Innovative Genomics Institute Next-Genration Sequencing Core using MiSeq V2 Micro 2×150 bp kit (Illumina). Reads were trimmed and merged (Geneious Prime, version 2022.0.1) and analyzed with CRISPResso2 (http://crispresso.pinellolab.partners.org/login).

### Statistical Analysis

Statistical analysis was performed using Prism v9. Statistical details for all experiments, including value and definition of N, and error bars can be found in the Figure Legends.

### Results

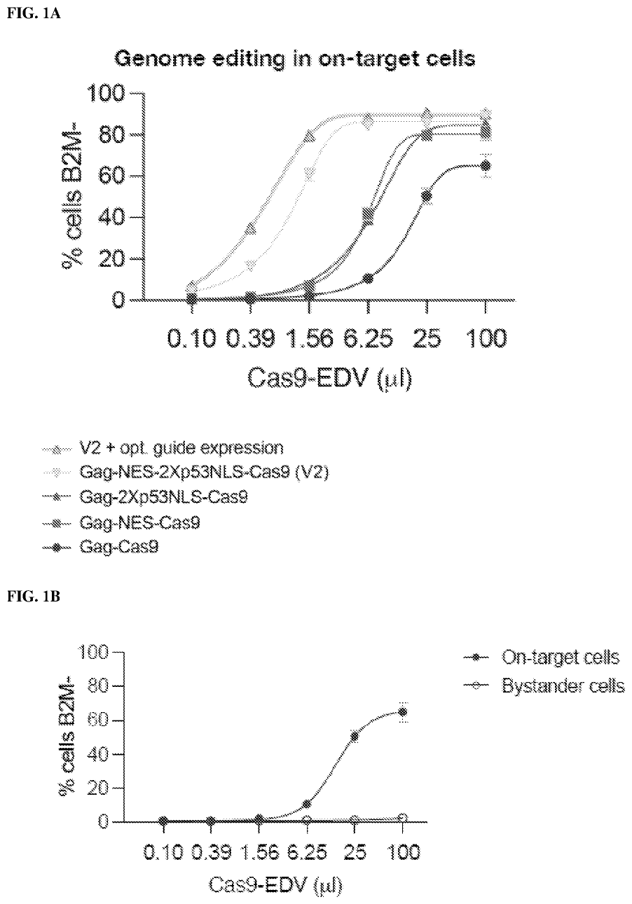

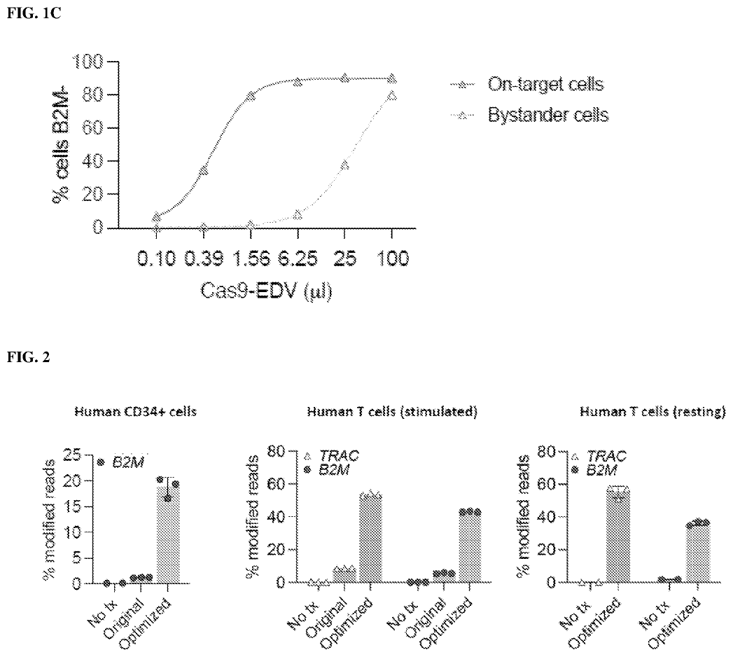

The results are depicted in FIG. 1-14.

FIG. 1A: A panel of CD19-scFv targeted EDVs (packaging Cas9 RNP complexes targeting the β-2 microglobulin (“B2M”) locus for genome editing) were produced using various Gag-Cas9 polypeptide compositions and concentrated approximately 15-fold using LentiX Concentrator (˜10 ml EDV-containing supernatant into 0.65 ml Opti-MEM). A mixture of CD19-(˜80%) and CD19⁺EGFP+(20%) 293T cells were treated with the panel of EDVs at the indicated volumes (15k cells/96-well, 150ul total well volume). The loss of B2M expression (indicative of genome editing at the B2M locus) was assessed three days post-treatment in CD19⁺EGFP+on-target cells.

FIG. 1B: A mixture of CD19-(˜80%) and CD19⁺EGFP+(20%) 293T cells were treated with “Gag-Cas9” EDVs (packaging Cas9 RNP complexes targeting the B2M locus for genome editing) at the indicated volumes (15k cells/96-well, 150ul total well volume). The loss of B2M expression (indicative of genome editing at the B2M locus) was assessed three days post-treatment in CD19⁺EGFP+on-target cells and CD19-EGFP-bystander cells.

FIG. 1C: A mixture of CD19-(˜80%) and CD19⁺EGFP+(20%) 293T cells were treated with “V2⁺opt. guide expression” EDVs (packaging Cas9 RNP complexes targeting the B2M locus for genome editing) at the indicated volumes (15k cells/96-well, 150ul total well volume). The loss of B2M expression (indicative of genome editing at the B2M locus) was assessed three days post-treatment in CD19⁺EGFP+on-target cells and CD19-EGFP-bystander cells.

FIG. 2: VSVG-pseudotyped original (“Gag-Cas9”) and optimized (“V2⁺opt. guide expression”) EDVs (packaging Cas9 RNP complexes targeting the B2M locus for genome editing) were produced and concentrated approximately 50-fold using LentiX Concentrator (˜10 ml EDV-containing supernatant into 0.2 ml). Next generation sequencing (NGS) was performed to assess genome editing at 3 days (human CD43⁺ cells) or 4 days (human T cells) post-treatment.

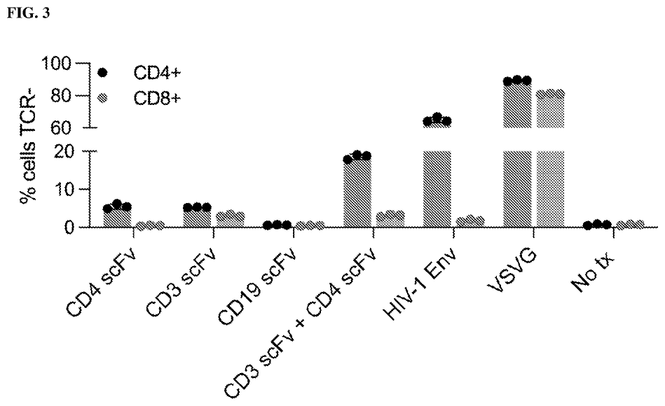

FIG. 3: Cas9-EDV genome editing activity can be directed to specific primary human cells using scFv-targeting molecules. A panel of optimized Cas9-EDVs (packaging TRAC-targeting Cas9 RNP complexes) were produced with various indicated pseudotypes and concentrated approximately 200-fold (30 ml supernatant (sup) into 150 μl T cell media). 50 μl Cas9-EDVs were used to treat 15k pre-stimulated primary human cells in a final well volume of 100ul. Flow cytometry was performed at five days post treatment to assess the loss of T cell receptor (TCR) expression in CD4⁺ and CD8⁺ cells. As expected, CD4 scFv-targeted Cas9-EDVs mediated TCR knockout in CD4⁺ cells but not in co-cultured CD8⁺bystander cells. CD3 scFv-targeted Cas9-EDVs mediated editing in both CD4⁺ and CD8⁺. Cas9-EDVs displaying CD19 scFv targeting molecules (which should not bind T cells) did not result in editing of either population. Multiplexing the display of CD3 and CD4 scFv-targeting molecules enhances genome editing over displaying either CD3 or CD4 scfvs alone. Cas9-EDVs pseudotyped with HIV-1 and VSVG are presented as controls-HIV-1 Env pseudotyped EDVs mediate genome editing specifically in CD4⁺ T cells, while VSVG pseudotyped EDVs mediate genome editing in CD4⁺ and CD8⁺ T cells.

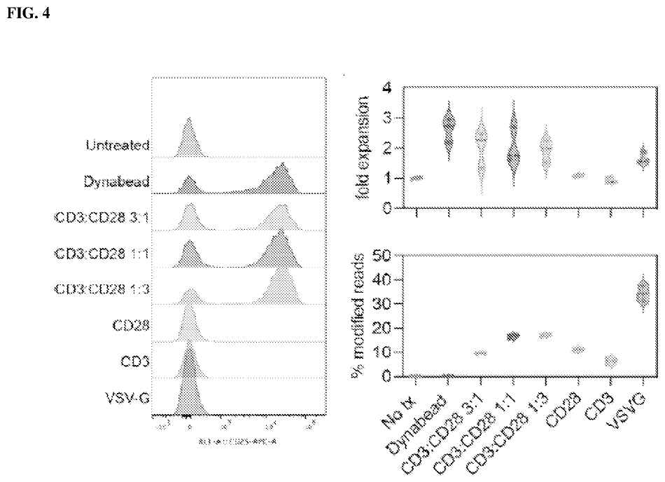

FIG. 4: Multiplexing CD3 and CD28 scFv targeting molecules on Cas9-EDVs enables T cell activation and proliferation. A panel of optimized Cas9-EDVs (packaging PDCD1-targeting Cas9 RNP complexes) were produced with various indicated pseudotypes and concentrated approximately 62.5-fold (10 ml sup into 160 μl T cell media). 50ul were used to treat 30k pre-stimulated primary human cells in a final well volume of 100 μl. Three days post treatment, cell count and expression of CD25 (a marker of T cell activation) was analyzed by flow cytometry. Cell count was used to calculate a fold expansion, normalized to untreated “no tx” cells. Additionally, DNA was extracted and genome editing at the PDCD1 locus was assessed by next-generation amplicon sequencing.

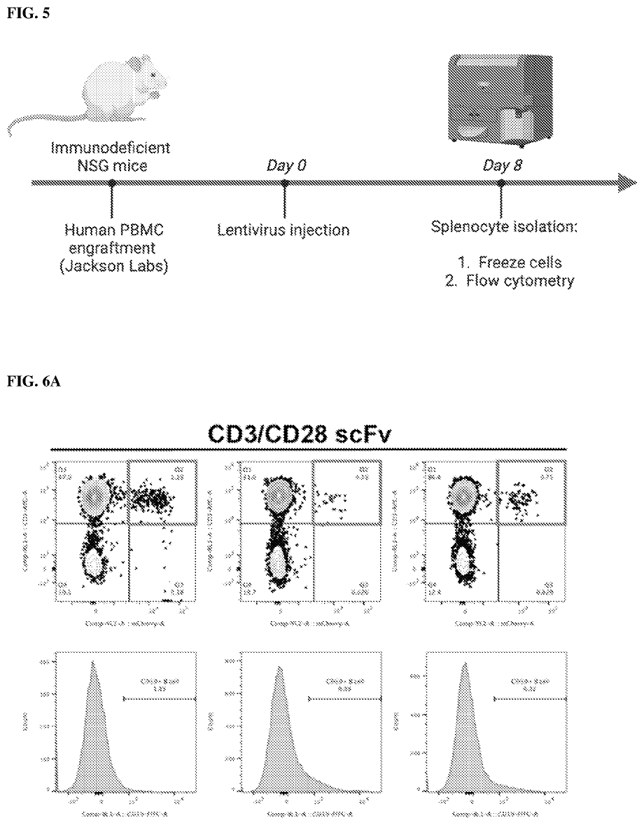

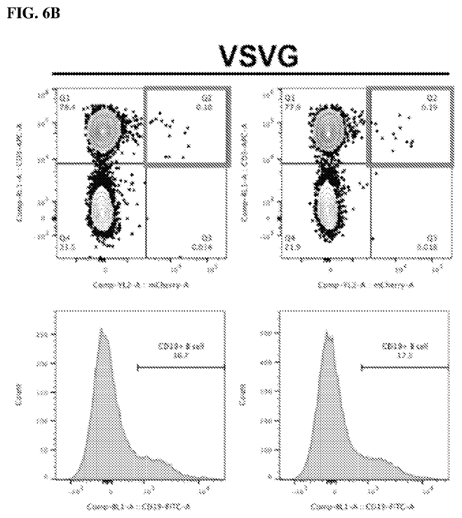

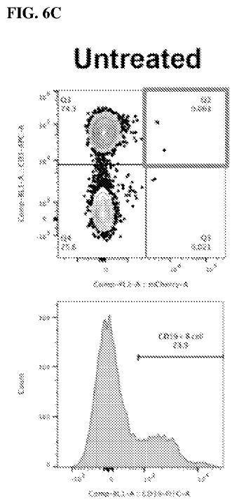

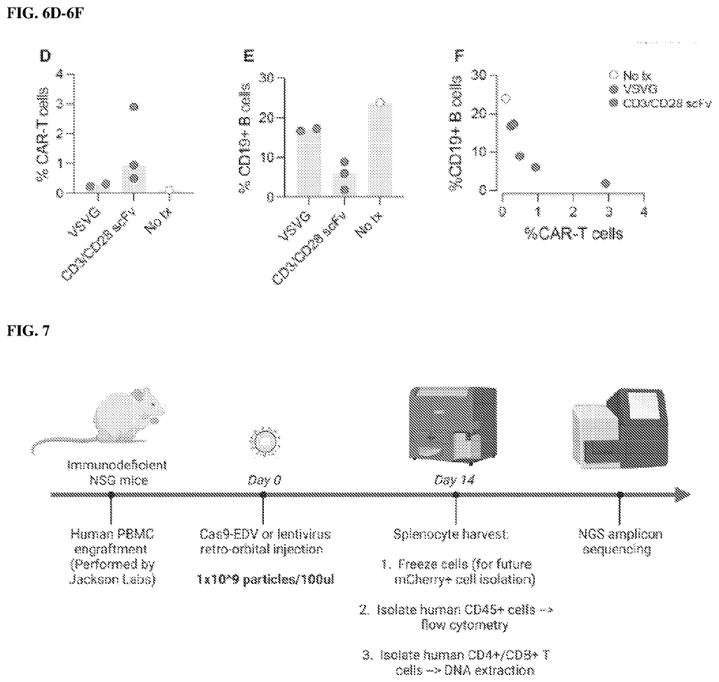

FIG. 5: Experimental overview schematic (refers to FIG. 6A-6F). Immunodeficient NSG mice engrafted with human PBMCs were procured from Jackson Laboratories and treated with lentiviral vectors encoding an expression cassette for an anti-CD19 chimeric antigen receptor (CAR) and mCherry fluorescent protein. The lentiviral vectors were either pseudotyped with VSVG or with a 1:1 mix of CD3 and CD28 scFv targeting molecules. 100 μl of 1.79×10\hat{ }11 particles/ml in PBS were administered per mouse. Mice were sacrificed 8 days post treatment and splenocytes were immunophenotyped via flow cytometry to assess CAR/mCherry expression.

FIG. 6A: Top: Lentivirus pseudotyped with CD3 and CD28 scFv targeting molecules enables in vivo generation of human CAR-T cells. CAR+/mCherry+human CD45⁺CD3⁺ T cells are observed. Bottom: In vivo CAR-T generation results in reduced percentages of B cells present in the spleen (% of human CD45⁺ cells that are CD19⁺ B cells), compared to the untreated mouse (FIG. 6C).

FIG. 6B: Top: Lentivirus pseudotyped with VSVG enables in vivo generation of human CAR-T cells, but with lower efficiency compared to lentivirus pseudotyped with CD3 and CD28 scFv targeting molecules (FIG. 6A). CAR+/mCherry+human CD45⁺CD3⁺ T cells are observed. Bottom: In vivo CAR-T generation results in reduced percentages of B cells present in the spleen (% of human CD45⁺ cells that are CD19⁺ B cells), compared to the untreated mouse (FIG. 6C).

FIG. 6C: Top: No CAR+/mCherry+human CD45⁺CD3⁺ T cells are observed in splenocytes isolated from an untreated mouse. Bottom: The percentages of B cells present in the spleen (% of human CD45⁺ cells that are CD19⁺ B cells) of an untreated mouse.

FIG. 6D-6F. Summary of flow cytometry data presented in FIG. 6A-6C.

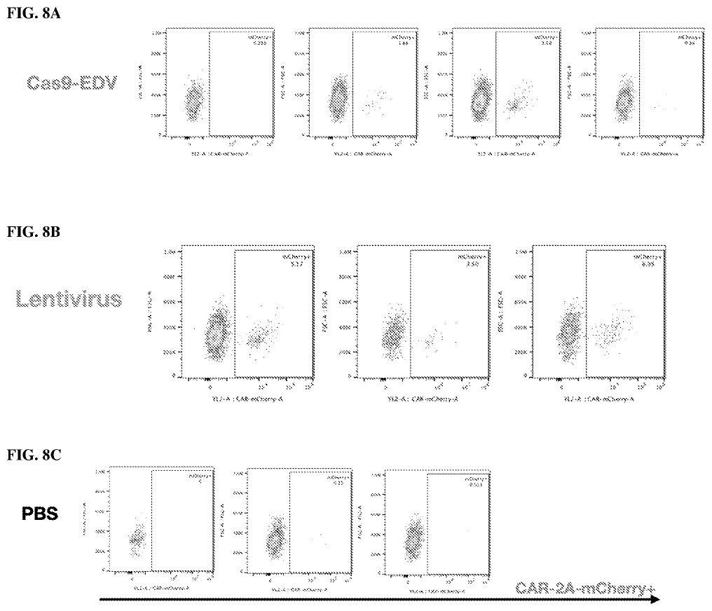

FIG. 7: Experimental overview schematic (refers to FIG. 8-14). Immunodeficient NSG mice engrafted with human PBMCs were procured from Jackson Laboratories. On day 0, mice were treated with Cas9-EDV or lentiviral vectors encoding an expression cassette for an anti-CD19 chimeric antigen receptor (CAR) and mCherry fluorescent protein (with the Cas9-EDVs additionally packaging Cas9 RNPs targeting TRAC for disruption). Both vectors were pseudotyped with a 1:1 mix of CD3 and CD28 scFv targeting molecules. 100ul of 1×10\hat{ }10 particles/ml in PBS were administered per mouse. Mice were sacrificed 14 days post treatment, subjected to the indicated cell isolation protocols, and cells were immunophenotyped via flow cytometry to assess CAR/mCherry expression.

FIG. 8A: Cas9-EDVs pseudotyped with CD3 and CD28 scFv targeting molecules enable in vivo generation of human CAR-T cells. Each plot depicts cells from an individual mouse. CAR+/mCherry+human CD45⁺CD19-T cells are observed in 3 of 4 mice treated with T cell-targeted Cas9-EDVs.

FIG. 8B: Lentivirus pseudotyped with CD3 and CD28 scFv targeting molecules enable in vivo generation of human CAR-T cells. Each plot depicts cells from an individual mouse. CAR+/mCherry+human CD45⁺CD19-T cells are observed in 3 of 3 mice treated with T cell-targeted lentivirus.

FIG. 8C: No CAR+/mCherry+human CD45⁺CD19-T cells are observed in PBS-treated control mice. Each plot depicts cells from an individual mouse.

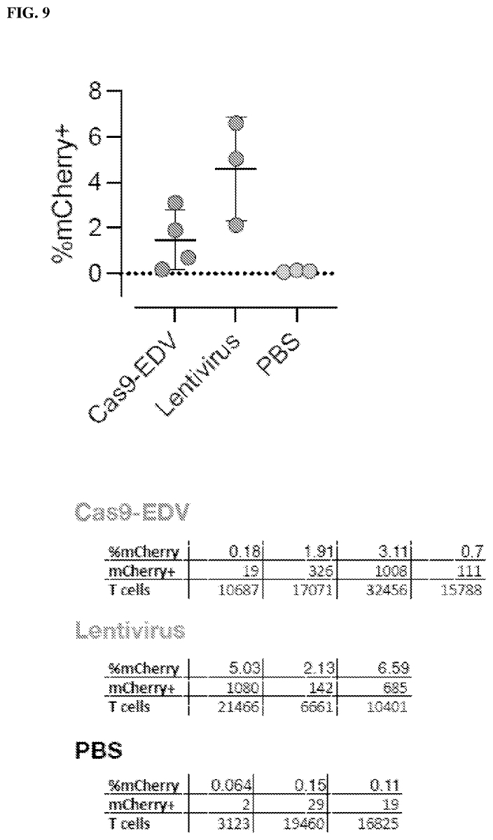

FIG. 9: Summary data quantifying in vivo-generated CAR+/mCherry+CD45⁺human T cells (either CD4⁺ or CD8⁺) using T cell-targeted Cas9-EDVs or lentivirus.

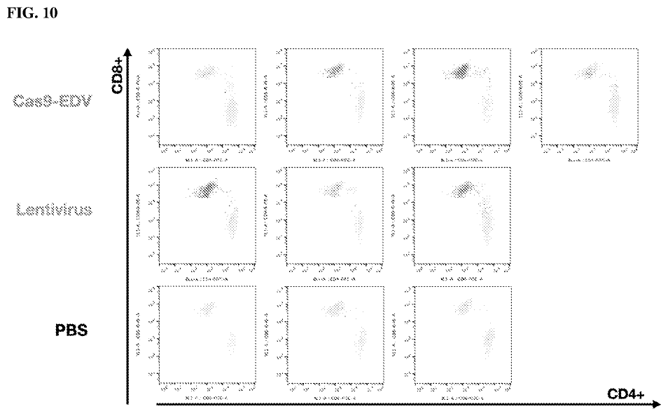

FIG. 10: Plot depicting CAR+/mCherry+T cells. The majority of CAR+/mCherry+ cells are CD8⁺ T cells.

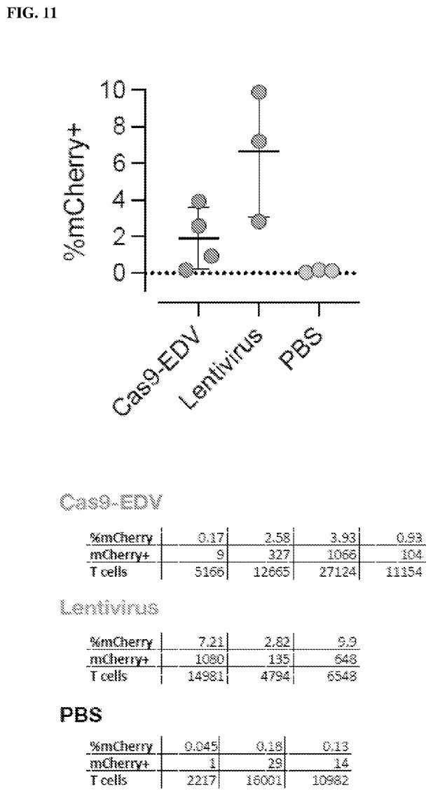

FIG. 11: Summary data quantifying in vivo-generated CAR+/mCherry+CD45⁺human CD8⁺ T cells using T cell-targeted Cas9-EDVs or lentivirus.

FIG. 12A: Assessment of T cell-targeted in vivo genome editing. Cas9-EDVs package Cas9 RNPs that target the TRAC locus to disrupt T cell receptor (TCR) expression. TCR expression was assessed in T cells either expressing CD4 or CD8.

FIG. 12B: Summary data quantifying the percent of TCR-negative T cells (from FIG. 12A).

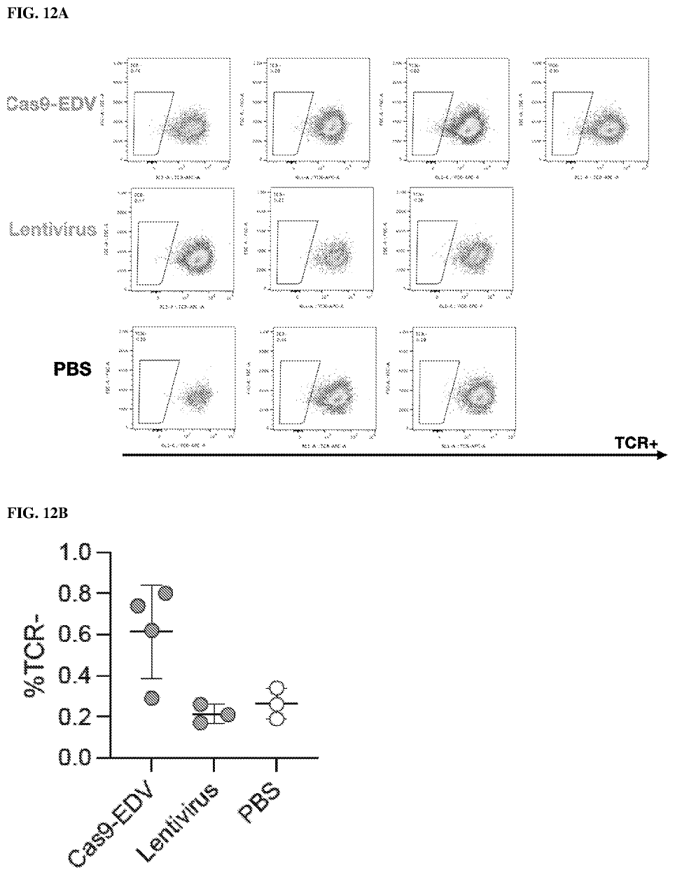

FIG. 13A: Assessment of T cell-targeted in vivo genome editing. Cas9-EDVs package Cas9 RNPs that target the TRAC locus to disrupt T cell receptor (TCR) expression. TCR expression was assessed in T cells expressing CD8.

FIG. 13B: Summary data quantifying the percent of TCR-negative CD8⁺ T cells (from FIG. 13A).

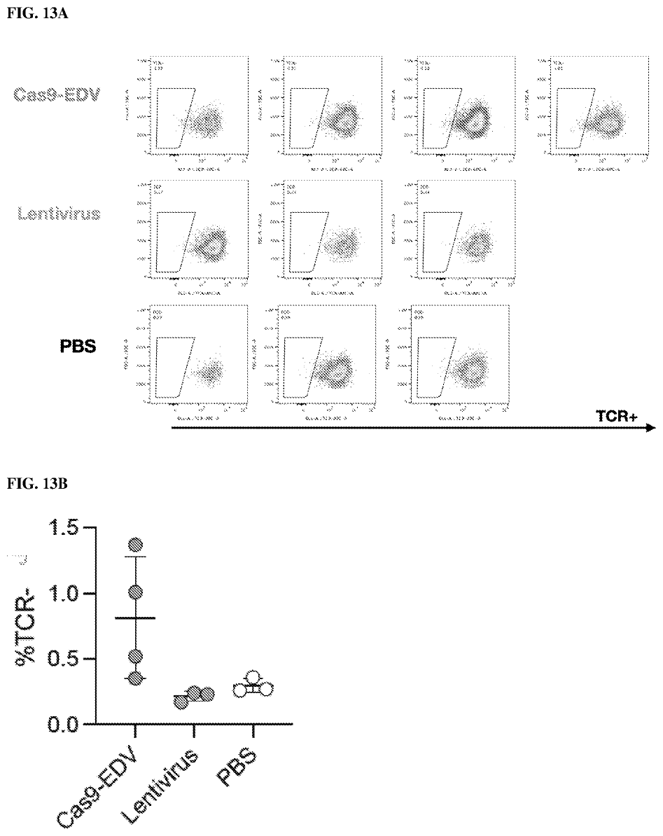

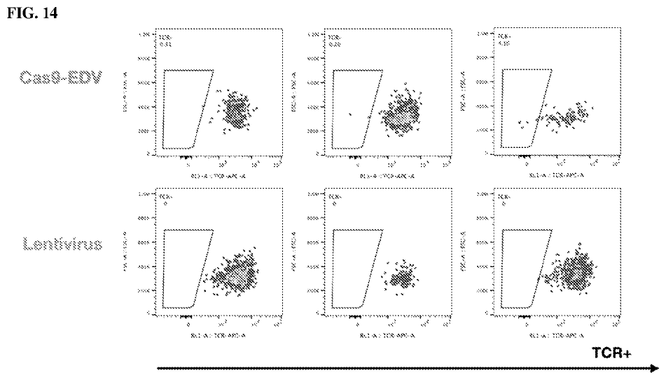

FIG. 14: Assessment of T cell-targeted in vivo genome editing. Cas9-EDVs package Cas9 RNPs that target the TRAC locus to disrupt T cell receptor (TCR) expression. TCR expression was assessed in CAR+/mCherry+T cells either expressing CD4 or CD8.

### Example 3

The results demonstrate cell-specific genome editing can be achieved both ex vivo and in vivo by pairing the display of VSVGmut with single-chain antibody fragments (scFvs) on EDVs that package Cas9 ribonucleoprotein (RNP) complexes (Cas9-EDVs). EDVs described herein leverage retroviral VLP assembly for the transient delivery of Cas9 RNP. Cas9-EDVs achieved targeted genome editing within in vivo-generated CAR T cells in humanized mice, with no off-target delivery to liver hepatocytes. These data show that EDVs are a programmable, cell-specific delivery platform for complex genome engineering in vivo.

### Results

Receptor-Mediated Delivery of Genome Editors with Cas9-EDVs

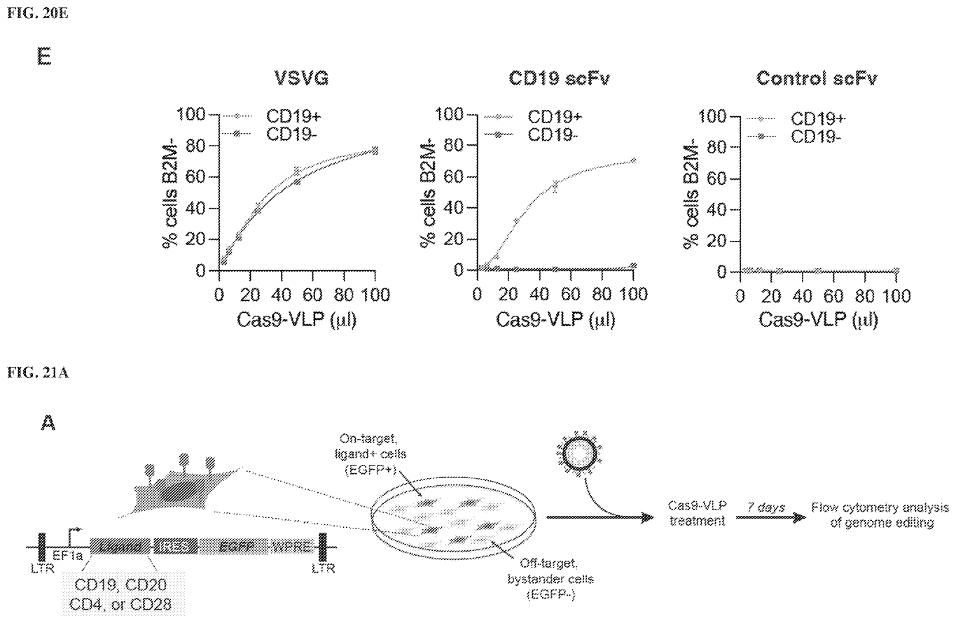

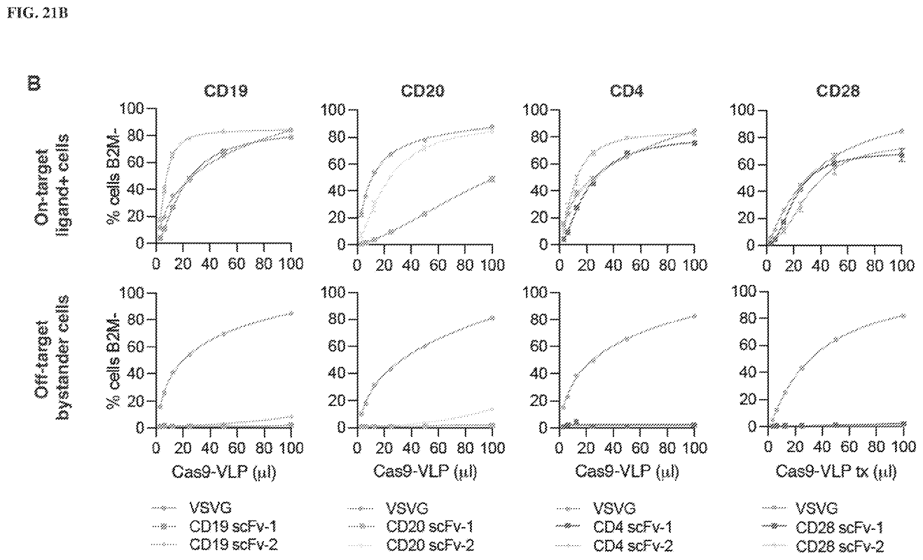

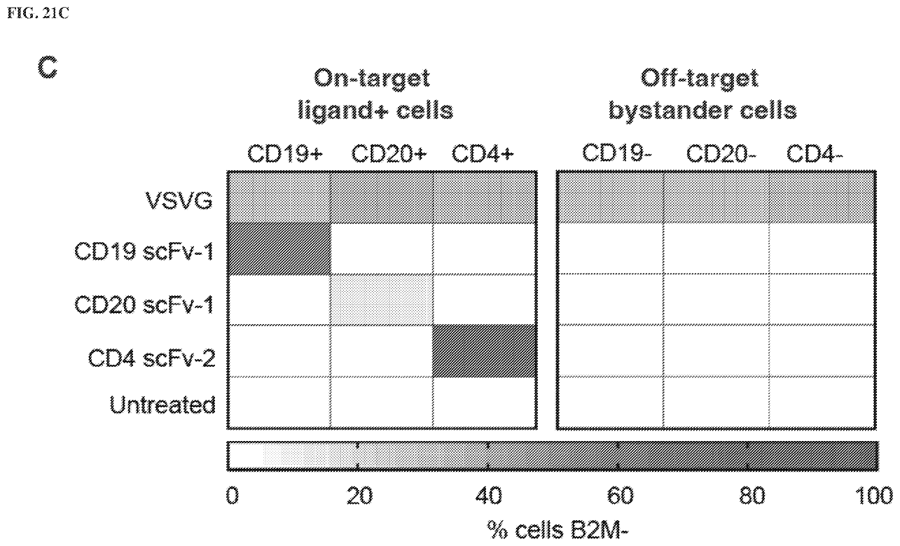

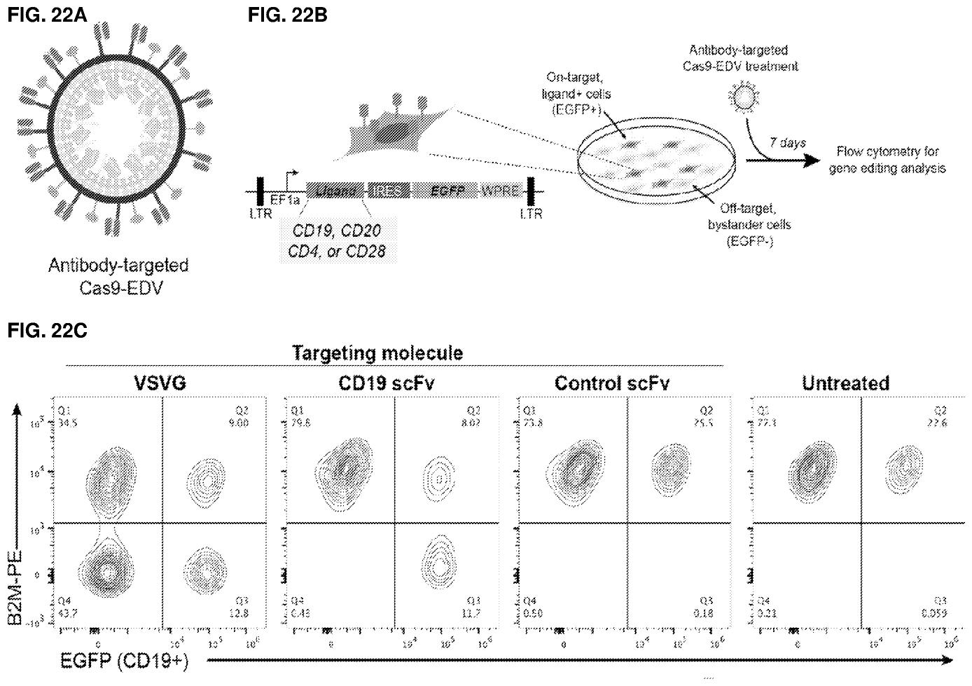

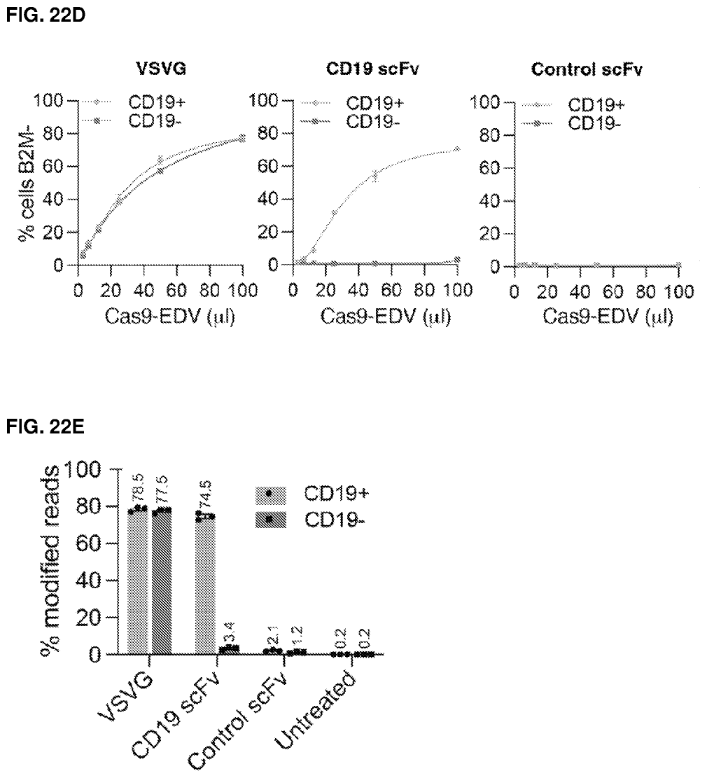

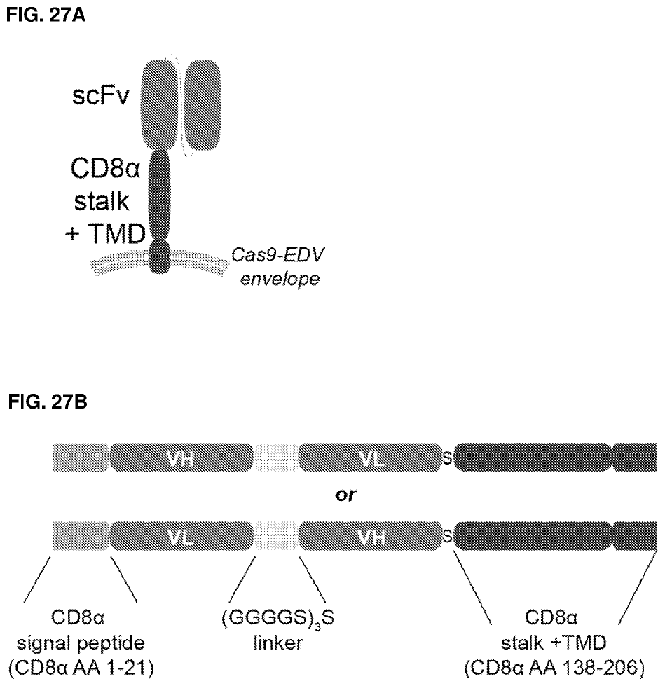

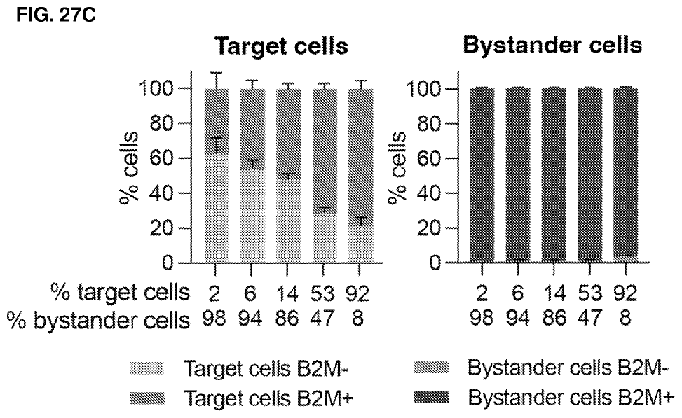

A major challenge for in vivo delivery of editing enzymes is the lack of vehicles capable of targeting specific cell types. VLPs can package Cas9 RNP complexes produced by over-expressing Cas9 fused to the C-terminal end of the viral Gag polyprotein during VLP production, but cell-selective VLP targeting has relied on cell infection strategies evolved by enveloped viruses. To test whether VLPs could be reformulated as programmable EDVs, an anti-CD19 scFv fused to the stalk and transmembrane domain of CD8a was first cloned (FIG. 22A; FIG. 27A, B). It was hypothesized that co-expression of a scFv targeting molecule and VSVGmut together with lentiviral components necessary for Cas9 RNP encapsulation would generate Cas9-EDVs possessing both receptor specificity and endosomal escape capability.

To analyze the receptor-mediated function of Cas9-EDVs, a HEK293T cell line that co-expresses both the B-cell ligand CD19 and EGFP was generated, facilitating assessment of genome editing in on-target, EGFP+ cells, and off-target, EGFP-bystander cells (FIG. 22B). Cas9-EDVs were produced containing single guide RNA (sgRNA) targeting the B2M gene and outwardly displaying either VSVG, CD19 scFv+VSVGmut, or a control scFv+VSVGmut that should not recognize the target cells in this experiment. In a-3:1 mixture of HEK293T and CD19 HEK293T cells, VSVG Cas9-EDVs mediated genome editing in both CD19⁺ and CD19-populations, whereas the CD19 scFv Cas9-EDVs induced the knockout of B2M only in CD19⁺ cells (FIG. 22C). No knockout was observed in either the CD19⁺ or CD19-cells treated with control scFv+VSVGmut Cas9-EDVs. Antibody-targeted Cas9-EDV activity was titratable, with up to 74% of target cells exhibiting genome editing with little to no editing detected in bystander cells (FIG. 22D, E). Antibody-targeted Cas9-EDVs produced genome edits in target cells present at 2-92% of a cell mixture, whereas bystander cell editing was unchanged (FIG. 27C). Together these results demonstrate the ability of EDVs to deliver functional molecular cargo in a receptor-mediated fashion.

Programmable Cell-Specific Genome Editing with Cas9-EDVs

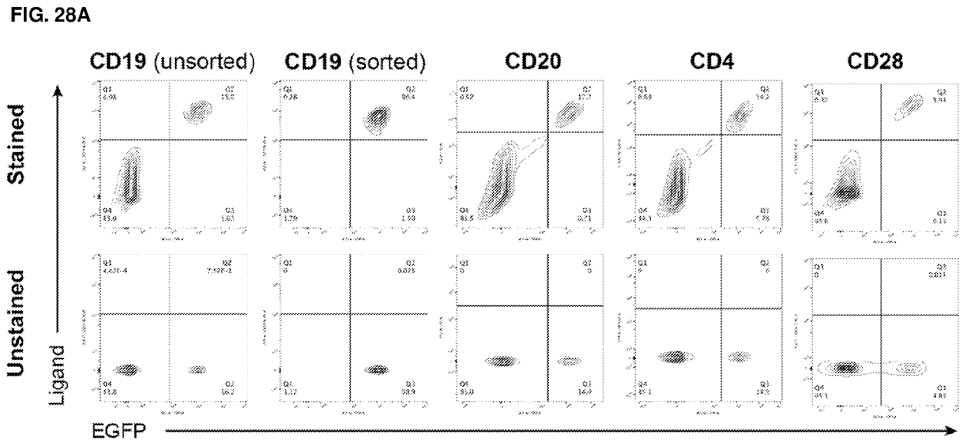

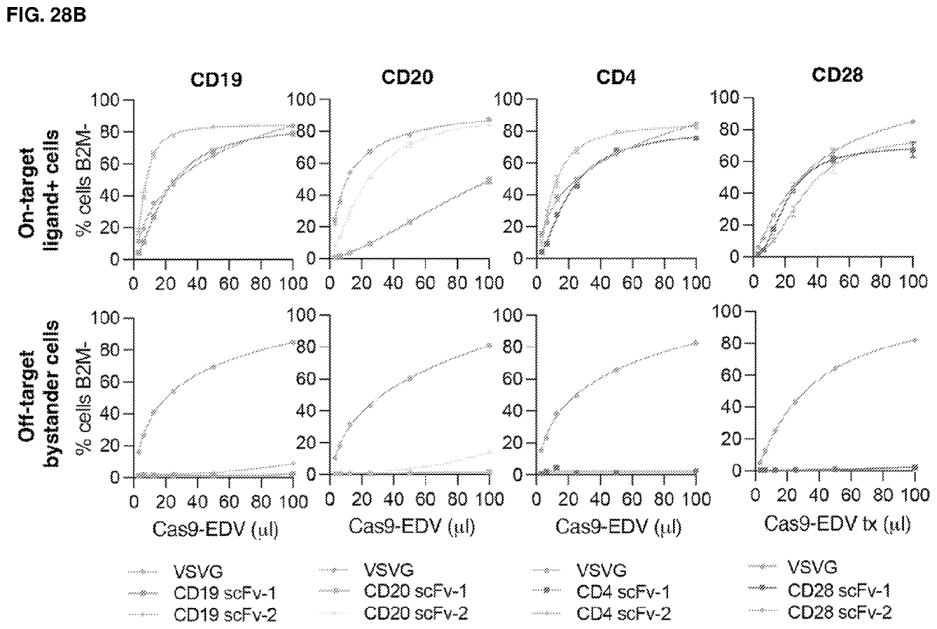

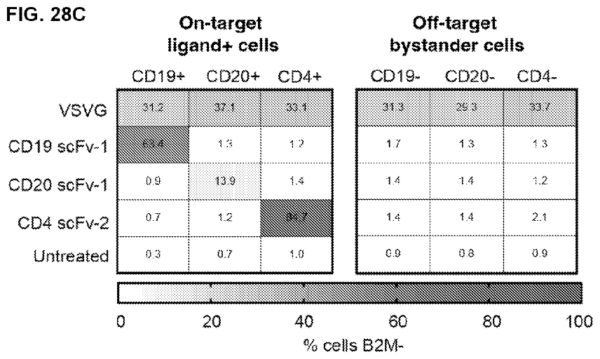

Receptor-mediated delivery of genome editing molecules could enable targeted engineering of any cell type as a function of its surface antigens. To test this possibility, the modularity and programmability of Cas9-EDVs to direct genome editing in HEK293T cells displaying various plasma membrane proteins normally expressed by human immune cells, including CD20, CD4 and CD28 was investigated (FIG. 22B; FIG. 28A). Cas9-EDVs displaying VSVGmut and scFv-based CD20, CD4 and CD28 targeting molecules, generated in both VH-linker-VL and VL-linker-VH orientations (Table S1) (29), induced up to 80% genome editing that was titratable and selective for ligand(+) over ligand(−) cells (FIG. 28B). Not all scFvs produced the same level of on-target cell editing, but in no case was an scFv-displaying EDV able to induce more than minimal editing in off-target cells lacking the cognate ligand. Furthermore, in all engineered cell mixtures, ligand(+) and ligand(−) cells were similarly susceptible to genome editing when treated with Cas9-EDVs displaying the VSVG fusogen (FIG. 28B). A panel of CD19, CD20 and CD4 antibody-targeted EDVs only mediated genome editing in cells expressing their matched ligand and not in mismatched ligand-expressing cells, demonstrating that delivery requires antibody-antigen interactions (FIG. 28C).

### Cas9-EDV Optimization Enhances Genome Editing and Reveals Nonessential Components

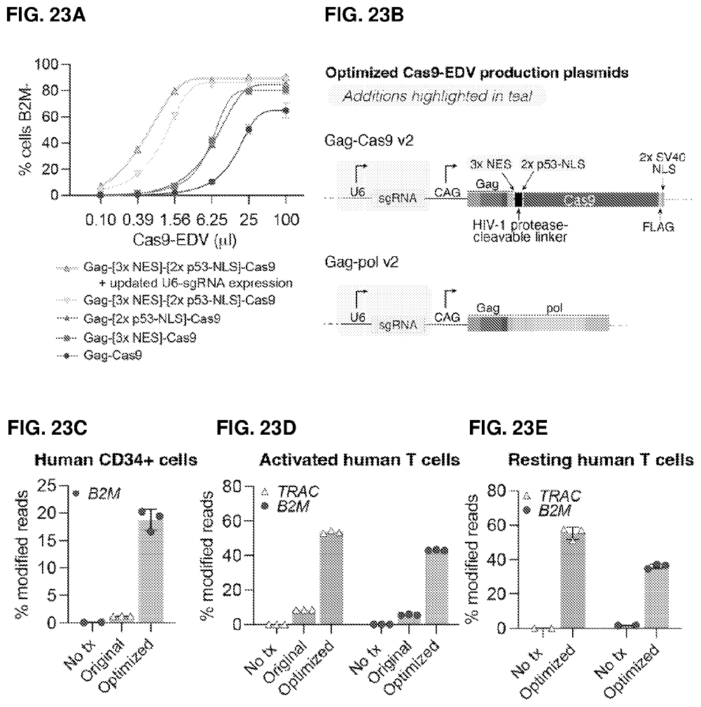

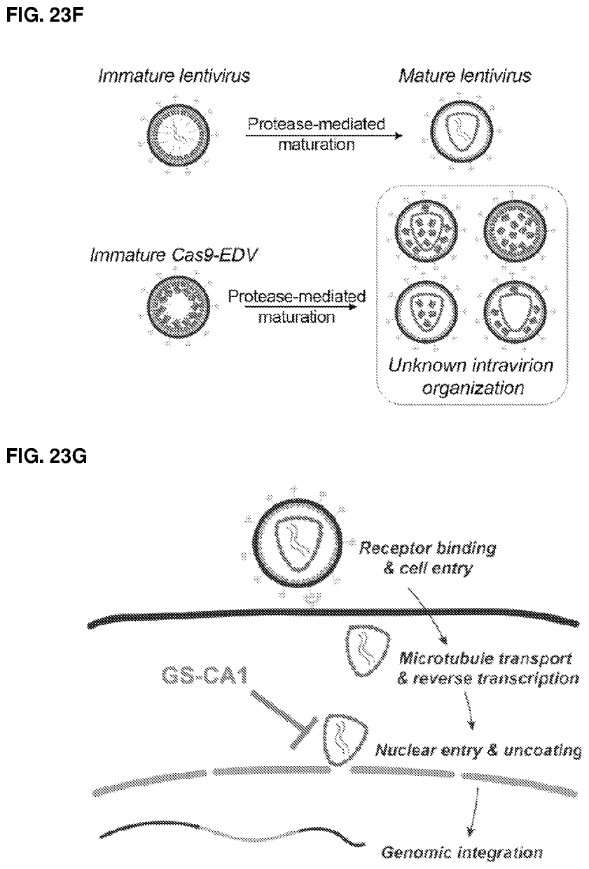

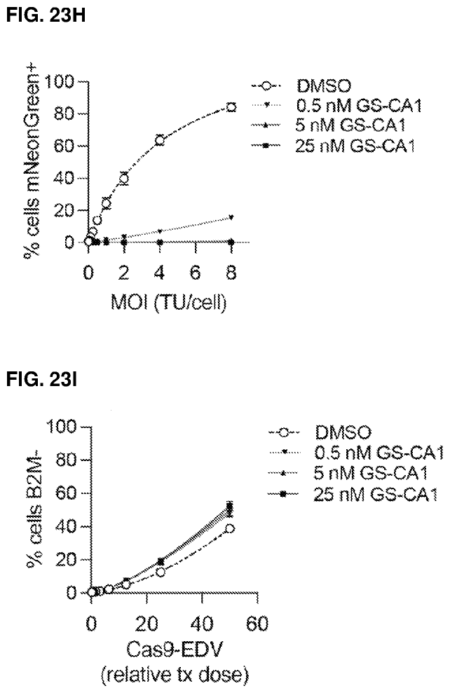

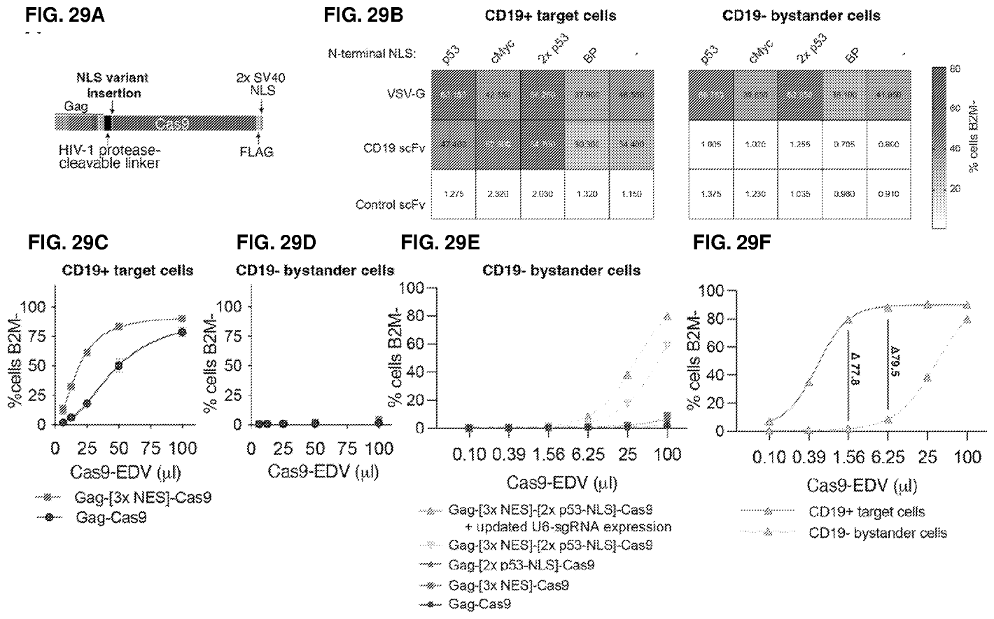

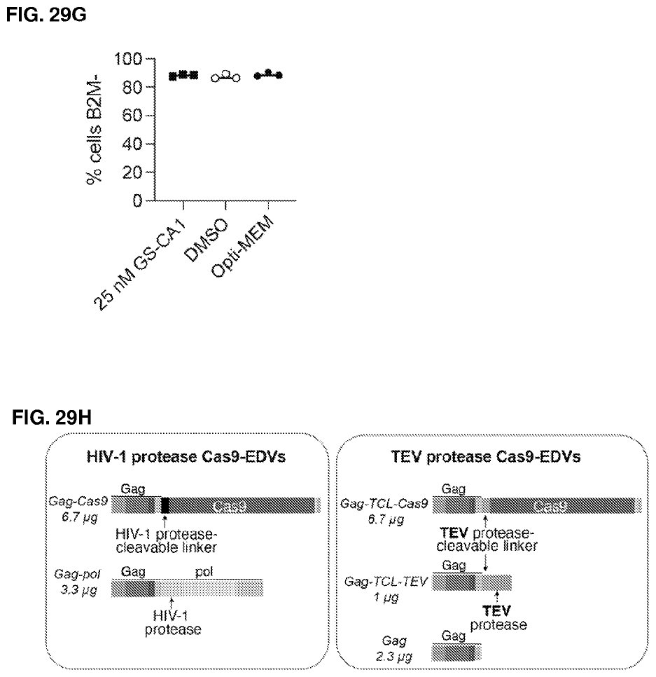

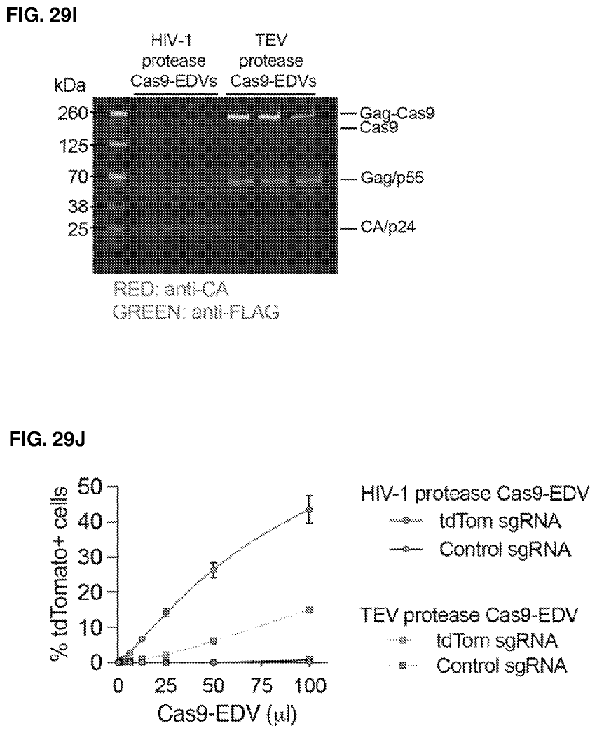

To enhance Cas9-EDV yield and per-particle editing efficiency ahead of in vivo administration, multiple N-terminal nuclear localization signal (NLS) additions to Cas9 were screened and it was found that 2× p53-derived NLS together with 3× nuclear export sequences (NESs) appended to the C-terminal end of Gag, most improved the editing efficiency of antibody-targeted Cas9-EDVs (FIG. 29A-D). Editing efficiency was further increased by expressing sgRNA from both the Gag-NES-NLS-Cas9 and Gag-pol plasmids, as opposed to expression from a separate plasmid (FIG. 23A, B). Optimized Cas9-EDVs maintained receptor-mediated delivery specificity except at the highest doses tested (FIG. 29E), and Cas9-EDV titration resulted in a 36-fold preference for genome editing in on-target over bystander cells (79.7% vs. 2.2%) (FIG. 29F). Improved editing efficiency was observed for optimized, broadly transducing VSVG Cas9-EDVs when tested on human CD34⁺ cells and cytokine-stimulated and activated human T cells ex vivo (FIG. 23C, D). Surprisingly, optimized Cas9-EDVs mediated genome editing in resting human T cells (FIG. 23E), which are difficult to edit using standard electroporation approaches. This suggests Cas9-EDVs may be leveraged for editing T cells in the absence of cellular activation, stimulation, and expansion.

Whether the internal composition of Cas9-EDVs affects editing efficiency was next investigated, focusing on the lentiviral capsid that forms during proteolytic virion maturation (FIG. 23F). To probe the role of the capsid in Cas9-EDV delivery, GS-CA1, a small-molecule inhibitor of nuclear import and/or subsequent uncoating of HIV-1 capsid cores was employed (FIG. 23G). Treatment of target cells with increasing concentrations of GS-CAI blocked the integration of a lentiviral transgene, which relies on nuclear import from the capsid (FIG. 23H), but did not negatively impact the genome editing efficiency of Cas9-EDVs (FIG. 231) or electroporated Cas9 RNPs (FIG. 29G). In a separate experiment, a Cas9-EDV variant that relies on TEV protease to release Cas9 from Gag (“TEVp-Cas9-EDVs,” FIG. 29H) was generated, preventing the HIV-1 protease-dependent virion maturation required for capsid assembly. Despite the demonstrated loss of Gag proteolytic processing (FIG. 29I), TEVp-Cas9-EDVs maintained genome editing activity in treated cells proportional to the amount of Cas9 generated (FIG. 29J). These results suggest that the capsid is not required for packaging and delivering Cas9 RNP complexes into target cell nuclei.

### Optimized Cas9-EDV Characterization

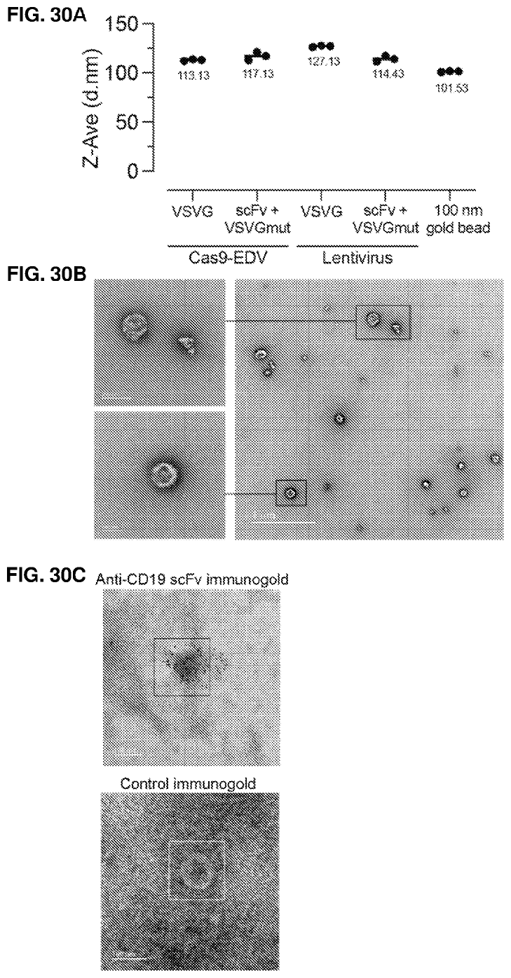

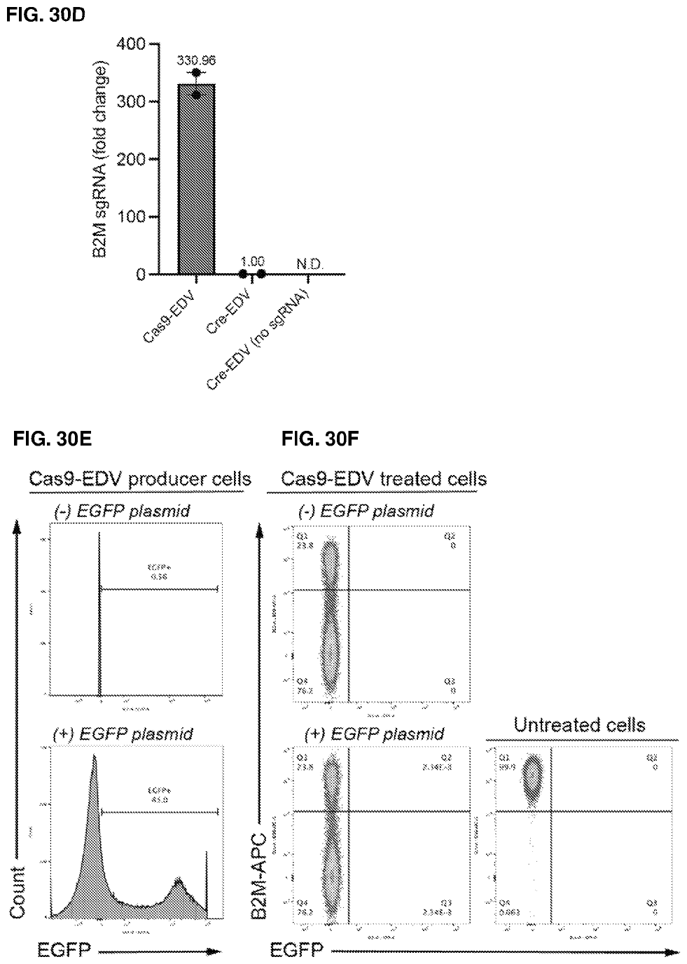

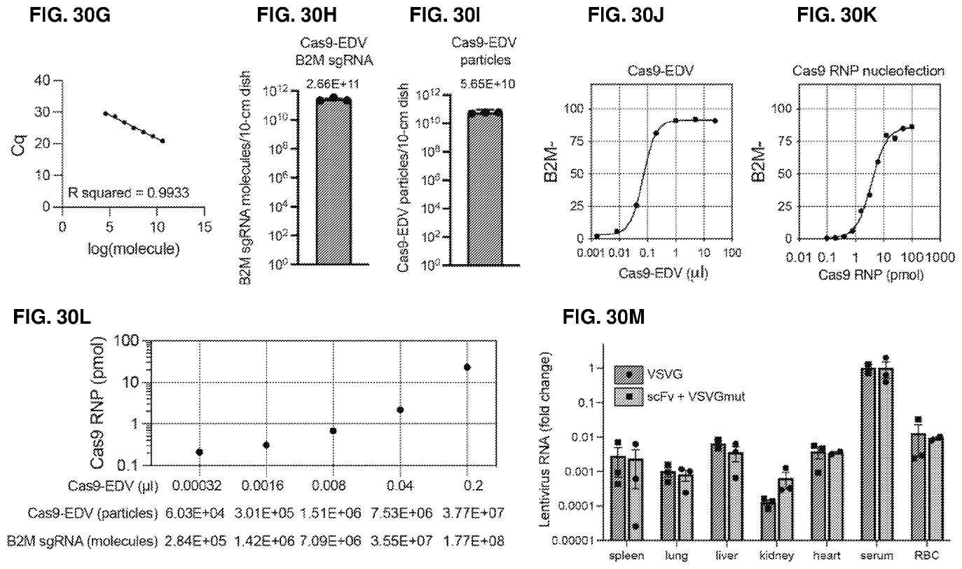

Cas9-EDVs characterization was next performed to understand particle composition and genome editing activity. Cas9-EDVs are similar in diameter to lentiviral vectors (FIG. 30A, 30B), and multiple scFvs are observable on the surface of antibody-targeted particles (FIG. 30C). Interestingly, Cas9-independent packaging of over-expressed sgRNA into Cre Recombinase-EDVs was detected, but sgRNA packaging was enhanced-330-fold in Cas9-containing particles (FIG. 30D). While others have shown the unintended packaging of cellular RNAs and proteins into retroviral vectors, which likely occurs in Cas9-EDVs, unintended Cas9-EDV-mediated delivery of plasmids from producer cells to treated cells was not detected (FIG. 30E, F).

Using synthetic sgRNA as a standard curve (FIG. 30G), it was estimated that Cas9-EDVs produced in one 10-cm plate contain-2.66E11 sgRNA molecules (FIG. 30G) distributed among -5.65E10 Cas9-EDVs (FIG. 30I). Benchmarking Cas9-EDVs against Cas9 RNP nucleofection, it was found that treatment with 0.2 μl of 30-fold concentrated Cas9-EDVs resulted in the equivalent amount of genome editing as-23 pmol of nucleofected RNP (FIG. 30J-L). Finally, the biodistribution of wild-type VSVG vs. VSVGmut pseudotyped lentiviral vectors was tested. It was found that, relative to the amount of vector in the serum, both pseudotypes exhibited similar biodistribution patterns shortly after systemic administration, suggesting that VSVGmut display does not hinder in vivo particle trafficking (FIG. 30M).

### A Multiplexed Targeting Approach for Human T Cell Engineering

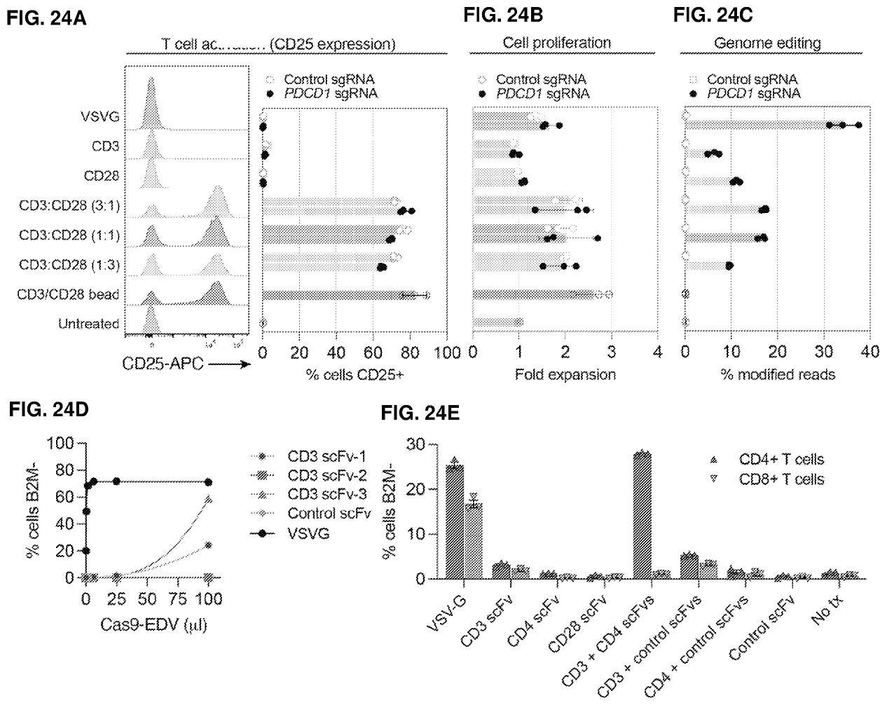

Human T cells are important targets for in vivo genome engineering applications due to their use in treating cancer and other diseases. Using CD25 expression as a marker, the Cas9-EDV co-display of CD3 and CD28 targeting molecules triggered T cell activation and cellular expansion similar to T cells pretreated with commercially-available CD3/CD28 coated magnetic beads or engineered lentiviruses (FIG. 24A, B). CD3⁺CD28 scFv Cas9-EDV treatment also led to robust levels of genome editing (FIG. 24C).

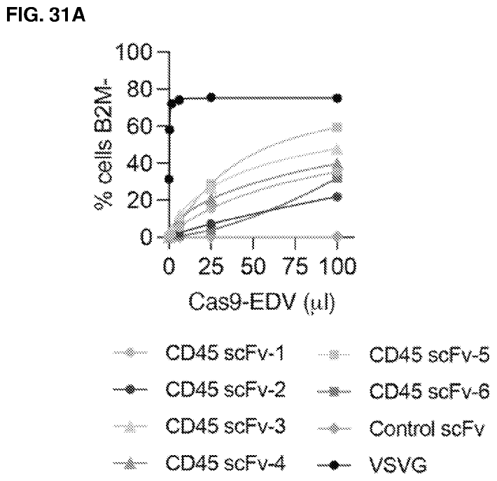

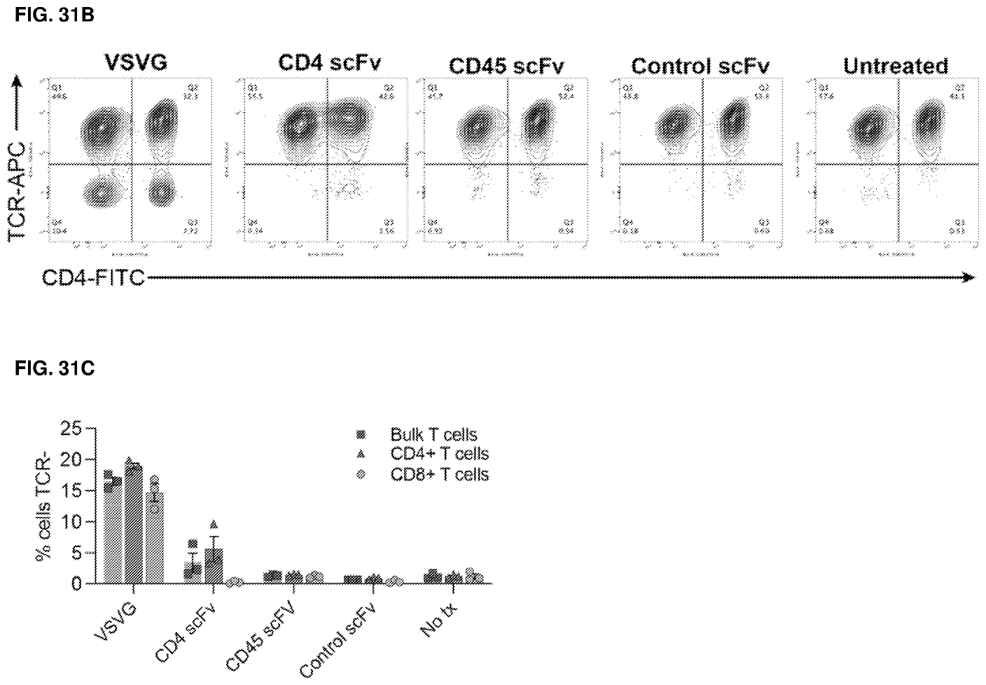

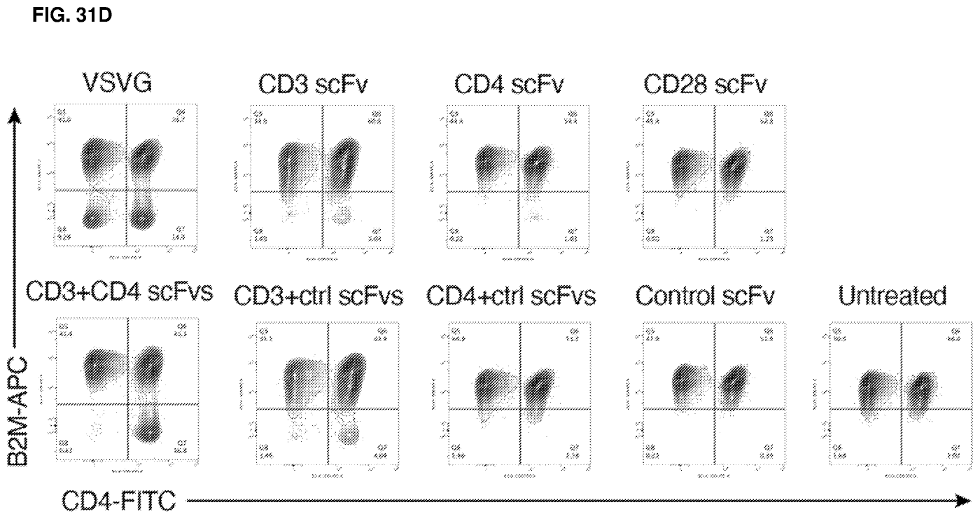

Further screening of CD3 and CD45 scFvs revealed additional Cas9-EDV targeting molecules that enabled genome editing of the human Jurkat T cell line (FIG. 24D; FIG. 31A). Relatively lower human T cell editing observed with CD45-targeted Cas9-EDVs may result from a lack of CD45 internalization from the plasma membrane following monoclonal antibody engagement (FIG. 31B, C). Primary human T cells were susceptible to genome editing using CD3-targeted Cas9-EDVs and, to a lesser extent, CD4-targeted Cas9-EDVs, but not Cas9-EDVs pseudotyped with off-target control scFvs (FIG. 24E; FIG. 31D). Immunophenotyping of T cells post-Cas9-EDV treatment showed that CD3-targeted Cas9-EDVs direct genome editing in CD4⁺ and CD8⁺ subsets of T cells as expected, whereas CD4 scFv-targeted Cas9-EDVs specifically mediated genome editing in the CD4⁺ subset population (FIG. 24E; FIG. 31D). Interestingly, multiplexing CD3 and CD4 scFv targeting molecules on the same Cas9-EDVs led to higher levels of editing than Cas9-EDVs displaying either CD3 or CD4 scFv targeting molecules alone (FIG. 24E; FIG. 31D). This observation was antigen-specific, as multiplexing CD3 targeting molecules with off-target control scFvs did not enhance genome editing. Receptor cross-linking or aggregation can lead to endocytosis and subsequent lysosomal trafficking. Because the fusogenic activity of VSVGmut is triggered in the low pH of the late endosome, this may explain the synergistic increase in genome editing by engaging both CD3 and CD4 receptors.

### T-Cell Targeted Cas9-EDVs Enable Complex Genome Engineering in Humanized Mice

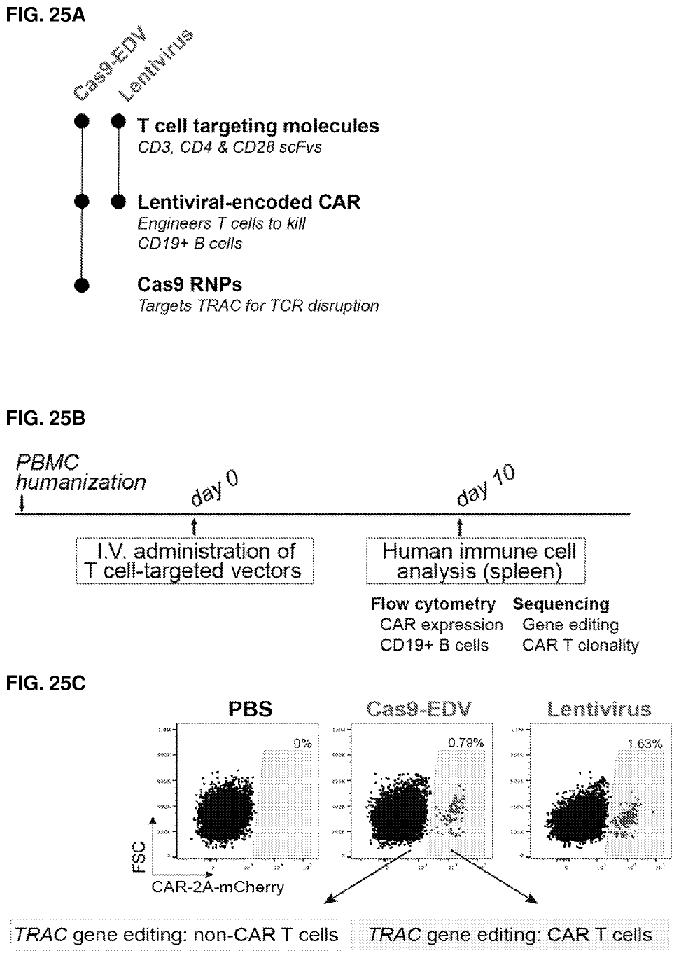

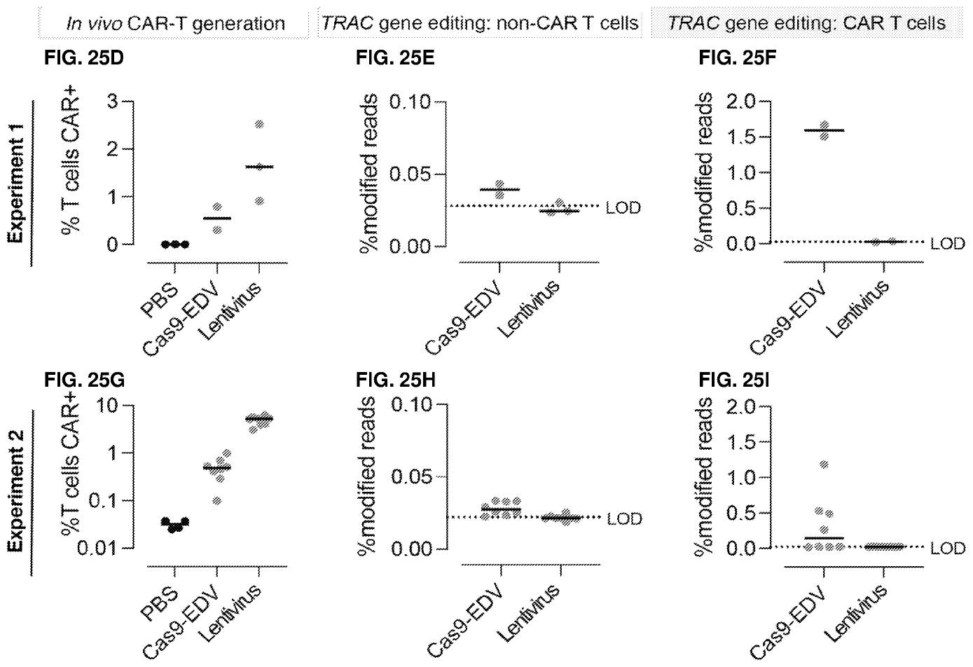

The ability of Cas9-EDVs to deliver both genome editors and transgenes in a cell-type-specific manner in vivo was next investigated. As a first application, Cas9-EDVs were tested for their ability to generate gene-edited human CAR T cells in vivo, an advance that could negate the delays and costs associated with current ex vivo approaches. Using immunodeficient mice engrafted with human peripheral blood mononuclear cells (PBMCs) to mimic a humanized immune system, two T cell-targeted vectors were tested for human CAR T cell engineering in vivo. Both vectors package a lentiviral-encoded a-CD19-4-1BBz CAR-P2A-mCherry transgene, with the Cas9-EDVs additionally packaging Cas9 RNP complexes to disrupt the T cell receptor alpha constant (TRAC) gene (FIG. 25A). Both vectors rely on semi-random integration of the transgene for CAR expression and both co-display CD3, CD4 and CD28 scFvs to trigger enhanced cell entry (CD4⁺CD3) as well as cell activation and proliferation (CD3⁺CD28).

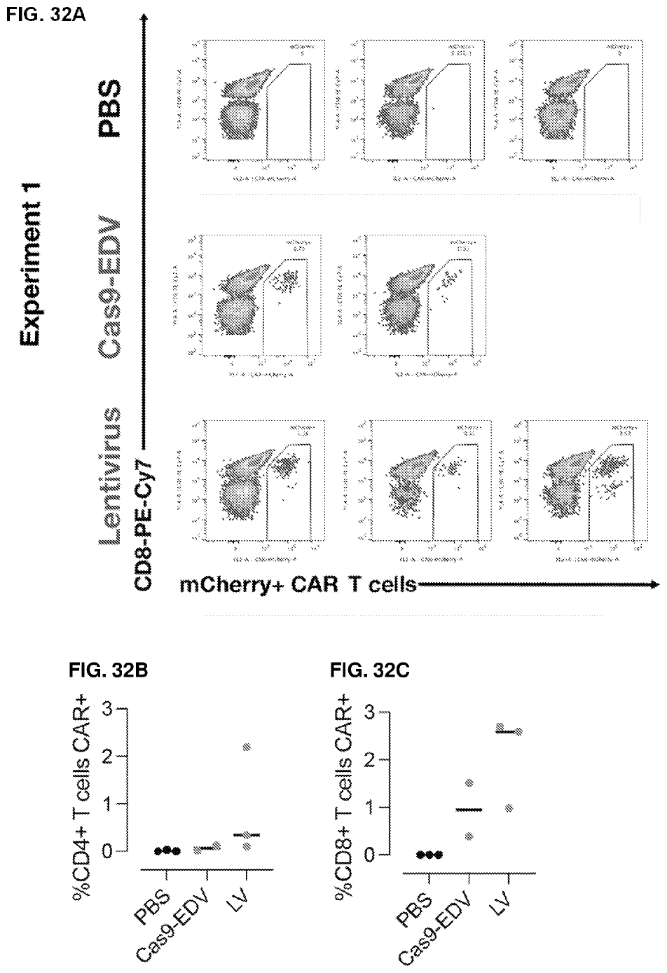

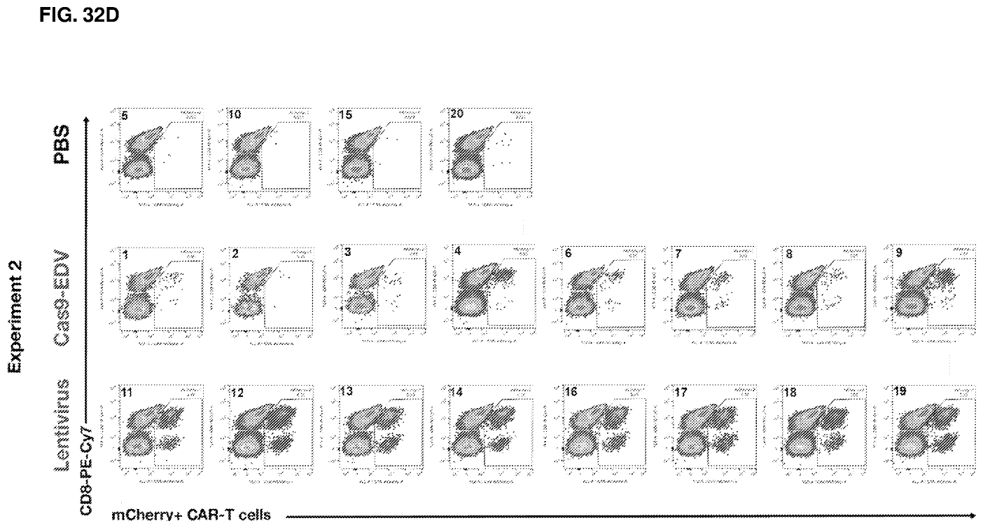

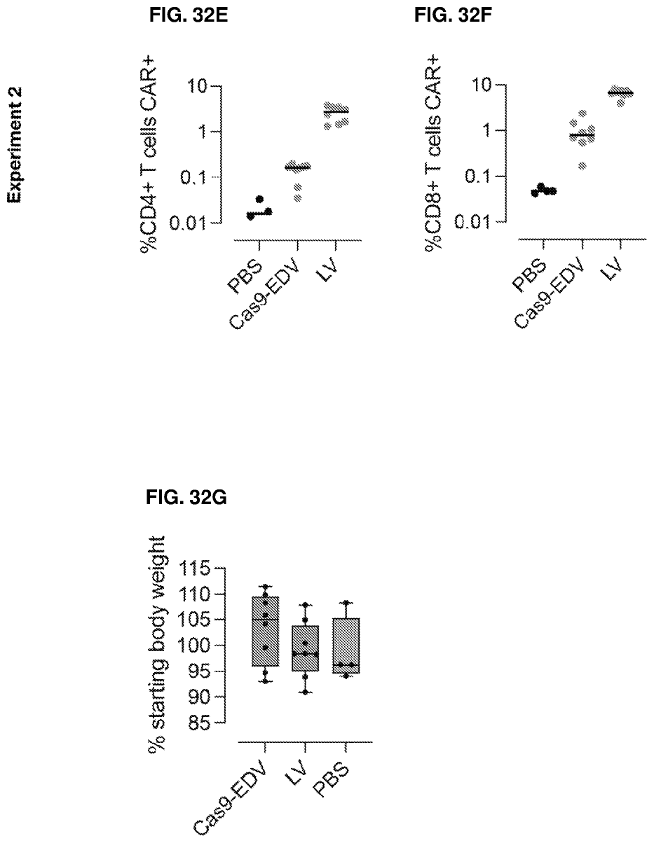

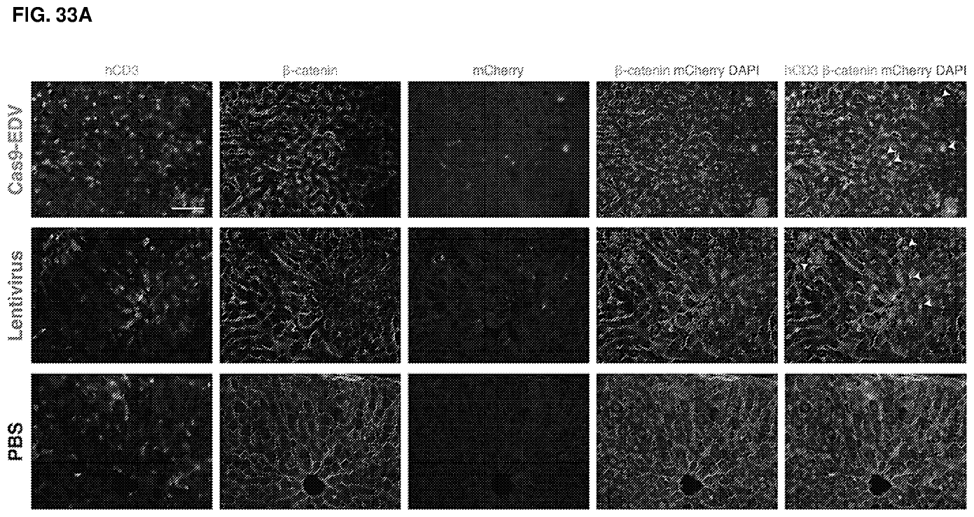

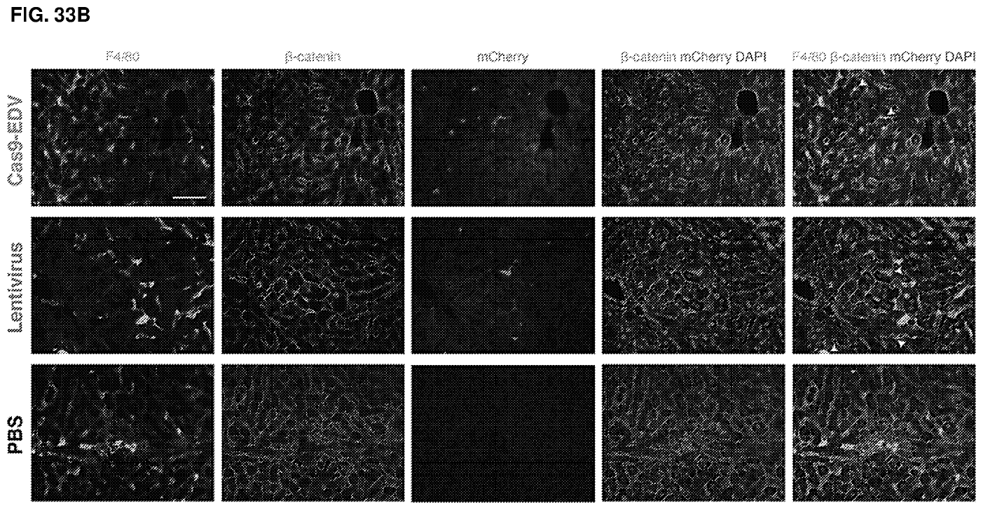

T cell-targeting Cas9-EDVs (N=4) or T cell-targeting lentivirus (N=3) were systemically administered and in vivo cell engineering was assessed 10 days post-treatment (FIG. 25B). CAR-transduced, mCherry+T cells were observed in all mice in which human cells successfully engrafted (FIG. 25C, D; FIG. 32A-C). In the Cas9-EDV-treated mice that successfully engrafted with human T cells (N=2 of 4), 1.67% and 1.51% modified alleles in the CAR-transduced T cells were observed, compared to 0.04% and 0.04% in the CAR-negative T cells isolated from the same mice (FIG. 25E, F). As expected, no modified alleles were observed in cells isolated from mice treated with the T-cell-targeted lentivirus. Repeating this experiment with mice humanized with PBMCs from a different donor and with more mice per treatment group, CAR T cells generated in vivo in 8/8 mice treated with T cell-targeted Cas9-EDVs and 8/8 mice treated with T cell-targeted lentivirus (˜0.5% vs.-5% CAR+T cells, respectively) (FIG. 25G; FIG. 32D-F). Genome editing was observed only in mice treated with Cas9-EDVs (N=4 of 8), with higher levels of genome editing in CAR-transduced T cells over CAR-negative T cells (FIG. 25H, I). Treatment with the T cell-targeted Cas9-EDV and lentivirus was well tolerated, with no weight loss observed (FIG. 32G). While mCherry+F4/80⁺Kupffer cells/macrophages were observed, no mCherry+β-catenin-expressing hepatocytes were detected in the liver (FIG. 33A, B). Together, these results indicate that antibody-based targeting of Cas9-EDVs is a strategy that maintains cell-selective and tissue-specific delivery of transgenes and genome editors in vivo.

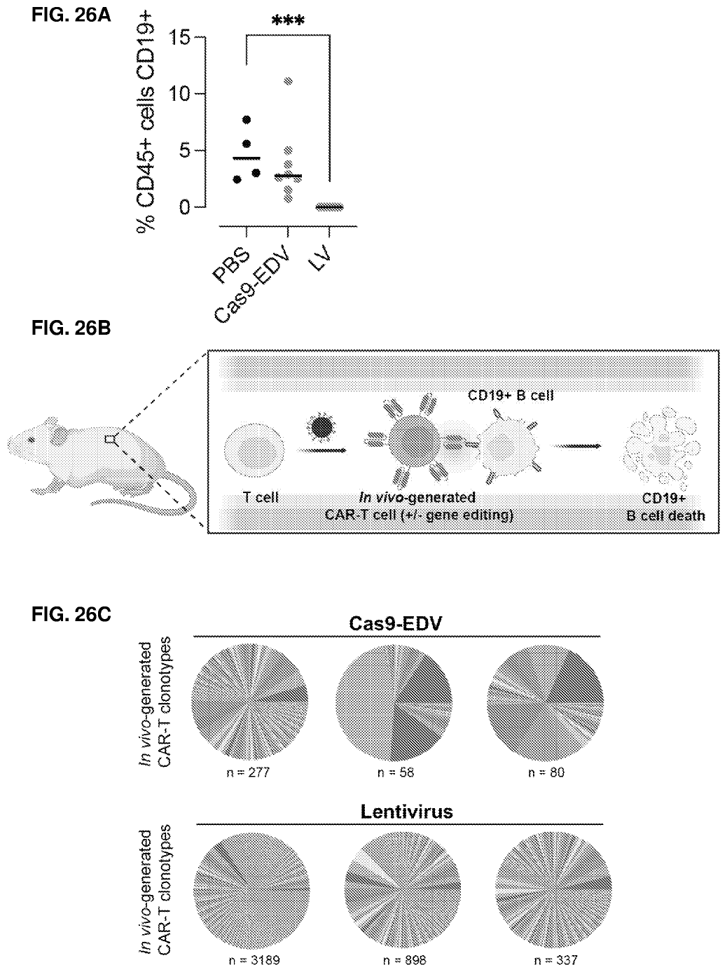

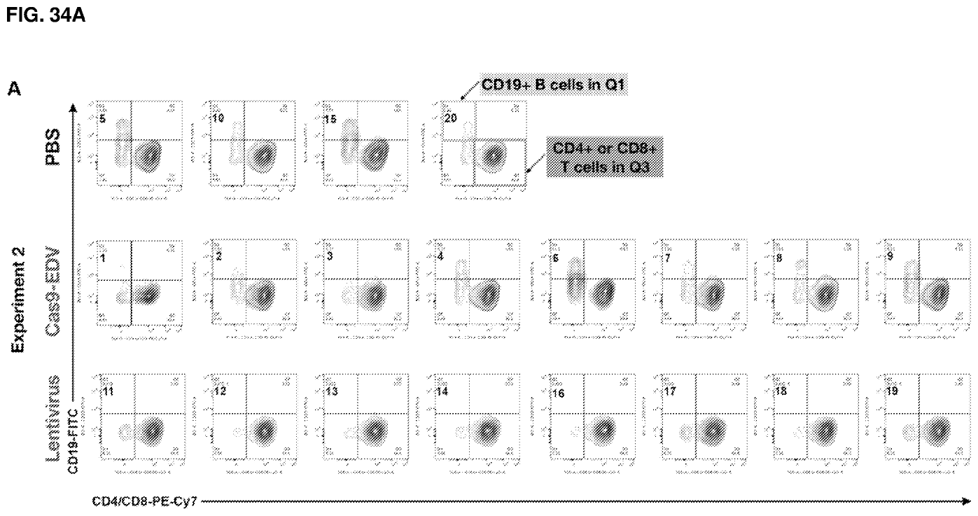

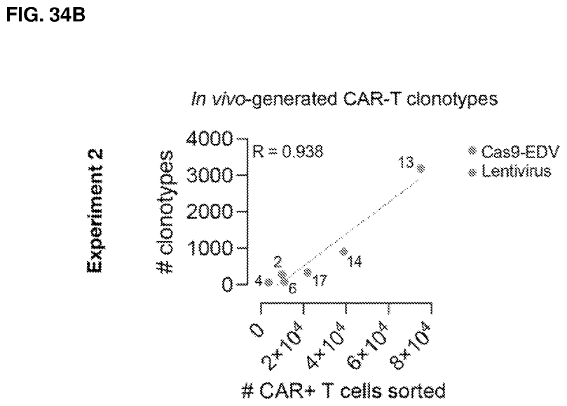

The primary objective of testing Cas9-EDVs in humanized mice was to assess cell-targeted genome editor and transgene delivery in vivo. Because human CD19⁺ B cells, in addition to T cells, engrafted in the second mouse cohort, in vivo CAR T killing activity was additionally assessed. Variable levels of CD19⁺ B cells were observed in Cas9-EDV-treated mice, and no CD19⁺ B cells were detected in mice treated with antibody-targeted lentivirus, demonstrating in vivo CAR T mediated cytotoxicity (FIG. 26A; FIG. 34A). This analysis suggests a model where antibody-derived targeting molecules can direct molecular cargo to specific cells in vivo to successfully reprogram cell activity (FIG. 26B). Diverse T cell clonotypes were observed for CAR-transduced T cells isolated from mice in both groups (FIG. 26C), suggesting that multiple cells were engineered in vivo, and did not arise solely through expansion of a single engineered cell. Because clonotype diversity correlated with the number of CAR T cells analyzed (FIG. 34B), the clearance of B cells in the lentiviral group was likely attributable to a higher number of CAR T cells generated during the initial in vivo transduction. Taken together, these findings offer an approach for generating genome-engineered cells with complex edits that could prove valuable for a wide range of clinical applications in the future.

Enveloped delivery vehicles link the specificity of antibody binding to the delivery of Cas9 protein, sgRNAs and transgenes to enable the engineering of specific human cell types both ex vivo and in vivo. As shown here, Cas9-EDVs facilitate the in vivo generation of gene-edited human CAR T cells, with important advantages relative to other in vivo delivery methods. First, EDVs can be administered systemically for cell-type specific receptor-mediated delivery of multiple cargo molecules including protein, RNA and DNA. Second, by contrast to viral vector-based methods for delivering DNA-encoded molecules, EDVs provide transient delivery of preassembled genome editors whose short lifetime limits off-target editing. In addition, both AAV and lentiviral delivery can involve random transgene integration that could be avoided in the future using Cas9 RNP-mediated genome editing for targeted transgene knock-in. Third, distinct from viral vectors and lipid nanoparticles, EDVs do not induce detectable transduction in liver hepatocytes, which could help avoid toxicity by minimizing the effective concentration necessary for therapeutic benefit. Finally, in contrast to previous in vivo CAR T cell generation reports using retargeted retroviruses, there is no need for T cell activation prior to vector administration because Cas9-EDVs activate T cells during delivery.

### Materials and Methods

Plasmid construction

VSVGmut (K47A R354A VSVG) sequence was human codon-optimized and synthesized as a gBlock (Integrated DNA Technologies) and cloned into the pCAGGS expression plasmid. To generate the CD19 scFv-1 expression plasmid, the sequence encoding the CD8a signal peptide, myc epitope tag, scFv, and CD8a stalk and transmembrane domain of a-CD19-4-1BBi;-P2A-mCherry was subcloned into pCAGGS. This plasmid was subsequently used as an entry plasmid for cloning all other scFv antibody fragments: the CD8a signal peptide, myc tag, and scFv sequences were dropped out by EcoRI/Esp3L restriction digest (New England Biolabs) and new DNA sequences encoding CD8a signal peptide and scFv were inserted. This cloning strategy resulted in removing the n-terminal myc epitope tag and adding a serine amino acid residue between the scFv and CD8a hinge domains. A flexible linker (GGGGSGGGGSGGGGSS; SEQ ID NO:203) was used to link VH and VL domains of source monoclonal antibody sequences. If the antibody source sequence was already an scFv, the linker from the source sequence was used. Except for CD19 scFv-1, all antibody fragment sequences were human codon-optimized and synthesized as eBlock Gene Fragments (Integrated DNA Technologies). Lastly, a CD19 scFv expression plasmid with 2× strep-tag was generated by removing the myc tag from CD19 scFv-1 and inserting the 2× strep tag. InFusion cloning (Takara Bio) was used to generate all plasmids. Additional information on the scFv targeting molecules and sequence sources can be found in FIG. 35.

A second-generation lentiviral transfer plasmid encoding expression of EFl a promoter-CAR-P2A-mCherry was digested with XbaI and MluI (New England Biolabs) to drop out the CAR-P2A-mCherry transgene. Human CD19 (Uniprot #Q71UWO) DNA was ordered as a gBlock (Integrated DNA Technologies) and IRES-EGFP (amplified from the Xlone TRE3G MCS-TEV-Halo-3XF IRES EGFP-Nuc-Puro plasmid, a gift from the Darzacq/Tijan Lab) sequences were inserted using InFusion cloning (Takara Bio). This cloning strategy inserted a MluI restriction digest site 3′ of the CD19 stop codon and removed the MluI restriction digest site 3′ of the EGFP stop codon. Human CD4 (Uniprot #P01730), CD20 (Uniprot #P11836), and CD28 (Uniprot #P10747) amino acid sequences were human codon-optimized for synthesis and ordered as an eBlock (CD28) or gBlocks (CD20, CD4) (Integrated DNA Technologies). Ligand-encoding sequences were cloned by restriction digest removal of CD19-encoding sequence from the EFla-CD19 IRES-EGFP lentiviral plasmid using XbaI & MIuI (New England Biolabs) and inserted with InFusion cloning (Takara Bio). VSVGmut and scFv targeting plasmids were prepared using the HiSpeed Plasmid Maxi or Plasmid Plus Midi kits (QIAGEN). Lentiviral plasmids were prepared with the QIAprep Spin Miniprep Kit (QIAGEN). All plasmids were sequence-confirmed (UC Berkeley DNA Sequencing Facility, Quintara Bio, or Primordium Labs) before use.

All Gag-fusion constructs were cloned using InFusion cloning (Takara Bio). The p53-NLS aa 305-322 sequence was obtained by reverse transcription PCR using RNA extracted from Raji cells as a template with the SuperScript™ III One-Step RT-PCR System with Platinum™ Taq DNA Polymerase (Thermo Fisher). 2×-p53 NLS was constructed by linking two p53 NLS sequences by a flexible linker (GGSGG; SEQ ID NO: 43) and the 2×-p53 NLS sequence was inserted into Gag-Cas9 (Addgene plasmid #171060) with InFusion cloning (Takara Bio). Gag-Cas9 and Gag-[2× p53-NLS]-Cas9 were digested with MfeI-HF and AgeI-HF (New England Biolabs). 3× NES sequence was human codon-optimized, synthesized as a gBlock (IDT), and inserted with InFusion cloning (Takara Bio) to generate Gag-[3× NES]-[2× p53-NLS]-Cas9.

The U6-sgRNA expression cassette was cloned into the plasmid backbones of Gag-[3× NES]-[2× p53-NLS]-Cas9 and psPax2 as follows: Gag-[3× NES]-[2× p53-NLS]-Cas9 was digested with SalI-HF (New England Biolabs). The Gag-pol expression plasmid psPax2 (Addgene plasmid #12260) was first digested with AflII and SacI (New England Biolabs) to remove a SalI restriction site. The modified psPax2 was then digested with SalI-HF (New England Biolabs). The U6-sgRNA expression cassette was amplified from the spyCas9 sgRNA-BsmBI-Destination plasmid (Addgene plasmid #171625) and inserted into the digested Gag-[3× NES]-[2× p53-NLS]-Cas9 and psPax2 with InFusion cloning (Takara Bio). Oligos encoding guide RNA spacers (B2M: 5′-GAGTAGCGCGAGCACAGCTA [SEQ ID NO:208]; TRAC: 5′-AGAGTCTCTCAGCTGGTACA [SEQ ID NO:209]; PDCD-1: 5′-CGACTGGCCAGGGCGCCTGT [SEQ ID NO:210]; tdTomato: 5′-AAGTAAAACCTCTACAAATG [SEQ ID NO:213]; control: 5′-GTATTACTGATATTGGTGGG [SEQ ID NO:214]) were ordered from IDT, phosphorylated, annealed and ligated into BsmBI-digested sgRNA expression vectors.

The U6-B2M Gag-[3× NES]-[2× p53-NLS]-Cre plasmid was generated as follows: Gag-Cre was digested with MfeI-HF and NheI. The [3× NES]-[2× p53-NLS]-Cre sequence was synthesized as a gBlock (IDT) and inserted downstream of Gag with InFusion cloning (Takara Bio). Then, the Gag-[3× NES]-[2× p53-NLS]-Cre plasmid was digested with XbaI and PvuI-HF (New England Biolabs). The U6-B2M expression cassette was isolated by digesting U6-B2M Gag-[3× NES]-[2× p53-NLS]-Cas9 with XbaI and PvuI-HF (New England Biolabs) and inserted into the digested Gag-[3× NES]-[2× p53-NLS]-Cre with T4 DNA ligase (New England Biolabs).

### Tissue Culture and Cell Line Generation

Lenti-X and HEK293T cells, obtained and authenticated by the UC Berkeley Cell Culture Facility, were cultured in DMEM (Corning) supplemented with 10% fetal bovine serum (VWR) and 100 U/nil penicillin-streptomycin (Gibco) (“cDMEM”). To generate lentiviruses encoding EFla-ligand IRES-EGFP, 3.5-4 million Lenti-X cells were plated in a 10 cm tissue culture dish (Corning) and transfected with 1 μg pCMV-VSV-G (Addgene plasmid #8454), 10 μg psPax2 (Addgene plasmid #12260), and 10 μg of EFla-ligand IRES-EGFP lentiviral transfer plasmid using polyethylenimine (Polysciences Inc.) at a 3:1 PEI:plasmid ratio. Lentiviral-containing supernatants were harvested two days post-transfection and passed through a 0.45 pm PES syringe filter (VWR). Ligand-expressing cells were generated by transducing HEK293T cells (100,000 per well in a 12-well dish) with lentivirus (0.15-1 ml) in a total well volume of 1 ml. Four days post-transduction, flow cytometry was used to identify cell mixtures where <25% of cells were expressing EGFP. Following expansion, CD19 EGFP HEK293T cells were additionally sorted for EGFP expression using an SH800S cell sorter (Sony Biotechnology) to generate a population of cells-100% CD19⁺EGFP+ cells.

### T Cell Culture

Cryopreserved human peripheral blood mononuclear cells (PBMCs, AllCells) were thawed in X-VIVO 15 (Lonza) with 50 μM 2-Mercaptoethanol (Gibco), 5% fetal bovine serum (VWR), and 10 mM N-acetyl L-cysteine (Sigma-Aldrich). This media was also used to culture the T cells isolated from PBMCs. To isolate T cells, PBMCs were incubated in 100 μg/mL DNase I solution (Stem Cell Technologies) at room temperature for 15 minutes and resuspended in EasySep™ Buffer (Stem Cell Technologies). Aggregated suspensions were filtered through a 37 pm cell strainer (Stem Cell Technologies) and incubated with EasySep™ Human T Cell Isolation Cocktail (Stem Cell Technologies). Then, EasySep™ Dextran RapidSpheres™ (Stem Cell Technologies) were used to separate T cells via an EasySep™ Magnet (Stem Cell Technologies). Dynabeads™ Human T-Activator CD3/CD28 (Gibco) and recombinant human 500 U/mL IL-2 (Peprotech), 5 ng/mL IL-7 (Peprotech), and 5 ng/mL IL-15 (R&D Systems) were used to stimulate and activate T cells for two days before treatment. Pre-activated T cells were cultured in media containing 500 U/mL IL-2 (Peprotech).

### CD34⁺ Cell Culture

Cryopreserved G-CSF-mobilized human CD34⁺ cells were acquired from AllCells. Cells were thawed, resuspended inIMDM (Gibco) spun, and then were cultured in StemSpan™ SFEM II media (Stem Cell Technologies) with 100 U/ml penicillin-streptomycin (Gibco) and the cytokine cocktail StemSpan™ CC 110 (Stem Cell Technologies). Cells were treated with 8 μM cyclosporine H (Sigma-Aldrich) for 24 hours before treatment. Additionally, cells were treated with 1 μg/ml poloxamer (BASF) at the time of treatment. Cas9-EDVs were concentrated 50-fold using Lenti-X Concentrator (Takara Bio), resuspended in SFEM II media, and mixed with 40k CD34⁺ cells in a final well volume of 100 μl. Transduction was performed in a U-bottom 96-well plate for 24 hours on an orbital shaker before cells were spun, expanded 1:1, and cultured stationary until analysis.

### Cas9-EDV Production

Cas9-EDVs (formerly known as “Cas9-VLPs”) were produced as previously described (22). Briefly, VSVG-pseudotyped Cas9-EDVs were produced by seeding 3.5-4 million Lenti-X cells (Takara Bio) into 10 cm tissue culture dishes (Corning) and transfecting the next day with 1 μg pCMV-VSV-G (Addgene plasmid #8454), 6.7 μg Gag-Cas9 (Addgene plasmid #171060), 3.3 μg psPax2 (Addgene plasmid #12260), and 10 μg U6-sgRNA (Addgene plasmid #171635 or Addgene plasmid #171634) using polyethylenimine (Polysciences Inc.) at a 3:1 PEI:plasmid ratio. Antibody-targeted Cas9-EDVs were produced in the same way as VSVG Cas9-EDVs, except that the pCMV-VSV-G plasmid was omitted, and 7.5 μg of scFv targeting plasmid and 2.5 μg of VSVGmut were included during transfection. For scFv multiplexing, a total of 7.5 μg scFv plasmids was used, split 1:1 or 1:1:1, unless otherwise described in the figure legend. For Cas9-EDVs used to treat primary human cells (T cells, CD34⁺ cells) or humanized mice, media was changed 6-18 hours post transfection into Opti-MEM (Gibco). Two days post-transfection (or media change), Cas9-EDV-containing supernatants were harvested, passed through a 0.45 pm PES syringe filter (VWR), and concentrated with Lenti-X Concentrator (Takara Bio) according to the manufacturer's instructions. Concentrated Cas9-EDVs were resuspended in Opti-MEM (Gibco) at a final concentration of 10× unless otherwise noted in the figure legend. Cas9-EDVs were stored at 4° C. or frozen at −80° C. within an isopropanol-filled freezing container until use.

To optimize Cas9-EDVs, variants with different Gag-Cas9 polypeptides were produced in the same way as above, except that 6.7 μg of each Gag-Cas9 variant (Gag-[3× NES]-Cas9, Gag-[2× p53-NLS]-Cas9, and Gag-[3× NES]-[2× p53-NLS]-Cas9) was used instead of Gag-Cas9 during transfection. Gag-[3× NES]-[2× p53-NLS]-Cas9 EDVs with U6-sgRNA expression from plasmid backbones were produced similarly, except that the U6-B2M plasmid was omitted, and 6.7 μg of U6-sgRNA Gag-[3× NES]-[2× p53-NLS]-Cas9 and 3.3 μg of U6-sgRNA psPax2 were included instead of the Gag-Cas9 and psPax2 plasmids during transfection. To capture both improvements in Cas9-EDV particle production and the per-particle editing efficiency of Cas9 EDVs, equivalent numbers of transfected 10 cm tissue culture dishes (Coming) of each Cas9-EDV variant tested were produced.

For humanized mouse experiments, Cas9-EDVs were produced by transfecting Lenti-X cells with 2.5 μg of each scFv targeting plasmid (CD3 scFv-3, CD4 scFv-1, and CD28 scFv-2), 2.5 μg of VSVGmut, 3.3 μg of U6-TRAC Gag-[3× NES]-[2× p53-NLS]-Cas9, 6.7 μg of U6-TRAC psPax2, and 2.5 μg of the lentiviral transfer plasmid encoding an a-CD19-4-1BBz CAR-P2A-mCherry transgene, as optimized in. Lentivirus was produced in the same way, except that U6-TRAC Gag-[3× NES]-[2× p53-NLS]-Cas9 and U6-TRAC psPax2 were omitted, and 10 μg psPax2 was included. Cas9-EDV- and LV-containing supernatants were passed through a 0.45 pm PES filter bottle (Thermo Fisher) and concentrated via ultracentrifugation by floating the supernatant on top of a cushioning buffer of 30% (w/v) sucrose in 100 mM NaCl, 10 mM Tris-HCl pH 7.5, 1 mM EDTA pH 8.0, at 25,000 rpm with a SW28 Ti rotor (Beckman Coulter) for 2 hours at 4° C. in polypropylene tubes (Beckman Coulter). After ultracentrifugation, the Cas9-EDV pellet was resuspended in sterile Dulbecco's phosphate-buffered saline (DPBS) (Gibco).

### Cas9 RNP Electroporation

B2M-targeting crRNA (IDT) and tracrRNA (IDT) were resuspended in IDT duplex buffer to 160 μM, combined at a ratio of 1:1, and annealed at 37° C. for 30 minutes. Cas9 RNPs were formed by combining the annealed crRNA and tracrRNA and 40 μM Cas9-NLS (UC Berkeley QB3 MacroLab) at a molar ratio of 2:1 and incubating at 37° C. for 15 minutes. Electroporation was performed using a 96-well format 4D nucleofector (Lonza) with 200 000 cells per well. HEK293T cells were electroporated with the SF buffer and the CM-130 pulse code, and primary human T cells with the P3 buffer and the EH-115 pulse code. Cells were immediately resuspended in pre-warmed media, incubated for 20 minutes, and transferred to culture plates.

### Cas9-EDV Titer Quantification

The QuickTiter™ Lentivirus Titer Kit (Lentivirus-Associated HIV p24) (Cell Biolabs, INC) was used to quantify Cas9-EDV particle number. Cas9-EDVs were diluted 1:1,00-100,000 and the ELISA was performed according to the manufacturer's directions. 450 nm absorbance was measured plate reader (BioTek). Cas9-EDV p24 content was calculated by comparison to serial dilution of a p24 standard and guidance from the manufacturer was used to convert p24 quantity into particle number (Cell Biolabs, INC).

### Western Blot Analysis

Cas9-EDVs were mixed with Laemmli buffer containing 10% 2-mercaptoethanol and heating at 90C for 5 minutes. Proteins from whole cell lysates were separated by 4%-20% SDS-PAGE gel (Bio-Rad) and transferred to an Immun-Blot® low fluorescence PVDF membrane (Bio-Rad). Membranes were blocked and incubated with primary antibodies at 4° C. overnight followed by secondary antibodies at room temperature for 1 hour. Primary antibodies were anti-FLAG M2 (F1804, RRID:AB_262044, Sigma-Aldrich) and anti-HIVI p24 (ab9071, RRID:AB_306981, abcam). Secondary antibodies were anti-mouse Alexa Fluor™ 488 (A28175, RRID:AB_2536161, Thermo Fisher) and anti-rabbit Alexa FluorT^{M} 647 (A-31573, RRID:AB_2536183, Thermo Fisher). Imaging was performed using the Odyssey imaging system (LI-COR).

### GS-CA1 Experiments

To generate lentiviruses encoding EFla-mNeonGreen, 3.5-4 million Lenti-X cells were plated in a 10 cm tissue culture dish (Corning) and transfected with 1 μg pCMV-VSV-G (Addgene plasmid #8454), 10 μg psPax2 (Addgene plasmid #12260), and 2.5 μg of EFla-mNeonGreen lentiviral transfer plasmid using polyethylenimine (Polysciences Inc.) at a 3:1 PEL:plasmid ratio. Lentiviral-containing supernatants were harvested two days post-transfection and passed through a 0.45 pm PES syringe filter (VWR). Lentiviral supernatants were concentrated 20× with Lenti-X Concentrator (Takara Bio) according to the manufacturer's instructions, resuspended in Opti-MEM (Gibco), aliquoted, and frozen at −80° C. for future use. B2M-targeted VSVG Cas9-EDVs were produced, as described above. The mNeonGreen lentivirus stock was pre-titered on HEK293T cells: Cas9-EDV and mNeonGreen lentivirus samples were diluted in Opti-MEM in a 2-fold dilution series. 50 μl of each dilution series was mixed with 15,000 HEK293T cells in 50 μl cDMEM in triplicate in a 96-well plate. For the lentiviral sample, the percentage of mNeonGreen-positive cells was assessed by flow cytometry three days post-transduction. Wells where the percent of mNeonGreen+ cells was <25% were used to calculate the transducing units (TU) per ml. The genome editing activity of the Cas9-EDV stock was pre-titered similarly, except that B2M expression was assessed by flow cytometry at three days post-treatment to calculate Cas9-EDV volume that resulted in approximately 50% cells negative for B2M expression.

The HIV-1 capsid inhibitor GS-CA1 (Gilead Sciences, Inc.) was diluted to a working stock of 100 μM using DMSO. 15,000 HEK293T cells were transduced with mNeonGreen lentivirus or B2M-targeting Cas9-EDVs and simultaneously treated with 0, 0.5, 5, or 25 nM GS-CAJ in a total well volume of 100 μl (0.05% DMSO final). mNeonGreen and B2M expression were assessed by flow cytometry three days post-treatment. As a control, B2M sgRNA and Cas9 protein (Integrated DNA Technologies) were complexed at a 2:1 ratio for 15 minutes at 37° C., and 50 pmol Cas9 RNPs were nucleofected into 200,000 HEK293T cell using the SF Cell Line 4D-Nucleofector Kit and 4D-Nucleofector instrument (Lonza) using pulse code CM-130. Post nucleofection, cells were brought up to 100 μl with pre-warmed cDMEM and incubated at 37° C. for 15 minutes. Nucleofected cells were plated at 15,000 per well of a 96-well plate and treated with 25 nM GS-CA1, 0.05% DMSO, or Opti-MEM. B2M expression was assessed by flow cytometry three days post-nucleofection.

### Humanized Mouse Experiments

All studies and procedures were in accordance with the established NIH guidelines for animal care and use and were approved by the UC Berkeley, Animal Care and Use Conmnittee (ACUC). All experimental and control animals were housed under the same conditions as approved by the Berkeley Office of Laboratory Animal Care (OLAC). Human peripheral blood mononuclear cell-engrafted NSG™ mice (745557) were purchased from Jackson Laboratory. “Experiment 1” and “experiment 2” mouse cohorts were engrafted with cells from unique human donors. Following anesthesia induction with 2-3% isofluorane, 100 μl of Cas9-EDVs, lentivirus, or phosphate-buffered saline (PBS) was administered by retro-orbital injection. 10 days post-treatment, mice were euthanized with CO2 to harvest spleen and liver. See “Immunofluorescent staining and imaging” for downstream liver sample processing. Single-cell suspensions of spleen were prepared by gently bursting the organ in 4 ml DMEM (Corning) in a well of a 6-well dish using the back of 3 ml syringe (Thermo Fisher). Splenocytes were passed through a 100 m cell mesh (Corning), brought up to 25 ml with DPBS (Gibco) and spun at 300×g for 10 minutes. Erythrocytes were lysed by resuspending splenocytes in 5 ml ix BD Pharm Lyse™ lysing solution (BD Biosciences) for 5 minutes before being brought up to 25 ml with DPBS. Cells were then pelleted at 300xg for 10 minutes, resuspended in 10 ml DPBS and counted using a Countess 3 automated cell counter (Thermo Fisher) Cells were cryopreserved in freeze media (Bambanker) for downstream cell sorting. For flow cytometry analysis, CD45⁺ cells were isolated from splenocytes using the EasySep™ Release Human CD45 Positive Selection Kit for Humanized Mouse Samples (Stem Cell Technologies) on an EasySep™ EasyEights Magnet (Stem Cell Technologies) according to the manufacturer's instructions. Immunophenotyping was performed using anti-human CD4-FITC (300538, RRID:AB_2562052, Biolegend), anti-human CD4-PE-Cyanine7 (300512, RRID:AB_314080, Biolegend), anti-human CD8-PE-Cyanine7 (557746, RRID:AB_396852, BD Biosciences), and anti-human CD19-FITC (302206, RRID:AB_314236, Biolegend), and cells were assessed for CAR-2A-mCherry expression using an Attune N×T flow cytometer with 96-well autosampler (Thermo Fisher). For cell sorting, cryopreserved splenocytes were thawed and stained with anti-human CD4-FITC (300538, RRID:AB_2562052, Biolegend) and anti-human CD8-FITC (557085, RRID:AB_396580, BD Biosciences) in PBS containing 1% bovine serum albumin, and an SH800S cell sorter (Sony Biotechnology) was used to sort mCherry+ and mCherry-primary human T cells that were either expressing CD4 or CD8.

### Flow Cytometry

Cells were stained with anti-human B2M-APC (316312, RRID:AB_10641281, Biolegend) or anti-human B2M-PE (316306, RRID:AB_492839, Biolegend) in PBS containing 1% bovine serum albumin, and an Attune N×T flow cytometer with 96-well autosampler (Thermo Fisher) was used for flow cytometry analysis. Ligand expression was confirmed for engineered HEK293T cells using anti-human CD28-PE (302907, RRID:AB_314309, Biolegend), anti-human CD20-PE (302306, RRID:AB_314254, Biolegend), anti-human CD4-PE-Cyanine7 (300512, RRID:AB_314080, Biolegend) and anti-human CD19-PE (302208, RRID:AB_314238, Biolegend). T cell immunophenotyping was performed using anti-human CD3-FITC (317306, RRID:AB_571907, Biolegend), anti-human CD4-FITC (300538, RRID:AB_2562052, Biolegend), and anti-human CD8-PE-Cyanine7 (557746, RRID:AB_396852, BD Biosciences). CD25 expression was assessed using anti-human CD25-APC (302610, RRID:AB_314280, Biolegend). Data analysis was performed using FlowJo v10 10.7.1 (FlowJo, LLC, Ashland OR).

### Amplicon Sequencing to Assess Genome Editing

Next-generation sequencing was used to detect on-target genome editing in EGFP-positive, and EGFP-negative sorted HEK293T cells. Genomic DNA was extracted using QuickExtract (Lucigen) as previously described. PrimeStar GXL DNA polymerase (Takara Bio) was used to amplify the Cas9-RNP target site using the following primers: B2M: 5′-GCTCTTCCGATCTaagctgacagcattcgggc (SEQ ID NO:211) and 5′-GCTCTTCCGATCTgaagtcacggagcgagagag (SEQ ID NO:212); PDCD-1: 5′-GCTCTTCCGATCTccgaccccacctacctaaga (SEQ ID NO:282) and 5′-GCTCTTCCGATCTgacagtttcccttccgctca (SEQ ID NO:283). The resulting PCR products were cleaned up using magnetic SPRI beds (UC Berkeley DNA Sequencing Facility). The Innovative Genomics Institute Next-Generation Sequencing Core performed library preparation and sequencing using a MiSeq V2 Micro 2×150 bp kit (Illumina). Reads were trimmed and merged (Geneious Prime, version 2022.0.1) and analyzed with CRISPResso2

### NextSeq P2 Sequencing of Sorted Mouse Cells

Genomic DNA was extracted from sorted mCherry+ and mCherry-primary human T cells using the QIAamp DNA Mini Kit (Qiagen) according to the manufacturer's instructions. PrimeStar GXL DNA polymerase (Takara Bio) was used to amplify the TRAC Cas9-RNP target site using primers 5′-GCTCTTCCGATCTggggcaaagagggaaatgaga (SEQ ID NO:284) and 5′-GCTCTTCCGATCTactttgtgacacatttgtttgag (SEQ ID NO:285). The resulting PCR products were cleaned up using magnetic SPRI beds (UC Berkeley DNA Sequencing Facility). The Innovative Genomics Institute Next-Generation Sequencing Core performed library preparation and sequencing using a NextSeq 1000/2000 P2 V3 2×150 bp kit (Illumina). Reads were trimmed and merged (Geneious Prime, version 2022.0.1) and analyzed with CRISPResso2.

### TCR Sequencing

Splenocytes from three Cas9-EDV-treated mice and three lentivirus-treated mice in humanized mouse experiment 2 were sorted into mCherry+ and mCherry-primary human T cells using an SH800S cell sorter (Sony Biotechnology). RNA was extracted from sorted cells using the RNeasy Mini Kit (Qiagen) according to the manufacturer's instructions. TCR a/b libraries were prepared from each sample using the SMART-Seq Human TCR (with UMIs) kit (Takara Bio) according to the manufacturer's instructions. Samples were pooled and sequenced on an Illumina MiSeq using 2×300 bp paired-end chemistry and MiSeq Reagent Kit v3 (MS-102-3003, Illumina), yielding an average sequencing depth of 1.75 million reads. FASTQ files were analyzed with Cogent NGS Immune Profiler Software (Version 1.5), using a UMI-cutoff of 3. Clonotypes were visualized using Cogent NGS Immune Viewer (Version 1.0) and a custom python script.

### Immunofluorescent Staining and Imaging

Humanized mice were euthanized with CO2, and livers were dissected. The livers were fixed in 4% paraformaldehyde (PFA, Electron Microscopy Sciences, 15710, Hatfield, PA, USA) for 24 hours and transferred to 30% sucrose (Fisher Chemical, S5-500, Fairlawn, NJ, USA). After 3 days, the livers were embedded into cryoblocks (Tissue Plus O.C.T. Compound, Fisher HealthCare, #4548, Houston, TX, USA). 20 pm liver sections were cut with Cryostat (Cryostat, Leica, CM 3050 S, Buffalo Grove, IL, USA) and placed on microscope slides (Superfrost Plus™, Fisher Scientific, #1255015, Pittsburgh, PA, USA), and kept at −80° C. until further use.

The sections were blocked with a buffer including 5% normal goat serum (Sigma-Aldrich, G9023-1OmL, St. Louis, MO, USA), 2% bovine serum albumin (BSA, Sigma-Aldrich, A9418-50G, St. Louis, MO, USA), 0.03% triton X-100 (Fisher Scientific, BP151-100, Eugene, OR, USA), 0.05% sodium azidc (Sigma-Aldrich, 71289-5G, St. Louis, MO, USA) for 1 hour at room temperature (RT). Sections were stained for 2 hours at RT with the combination of primary antibodies as follows 1) mouse anti-p-catenin (Thermo Fisher, 14-2567-82, RRID:AB_1724004, Eugene, OR, USA, 1:100) and rat anti-F4/80 (Novus, NB600-404, RRID:AB_10003219, Centennial, CO, USA, 1:100); 2) mouse anti-0-catenin (Thermo Fisher, 14-2567-82, RRID:AB_1724004, Eugene, OR, USA, 1:100) and rabbit anti-hCD3 (Abcam, ab5690, RRID:AB_305055, Waltham, MA, USA, 1:100). Tissue sections were washed 3 times with lx PBS (Thermo Fisher, 10010023, Eugene, OR, USA) and stained with the secondary antibodies for 1 hour as follows; AlexaFluor 647 goat anti-mouse (Thermo Fisher, A21236, RRID:AB_2535805, Eugene, OR, USA, 1:200), AlexaFluor 488 goat anti-Rat (Thermo Fisher, Al 1006, RRID:AB_2534074, Eugene, OR, USA, 1:200) and AlexaFluor 488 goat anti-rabbit (Thermo Fisher, Al 1034, RRID:AB_2576217, Eugene, OR, USA, 1:200). Tissue sections were washed again three times with lx PBS and treated with DAPI (Sigma-Aldrich, 10236276001, St. Louis, MO, USA, 0.5 mg/mL) for 10 minutes. Then the sections were covered with cover glass slip (Micro cover glass, VWR, 48393-106, Radnor, PA, USA) with Fluoromount-G®(Southern Biotech, 0100-01, Birmingham, AL, USA). For the negative control, the sections were treated with the secondary antibodies only and DAPI (Sigma-Aldrich, 10236276001, St. Louis, MO, USA, 0.5 mg/mL).

The images of the stained livers were taken at 20× magnification with the Echo Revolve fluorescent microscope (Echo, RVL-100-G, San Diego, CA, USA) and obtained using the associated software (ECHOPro) through DAPI, FITC, Texas Red and CY5 channels.

### Immunogold Staining

Cas9-EDVs with CD19 scFvs with either strep or myc epitope tags were produced as stated above. 25 mL of the Cas9-EDVs were concentrated using ultracentrifugation at 100,000×g for 75 minutes through 9 mL of a 10 v/v % iodixanol cushion (Stemcell™ Technologies) in 1× PBS. The supernatant was removed, and the Cas9-EDVs were resuspended in 100 μL of 10 mM Tris HCl pH 7.5, 150 mM NaCl. Cas9-EDVs were stored at 4° C. and used within 48 hours. Carbon Type-B copper transmission electron microscopy (TEM) grids (Ted Pella, Inc.) were glow-discharged. 5 μL of the EDVs were applied to the carbon-side of the grid and incubated for 3 minutes. Excess sample was removed by blotting with Kimwipes. The grids were blocked for 2 minutes using 15 μL of 10 mM Tris-HCl pH 7.5, 150 mM NaCl, 1 w/v % bovine serum albumin (Sigma Aldrich) (blocking buffer). The excess blocking buffer was removed by blotting with Kimwipe. 15 μL of 50 g/mL of mouse Anti-myc antibody (Abeam, ab32, RRID:AB_303599, Waltham, MA, USA) was applied to grid and incubated for 30 minutes at room temperature. To prevent grids from drying out, a lid was used to cover the grids. The grids were subsequently washed five times with 15 μL of blocking buffer. 12 μL of 1/10 diluted goat anti-mouse 6-nm immunogold conjugates (25124, Electron Microscopy Sciences) was applied to the grids and incubated for 30 minutes at room temperature. The grids were subsequently washed with 15 μL of blocking buffer four times, and Ultrapure water once. 5 μL of 0.7% Uranyl formate (Ted Pella) was applied to stain and fix the samples. Excess stain was removed by blotting with Kimwipe. The EDVs were visualized using a FEI Tecnai T12 TEM operating at 120 kV and a Gatan UltraScan 895 4k CCD (UC San Francisco EM Core).

### Dynamic Light Scattering (DLS)

Cas9-EDV and LV were assessed using a Zetasizer Nano ZS (Malvern Panalytical) instrument with plastic micro cuvettes (Malvern Panalytical). Cas9-EDV and LV were produced as described above, except that 6-18 hours post-transfection of lentiX-cells, media was changed into 5 mL Opti-MEM instead of 10 mL per 10 cm tissue culture dish. Two days post-media change, Cas9-EDV or LV-containing supernatants were harvested and passed through a 0.45 pm PES syringe filter (VWR) without further concentration. EDV and LV particle numbers were measured and normalized using the QuickTiter™ Lentivirus Titer Kit (Lentivirus-Associated HIV p24) as described above. For DLS measurements, 40 1 of normalized Cas9-EDV and LV were prepared, and particle size was measured at 25° C. 100 nm diameter NanoXact Gold Nanospheres-Bare (Citrate) (nanoComposix) were included as a control. Data were analyzed by intensity using the Zetasizer analysis software.

Quantitative Reverse Transcription-PCR (RT-qPCR) of B2M sgRNA

Cas9-EDVs containing guide RNA targeting the B2M gene were produced and concentrated as described above, and RNA was extracted from 150pd of Cas9-EDVs using the NucleoSpin RNA Virus Kit (Takara Bio) according to the manufacturer's instructions. RT-qPCR was performed using the PrimeTime™ One-Step RT-qPCR Master Mix (IDT) according to the manufacturer's instructions on a QuantStudio 6 Flex Real-Time PCR System (Thermo Fisher). qPCR primers were ordered as a custom TaqMan Small RNA Assay to detect the B2M sgRNA target sequence

(SEQ ID NO: 286)
GAGUAGCGCGAGCACAGCUAGUUUAAGAGCUAUGCUGGAAACAGCAUAG
CAAGUUUAAAUAAGGCUAGUCCGUUAUCAACUUGAAAAAGUGGCACCGA
GUCGGUGC (Thermo Fisher, Assay ID CTWCW3V).

### Biodistribution Experiment

All studies and procedures were in accordance with the established NIH guidelines for animal care and use and were approved by the UC Berkeley Animal Care and Use Committec (ACUC). All experimental and control animals were housed under the same conditions as approved by the Berkeley Office of Laboratory Animal Care (OLAC). Following anesthesia induction with 2-3% isofluorane, 100 μl of lentivirus displaying either VSVG or CD19⁺VSVGmut, or phosphate-buffered saline (PBS) was administered to C57BL/6 mice (000664, Jackson Laboratory) by retro-orbital injection. 30 minutes post-treatment, mice were euthanized with CO2 to harvest spleen, kidney, heart, liver, lung, and blood. 15 mg of spleen, kidney, heart, liver, and lung samples were harvested and stored in 1×DNA/RNA Shield (Zymo Research). Blood samples were drawn into tubes pre-coated with 0.5M EDTA and were then separated into plasma and red blood cells (RBCs) by carefully layering blood diluted with PBS containing 2% FBS on top of Percoll density gradient media (Cytiva) and centrifuging at 800xg for 20 minutes. Plasma and RBC layers were extracted and mixed with an equal volume of 2×DNA/RNA Shield (Zymo Research). RNA was extracted from tissue samples using the Quick-RNA Viral Kit (Zymo Research) according to the manufacturer's instructions. Lentivirus titers were measured using the Lenti-X™ qRT-PCR Titration Kit (Takara Bio) on a QuantStudio 3 Real-Time PCR System (Thermo Fisher).

### Statistical Analysis

Statistical analysis was performed using Prism v9. Statistical details for all experiments, including value and definition of N, and error bars can be found in the figure legends

### Example 4: Improving Genome Editing Activity of Cas9-EDVs

The targeted delivery of either transgenes and/or genome editor complexes to specific cell types inside the body would facilitate genome engineering-based therapeutics that are not currently used. In an in vivo context, where the ratio of Cas9-EDV:target cell ratio is low, each Cas9-EDV particle should be maximally potent, and/or that high enough Cas9-EDV titers can be administered such that multiple EDVs are delivered per target cell. Therefore, the next goal was to improve the titer and/or the per-particle editing efficiency of Cas9-EDVs. A screen was performed of nuclear localization sequences (NLS) appended to the N-terminal end of Cas9 and identified a 2× p53 NLS variant most potently enhanced genome editing efficiency (FIG. 29). As has been reported previously, it was also confirmed that the inclusion of nuclear export sequence (NES) on the C-terminal end of Gag enhanced the editing efficiency of antibody-targeted Cas9-EDVs (FIG. 29). These different Gag-Cas9 polypeptide variants in the context of CD19 antibody-targeted Cas9 EDVs were next compared and screened for editing efficiency in both on-target CD19⁺EGFP+target cells as well as bystander CD19-EGFP-cells. Both Gag-NES-Cas9 and Gag-2xp53NLS-Cas9 variants resulted in improved editing efficiency compared to the original Gag-Cas9 polypeptide, but incorporating both NLS and NES improved the genome editing efficiency of Cas9-EDVs to a greater extent than either modification alone (FIG. 23). Lastly, the editing efficiency of optimized Gag-NES-NLS-Cas9 EDVs could be pushed even higher by expressing the sgRNA from the Gag-NES-NLS-Cas9 and Gag-pol plasmid backbones, opposed to a separate plasmid (“V2⁺opt. guide expression,” FIG. 23). This strategy ensured that sgRNA over-expression occurred in the cells successfully transfected with Gag-Cas9 and reduced the amount of transfection reagent needed per plate for Cas9-EDV production, likely improving the health of the producer cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An enveloped delivery vehicle (EDV) comprising:
a) a nucleic acid-binding effector polypeptide; and
b) one or more fusion polypeptides comprising:
i) a viral envelop protein; and
ii) a targeting polypeptide that provides for binding to a target cell.

2. The EDV of claim 1, wherein the targeting polypeptide comprises one or more antibodies or antibody analogs.

3. The EDV of claim 2, wherein the one or more antibody analogs is an affibody, an affilin, an affimer, an affitin, an alphabody, an anticalin, an avimer, a DARPin, a Fynomer, a Kunitz domain peptide, a monobody, a repebody, a VLR, or a nanoCLAMP.

4. The EDV of claim 2, wherein the one or more antibodies is a single chain Fv (scFv) polypeptide, a diabody, a bispecific antibody, a triabody, or a nanobody.

5. The EDV of any one of claim 1-4, wherein the target cell is a cancer cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.

6. The EDV of any one of claims 1-4, wherein the target cell is a cancer cell.

7. The EDV of any one of claims 1-4, wherein the target cell is a CD8⁺ T cell or a CD4⁺ T cell.

8. The EDV of any one of claims 1-4, wherein the targeting polypeptide comprises an anti-CD19, anti-CD20, anti-CD4, anti-CD28, or anti-CD3 antibody.

9. The EDV of any one of claims 1-4, wherein the targeting polypeptide comprises: (a) an anti-CD3 and an anti-CD4 antibody; (b) an anti-CD3 and an anti-CD28 antibody; or (c) an anti-CD3, an anti-CD4, and an anti-CD28 antibody.

10. The EDV of any one of claims 2-9, wherein the targeting polypeptide is a fusion polypeptide comprising:
(i) the one or more antibodies or antibody analogs; and
(ii) one or more heterologous polypeptides.

11. The EDV of claim 10, wherein the one of more heterologous polypeptides comprises a stalk portion of a transmembrane polypeptide.

12. The EDV of claim 10, wherein the one of more heterologous polypeptides comprises a stalk portion and a transmembrane portion of a transmembrane polypeptide.

13. The EDV of claim 12, wherein the transmembrane polypeptide is a CD8a chain polypeptide or a platelet-derived growth factor polypeptide.

14. The EDV of claim 11, wherein the one or more heterologous polypeptides comprises the stalk portion of a CD8a chain polypeptide.

15. The EDV of claim 11, wherein the one or more heterologous polypeptides comprises the stalk portion and the transmembrane domain of a CD8a chain polypeptide.

16. The EDV of claim 15, wherein the stalk portion and the transmembrane domain comprises the amino acid sequence TTTPAPRPPTPAPTIASQPLSLRPEACRPAAGGAVIHTRGLDFACDIYIWAPLAGTCGVLLLSLVIT LYC (SEQ ID NO:20).

17. The EDV of any one of claims 1-16, wherein the viral envelope protein is selected from a Hepatitis B virus (HBV) glycoprotein, a Hepatitis C virus (HCV) glycoprotein, a Marburg virus glycoprotein, an Ebola virus glycoprotein, a vesicular stomatitis virus (VSV) glycoprotein, an influenza virus hemagglutinin, a SARS-CoV glycoprotein, a respiratory syncytial virus (RSV) glycoprotein, a human parainfluenza virus glycoprotein, a measles virus hemagglutinin and/or a measles virus fusion glycoprotein, an HTLV-1 glycoprotein, a Ross river virus glycoprotein, a rabies virus glycoprotein, a Mokola virus glycoprotein, a Semliki Forest virus glycoprotein, a Sindbis virus glycoprotein, a Venezuelan equine encephalitis virus glycoprotein.

18. The EDV of any one of claims 1-16, wherein the viral envelope protein is a variant viral envelope protein that comprises one or more amino acid substitutions that reduce binding of the viral envelope protein to its receptor.

19. The EDV of claim 18, wherein the viral glycoprotein is a variant vesicular stomatitis virus glycoprotein (VSVG) that comprises a substitution of K47 and/or R354, wherein the amino acid numbering is based on the amino acid sequence depicted in FIG. 16A.

20. The EDV of any one of claims 1-19, wherein the nucleic acid binding effector polypeptide is a CRISPR-Cas effector polypeptide, a Zinc Finger Nuclease (ZFN) or a Transcription activator-like effector nuclease. CRISPR-Cas effector polypeptide is a type 11 CRISPR-Cas effector polypeptide, a type V CRISPR-Cas effector polypeptide, or a type VI CRISPR-Cas effector polypeptide.

21. The EDV of claim 20, wherein the CRISPR-Cas effector polypeptide is a type II CRISPR-Cas effector polypeptide, a type V CRISPR-Cas effector polypeptide, or a type VI CRISPR-Cas effector polypeptide.

22. The EDV of any one of claims 1-21, wherein the nucleic acid-binding effector polypeptide is a fusion polypeptide comprising: i) a CRISPR-Cas effector polypeptide; and ii) one or more heterologous polypeptides.

23. The EDV of claim 22, wherein the CRISPR-Cas effector polypeptide exhibits reduced catalytic activity compared to a wild-type CRISPR-Cas effector polypeptide, wherein the CRISPR-Cas effector polypeptide retains the ability to bind to a target nucleic acid when the CRISPR-Cas effector polypeptide is complexed with a guide nucleic acid.

24. The EDV of claim 22 or 23, wherein at least one of the one or more heterologous polypeptides comprises a deaminase, a reverse transcriptase, a transcription modulator, or an epigenetic modulator.

25. The EDV of any one of claims 22-24, wherein at least one of the one or more heterologous polypeptides is a lentiviral Gag polypeptide.

26. The EDV of any one of claims 22-25, wherein the one or more heterologous polypeptides comprises one or more nuclear localization signals.

27. The EDV of any one of claims 22-26, wherein the one or more heterologous polypeptides comprises a nuclear export signal (NES) polypeptide.

28. The EDV of any one of claims 1-27, comprising one or more CRISPR-Cas guide RNAs, or one or more nucleic acids comprising nucleotide sequences encoding the one or more CRISPR-Cas guide RNAs.

29. The EDV of any one of claims 1-28, further comprising a donor template nucleic acid, or a nucleotide sequence encoding the donor template nucleic acid.

30. The EDV of any one of claims 1-29, further comprising a therapeutic polypeptide, or a nucleic acid comprising a nucleotide sequence encoding a therapeutic polypeptide.

31. The EDV of claim 30, wherein the therapeutic polypeptide is a chimeric antigen receptor (CAR).

32. The EDV of claim 31, wherein the CAR comprises one or more scFv or one or more nanobodies specific for a cancer-associated antigen.

33. The EDV of claim 32, wherein:
a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Ra2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin aV03, IGF-R1, IL6, KIR, PD-1, PD-Li, TRAILRI, TRAILR2, transferrin receptor, and VEGF.

34. The EDV of any one of claims 28-33, wherein at least one of the one or more guide RNAs comprises a nucleotide sequence that hybridizes with a target nucleic acid and provides for knockout of the target nucleic acid.

35. The EDV of claim 34, wherein the target nucleic acid that is knocked out encodes an immune checkpoint.

36. The EDV of claim 35, wherein the immune checkpoint is PD-1.

37. The EDV of claim 34, wherein the target nucleic acid that is knocked out encodes a T-cell receptor alpha constant (TRAC) polypeptide.

38. A composition comprising the EDV of any one of claims 1-37.

39. The composition of claim 38, comprising a pharmaceutically acceptable excipient.

40. A method of delivering a nucleic acid-binding effector polypeptide to a eukaryotic cell, the method comprising contacting a eukaryotic cell with the EDV of any one of claims 1-37, or the composition of claim 38 or claim 39.

41. The method of claim 40, wherein the eukaryotic cell is in vivo.

42. The method of claim 40, wherein the eukaryotic cell is in vitro.

43. The method of any one of claims 40-42, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.

44. A method for modifying a target nucleic acid in a eukaryotic cell, the method comprising contacting a eukaryotic cell with the EDV of any one of claims 1-37, or the composition of claim 38 or claim 39, wherein said contacting results in delivery of the nucleic acid-binding effector polypeptide into the cell and modification of a target nucleic acid within the cell.

45. The method of claim 44, wherein the eukaryotic cell is in vivo.

46. The method of claim 44, wherein the eukaryotic cell is in vitro.

47. The method of any one of claims 44-46, wherein the eukaryotic cell is a cancer cell, a stem cell, a hematopoietic stem cell, a lung cell, a neuron, an adipocyte, a hepatocyte, an endothelial cell, a muscle cell, a cardiomyocyte, a retinal cell, a tissue-resident stem cell, a monocyte, a macrophage, a B cell, or a T cell.

48. A method of modifying a target nucleic acid in a target eukaryotic cell in vivo, the method comprising administering to an individual in need thereof an effective amount of the EDV of any one of claims 1-37, or the composition of claim 38 or claim 39, wherein the EDV enters a target eukaryotic cell in the individual and modifies a target nucleic acid in the target eukaryotic cell.

49. The method of claim 48, wherein the target eukaryotic cell is a CD4⁺ T cell or a CD8′ T cell.

50. The method of claim 49, wherein the targeting polypeptide comprises an anti-CD3 antibody and an anti-CD28 antibody.

51. The method of claim 50, wherein the EDV comprises:
a) a CRISPR-Cas effector polypeptide, or a nucleic acid encoding the CRISPR-Cas effector polypeptide; and
b) one or more CRISPR-Cas guide RNAs, or one or more nucleic acids encoding the one or more CRISPR-Cas guide RNAs.

52. The method of claim 48, wherein the targeting polypeptide is an antibody, antibody analog, single chain Fv, diabody, triabody, nanobody or a bi-specific antibody.

53. The method of claim 52, wherein the targeting polypeptide binds to a surface antigen on a T-cell.

54. The method of claim 52, wherein the targeting polypeptide binds to CD19, CD20, CD4, CD28, or CD3.

55. The method of any one of claims 48-54, wherein the EDV comprises a nucleic acid comprising a nucleotide sequence encoding a chimeric antigen receptor (CAR).

56. The method of claim 55, wherein the CAR comprises one or more scFv or one or more nanobodies specific for a cancer-associated antigen.

57. The method of claim 56, wherein:
a) the cancer-associated antigen is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Ra2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) the cancer-associated antigen is an antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILRI, TRAILR2, transferrin receptor, and VEGF.

58. The method of any one of claims 51-57, wherein at least one of the one or more guide RNAs comprises a nucleotide sequence that hybridizes with a target nucleic acid and provides for knockout of the target nucleic acid.

59. The method of claim 58, wherein the target nucleic acid that is knocked out encodes an immune checkpoint.

60. The method of claim 59, wherein the immune checkpoint is PD-1.

61. The method of claim 58, wherein the target nucleic acid that is knocked out encodes a T-cell receptor alpha constant (TRAC) polypeptide.

62. The method of any one of claims 57-61, wherein said administering treats a cancer in the individual, wherein the cancer comprises cells that express the cancer-associated antigen.

63. The method of any one of claims 51-62, wherein said administering is via intravenous administration.


SEQUENCE LISTING (286 sequences)

SEQ ID NO: 1 - protein, 7 aa, synthetic construct
PKKKRKV

SEQ ID NO: 2 - protein, 16 aa, synthetic construct
KRPAATKKAGQAKKKK

SEQ ID NO: 3 - protein, 9 aa, synthetic construct
PAAKRVKLD

SEQ ID NO: 4 - protein, 11 aa, synthetic construct
RQRRNELKRSP

SEQ ID NO: 5 - protein, 38 aa, synthetic construct
NQSSNFGPMKGGNFGGRSSGPYGGGGQYFAKPRNQGGY

SEQ ID NO: 6 - protein, 42 aa, synthetic construct
RMRIZFKNKGKDTAELRRRRVEVSVELRKAKKDEQILKRRNV

SEQ ID NO: 7 - protein, 8 aa, synthetic construct
VSRKRPRP

SEQ ID NO: 8 - protein, 8 aa, synthetic construct
PPKKARED

SEQ ID NO: 9 - protein, 8 aa, synthetic construct
PQPKKKPL

SEQ ID NO: 10 - protein, 12 aa, synthetic construct
SALIKKKKKMAP

SEQ ID NO: 11 - protein, 5 aa, synthetic construct
DRLRR

SEQ ID NO: 12 - protein, 10 aa, synthetic construct
RKLKKKIKKL

SEQ ID NO: 13 - protein, 10 aa, synthetic construct
REKKKFLKRR

SEQ ID NO: 14 - protein, 20 aa, synthetic construct
KRKGDEVDGVDEVAKKKSKK

SEQ ID NO: 15 - protein, 17 aa, synthetic construct
RKCLQAGMNLEARKTKK

SEQ ID NO: 16 - protein, 7 aa, synthetic construct
PKQKKRK

SEQ ID NO: 17 - protein, 30 aa, synthetic construct
MDSLLMNRRKFLYQFKNVRWAKGRRETYLC

SEQ ID NO: 18 - protein, 512 aa, synthetic construct
IMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 21 - protein, 45 aa, synthetic construct
TTTPAPRPPTPAPTIASQPLSLRPEACRPAAGGAVHTRGLDFACD

SEQ ID NO: 22 - protein, 24 aa, synthetic construct
IYIWAPLAGTCGVLLLSLVITLYC

SEQ ID NO: 23 - skipped

SEQ ID NO: 24 - protein, 20 aa, synthetic construct
  VARIANT 4: note = wherein each X at position 4 is selected from lysine, histidine, and arginine
  VARIANT 8: note = wherein each X at position 8 is selected from lysine, histidine, and arginine
  VARIANT 11: note = wherein each X at position 11 is selected from lysine, histidine, and arginine
  VARIANT 15: note = wherein each X at position 15 is selected from lysine, histidine, and arginine
  VARIANT 19: note = wherein each X at position 19 is selected from lysine, histidine, and arginine
GLFXALLXLLXSLWXLLLXA

SEQ ID NO: 25 - protein, 20 aa, synthetic construct
GLFHALLHLLHSLWHLLLHA

SEQ ID NO: 26 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 27 - protein, 178 aa, synthetic construct
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 28 - protein, 160 aa, synthetic construct
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 29 - protein, 161 aa, synthetic construct
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 30 - protein, 183 aa, synthetic construct
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 31 - protein, 164 aa, synthetic construct
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 32 - protein, 173 aa, synthetic construct
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 33 - protein, 161 aa, synthetic construct
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 34 - protein, 179 aa, synthetic construct
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 35 - protein, 198 aa, synthetic construct [sequence omitted]

SEQ ID NO: 36 - protein, 188 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 37 - skipped

SEQ ID NO: 38 - protein, 50 aa, synthetic construct
  VARIANT 6..50: note = Wherein up to 9 copies of GGGGS may be omitted
GGGGSGGGGSGGGGSGGGGSGGGGSGGGGSGGGGSGGGGSGGGGSGGGGS

SEQ ID NO: 39 - protein, 4 aa, synthetic construct
GGSG

SEQ ID NO: 40 - protein, 50 aa, synthetic construct
  VARIANT 6..50: note = Wherein up to 9 copies of GSGGS may be omitted
GSGGSGSGGSGSGGSGSGGSGSGGSGSGGSGSGGSGSGGSGSGGSGSGGS

SEQ ID NO: 41 - protein, 60 aa, synthetic construct
  VARIANT 7..60: note = Wherein up to 9 copies of GGSGGS may be omitted
GGSGGSGGSGGSGGSGGSGGSGGSGGSGGSGGSGGSGGSGGSGGSGGSGGSGGSGGSGGS

SEQ ID NO: 42 - protein, 40 aa, synthetic construct
  VARIANT 5..40: note = Wherein up to 9 copies of GGGS may be omitted
GGGSGGGSGGGSGGGSGGGSGGGSGGGSGGGSGGGSGGGS

SEQ ID NO: 43 - protein, 5 aa, synthetic construct
GGSGG

SEQ ID NO: 44 - protein, 5 aa, synthetic construct
GSGSG

SEQ ID NO: 45 - protein, 5 aa, synthetic construct
GSGGG

SEQ ID NO: 46 - protein, 5 aa, synthetic construct
GGGSG

SEQ ID NO: 47 - protein, 5 aa, synthetic construct
GSSSG

SEQ ID NO: 48 - protein, 422 aa, synthetic construct
SVSQHFNVYKATRPYIAYCADCGAGHSCHSPVAIEAVRSEATDGMLKIQFSAQIGIDKSDNHDYTKIRYADGHAIENAVRSSLKVATSGDCFVHGTMGHFILAKCPPGEFLQVSIQDTRNAVRACRIQYHHDPQPVGREKFTIRPHYGKEIPCTTYQQTTAETVEEIDMHMPPDTPDRTLLSQQSGNVKITVGGKKVKYNCTCGTGNVGTTNSDMTINTCLIEQCHVSVTDHKKWQFNSPFVPRADEPARKGKVHIPFPLDNITCRVPMAREPTVIHGKREVTLHLHPDHPTLFSYRTLGEDPQYHEEWVTAAVERTIPVPVDGMEYHWGNNDPVRLWSQLTTEGKPHGWPHQIVQYYYGLYPAATVSAVVGMSLLALISIFASCYMLVAARSKCLTPYALTPGAAVPWTLGILCCAPRAHA

SEQ ID NO: 49 - protein, 563 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 50 - protein, 566 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRXXEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 51 - protein, 566 aa, synthetic construct
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 52 - protein, 568 aa, synthetic construct
MEKIVLLLAIVSLVKSDQICIGYHANNSTEQVDTIMEKNVTVTHAQDILEKTHNGKLCDLNGVKPLILRDCSVAGWLLGNPMCDEFINVPEWSYIVEKASPANDLCYPGDFNDYEELKHLLSRTNHFEKIQIIPKSSWSNHDASSGVSSACPYHGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIENLNKQMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 53 - protein, 560 aa, synthetic construct
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 54 - protein, 226 aa, synthetic construct
MENTTSGFLGPLLVLQAGFFLLTRNLTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 55 - protein, 281 aa, synthetic construct
MQWNSTAFHQALQDPKVRGLYFPAGGSSSGTVNPAPNIASHISSISARTGDPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 56 - protein, 399 aa, synthetic construct
MGLSWTVPLEWGKNHSTTNPLGFFPDHQLDPAFRANTRNPDWDHNPNKDHWTEANKVGVGAFGPGFTPPHGGLLGWSPQAQGMLKTLPADPPPASTNRQSGRQPTPITPPLRDTHPQAMQWNSTTFHQALQDPKVSALYLPAGGSSSGTVNPVPTTASLISSIFSRIGDPAPNMESITSGFLGPLLVLQAGFFLLTKILTIPQSLDSWWTSLNFLGGAPVCLGQNSQSPTSSHSPTSCPPICPGYRWMCLRRFIIFLFILLLCLIFLLVLLDYQGMLPVCPLIPGSSTTSTGPCRTCTTLAQGTSMFPSCCCSKPSDGNCTCIPIPSSWAFGKFLWEWASARFSWLSLLVPFVQWFAGLSPTVWLSVIWMMWYWGPSLYNILSPFIPLLPIFFCLWVYI

SEQ ID NO: 57 - protein, 226 aa, synthetic construct
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWAPNLHNILSPFLPLLPIFLCLWVYI

SEQ ID NO: 58 - protein, 400 aa, synthetic construct
MGGWSSKPRKGMGTNLAVPNPLGFFPDHQLDPAFKANSDNPDWDLNTHKDYWPDAWKVGVGAFGPGFTPPHGGLLGWSPQAQGLLTTVPAAPPPASTNRQSGRQPTPLSPPLRDTHPQAMKWNSTTFHQTLQDPRVRALYLPAGGSSSGTVSPAQNTVSAISSILSKTGDPVPNMESIASGLLGPLLVLQAGFFLLTKILTIPQSLDSWWTSLNFLGGTPVCLGQNSQSQISSHSPTCCPPTCPGYRWMCLRRFIIFLCILLLCLIFLLVLLDYQGMLPVCPLIPGSSTTSTGPCKTCTAPAQGTSMFPSCCCTKPTDGNCTCIPIPSSWAFAKYLWEWASVRFSWLSLLVPFVQWFVGLSPTVWLSVIWMMWFWGPSLYNILSPFIPLLPIFFCLWVYI

SEQ ID NO: 59 - protein, 281 aa, synthetic construct
MQWNSTTFHQTLQDPRVRGLYFPAGGSSSGTVNPVPTTVSHISSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLSVCPLIPGSTTTSTGPCKTCTTPAQGTSIHPSCCCTKPSDGNCTWIPIPSSWAFGKFLWEWASARFSWLSLLVPFVQWFVGLSPTVWLSVIWIMWYWGPSLYSILSPFLPLLPIFFCLWVYI

SEQ ID NO: 60 - protein, 524 aa, synthetic construct
MVPQALLFVPLLVFPLCFGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESLVIISPSVADLDPYDKSLHSRVFPSGKCSGITVSSTYCSTNHDYTIWMPENLRLGTSCDIFINSRGKRASKGSQTCGFIDERGLYKSLKGACKLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPEGHVLIPEMQSSLLQQHMELLESSVIPLMHPLADPSTVFKEGDEAEDFVEVHLPDVHKQVSGVNLGLPNWGKYVLLSAGALIALMLIIFLLTCCRRVNRPESTQHSLGGKRRKVSITSQSGKIISSWESYKSGGETRL

SEQ ID NO: 61 - protein, 522 aa, synthetic construct
MNIPCFVVILSLATTHSLGEFPLYTIPEKIEKWTPIDMIHLSCPNNLLSEEEGCNAESSFTYFELKSGYLAHQKVPGFTCTGVVNEAETYTNFVGYVTTTFKRKHFRPTVAACRDAYNWKVSGDPRYEESLHTPYPDSSWLRTVTTTKESLLIISPSIVEMDIYGRTLHSPMFPSGVCSNVYPSVPSCETNHDYTLWLPEDPSLSLVCDIFTSSNGKKAMNGSRICGFKDERGFYRSLKGACKLTLCGRPGIRLFDGTWVSFTKPDVHVWCTPNQLINIHNDRLDEIEHLIVEDIIKKREECLDTLETILMSQSVSFRRLSHFRKLVPGYGKAYTILNGSLMETNVYYKRVDKWADILPSKGCLKVGQQCMEPVKGVLFNGIIKGPDGQILIPEMQSEQLKQHMDLLKAAVFPLRHPLISREAVFKKDGDADDFVDLHMPDVHKSVSDVDLGLPHWGFWMLIGATIVAFVVLVCLLRVCCKRVRRRRSGRATQEIPLSFPSAPVPRAKVVSSWESYKGLPGT

SEQ ID NO: 62 - protein, 498 aa, synthetic construct
MGQIVTMFEALPHIIDEVINIVIIVLIIITSIKAVYNFATCGILALISFLLLAGRSCGLYGLDGPDIYKGIYQFKSVEFDMSHLNLTMPNACSANNSHHYISMGNSGLELTFTNDSIISHNFCNLTSAFNKKTFDHTLMSIVSSLHLSIRGNSNYKAVSCDFNSGITIQYNLSFSDAQSALSQCKTFRGRVLDMFRTAFGGKYMRSGWGWTGSDGKTTWCSQTSYQYLIIQNRTWENHCRYAGPFGMARILFAQEKTKFLTRRLAGTFTWTLSDSSGVDNPGGYCLTRWMILAADLKCFGNTAVAKCNMNHDEEFCDMLRLIDYNKAALSKFKEDVESALHLFKVTVNSLVSDQLLMRNHLRDLMGVPYCNYSRFWYLEHTKTGETSVPKCWLVTNGSYLNETHFSDQIEQEADNMITDMLRKDYIKRQGSTPLALMDLLMFSTSAYLVSVFLHLVKIPTHRHIKGGSCPKPHRLTNKGICSCGAFKVPGVKTVWKRR

SEQ ID NO: 63 - protein, 498 aa, synthetic construct
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 64 - protein, 498 aa, synthetic construct
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFFTRRLAGTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 65 - protein, 207 aa, synthetic construct
MYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFFTRRLA

SEQ ID NO: 66 - protein, 233 aa, synthetic construct
GTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 67 - protein, 438 aa, synthetic construct
YEHTATIPNVVGFPYKAHIERNXFSPMTLQLEVVXXSLEPTLNLEYITCEYKTVVPSPFIKCCGTSECSSKEQPDYQCKVYTGVYPFMWGGAYCFCDSENTQLSEAYVDRSDVCKHDHALAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTQTPSGFKYWLKEKGSSLNTKAPFGCKIKTNPVRAMDCAVGSIPVSMDIPDSAFTRVVDAPAVTDLSCQVAVCTHSSDFGXVATLSYKTDKPGKCAVHSHSNVATLQEATVDVKEDGKVTVHFSXXSASPAFKVSVCDAKTTCTAACEPPKDHIVPYGASHNNQVFPDMSGTAMTWVQRMASGLGGLALIAVVVLVLVTCITMRR

SEQ ID NO: 68 - protein, 422 aa, synthetic construct
SVIEHFNVYKATRPYLAXCADCGDGYFCYSPVAIEKIRDEASDGMLKIQVSAQIGLDKAGTHAHTKMRYMAGHDVQESKRDSLRVYTSAACSIHGTMGHFIVAHCPPGDYLKXSFEDANSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWXFTSPFVPRADQTARKGKVHVPFPLTNVTCRVPLARAPDVTYGKKEVTLRLHPDHPTXFSYRSLGAVPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLXCAPRANA

SEQ ID NO: 69 - protein, 438 aa, synthetic construct
YEHSTVMPNVVGFPYKAHIERPGYSPLTLQMQVVETSLEPTLNLEYITCEYKTVVPSPYVKCCGASECSTKEKPDYQCKVYTGVYPFMWGGAYCFCDSENTQLSEAYVDRSDVCRHDHASAYKAHTASLKAKVRVMYGNVNQTVDVYVNGDHAVTIGGTQFIFGPLSSAWTPFDNKIVVYKDEVFNQDFPPYGSGQPGRFGDIQSRTVESNDLYANTALKLARPSPGMVHVPYTQTPSGFKYWLKEKGTALNTKAPFGCQIKTNPVRAMNCAVGNIPVSMNLPDSAFTRIVEAPTIIDLTCTVATCTHSSDFGGVLTLTYKTNKNGDCSVHSHSNVATLQEATAKVKTAGKVTLHFSTASASPSFVVSLCSARATCSASCEPPKDHIVPYAASHSNVVFPDMSGTALSWVQKISGGLGAFAIGAILVLVVVTCIGLRR

SEQ ID NO: 70 - protein, 439 aa, synthetic construct
YEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 71 - protein, 423 aa, synthetic construct
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 72 - protein, 676 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 73 - protein, 644 aa, synthetic construct
IPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 74 - protein, 677 aa, synthetic construct
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFAEGVVAFLILSEPKKHFWKATPAHEPVNTTDDSTSYYMTLTLSYEMSNFGGNESNTLFKVDNHTYVQLDRPHTPQFLVQLNETLRRNNRLSNSTGRLTWTLDPKIEPDVGEWAFWETKKNFSQQLHGENLHFQIPSTHTNNSSDQSPAGTVQGKISYHPPANNSELVPTDSPPVVSVLTAGRTEEMSTQGLTNGETITGFTANPMTTTIAPSPTMTSEVDNNVPSEQPNNTASIEDSPPSASNETIYHSEMDPIQGSNNSAQSPQTKTTPAPTTSPMTQDPQETANSSKPGTSPGSAAGPSQPGLTINTVSKVADSLSPTRKQKRSVRQNTANKCNPDLYYWTAVDEGAAVGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 75 - protein, 681 aa, synthetic construct
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 76 - protein, 669 aa, synthetic construct
MESTTLSKPFKNQVNPWGPLIVLLILGGVNPVALGNSPHQVFNLTWEVTNGDRETVWAIAGNHPLWTWWPDLTPDLCMLALHGPSYWGLEYRAPFSPPPGPPCCSGSSDSTPGCSRDCEEPLTSYTPRCNTAWNRLKLSKVTHAHNEGFYVCPGPHRPRWARSCGGPESFYCASWGCETTGRASWKPSSSWDYITVSNNLTSDQATPVCKGNEWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILDLTTDYCVLVELWPRVTYHSPSYVYHQFEGRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMGPLIILLLILLFGPCILNRLVQFIKDRISVVQALVLTQQYHQLKTIRDCKSRE

SEQ ID NO: 77 - protein, 669 aa, synthetic construct
MESTTLSKPFKNQVNPWGPLIVLLILRGVNPVTLGNSPHQVFNLTWEVTNGDRETVWAITGNHPLWTWWPDLTPDLCMLALHGPSYWGLEYRAPFSPPPGPPCCSGSSDSTPGCSRDCEEPLTSYTPRCNTAWNRLKLSKVTHAHNGGFYVCPGPHRPRWARSCGGPESFYCASWGCETTGRASWKPSSSWDYITVSNNLTSDQATPVCKGNKWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILDLTTDYCVLVELWPRVTYHSPSYVYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMGPLIILLLILLFGPCILNRLVQFIKDRISVVQALVLTQQYHQLKIIEDCKSRE

SEQ ID NO: 78 - protein, 654 aa, synthetic construct
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 79 - protein, 645 aa, synthetic construct
MEGPAFSKPLKDKINPWKSLMVMGVYLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYQALNLTNPDKTQECWLCLVSGPPYYEGVAVVGTYTNHSTAPASCTATSQHKLTLSEVTGQGLCMGALPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGQFNRSPWFTTLISTIMGPLIVLLLILLFGPCILNRLVQFVKDRISVVQALVLTQQYHQLKPIEYEP

SEQ ID NO: 80 - protein, 644 aa, synthetic construct
MEGSAFSKPLKDKINPWGPLIVMGILVRAGASVQRDSPHQIFNVTWRVTNLMTGQTANATSLLGTMTDTFPKLYFDLCDLVGDYWDDPEPDIGDGCRTPGGRRRTRLYDFYVCPGHTVPIGCGGPGEGYCGKWGCETTGQAYWKPSSSWDLISLKRGNTPKDQGPCYDSSVSSGVQGATPGGRCNPLVLEFTDAGRKASWDAPKVWGLRLYRSTGADPVTRFSLTRQVLNVGPRVPIGPNPVITDQLPPSQPVQIMLPRPPHPPPSGTVSMVPGAPPPSQQPGTGDRLLNLVEGAYQALNLTSPDKTQECWLCLVSGPPYYEGVAVLGTYSNHTSAPANCSVASQHKLTLSEVTGQGLCVGAVPKTHQALCNTTQKTSDGSYYLAAPAGTIWACNTGLTPCLSTTVLNLTTDYCVLVELWPKVTYHSPDYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLLGPCILNRLVQFVKDRISVVQALILTQQYHQLKSIEPEEVESRE

SEQ ID NO: 81 - protein, 408 aa, synthetic construct
VQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSLWDLISLKRGNTPQNQGPCYDSSAVSSDIKGATPGGRCNPLVLEFTDAGKKASWDGPKVWGLRLYRSTGTDPVTRFSLTRRVLNIGPRVPIGPNPVIIDQLPPSRPVQIMLPRPPQPPPPGAASIVPETAPPSNQPGTGDRLLNLVDGAYQALNLTSPDKTQECWLCLVAEPPYYEGVAVLGTYSNHTSAPANCSVASQHKLTLSEVTGRGLCIGTVPKTHQALCNTTLKTNKGSYYLVAPAGTTWACNTGLTPCLSATVLNRTTDYCVLVELWPRVTYHPPSYVYSQFEKSYRHKR

SEQ ID NO: 82 - protein, 685 aa, synthetic construct
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 83 - protein, 644 aa, synthetic construct
TSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 84 - protein, 448 aa, synthetic construct
TSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKR

SEQ ID NO: 85 - protein, 181 aa, synthetic construct
EAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKIL

SEQ ID NO: 86 - protein, 565 aa, synthetic construct
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVVHAMVLAQQYQALKAEEEAQD

SEQ ID NO: 87 - protein, 565 aa, synthetic construct
MTAYIQRSQCISTSLLVVLTTLVSCQIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAKER

SEQ ID NO: 88 - protein, 540 aa, synthetic construct
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR

SEQ ID NO: 89 - protein, 91 aa, synthetic construct
QIPRDRLSNIGVIVDEGKSLKIAGSHESRYIVLSLVPGVDFENGCGTAQVIQYKSLLNRLLIPLRDALDLQEALITVTNDTTQNAGAPQSR

SEQ ID NO: 90 - protein, 449 aa, synthetic construct
FFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAKER

SEQ ID NO: 91 - protein, 575 aa, synthetic construct
MDGDRSKRDSYWSTSPGGSTTKLVSDSERSGKVDTWLLILAFTQWALSIATVIICIVIAARQGYSMERYSMTVEALNTSNKEVKESLTSLIRQEVITRAANIQSSVQTGIPVLLNKNSRDVIRLIEKSCNRQELTQLCDSTIAVHHAEGIAPLEPHSFWRCPAGEPYLSSDPEVSLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPIVDERTDYSSDGIEDLVLDILDLKGRTKSHRYSNSEIDLDHPFSALYPSVGSGIATEGSLIFLGYGGLTTPLQGDTKCRIQGCQQVSQDTCNEALKITWLGGKQVVSVLIQVNDYLSERPRIRVTTIPITQNYLGAEGRLLKLGDQVYIYTRSSGWHSQLQIGVLDVSHPLTISWTPHEALSRPGNEDCNWYNTCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 92 - protein, 611 aa, synthetic construct
MPKRRAGFRKGWYARQRNSLTHQMQRMTLSEPTSELPTQRQIEALMRYAWNEAHVQPPVTPTNILIMLLLLLQRIQNGAAATFWAYIPDPPMLQSLGWDKETVPVYVNDTSLLGGKSDIHISPQQANISFYGLTTQYPMCFSYQSQHPHCIQVSADISYPRVTISGIDEKTGMRSYRDGTGPLDIPFCDKHLSIGIGIDTPWTLCRARIASVYNINNANTTLLWDWAPGGTPDFPEYRGQHPPISSVNTAPIYQTELWKLLAAFGHGNSLYLQPNISGSKYGDVGVTGFLYPRACVPYPFMVIQGHMEITPSLNIYYLNCSNCILTNCIRGVAKGEQVIIVKQPAFVMLPVEITEEWYDETALELLQRINTALSRPKRGLSLIILGIVSLITLIATAVTASVSLAQSIQVAHTVDSLSSNVTKVMGTQENIDKKIEDRLPALYDVVRVLGEQVQSINFRMKIQCHANYKWICVTKKPYNTSDFPWDKVKKHLQGIWFNTTVSLDLLQLHNEILDIENSPKATLNIADTVDNFLQNLFSNFPSLHSLWRSIIAMGAVLTFVLIIICLAPCLIRSIVKEFLHMRVLIHKNMLQHQHLMELLNNKERGAAGDDP

SEQ ID NO: 93 - protein, 505 aa, synthetic construct
MFHLLTLLLLLFINMNLYLAGEHCNVQMKNGPYRIKNLAITPPRETLKKDVTVTIVETDYEENVLIGYKGYYQAYGYNGGSLDANTRLEETMESLPLTKEDLLTWTYRQECEVGEELIDRWGSDSDDCYRNKDGRGVWVKTKELVKRQNNNHFAHHTCNRSWRCGFSTAKMYSKLVCDDETNDCKVFILDNTGKPINITTNEVLYRDGVNMMLKSKPTFTRREEKVACLLVKDELNPDKTREHCLIDSDIYDLSNNNWFCMFNKCIKRNVDSVVKKRPNKWMHNLAPKYSEGATATKGDMMHIQEELMYENDLLKMNIELVHAHMNKLNNIIHDLIVSIAKVDERLIGNLMNISVSSVFLSDDTFLLMPCTNPPQHTSNCYNNSIYREGRWVFNEDTSECIDFNNYRELSIDDDIEFWIPTIGNTTYHDSWKDASGWSFVAQQKSNLIMTMENTKFGGVGTSLSDITSMSEGELTAKLTTFVFSHIVTFILIIILIILCICLLKK

SEQ ID NO: 94 - protein, 509 aa, synthetic construct
MLRITLLILFLVRFVSGAEHCNAQMKSGPWRIKNLPIAPPKETLQKDVDVEIVETDLDENVIIGYKGYYQAYAYNGGSLDPNTSVDETTQTLNIDKDDLITWGDRRKCEVGEELIDQWGSDSDSCFKDKLGRGVWVAGKELVKRKNNNHFAHHTCNRSWRCGVSTAKMYTRLECDNETDDCKVTILDINGTVINVTENEVLHRDGVSMILKQKSTFTRRTEKVACLLIKDDKSDPYSITREHCLIDNDIFDLSKNTWNCKFNRCIKRRSENVVKKRPPTWRHNEPPKHSEGTTATKGDLMHIQEELMYENDLLRMNLELLHAHINKLNNMMHDLIVSVAKVDERLIGNLMNNSVSSTFLSDDTFLLMPCTNPPPHTSNCYNNSIYKEGRWVANTDSSQCIDFRNYKELAIDDDIEFWIPTIGNTSYHESWKDASGWSFIAQQKSNLISTMENTKFGGHTTSLSDIGDMAKGELNATLYSFMLGHGFSFFLIIGVIVFLICMVRSRVRAF

SEQ ID NO: 95 - protein, 530 aa, synthetic construct
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 96 - protein, 1251 aa, synthetic construct
MFPFQPMYPMQPMPYRNPFAAPRRPWFPRTDPFLAMQVQELTRSMANLTFKQRRDAPPEGPSAKKPKKEASQKQKGGGQGKKKKNQGKKKAKTGPPNPKAQNGNKKKTNKKPGKRQRMVMKLESDKTFPIMLEGKINGYACVVGGKLFRPMHVEGKIDNDVLAALKTKKASKYDLEYADVPQNMRADTFKYTHEKPQGYYSWHHGAVQYENGRFTVPKGVGAKGDSGRPILDNQGRVVAIVLGGVNEGSRTALSVVMWNEKGVTVKYTPENCEQWSLVTTMCLLANVTFPCAQPPICYDRKPAETLAMLSVNVDNPGYDELLEAAVKCPGSTEELFKEYKLTRPYMARCIRCAVGSCHSPIAIEAVKSDGHDGYVRLQTSSQYGLDSSGNLKGRTMRYDMHGTIKEIPLHQVSLHTSRPCHIVDGHGYFLLARCPAGDSITMEFKKDSVTHSCSVPYEVKFNPVGRELYTHPPEHGVEQACQVYAHDAQNRGAYVEMHLPGSEVDSSLVSLSGSSVTVTPPVGTSALVECECGGTKISETINKTKQFSQCTKKEQCRAYRLQNDKWVYISDKLPKAAGATLKGKLHVPFLLADGKCTVPLAPEPMITFGFRSVSLKLHPKNPTYLTTRQLADEPHYTHELISEPAVRNFTVTGKGWEFVWGNHPPKRFWAQETAPGNPHGLPHEVITHYYHRYPMSTILGLSICAAIATVSVAASTWLFCRSRVACLTPYRLTPNARIPFCLAVLCCARTARAETTWESLDHLWNNNQQMFWIQLLIPLAALIVVTRLLRCVCCVVPFLVMAGAAGAGAYEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKLTAGPLSTAWTPFDRKIVQYAGEIYNYDFPEYGAGQPGAFGDIQSRTVSSSDLYANTNLVLQRPKAGAIHVPYTQAPSGFEQWKKDKAPSLKSTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 97 - protein, 423 aa, synthetic construct
STEELFKEYKLTRPYMARCIRCAVGSCHSPIAIEAVKSDGHDGYVRLQTSSQYGLDSSGNLKGRTMRYDMHGTIKEIPLHQVSLHTSRPCHIVDGHGYFLLARCPAGDSITMEFKKDSVTHSCSVPYEVKFNPVGRELYTHPPEHGVEQACQVYAHDAQNRGAYVEMHLPGSEVDSSLVSLSGSSVTVTPPVGTSALVECECGGTKISETINKTKQFSQCTKKEQCRAYRLQNDKWVYISDKLPKAAGATLKGKLHVPFLLADGKCTVPLAPEPMITFGFRSVSLKLHPKNPTYLTTRQLADEPHYTHELISEPAVRNFTVTGKGWEFVWGNHPPKRFWAQETAPGNPHGLPHEVITHYYHRYPMSTILGLSICAAIATVSVAASTWLFCRSRVACLTPYRLTPNARIPFCLAVLCCARTARA

SEQ ID NO: 98 - protein, 442 aa, synthetic construct
YEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKLTAGPLSTAWTPFDRKIVQYAGEIYNYDFPEYGAGQPGAFGDIQSRTVSSSDLYANTNLVLQRPKAGAIHVPYTQAPSGFEQWKKDKAPSLKSTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 99 - protein, 491 aa, synthetic construct
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 100 - protein, 607 aa, synthetic construct
MEAVIKMRRALFLQAFLTGRPGKASKKDPKKNPLATSKKDPEKTPLLPTRVNYILIIGVLVLCEVTGVRADVHLLEQPGNLWITWANRTGQTDFCLSTQSATSPFQTCLIGIPSPISEGDFKGYVSDNCTTLGTDRLVSSASITGGPDNSTTLTYRKVSCLLLKLNVSMWNEPPELQLLGSQSLPNITDITQISGVAGGCVGFRPKGVPWYLGWSQGEATRFLLRHPSFSNLTGPFTVVTADRHNLFMGSEYCGAYGYRFWEIYNCSQEGQQYRCGKARRPRPQSPETQCTRQGGIWVNRSKEINETEPFSFTVNCTASNLGNASGCCGKAGTILPGIWVDSTQGNFTKPKALPPAIFLICGDRAWQGIPSRPVGGPCYLGKLTMLAPNHTDILKILANSSRTGIRRRRSVSHLDDTCSDEVQLWGPTARIFASILAPGVAAAQALREIERLACWSVKQANLTTSLLGDLLDDVTSIRHAVLQNRAAIDFLLLAHGHGCEDIAGMCCFNLSDHSESIQKKFQLMKEHVNKIGVDSDPIGSWLRGLFGGIGGWAVHLLKGLLLGLVVILLLVVCLPCFLQFVSSSIRKMINNSVSYHTEYRKMQGGAV

SEQ ID NO: 101 - protein, 614 aa, synthetic construct
MEAVIKMRRALFLQAFLTGHPGKVSKKDSKKKPPATGKRDPEKTPLLPTRVNYILIIGVLVLCEVTGVRADVHLLEQPGNLWITWANRTGQTDFCLSTQSATSPFQTCLIGIPSPISEGDFKGYVSGNCTALGTHRLVSSGIHGGPDNSTTLTYRKVSCLLLKLNVSLLDEPSELQLLGSQSLPNITNITQIPSVAGGCIGFTPYGSPAGVYGWDRRQVTHILLTDPGSNPFFNKASNSSKPFTVVTADRHNLFMGSEYCGAYGYRFWEMYNCSQMRQNWSICMDVWGRGLPESWCTSTGGIWVNQSKEINETEPFSFTANCTGSNLGNVSGCCGESITILPPGAWVDSTQGSFTKPKALPPGIFLICGDRAWQGIPSRPVGGPCYLGKLTMLAPNHTDILKILANSSQTGVRHKRSVTHLDDTCSDEVQLWGPTARIFASILAPGVAAAQALREIERLACWSVKQANLTTSLLGDLLDDVTSIRHAVLQNRAAIDFLLLAHGHGCEDIAGMCCFNLSDHSESIQKKFQLMKEHVNKIGVDSDPIGSWLRGLFGGIGEWAVHLLKGLLLGLVVILLLVVCLPCFLQFVSSSIRKMINNSISYHTEYRKMQGGAV

SEQ ID NO: 102 - protein, 569 aa, synthetic construct
MEAVIKAFLTGHPGKVSKKDSKKKPPATSKKDPEKTPLLPSRGYFFFPTILVCVVIISVVPGVGGVHLLRQPGNVWVTWANKTGRTDFCLSLQSATSPFRTCLIGIPQYPLNTFKGYVTNVTACDNDADLASQTACLIKALNTTLPWDPQELDILGSQMIKNGTTRTCVTFGSVCYKENNRSRVCHNFDGNFNGTGGAEAELRDFIAKWKSDDLLIRPYVNQSWTMVSPINVESFSISRRYCGFTSNETRYYRGDLSNWCGSKRGKWSAGYSNRTKCSSNTTGCGGNCTTEWNYYAYGFTFGKQPEVLWNNGTAKALPPGIFLICGDRAWQGIPRNALGGPCYLGQLTMLSPNFTTWITYGPNITGHRRSRRAIRGLSPDCSDEVQLWSATARIFASFFAPGVAAAQALKEIERLACWSVKQANLTSLILNAMLEDMNSIRHAVLQNRAAIDFLLLAQGHGCQDVEGMCCFNLSDHSESIHKALQAMKEHTEKIQVEDDPIGDWFTRTFGDLGRWLAKGVKTLLFALLVIVCLLAIIPCIIKCFQDCLSRTMNQFMDERIRYHRIREQL

SEQ ID NO: 103 - protein, 488 aa, synthetic construct
MGKFLATLILFFQFCPLILGDYSPSCCTLTVGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSHLNINLHFSKCGFPFSLLVDAPGYDPIWFLNTEPSQLPPTAPPLLSHSNLDHILEPSIPWKSKLLTLVQLTLQSTNYTCIVCIDRASLSTWHVLYSPNVSVPSLSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCILRQLRHLPSRVRYPHYSLINPESSL

SEQ ID NO: 104 - protein, 989 aa, synthetic construct
MAPPMTLQQWIIWKKMNKAHEALQNTTTVTEQQKEQIILDIQNEEVQPTRRDKFRYLLYTCCATSSRVLAWMFLVCILLIIVLVSCFVTISRIQWNKDIQVLGPVIDWNVTQRAVYQPLQTRRIARSLRMQHPVPKYVEVNMTSIPQGVYYEPHPEPIVVKERVLGLSQILMINSENIANNANLTQEVKKLLTEMVNEEMQSLSDVMIDFEIPLGDPRDQEQYIHRKCYQEFANCYLVKYKEPKPWPKEGLIADQCPLPGYHAGLTYNRQSIWDYYIKVESIRPANWTTKSKYGQARLGSFYIPSSLRQINVSHVLFCSDQLYSKWYNIENTIEQNERFLLNKLNNLTSGTSVLKKRALPKDWSSQGKNALFREINVLDICSKPESVILLNTSYYSFSLWEGDCNFTKDMISQLVPECDGFYNNSKWMHMHPYACRFWRSKKNEKEETKCRDGETKRCLYYPLWDSPESTYDFGYLAYQKNFPSPICIEQQKIRDQDYEVYSLYQERKIASKAYGIDTVLFSLKNFLNYTGTPVNEMPNARAFVGLIDPKFPPSYPNVTREHYTSCNNRKRRSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 105 - protein, 446 aa, synthetic construct
SLRMQHPVPKYVEVNMTSIPQGVYYEPHPEPIVVKERVLGLSQILMINSENIANNANLTQEVKKLLTEMVNEEMQSLSDVMIDFEIPLGDPRDQEQYIHRKCYQEFANCYLVKYKEPKPWPKEGLIADQCPLPGYHAGLTYNRQSIWDYYIKVESIRPANWTTKSKYGQARLGSFYIPSSLRQINVSHVLFCSDQLYSKWYNIENTIEQNERFLLNKLNNLTSGTSVLKKRALPKDWSSQGKNALFREINVLDICSKPESVILLNTSYYSFSLWEGDCNFTKDMISQLVPECDGFYNNSKWMHMHPYACRFWRSKKNEKEETKCRDGETKRCLYYPLWDSPESTYDFGYLAYQKNFPSPICIEQQKIRDQDYEVYSLYQERKIASKAYGIDTVLFSLKNFLNYTGTPVNEMPNARAFVGLIDPKFPPSYPNVTREHYTSCNNRKRR

SEQ ID NO: 106 - protein, 417 aa, synthetic construct
SVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 107 - protein, 983 aa, synthetic construct
MASKESKPSRTTRRGMEPPLRETWNQVLQELVKRQQQEEEEQQGLVSGKKKSWVSIDLLGTEGKDIKKVNIWEPCEKWFAQVVWGVLWVLQIVLWGCLMWEVRKGNQCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKRGIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 108 - protein, 877 aa, synthetic construct
QCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKRGIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 109 - protein, 550 aa, synthetic construct
QCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKR

SEQ ID NO: 110 - protein, 327 aa, synthetic construct
GIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 111 - protein, 1255 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 112 - protein, 605 aa, synthetic construct
CDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIVIVMVTILLCCMTSCCSCLKGACSCGSCCKFDEDDSEPVLKGVKL

SEQ ID NO: 113 - protein, 253 aa, synthetic construct
PNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSE

SEQ ID NO: 114 - protein, 298 aa, synthetic construct
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 115 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 116 - protein, 549 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 117 - protein, 111 aa, synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRR

SEQ ID NO: 118 - protein, 438 aa, synthetic construct
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 119 - protein, 572 aa, synthetic construct
MEYWKHTNHGKDAGNELETSMATHGNKLTNKITYILWTIILVLLSIVFIIVLINSIKSEKAHESLLQNINNEFMEITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASPGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTLQPMLFKTEIPKSCS

SEQ ID NO: 120 - protein, 539 aa, synthetic construct
MPISILLIITTMIMASHCQIDITKLQHVGVLVNSPKGMKISQNFETRYLILSLIPKIDDSNSCGDQQIKQYKRLLDRLIIPLYDGLRLQKDVIVANQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 121 - protein, 192 aa, synthetic construct
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDA

SEQ ID NO: 122 - protein, 363 aa, synthetic construct
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 123 - protein, 563 aa, synthetic construct
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCYPGKFVNEEALRQILRGSGGIDKETMGFTYSGIRTNGTTSACRRSGSSFYAEMEWLLSNTDNASFPQMTKSYKNTRRESALIVWGIHHSGSTTEQTKLYGSGNKLITVGSSKYHQSFVPSPGTRPQINGQSGRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKREKRGLFGAIAGFIENGWEGLVDGWYGFRHQNAQGEGTAADYKSTQSAIDQITGKLNRLIEKTNQQFELIDNEFTEVEKQIGNLINWTKDFITEVWSYNAELLVAMENQHTIDLADSEMNKLYERVRKQLRENAEEDGTGCFEIFHKCDDDCMASIRNNTYDHSKYREEAMQNRIQIDPVKLSSGYKDVILWFSFGASCFLLLAIAVGLVFICVKNGNMRCTICI

SEQ ID NO: 124 - protein, 492 aa, synthetic construct
AEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 125 - skipped

SEQ ID NO: 126 - protein, 553 aa, synthetic construct
MSIMGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIETIRQAGQEMILAVQGVQDYINNELIPSMNQLSCDLIGQKLGLKLLRYYTEILSLFGPSLRDPISAEISIQALSYALGGDINKVLEKLGYSGGDLLGILESGGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGYTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 127 - protein, 11 aa, synthetic construct
YGRKKRRQRRR

SEQ ID NO: 128 - protein, 12 aa, synthetic construct
RRQRRTSKLMKR

SEQ ID NO: 129 - protein, 27 aa, synthetic construct
GWTLNSAGYLLGKINLKALAALAKKIL

SEQ ID NO: 130 - protein, 33 aa, synthetic construct
KALAWEAKLAKALAKALAKHLAKALAKALKCEA

SEQ ID NO: 131 - protein, 16 aa, synthetic construct
RQIKIWFQNRRMKWKK

SEQ ID NO: 132 - protein, 9 aa, synthetic construct
RKKRRQRRR

SEQ ID NO: 133 - protein, 8 aa, synthetic construct
RKKRRQRR

SEQ ID NO: 134 - protein, 11 aa, synthetic construct
YARAAARQARA

SEQ ID NO: 135 - protein, 11 aa, synthetic construct
THRLPRRRRRR

SEQ ID NO: 136 - protein, 11 aa, synthetic construct
GGRRARRRRRR

SEQ ID NO: 137 - protein, 1368 aa, Streptococcus pyogenes [sequence omitted]

SEQ ID NO: 138 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 139 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 140 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 141 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 142 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 143 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 144 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 145 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 146 - protein, 1306 aa, synthetic construct
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 147 - protein, 1120 aa, synthetic construct
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 148 - protein, 1159 aa, synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 149 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 150 - protein, 1285 aa, synthetic construct
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 151 - protein, 1175 aa, synthetic construct
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 152 - protein, 1285 aa, synthetic construct
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 153 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 154 - protein, 495 aa, synthetic construct
KFTIVFPHNQKGNWKNVPSNYHYCPSSSDLNWHNDLIGTALQVQMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 155 - protein, 617 aa, synthetic construct
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKGSELSQLSMYRVFEVGVIRNPGLGAPVFHMTNYFEQPVSNDLSNCMVALGELKLAALCHGGDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPSRSFSYFYPFRLPIKGIPIELQVECFTWDQKLWCRHFCVLADSESGGHITHSGMVGMGVSCTVTREDGTNSR

SEQ ID NO: 156 - protein, 617 aa, synthetic construct
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKGSELSQLSMYRVFEVGVIRNPGLGAPVFHMTNYFEQPVSNDLSNCMVALGELKLAALCHGGDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFTVPIKEAGEDCHAPTYLPAEVDGDVKLSSNLVILPGQDLQYVLATYDTSAVEHAVVYYVYSPSRLSSYFYPFRLPIKGIPIELQVECFTWDQKLWCRHFCVLADSESGGHITHSGMVGMGVSCTVTREDGTNSR

SEQ ID NO: 157 - protein, 671 aa, synthetic construct
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 158 - protein, 512 aa, synthetic construct
MVSAIVLYVLLAAAAHSAFAAEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 159 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 107 aa, synthetic construct [sequence omitted]

SEQ ID NO: 162 - protein, 120 aa, synthetic construct [sequence omitted]

SEQ ID NO: 163 - skipped

SEQ ID NO: 164 - protein, 11 aa, synthetic construct
RASQDVNTAVA

SEQ ID NO: 165 - protein, 6 aa, synthetic construct
SASFLY

SEQ ID NO: 166 - protein, 8 aa, synthetic construct
QQHYTTPP

SEQ ID NO: 167 - protein, 8 aa, synthetic construct
GFNIKDTY

SEQ ID NO: 168 - protein, 8 aa, synthetic construct
IYPTNGYT

SEQ ID NO: 169 - protein, 13 aa, synthetic construct
SRWGGDGFYAMDY

SEQ ID NO: 170 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 171 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 107 aa, synthetic construct [sequence omitted]

SEQ ID NO: 174 - protein, 119 aa, synthetic construct [sequence omitted]

SEQ ID NO: 175 - protein, 11 aa, synthetic construct
KASQDVSIGVA

SEQ ID NO: 176 - protein, 6 aa, synthetic construct
SASYRY

SEQ ID NO: 177 - protein, 8 aa, synthetic construct
QQYYIYPY

SEQ ID NO: 178 - protein, 10 aa, synthetic construct
GFTFTDYTMD

SEQ ID NO: 179 - protein, 18 aa, synthetic construct
ADVNPNSGGSIYNQRFKG

SEQ ID NO: 180 - protein, 12 aa, synthetic construct
ARNLGPSFYFDY

SEQ ID NO: 181 - protein, 15 aa, synthetic construct
KASQSVDYDGDSYLN

SEQ ID NO: 182 - protein, 7 aa, synthetic construct
DASNLVS

SEQ ID NO: 183 - protein, 9 aa, synthetic construct
QQSTEDPWT

SEQ ID NO: 184 - protein, 5 aa, synthetic construct
SYWMN

SEQ ID NO: 185 - protein, 17 aa, synthetic construct
QIWPGDGDTNYNGKFKG

SEQ ID NO: 186 - protein, 15 aa, synthetic construct
RETTTVGRYYYAMDY

SEQ ID NO: 187 - protein, 249 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 188 - protein, 9 aa, synthetic construct
LPPLERLTL

SEQ ID NO: 189 - protein, 10 aa, synthetic construct
LALKLAGLDL

SEQ ID NO: 190 - protein, 11 aa, synthetic construct
LSQALASSFSV

SEQ ID NO: 191 - protein, 12 aa, synthetic construct
NELALKLAGLDI

SEQ ID NO: 192 - protein, 16 aa, synthetic construct
TGGGGSGGGGSGGGGS

SEQ ID NO: 193 - protein, 19 aa, synthetic construct
TGSTSGSGKPGSGEGSTKG

SEQ ID NO: 194 - protein, 15 aa, synthetic construct
GGGGSGGGGSGGGGS

SEQ ID NO: 195 - DNA, 5778 bp, synthetic construct
  source 1..5778: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaaaactttagccagaactatccgattgtgcagaccggtatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgccgactataaggaccacgacggagactacaaggatcatgatattgattacaaagacgatgacgataagagcctgggcagcggctcccccaagaaaaaacgcaaggtggaagatcctaagaaaaagcggaaagtgtaa

SEQ ID NO: 196 - DNA, 5877 bp, synthetic construct
  source 1..5877: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaactcccccctctggagcgccttactcttggctccctgcagttgcccccactggagcggctgaccctggggtcattgcagttgcctccactcgaacgactgacgttgagccagaactatccgattgtgcagaccggtatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgccgactataaggaccacgacggagactacaaggatcatgatattgattacaaagacgatgacgataagagcctgggcagcggctcccccaagaaaaaacgcaaggtggaagatcctaagaaaaagcggaaagtgtaa

SEQ ID NO: 197 - DNA, 5919 bp, synthetic construct
  source 1..5919: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaaaactttagccagaactatccgattgtgcagaccggtatgaagcgagcactgcccaacaacaccagctcctctccccagccaaagaagaaaccaggagggtcagggggaaagcgagcactgcccaacaacaccagctcctctccccagccaaagaagaaaccaggagggtcagggggaatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgccgactataaggaccacgacggagactacaaggatcatgatattgattacaaagacgatgacgataagagcctgggcagcggctcccccaagaaaaaacgcaaggtggaagatcctaagaaaaagcggaaagtgtaa

SEQ ID NO: 198 - DNA, 6018 bp, synthetic construct
  source 1..6018: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaactcccccctctggagcgccttactcttggctccctgcagttgcccccactggagcggctgaccctggggtcattgcagttgcctccactcgaacgactgacgttgagccagaactatccgattgtgcagaccggtatgaagcgagcactgcccaacaacaccagctcctctccccagccaaagaagaaaccaggagggtcagggggaaagcgagcactgcccaacaacaccagctcctctccccagccaaagaagaaaccaggagggtcagggggaatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgccgactataaggaccacgacggagactacaaggatcatgatattgattacaaagacgatgacgataagagcctgggcagcggctcccccaagaaaaaacgcaaggtggaagatcctaagaaaaagcggaaagtgtaa

SEQ ID NO: 199 - DNA, 105 bp, synthetic construct
  source 1..105: mol_type = other DNA
ctcccccctctggagcgccttactcttggctccctgcagttgcccccactggagcggctgaccctggggtcattgcagttgcctccactcgaacgactgacgttg

SEQ ID NO: 200 - DNA, 138 bp, synthetic construct
  source 1..138: mol_type = other DNA
aagcgagcactgcccaacaacaccagctcctctccccagccaaagaagaaaccaggagggtcagggggaaagcgagcactgcccaacaacaccagctcctctccccagccaaagaagaaaccaggagggtcaggggga

SEQ ID NO: 201 - protein, 2005 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQLPPLERLTLGSLQLPPLERLTLGSLQLPPLERLTLSQNYPIVQTGMKRALPNNTSSSPQPKKKPGGSGGKRALPNNTSSSPQPKKKPGGSGGMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDADYKDHDGDYKDHDIDYKDDDDKSLGSGSPKKKRKVEDPKKKRKV

SEQ ID NO: 202 - DNA, 1029 bp, synthetic construct
  source 1..1029: mol_type = other DNA
atggccttaccagtgaccgccttgctcctgccgctggccttgctgctccacgccgccaggccggaacagaaactgattagcgaagaagatcttgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctaa

SEQ ID NO: 203 - protein, 16 aa, synthetic construct
GGGGSGGGGSGGGGSS

SEQ ID NO: 204 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 205 - protein, 8 aa, synthetic construct
SQNYPIVQ

SEQ ID NO: 206 - protein, 18 aa, synthetic construct
KRALPNNTSSSPQPKKKP

SEQ ID NO: 207 - protein, 10 aa, synthetic construct
  VARIANT 1: note = Wherein L at position 1 may be any hydrophobic amino acid
  VARIANT 5: note = Wherein L at position 5 may be any hydrophobic amino acid
  VARIANT 8: note = Wherein L at position 8 may be any hydrophobic amino acid
  VARIANT 10: note = Wherein L at position 10 may be any hydrophobic amino acid
LXXXLXXLXL

SEQ ID NO: 208 - DNA, 20 bp, synthetic construct
  source 1..20: mol_type = other DNA
gagtagcgcgagcacagcta

SEQ ID NO: 209 - DNA, 20 bp, synthetic construct
  source 1..20: mol_type = other DNA
agagtctctcagctggtaca

SEQ ID NO: 210 - DNA, 20 bp, synthetic construct
  source 1..20: mol_type = other DNA
cgactggccagggcgcctgt

SEQ ID NO: 211 - DNA, 32 bp, synthetic construct
  source 1..32: mol_type = other DNA
gctcttccgatctaagctgacagcattcgggc

SEQ ID NO: 212 - DNA, 33 bp, synthetic construct
  source 1..33: mol_type = other DNA
gctcttccgatctgaagtcacggagcgagagag

SEQ ID NO: 213 - DNA, 20 bp, synthetic construct
  source 1..20: mol_type = other DNA
aagtaaaacctctacaaatg

SEQ ID NO: 214 - DNA, 20 bp, synthetic construct
  source 1..20: mol_type = other DNA
gtattactgatattggtggg

SEQ ID NO: 215 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 216 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 107 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 218 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 219 - protein, 14 aa, synthetic construct
TGTSSDIGGYNSVS

SEQ ID NO: 220 - protein, 11 aa, synthetic construct
LMIYGVNNRPS

SEQ ID NO: 221 - protein, 10 aa, synthetic construct
SSYDIESATP

SEQ ID NO: 222 - protein, 10 aa, synthetic construct
GYSFTSYWIG

SEQ ID NO: 223 - protein, 16 aa, synthetic construct
WMGIIDPGDSRTRYSP

SEQ ID NO: 224 - protein, 11 aa, synthetic construct
GQLYGGTYMDG

SEQ ID NO: 225 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 226 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 227 - protein, 10 aa, synthetic construct
GDSVSSNSAA

SEQ ID NO: 228 - protein, 9 aa, synthetic construct
TYYRSKWYN

SEQ ID NO: 229 - protein, 14 aa, synthetic construct
AREVTGDLEDAFDI

SEQ ID NO: 230 - protein, 6 aa, synthetic construct
QTIWSY

SEQ ID NO: 231 - protein, 9 aa, synthetic construct
QQSYSIPQT

SEQ ID NO: 232 - protein, 11 aa, synthetic construct
KASQDVSIAVA

SEQ ID NO: 233 - protein, 7 aa, synthetic construct
SASYRYT

SEQ ID NO: 234 - protein, 9 aa, synthetic construct
QQHYITPLT

SEQ ID NO: 235 - protein, 5 aa, synthetic construct
NYGMN

SEQ ID NO: 236 - protein, 17 aa, synthetic construct
WINTYTGEPTYTDDFKG

SEQ ID NO: 237 - protein, 12 aa, synthetic construct
GGFGSSYWYFDV

SEQ ID NO: 238 - protein, 5 aa, synthetic construct
TAGMQ

SEQ ID NO: 239 - protein, 17 aa, synthetic construct
WINTHSGVPKYAEDFKG

SEQ ID NO: 240 - protein, 12 aa, synthetic construct
SGFGSSYWYFDV

SEQ ID NO: 241 - protein, 11 aa, synthetic construct
KASQDVSTAVA

SEQ ID NO: 242-243 - skipped (2 entries)

SEQ ID NO: 244 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 245 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 246-247 - skipped (2 entries)

SEQ ID NO: 248 - protein, 217 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 249 - protein, 453 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 251 - protein, 131 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 252 - protein, 8 aa, synthetic construct
SSNIGSNT

SEQ ID NO: 253 - protein, 11 aa, synthetic construct
AAWDDSLNGWV

SEQ ID NO: 254 - protein, 8 aa, synthetic construct
GFTFGDYA

SEQ ID NO: 255 - protein, 10 aa, synthetic construct
SRSKAYGGTT

SEQ ID NO: 256 - protein, 14 aa, synthetic construct
ASSGYSSGWTPFDY

SEQ ID NO: 257 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 258 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 259 - protein, 11 aa, synthetic construct
SASQDISNYLN

SEQ ID NO: 260 - protein, 7 aa, synthetic construct
YTSNLHS

SEQ ID NO: 261 - protein, 9 aa, synthetic construct
QQYRKLPWT

SEQ ID NO: 262 - protein, 5 aa, synthetic construct
NYWMH

SEQ ID NO: 263 - protein, 17 aa, synthetic construct
ATYRGHSDTYYNQKFKG

SEQ ID NO: 264 - protein, 12 aa, synthetic construct
GAIYNGYDVLDN

SEQ ID NO: 265 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 266 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 267 - protein, 8 aa, synthetic construct
GFTFSNYY

SEQ ID NO: 268 - protein, 8 aa, synthetic construct
ISGRGSTI

SEQ ID NO: 269 - protein, 10 aa, synthetic construct
VKDRGGYSPY

SEQ ID NO: 270 - protein, 6 aa, synthetic construct
QSISTY

SEQ ID NO: 271 - protein, 10 aa, synthetic construct
QQSYSTPPIT

SEQ ID NO: 272 - protein, 8 aa, synthetic construct
GYTFTDYS

SEQ ID NO: 273 - protein, 8 aa, synthetic construct
INTETGVP

SEQ ID NO: 274 - protein, 8 aa, synthetic construct
ARRTGFDY

SEQ ID NO: 275 - protein, 11 aa, synthetic construct
KNLLHSDGITY

SEQ ID NO: 276 - protein, 9 aa, synthetic construct
VQVLELPFT

SEQ ID NO: 277 - protein, 8 aa, synthetic construct
GFTFSSYA

SEQ ID NO: 278 - protein, 8 aa, synthetic construct
ISDGGSYS

SEQ ID NO: 279 - protein, 14 aa, synthetic construct
ARDSYYDNSYVRDY

SEQ ID NO: 280 - protein, 6 aa, synthetic construct
QDINTF

SEQ ID NO: 281 - protein, 9 aa, synthetic construct
LQYDEFPLT

SEQ ID NO: 282 - DNA, 33 bp, synthetic construct
  source 1..33: mol_type = other DNA
gctcttccgatctccgaccccacctacctaaga

SEQ ID NO: 283 - DNA, 33 bp, synthetic construct
  source 1..33: mol_type = other DNA
gctcttccgatctgacagtttcccttccgctca

SEQ ID NO: 284 - DNA, 34 bp, synthetic construct
  source 1..34: mol_type = other DNA
gctcttccgatctggggcaaagagggaaatgaga

SEQ ID NO: 285 - DNA, 36 bp, synthetic construct
  source 1..36: mol_type = other DNA
gctcttccgatctactttgtgacacatttgtttgag

SEQ ID NO: 286 - RNA, 106 nt, synthetic construct
  source 1..106: mol_type = other RNA
gagtagcgcgagcacagctagtttaagagctatgctggaaacagcatagcaagtttaaataaggctagtccgttatcaacttgaaaaagtggcaccgagtcggtgc